(19) United States
(12) Patent Application Publication
(10) Pub. No.: US 2009/0186358 A1
(43) Pub. Date: Jul. 23, 2009

(54) Pathway Analysis of Cell Culture Phenotypes and Uses Thereof

(75) Inventors: Mark Melville, Melrose, MA (US); Niall Barron, Shankill (IE); Martin Clynes, Clontarf (IE); Padraig Doolan, Swords (IE); Patrick Gammell, Naas (IE); Paula Meleady, Ratoath (IE)

Correspondence Address: CHOATE, HALL & STEWART LLP, TWO INTERNATIONAL PLACE, BOSTON, MA, 02110, US

(73) Assignees: Wyeth, Madison, NJ (US); Dublin City University, Glasnevin (IE)

(21) Appl. No.: 12/340,629

(22) Filed: Dec. 19, 2008

Related U.S. Application Data
(60) Provisional application No. 61/016,390, filed on Dec. 21, 2007.

(51) Int. Cl. C12Q 1/68; C12P 21/00; C07K 14/00; C12Q 1/02; C12N 1/21; C12N 5/06; C12N 5/04

(52) U.S. Cl. 435/6; 435/69.1; 530/300; 435/29; 435/252.3; 435/325; 435/419; 435/254.2

(57) ABSTRACT

The present invention provides methods for systematically identifying genes, proteins and/or related pathways that regulate or indicative of cell phenotypes. The present invention further provides methods for manipulating the identified genes, proteins and/or pathways to engineer improved cell lines and/or to evaluate or select cell lines with desirable phenotypes.

### RELATED APPLICATIONS

This application claims priority to and the benefit of U.S. Application No. 61/016,390, filed on Dec. 21, 2007, the contents of which are hereby incorporated by reference in their entireties. This application also relates to U.S. application Ser. No. 11/788,872 and PCT/US2007/10002, both filed on Apr. 21, 2007, and U.S. application Ser. No. 12/139,294 and PCT/US2008/066845, both filed on Jun. 13, 2008, the contents of all of which are incorporated by reference herein.

### FIELD OF THE INVENTION

The present invention relates to methods for identifying genes, proteins and/or pathways that are involved in regulating cell culture phenotypes and the uses thereof.

### BACKGROUND OF THE INVENTION

Fundamental to the present-day study of biology is the ability to optimally culture and maintain cell lines. Cell lines not only provide an in vitro model for the study of biological systems and diseases, but are also used to produce organic reagents. Of particular importance is the use of genetically engineered prokaryotic or eukaryotic cell lines to generate mass quantities of recombinant proteins. A recombinant protein may be used in a biological study, or as a therapeutic compound for treating a particular ailment or disease.

The production of recombinant proteins for biopharmaceutical application typically requires vast numbers of cells and/or particular cell culture conditions that influence cell growth and/or expression. In some cases, production of recombinant proteins benefits from the introduction of chemical inducing agents (such as sodium butyrate or valeric acid) to the cell culture medium. Identifying the genes and related genetic pathways that respond to the culture conditions (or particular agents) that increase transgene expression may elucidate potential targets that can be manipulated to increase recombinant protein production and/or influence cell growth.

Research into optimizing recombinant protein production has been primarily devoted to examining gene regulation, cellular responses, cellular metabolism, and pathways activated in response to unfolded proteins. For example, currently available methods for detecting transgene expression include those that measure only the presence and amount of known proteins (e.g., Western blot analysis, enzyme-linked immunosorbent assay, and fluorescence-activated cell sorting), or the presence and amount of known messenger RNA (mRNA) transcripts (e.g., Northern blot analysis and reverse transcription-polymerase chain reaction). These and similar methods are not only limited in the number of known proteins and/or mRNA transcripts that can be detected at one time, but they also require that the investigator know or “guess” what genes are involved in transgene expression prior to experimentation (so that the appropriate antibodies or oligonucleotide probes are used). Another limitation inherent in blot analyses and similar protocols is that proteins or mRNA that are the same size cannot be distinguished. Considering the vast number of genes contained within a single genome, identification of even a minority of genes involved in a genetic pathway using the methods described above is costly and time-consuming. Additionally, the requirement that the investigator have some idea regarding which genes are involved does not allow for the identification of genes and related pathways that were either previously undiscovered or unknown to be involved in the regulation of transgene expression.

### SUMMARY OF THE INVENTION

The present invention provides, among other things, methods to identify genes, proteins and/or pathways that regulate and/or indicative of cell phenotypes of interest and the uses of such genes, proteins, and/or pathways to engineer improved cell lines, optimize cell culture conditions, evaluate and/or select cell lines.

In one aspect, the present invention provides engineered cell lines characterized by improved cell culture phenotypes as compared to a corresponding wild type or parental cell line. In some embodiments, an engineered cell line according to the invention includes a population of engineered cells, each of which contains an engineered construct modulating, i.e., up-regulating or down-regulating, one or more genes or proteins selected from Tables 1-35, wherein modulating (i.e., up-regulating or down-regulating) one or more genes or proteins confers the improved cell culture phenotype. In some embodiments, the improved cell culture phenotype is selected from the group consisting of improved peak cell density, improved cell growth rate, improved sustained high cell viability, improved maximum cellular productivity, improved sustained high cellular productivity, reduced lactate production, reduced ammonia production, and combinations thereof.

In some embodiments, the present invention provides an engineered cell line with improved peak cell density as compared to a corresponding wild type or parental cell line. In some embodiments, an engineered cell line of the present invention comprises a population of engineered cells, each of which containing an engineered construct modulating (i.e., up-regulating or down-regulating) one or more genes or proteins selected from Tables 10 and 11, wherein modulating (i.e., up-regulating or down-regulating) one or more genes or proteins confers the improved peak cell density.

In some embodiments, the present invention provides engineered cell lines with improved cell growth rate as compared to a corresponding wild type or parental cell line. In some embodiments, an engineered cell line of the present invention comprises a population of engineered cells, each of which containing an engineered construct modulating (i.e., up-regulating or down-regulating) one or more genes or proteins selected from Table 12, wherein modulating (i.e., up-regulating or down-regulating) one or more genes or proteins confers the improved cell growth rate.

In some embodiments, the present invention provides an engineered cell line with improved sustained high cell viability as compared to the corresponding wild type or parental cell line. In some embodiments, an engineered cell line of the present invention comprises a population of engineered cells, each of which containing an engineered construct modulating (i.e., up-regulating or down-regulating) one or more genes or proteins selected from Tables 1-9, wherein modulating (i.e., up-regulating or down-regulating) one or more genes or proteins confers the improved sustained high cell viability.

In some embodiments, the present invention provides engineered cell lines with improved maximum cellular productivity as compared to a corresponding wild type or parental cell line. In some embodiments, an engineered cell line of the present invention comprises a population of engineered cells, each of which containing an engineered construct modulating (i.e., up-regulating or down-regulating) one or more genes or proteins selected from Tables 13-20, wherein modulating (i.e., up-regulating or down-regulating) one or more genes or proteins confers the improved maximum cellular productivity.

In some embodiments, the present invention provides engineered cell lines with improved sustained high cellular productivity as compared to a corresponding wild type or parental cell line. In some embodiments, an engineered cell line of the present invention comprises a population of engineered cells, each of which containing an engineered construct modulating (i.e., up-regulating or down-regulating) one or more genes or proteins selected from Tables 21-24, wherein modulating (i.e., up-regulating or down-regulating) one or more genes or proteins confers the improved sustained high cellular productivity.

In some embodiments, the present invention provides engineered cell lines with reduced ammonium production as compared to a corresponding wild type or parental cell line. In some embodiments, an engineered cell line of the present invention comprises a population of engineered cells, each of which containing an engineered construct modulating (i.e., up-regulating or down-regulating) one or more genes or proteins selected from Tables 25-30, wherein modulating (i.e., up-regulating or down-regulating) one or more genes or proteins confers the reduced ammonium production.

In some embodiments, the present invention provides engineered cell lines with reduced lactate production as compared to a corresponding wild type or parental cell line. In some embodiments, an engineered cell line of the present invention comprises a population of engineered cells, each of which containing an engineered construct modulating (i.e., up-regulating or down-regulating) one or more genes or proteins selected from Tables 31-35, wherein modulating (i.e., up-regulating or down-regulating) one or more genes or proteins confers the reduced lactate production.

As used herein, “up-regulating” includes providing an exogenous nucleic acid (e.g., an over-expression construct) encoding a protein of interest or a variant retaining its activity (such as, for example, a mammalian homolog thereof, such as a primate or rodent homolog) or providing a factor or a molecule indirectly enhancing the protein or gene activity or expression level. As used herein, “down-regulating” includes knocking-out the gene encoding a protein of interest, providing an RNA interference construct, or providing an inhibitor or other factors indirectly inhibiting the protein or gene activity or expression level.

In some embodiments, an engineered construct suitable for the invention is an over-expression construct. In some embodiments, an engineered construct suitable for the invention is an RNA interfering construct.

In some embodiments, an engineered cell line is selected from BALB/c mouse myeloma line, human retinoblasts (PER.C6), monkey kidney cells, human embryonic kidney line (293), baby hamster kidney cells (BHK), Chinese hamster ovary cells (CHO), mouse sertoli cells, African green monkey kidney cells (VERO-76), human cervical carcinoma cells (HeLa), canine kidney cells, buffalo rat liver cells, human lung cells, human liver cells, mouse mammary tumor cells, TR1 cells, MRC 5 cells, FS4 cells, or human hepatoma line (Hep G2).

In another aspect, the present invention provides methods of producing a protein of interest using engineered cell lines of the invention. In some embodiments, a method of the invention include one or more of the following steps: (a) providing an engineered cell line described herein that carries a nucleic acid encoding a protein of interest; (b) culturing the engineered cell line under conditions that allow expression of the protein of interest; and (c) harvesting the protein of interest. In some embodiments, a protein of interest is a monoclonal antibody or a fragment thereof, a growth factor, a clotting factor, a cytokine, a vaccine, an enzyme, or a Small Modular ImmunoPharmaceuticals™ (SMIPs).

The present invention also provides proteins produced using methods described herein.

In another aspect, the present invention provides methods of improving a cell line by, e.g., modifying one or more pathways selected from any of the pathways shown in FIGS. 1-31.

In some embodiments, the present invention provides methods of improving a cell line including introducing at least one modification into one or more cells that alters alanine and aspartate metabolism, glutamate metabolism, or combinations thereof, wherein the at least one modification confers improved peak cell density as compared to the corresponding unmodified cell line.

In some embodiments, the present invention provides methods of improving a cell line including introducing at least one modification into one or more cells that alters G1/S checkpoint regulation, ATM signaling, Eda-A1 signaling, Eda-A2 signaling, p53 signaling, JNK-MAPK signaling pathway, mitochondrial control of apoptosis, Rb tumor suppressor signaling, or combinations thereof, wherein the at least one modification confers improved maximum cellular productivity as compared to the corresponding unmodified cell line.

In some embodiments, the present invention provides methods of improving a cell line including introducing at least one modification into one or more cells that alters synthesis and degradation of ketone bodies, wherein the at least one modification confers improved cell growth rate as compared to the corresponding unmodified cell line.

In some embodiments, the present invention provides methods of improving a cell line including introducing at least one modification into one or more cells that alters synthesis and degradation of ketone bodies, butanoate metabolism, valine, leucine, and isoleucine degradation, Eda-A1 signaling, Eda-A2 signaling, or combinations thereof, wherein the at least one modification confers reduced ammonia production as compared to the corresponding unmodified cell line.

In some embodiments, the present invention provides methods of improving a cell line including introducing at least one modification into one or more cells that alters oxidative phosphorylation, mitochondrial dysfunction, butanoate metabolism, synthesis and degradation of ketone bodies, Eda-A1 signaling, Eda-A2 signaling, or combinations thereof, wherein the at least one modification confers reduced lactate production as compared to the corresponding unmodified cell line.

In some embodiments, the present invention provides methods of improving a cell line including introducing at least one modification into one or more cells that alters citrate cycle, butanoate metabolism, glutathione metabolism, NRF2-mediated oxidative stress response, LPS-IL-1 mediated inhibition of RXR function, synthesis and degradation of ketone bodies, Eda-A1 signaling, Eda-A2 signaling, or combinations thereof, wherein the at least one modification confers improved sustained high cell viability as compared to the corresponding unmodified cell line.

In some embodiments, the present invention provides methods of improving a cell line including introducing at least one modification into one or more cells that alters inositol metabolism, glycolysis, gluconeogenesis, NRF2-mediated oxidative stress response, purine metabolism, or combinations thereof, wherein the at least one modification confers improved sustained high cellular productivity as compared to the corresponding unmodified cell line.

In some embodiments, the at least one modification comprises an over expression construct. In some embodiment, the at least one modification comprises an RNA interfering construct.

In some embodiments, the cell line is selected from BALB/c mouse myeloma line, human retinoblasts (PER.C6), monkey kidney cells, human embryonic kidney line (293), baby hamster kidney cells (BHK), Chinese hamster ovary cells (CHO), mouse sertoli cells, African green monkey kidney cells (VERO-76), human cervical carcinoma cells (HeLa), canine kidney cells, buffalo rat liver cells, human lung cells, human liver cells, mouse mammary tumor cells, TR1 cells, MRC 5 cells, FS4 cells, or human hepatoma line (Hep G2).

The present invention also provides cells or cell lines improved by the methods described herein.

In yet another aspect, the present invention provides methods of producing a protein of interest using improved cell lines of the invention. In some embodiments, methods of the invention include one or more steps of: (a) providing an improved cell line as described herein that carries a nucleic acid encoding a protein of interest; (b) culturing the improved cell line under conditions that allow expression of the protein of interest; and (c) harvesting the protein of interest.

In some embodiments, the protein of interest is a monoclonal antibody or a fragment thereof, a growth factor, a clotting factor, a cytokine, a vaccine, an enzyme, or a Small Modular ImmunoPharmaceuticals™ (SMIPs).

The present invention also provides proteins produced using the methods described herein.

In still another aspect, the present invention provides methods of evaluating a cell culture phenotype of a cell line using genes, proteins and/or pathways identified herein. In some embodiments, methods of the invention include one or more steps of: (a) detecting, in a sample of cultured cells, an expression level of at least one protein or gene selected from Tables 1-35; (b) comparing the expression level to a reference level, wherein the comparison is indicative of the cell culture phenotype.

In some embodiments, the cell culture phenotype is peak cell density and the at least one protein or gene is selected from Tables 10 and 11.

In some embodiments, the cell culture phenotype is high cell growth rate and the at least one protein or gene is selected from Table 12.

In some embodiments, the cell culture phenotype is sustained high cell viability and the at least one protein or gene is selected from Tables 1-9.

In some embodiments, the cell culture phenotype is maximum cellular productivity and the at least one protein or gene is selected from Tables 13-20.

In some embodiments, the cell culture phenotype is sustained high cellular productivity and the at least one protein or gene is selected from Tables 21-24.

In some embodiments, the cell culture phenotype is low ammonium production and the at least one protein or gene is selected from Tables 25-30.

In some embodiments, the cell culture phenotype is low lactate production and the at least one protein or gene is selected from Tables 31-35.

In some embodiments, methods of the invention include one or more steps of: (a) determining, in a sample of cultured cells, a signaling strength of at least one pathway selected from the pathways shown in FIGS. 1-31; (b) comparing the signaling strength to a reference; wherein the comparison is indicative of the cell culture phenotype.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitations.

FIG. 1 depicts exemplary pathways identified that may contribute to the regulation of relevant cell phenotypes.

FIG. 2 depicts an exemplary pathway, cholesterol biosynthetic pathway, identified by pathway analysis. Differential gene expression in the cholesterol biosynthetic pathway is indicated by black (upregulated by >1.5 fold) or gray (upregulated by <1.5 fold). Differential expression is represented as change in clone 19 compared to parent.

FIG. 3 depicts an exemplary butanoate metabolism pathway identified in the sustained high cell viability phenotype.

FIG. 4 depicts an exemplary citrate cycle pathway identified in the sustained high cell viability phenotype.

FIG. 5 depicts an exemplary glutathione metabolism pathway identified in the sustained high cell viability phenotype.

FIG. 6 depicts an exemplary LPS-IL-1 mediated inhibition of RXR function pathway identified in the sustained high cell viability phenotype.

FIG. 7 depicts an exemplary NRF-2 mediated oxidative stress response pathway identified in the sustained high cell viability phenotype.

FIG. 8 depicts an exemplary synthesis and degradation of ketone bodies pathway identified in the sustained high cell viability phenotype.

FIG. 9 depicts an exemplary Eda A1 pathway identified in connection with the sustained high cell viability phenotype, the high maximum cellular productivity phenotype, the low ammonium production phenotype, and the low lactate production phenotype.

FIG. 10 depicts an exemplary Eda A2 pathway identified in connection with the sustained high cell viability phenotype, the high maximum cellular productivity phenotype, the low ammonium production phenotype, and the low lactate production phenotype.

FIG. 11 depicts an exemplary alanine and aspartate metabolism pathway identified in the high cell density phenotype.

FIG. 12 depicts an exemplary glutamate metabolism pathway identified in the high cell density phenotype.

FIG. 13 depicts an exemplary synthesis and degradation of ketone bodies pathway identified in the high cell growth rate phenotype.

FIG. 14 depicts an exemplary G1/S checkpoint regulation pathway identified in the high maximum cellular productivity phenotype.

FIG. 15 depicts an exemplary ATM signaling pathway identified in the high maximum cellular productivity phenotype.

FIG. 16 depicts an exemplary Jnk-mapk pathway identified in the high maximum cellular productivity phenotype.

FIG. 17 depicts an exemplary mitochondrial control of apoptosis pathway identified in the high maximum cellular productivity phenotype.

FIG. 18 depicts an exemplary p53 signaling pathway identified in the high maximum cellular productivity phenotype.

FIG. 19 depicts an exemplary RB tumor suppressor pathway identified in the high maximum cellular productivity phenotype.

FIG. 20 depicts an exemplary inositol metabolism pathway identified in the high cellular productivity phenotype.

FIG. 21 depicts an exemplary glycolysis, gluconeogenesis pathway identified in the high cellular productivity phenotype.

FIG. 22 depicts an exemplary NRF-2 mediated oxidative stress response pathway identified in the sustained high cellular productivity phenotype.

FIG. 23 depicts an exemplary purine metabolism pathway identified in the sustained high cellular productivity phenotype.

FIG. 24 depicts an exemplary ER stress response pathway identified in the low ammonium production phenotype.

FIG. 25 depicts an exemplary synthesis and degradation of ketone bodies pathway identified in the low ammonium production phenotype.

FIG. 26 depicts an exemplary butanoate metabolism pathway identified in the low ammonium production phenotype.

FIG. 27 depicts an exemplary valine, leucine, and isoleucine degradation pathway identified in the low ammonium production phenotype.

FIG. 28 depicts an exemplary oxidative phosphorylation pathway identified in the low lactate production phenotype.

FIG. 29 depicts an exemplary mitochondrial dysfunction pathway identified in the low lactate production phenotype.

FIG. 30 depicts an exemplary butanoate metabolism pathway identified in the low lactate production phenotype.

FIG. 31 depicts an exemplary synthesis and degradation of ketone bodies pathway identified in the low lactate production phenotype.

FIG. 32 depicts an exemplary target validation workflow.

### DEFINITIONS

Antibody: The term “antibody” as used herein refers to an immunoglobulin molecule or an immunologically active portion of an immunoglobulin molecule, i.e., a molecule that contains an antigen binding site which specifically binds an antigen, such as a Fab or F(ab′)₂ fragment. In certain embodiments, an antibody is a typical natural antibody known to those of ordinary skill in the art, e.g., glycoprotein comprising four polypeptide chains: two heavy chains and two light chains. In certain embodiments, an antibody is a single-chain antibody. For example, in some embodiments, a single-chain antibody comprises a variant of a typical natural antibody wherein two or more members of the heavy and/or light chains have been covalently linked, e.g., through a peptide bond. In certain embodiments, a single-chain antibody is a protein having a two-polypeptide chain structure consisting of a heavy and a light chain, which chains are stabilized, for example, by interchain peptide linkers, which protein has the ability to specifically bind an antigen. In certain embodiments, an antibody is an antibody comprised only of heavy chains such as, for example, those found naturally in members of the Camelidae family, including llamas and camels (see, for example, U.S. Pat. Nos. 6,765,087 by Casterman et al., 6,015,695 by Casterman et al., 6,005,079 and by Casterman et al., each of which is incorporated by reference in its entirety). The terms “monoclonal antibodies” and “monoclonal antibody composition”, as used herein, refer to a population of antibody molecules that contain only one species of an antigen binding site and therefore usually interact with only a single epitope or a particular antigen. Monoclonal antibody compositions thus typically display a single binding affinity for a particular epitope with which they immunoreact. The terms “polyclonal antibodies” and “polyclonal antibody composition” refer to populations of antibody molecules that contain multiple species of antigen binding sites that interact with a particular antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Batch culture: The term “batch culture” as used herein refers to a method of culturing cells in which all the components that will ultimately be used in culturing the cells, including the medium (see definition of “Medium” below) as well as the cells themselves, are provided at the beginning of the culturing process. A batch culture is typically stopped at some point and the cells and/or components in the medium are harvested and optionally purified.

Bioreactor: The term “bioreactor” as used herein refers to any vessel used for the growth of a mammalian cell culture. A bioreactor can be of any size so long as it is useful for the culturing of mammalian cells. Typically, such a bioreactor will be at least 1 liter and may be 10, 100, 250, 500, 1000, 2500, 5000, 8000, 10,000, 12,000 liters or more, or any volume in between. The internal conditions of the bioreactor, including, but not limited to pH, dissolved oxygen and temperature, are typically controlled during the culturing period. A bioreactor can be composed of any material that is suitable for holding mammalian cell cultures suspended in media under the culture conditions of the present invention, including glass, plastic or metal. The term “production bioreactor” as used herein refers to the final bioreactor used in the production of the protein of interest. The volume of the production bioreactor is typically at least 500 liters and may be 1000, 2500, 5000, 8000, 10,000, 12,000 liters or more, or any volume in between. One of ordinary skill in the art will be aware of and will be able to choose suitable bioreactors for use in practicing the present invention.

Cell density and high cell density: The term “cell density” as used herein refers to the number of cells present in a given volume of medium. The term “high cell density” as used herein refers to a cell density that exceeds 5×10⁶/mL, 1×10⁷/mL, 5×10⁷/mL, 1×10⁸/mL, 5×10⁸/mL, 1×10⁹/mL, 5×10⁹/mL, or 1×10¹⁰/mL.

Cellular productivity and sustained high cellular productivity: The term “cellular productivity” as used herein refers to the total amount of recombinantly expressed protein (e.g., polypeptides, antibodies, etc.) produced by a mammalian cell culture in a given amount of medium volume. Cellular productivity is typically expressed in milligrams of protein per milliliter of medium (mg/mL) or grams of protein per liter of medium (g/L). The term sustained high cellular productivity as used herein refers to the ability of cells in culture to maintain a high cellular productivity (e.g., more than 5 g/L, 7.5 g/L, 10 g/L, 12.5 g/L, 15 g/L, 17.5 g/L, 20 g/L, 22.5 g/L, 25 g/L) under a given set of cell culture conditions or experimental variations.

Cell growth rate and high cell growth rate: The term “cell growth rate” as used herein refers to the rate of change in cell density expressed in “hr⁻¹” units as defined by the equation: (ln X2−ln X1)/(T2−T1) where X2 is the cell density (expressed in millions of cells per milliliter of culture volume) at time point T2 (in hours) and X1 is the cell density at an earlier time point T1. In some embodiments, the term “high cell growth rate” as used herein refers to a growth rate value that exceeds 0.023 hr⁻¹.

Cell viability and sustained high cell viability: The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time. The term “sustained high cell viability” as used herein refers to the ability of cells in culture to maintain a high cell viability (e.g., more than 60%, 65%, 70%, 75%, 80%, 85%, 90%, 95%, 98%, 99% of the total number of cells that are alive) under a given set of cell culture conditions or experimental variations.

Control and test: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested or monitored) is applied or present (e.g., a test cell line or culture with a desirable phenotype). In the second experiment, the “control,” the variable being tested is not applied or present (e.g., a control cell line or culture that does not have the desirable phenotype). In some embodiments, a control is a historical control (i.e., of a test or assay performed previously, or an amount or result that is previously known). In some embodiments, a control is or comprises a printed or otherwise saved record. A control may be a positive control or a negative control.

Culture: The term “cell culture” as used herein refers to a cell population that is suspended in a medium (see definition of “Medium” below) under conditions suitable to survival and/or growth of the cell population. As will be clear to those of ordinary skill in the art, in certain embodiments, these terms as used herein refer to the combination comprising the cell population and the medium in which the population is suspended. In certain embodiments, the cells of the cell culture comprise mammalian cells.

Differential expression profiling: The term “differential expression profiling” as used herein refers to methods of comparing the gene or protein expression levels or patterns of two or more samples (e.g., test samples vs. control samples). In some embodiments, differential expression profiling is used to identify genes, proteins or other components that are differentially expressed. A gene or protein is differentially expressed if the difference in the expression level or pattern between two samples is statistically significant (i.e., the difference is not caused by random variations). In some embodiments, a gene or protein is differentially expressed if the difference in the expression level between two samples is more than 1.2-fold, 1.5-fold, 1.75-fold, 2-fold, 2.25-fold, 2.5-fold, 2.75-fold, or 3-fold.

Fed-batch culture: The term “fed-batch culture” as used herein refers to a method of culturing cells in which additional components are provided to the culture at a time or times subsequent to the beginning of the culture process. Such provided components typically comprise nutritional components for the cells which have been depleted during the culturing process. Additionally or alternatively, such additional components may include supplementary components (see definition of “Supplementary components” below). In certain embodiments, additional components are provided in a feed medium (see definition of “Feed medium” below). A fed-batch culture is typically stopped at some point and the cells and/or components in the medium are harvested and optionally purified.

Feed medium: The term “feed medium” as used herein refers to a solution containing nutrients which nourish growing mammalian cells that is added after the beginning of the cell culture. A feed medium may contain components identical to those provided in the initial cell culture medium. Alternatively, a feed medium may contain one or more additional components beyond those provided in the initial cell culture medium. Additionally or alternatively, a feed medium may lack one or more components that were provided in the initial cell culture medium. In certain embodiments, one or more components of a feed medium are provided at concentrations or levels identical or similar to the concentrations or levels at which those components were provided in the initial cell culture medium. In certain embodiments, one or more components of a feed medium are provided at concentrations or levels different than the concentrations or levels at which those components were provided in the initial cell culture medium.

Fragment: The term “fragment” as used herein refers to a polypeptide that is defined as any discrete portion of a given polypeptide that is unique to or characteristic of that polypeptide. For example, the term as used herein refers to any portion of a given polypeptide that includes at least an established sequence element found in the full-length polypeptide. In certain fragments, the sequence element spans at least 4-5, 10, 15, 20, 25, 30, 35, 40, 45, 50 or more amino acids of the full-length polypeptide. Alternatively or additionally, the term as used herein refers to any discrete portion of a given polypeptide that retains at least a fraction of at least one activity of the full-length polypeptide. In certain embodiments, the fraction of activity retained is at least 10% of the activity of the full-length polypeptide. In certain embodiments, the fraction of activity retained is at least 20%, 30%, 40%, 50%, 60%, 70%, 80% or 90% of the activity of the full-length polypeptide. In certain embodiments, the fraction of activity retained is at least 95%, 96%, 97%, 98% or 99% of the activity of the full-length polypeptide. In certain embodiments, the fragment retains 100% of more of the activity of the full-length polypeptide.

Gene: The term “gene” as used herein refers to any nucleotide sequence, DNA or RNA, at least some portion of which encodes a discrete final product, typically, but not limited to, a polypeptide, which functions in some aspect of cellular metabolism or development. Optionally, the gene comprises not only the coding sequence that encodes the polypeptide or other discrete final product, but also comprises regions preceding and/or following the coding sequence that modulate the basal level of expression (sometimes referred to as “genetic control element”), and/or intervening sequences (“introns”) between individual coding segments (“exons”).

Low ammonium producer: The term “low ammonium producer” as used herein refers to a metabolic characteristic of cells that results in a low net ammonium concentration (brought about through a balance between ammonium production and ammonium depletion) in the culture medium. In some embodiments, the term “low ammonium producer” refers to a metabolic characteristic of cells that results in a net ammonium concentration in the culture medium of <3.0 millimolar.

Low lactate producer: The term “low lactate producer” as used herein refers to a metabolic characteristic of cells that results in a low net lactic acid concentration (brought about through a balance between lactic acid production and lactic acid consumption) in the culture medium. In some embodiments, the term “low lactate producer” refers to a metabolic characteristic of cells that results in a net lactic acid concentration in the culture medium of <3.0 g/L.

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Supplementary components: The term “supplementary components” as used herein refers to components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In certain embodiments, supplementary components may be added to the initial cell culture. In certain embodiments, supplementary components may be added after the beginning of the cell culture.

“Titer”: The term “titer” as used herein refers to the total amount of recombinantly expressed protein (e.g., polypeptides, antibodies) produced by a mammalian cell culture in a given amount of medium volume. Titer is typically expressed in units of milligrams of protein per milliliter of medium.

### DETAILED DESCRIPTION OF THE INVENTION

The present invention provides, among other things, methods for identifying genes, proteins, and/or pathways regulating and/or indicative of cell culture phenotypes. In particular, inventive methods according to the present invention involve pathway analysis. The present invention further provides methods of engineering cell lines, optimizing cell culture conditions, evaluating and/or selecting cell lines based on the genes, proteins and/or pathways of the invention.

Various aspects of the invention are described in further detail in the following subsections. The use of subsections is not meant to limit the invention. Each subsection may apply to any aspect of the invention. In this application, the use of “or” means “and/or” unless stated otherwise.

### Cell Lines and Cell Culture Phenotypes

Cells and cell lines of the present invention include cells and cells lines derived from a variety of organisms, including, but not limited to, bacteria, plants, fungi, and animals (the latter including, but not limited to, insects and mammals). For example, the present invention may be applied to Escherichia coli, Spodoptera frugiperda, Nicotiana sp., Zea mays, Lemna sp., Saccharomyces sp., Pichia sp., Schizosaccharomyces sp., mammalian cells, including, but not limited to, COS cells, CHO cells, 293 cells, A431 cells, 3T3 cells, CV-1 cells, HeLa cells, L cells, BHK21 cells, HL-60 cells, U937 cells, HEK cells, PerC6 cells, Jurkat cells, normal diploid cells, cell strains derived from in vitro culture of primary tissue, and primary explants. The list of organisms and cell lines are meant only to provide nonlimiting examples. In particular, the present invention can be applied to industrially relevant cell lines, such as, for example, CHO cells. CHO cells are a primary host for therapeutic protein production, such as, for example, monoclonal antibody production, receptor productions, and Fc fusion proteins because CHO cells provide fidelity of folding, processing, and glycosylation. CHO cells are also compatible with deep-tank, serum-free culture and have excellent safety records.

The present invention permits identification of pathways, genes and proteins that influence desired cell culture phenotypes or characteristics, for example, cell phenotypes that enable highly productive fed-batch processes. Such desired cell phenotypes include, but are not limited to, high cell growth rate, high peak cell density, sustained high cell viability, high maximum cellular productivity, sustained high cellular productivity, low ammonium production, and low lactate production. Desired phenotypes or characteristics may be inherent properties of established cell lines that have certain genomic backgrounds. Desired phenotypes or characteristics may also be conferred to cells by growing the cells in different conditions, e.g., temperatures, cell densities, the use of agents such as sodium butyrate, to be in different kinetic phases of growth (e.g., lag phase, exponential growth phase, stationary phase or death phase), and/or to become serum-independent, etc. During the period in which these phenotypes are induced, and/or after these phenotypes are achieved, a pool of target nucleic acid or protein samples can be prepared from the cells and analyzed with the oligonucleotide array to determine and identify which genes demonstrate altered expression in response to a particular stimulus (e.g., temperature, sodium butyrate), and therefore are potentially involved in conferring the desired phenotype or characteristic.

### Differential Expression Profiling Analysis

Genes and proteins regulating or indicative of cell culture phenotypes may be identified using differential expression profiling analysis.

In some embodiments, two or more pairs of different cell lines that display a different cell culture phenotype can be compared to identify genes and/or proteins regulating or indicative of the cell culture phenotype of interest. For example, a pair may include two cell lines, one displays high viability (test cell line) and the other displays low viability (control cell line). Comparison of each pair (e.g., high viability vs. low viability) identifies differentially expressed proteins or genes that may influence the cell culture phenotype of interest (e.g., high cell viability).

The cell phenotypes of a cell line may change over time under a cell culture condition. Typically, the change of cell phenotypes correlates with cell growth kinetics under a particular cell culture condition. For example, in the fed batch culture, cells undergo an initial phase of exponential growth. Typically, after several days, the culture temperature is lowered. Nutrient feeds are added to supplement growth and the cells are maintained for up to 14 days. At this time, the cells enter a lag phase, and in some cases, begin to decline in viability towards the end of the culture.

Therefore, in some embodiments, proteins or genes regulating or indicative of changes of cell phenotypes over time under a cell culture condition can be identified by examining the changes in gene or protein expression patterns over time in cells cultured under particular cell culture conditions. By observing these changes, we can gain an understanding of how a cell culture dynamically responds to its changing environment. For example, one cell line (referred to as test cell line) maintains a high viability throughout the fed batch, while the other cell line (referred to as control cell line) declines in viability relatively early. Replicate cultures of each cell line grown under similar fed batch conditions are sampled at multiple time points. Each is analyzed in order to characterize how the cells change their expression profiles over time. Differentially expressed proteins or genes are identified in each cell line. In some embodiments, differentially expressed proteins or genes in the test cell line are compared to the differentially expressed proteins or genes in the control cell line to classify the differentially expressed proteins or genes into three groups. The first group includes those that are unique to the test (e.g., high viability) cell line. The second group includes those unique to the control (e.g., low viability) cell line. The third group includes those in common between the two cell lines.

Each of the groups of differentially expressed genes or proteins provides insight into the cell lines and culture conditions. Those unique to the test cell line provide information regarding what may contribute to the ability of this cell line to maintain a desirable cell phenotype, for example, high viability. This group (test-only) of differentially expressed proteins or genes can be used to engineer cells to reproduce the desirable phenotype, or as indicate biomarkers to screen for or select the desirable phenotype. Conversely, those unique to the control cell line provide insights into what may contribute to a undesirable cell phenotype, for example, a decline in cell viability. This information can be used to engineer cells to avoid the undesirable phenotype, or as biomarkers to screen for or select against this phenotype. Finally, the differentially expressed genes and proteins that are in common between the cell lines provide insights into the process itself, that is, how cells generally respond to a cell culture condition, for example, a fed batch culture system.

In some embodiments, the change of the cell phenotype of interest over time under a cell culture condition in a test cell line is distinct from that in a control cell line. In some embodiments, a test cell line and a control cell line can be different cell lines with different genetic background or similar cell lines with modified genetic background. For example, a test cell line can be generated by over-expressing a protein, a gene or an inhibitory RNA in a control cell line to induce a desirable cell phenotype.

Differential Gene Expression Profiling Analysis

Methods used to detect the hybridization profile of target nucleic acids with oligonucleotide probes are well known in the art. In particular, means of detecting and recording fluorescence of each individual target nucleic acid-oligonucleotide probe hybrid have been well established and are well known in the art, described in, e.g., U.S. Pat. No. 5,631,734, U.S. Publication No. 20060010513, incorporated herein in their entirety by reference. For example, a confocal microscope can be controlled by a computer to automatically detect the hybridization profile of the entire array. Additionally, as a further nonlimiting example, the microscope can be equipped with a phototransducer attached to a data acquisition system to automatically record the fluorescence signal produced by each individual hybrid.

It will be appreciated by one of skill in the art that evaluation of the hybridization profile is dependent on the composition of the array, i.e., which oligonucleotide probes were included for analysis. For example, where the array includes oligonucleotide probes to consensus sequences only, or consensus sequences and transgene sequences only, (i.e., the array does not include control probes to normalize for variation between experiments, samples, stringency requirements, and preparations of target nucleic acids), the hybridization profile is evaluated by measuring the absolute signal intensity of each location on the array. Alternatively, the mean, trimmed mean (i.e., the mean signal intensity of all probes after 2-5% of the probesets with the lowest and highest signal intensities are removed), or median signal intensity of the array may be scaled to a preset target value to generate a scaling factor, which will subsequently be applied to each probeset on the array to generate a normalized expression value for each gene (see, e.g., Affymetrix (2000) Expression Analysis Technical Manual, pp. A5-14). Conversely, where the array further comprises control oligonucleotide probes, the resulting hybridization profile is evaluated by normalizing the absolute signal intensity of each location occupied by a test oligonucleotide probe by means of mathematical manipulations with the absolute signal intensity of each location occupied by a control oligonucleotide probe. Typical normalization strategies are well known in the art, and are included, for example, in U.S. Pat. No. 6,040,138 and Hill et al. (2001) Genome Biol. 2(12):research 0055.1-0055.13.

Signals gathered from oligonucleotide arrays can be analyzed using commercially available software, such as those provide by Affymetrix or Agilent Technologies. Controls, such as for scan sensitivity, probe labeling and cDNA or cRNA quantitation, may be included in the hybridization experiments. The array hybridization signals can be scaled or normalized before being subjected to further analysis. For instance, the hybridization signal for each probe can be normalized to take into account variations in hybridization intensities when more than one array is used under similar test conditions. Signals for individual target nucleic acids hybridized with complementary probes can also be normalized using the intensities derived from internal normalization controls contained on each array. In addition, genes with relatively consistent expression levels across the samples can be used to normalize the expression levels of other genes.

To identify genes that confer or correlate with a desired phenotype or characteristic, a gene expression profile of a sample derived from a test cell line is compared to a control profile derived from a control cell line that has a cell culture phenotype of interest distinct from that of the test cell line and differentially expressed genes are identified. For example, the method for identifying the genes and related pathways involved in cellular productivity may include the following: 1) growing a first sample of a first cell line with a particular cellular productivity and growing a second sample of a second cell line with a distinct cellular productivity; 2) isolating, processing, and hybridizing total RNA from the first sample to a first oligonucleotide array; 3) isolating, processing, and hybridizing total RNA from the second sample to a second oligonucleotide array; and 4) comparing the resulting hybridization profiles to identify the sequences that are differentially expressed between the first and second samples. Similar methods can be used to identify genes involved in other phenotypes.

Typically, each cell line was represented by at least three biological replicates. Programs known in the art, e.g., GeneExpress 2000 (Gene Logic, Gaithersburg, Md.), were used to analyze the presence or absence of a target sequence and to determine its relative expression level in one cohort of samples (e.g., cell line or condition or time point) compared to another sample cohort. A probeset called present in all replicate samples was considered for further analysis. Generally, fold-change values of 1.2-fold, 1.5-fold or greater were considered statistically significant if the p-values were less than or equal to 0.05.

The identification of differentially expressed genes that correlate with one or more particular cell phenotypes (e.g., cell growth rate, peak cell density, sustained high cell viability, maximum cellular productivity, sustained high cellular productivity, ammonium production or consumption, lactate production or consumption, etc.) can lead to the discovery of genes and pathways, including those which were previously undiscovered, that regulate or are indicative of the cell phenotypes.

The subsequently identified genes are sequenced and the sequences are blasted against various databases to determine whether they are known genes or unknown genes. If genes are known, pathway analysis can be conducted based on the existing knowledge in the art. Both known and unknown genes are further confirmed or validated by various methods known in the art. For example, the identified genes may be manipulated (e.g., up-regulated or down-regulated) to induce or suppress the particular phenotype by the cells.

More detailed identification and validation steps are further described in the Examples section.

Differential Protein Expression Profiling Analysis

The present invention also provides methods for identifying differentially expressed proteins by protein expression profiling analysis. Protein expression profiles can be generated by any method permitting the resolution and detection of proteins from a sample from a cell line. Methods with higher resolving power are generally preferred, as increased resolution can permit the analysis of greater numbers of individual proteins, increasing the power and usefulness of the profile. A sample can be pre-treated to remove abundant proteins from a sample, such as by immunodepletion, prior to protein resolution and detection, as the presence of an abundant protein may mask more subtle changes in expression of other proteins, particularly for low-abundance proteins. A sample can also be subjected to one or more procedures to reduce the complexity of the sample. For example, chromatography can be used to fractionate a sample; each fraction would have a reduced complexity, facilitating the analysis of the proteins within the fractions.

Three useful methods for simultaneously resolving and detecting several proteins include array-based methods; mass-spectrometry based methods; and two-dimensional gel electrophoresis based methods.

Protein arrays generally involve a significant number of different protein capture reagents, such as antibodies or antibody variable regions, each immobilized at a different location on a solid support. Such arrays are available, for example, from Sigma-Aldrich as part of their Panorama™ line of arrays. The array is exposed to a protein sample and the capture reagents selectively capture the specific protein targets. The captured proteins are detected by detection of a label. For example, the proteins can be labeled before exposure to the array; detection of a label at a particular location on the array indicates the detection of the corresponding protein. If the array is not saturated, the amount of label detected may correlate with the concentration or amount of the protein in the sample. Captured proteins can also be detected by subsequent exposure to a second capture reagent, which can itself be labeled or otherwise detected, as in a sandwich immunoassay format.

Mass spectrometry-based methods include, for example, matrix-assisted laser desorption/ionization (MALDI), Liquid Chromatography/Mass Spectrometry/Mass Spectrometry (LC-MS/MS) and surface enhanced laser desorption/ionization (SELDI) techniques. For example, a protein profile can be generated using electrospray ionization and MALDI. SELDI, as described, for example, in U.S. Pat. No. 6,225,047, incorporates a retention surface on a mass spectrometry chip. A subset of proteins in a protein sample are retained on the surface, reducing the complexity of the mixture. Subsequent time-of-flight mass spectrometry generates a “fingerprint” of the retained proteins.

In methods involving two-dimensional gel electrophoresis, proteins in a sample are generally separated in a first dimension by isoelectric point and in a second dimension by molecular weight during SDS-PAGE. By virtue of the two dimensions of resolution, hundreds or thousands of proteins can be simultaneously resolved and analyzed. The proteins are detected by application of a stain, such as a silver stain, or by the presence of a label on the proteins, such as a Cy2, Cy3, or Cy5 dye. To identify a protein, a gel spot can be cut out and in-gel tryptic digestion performed. The tryptic digest can be analyzed by mass spectrometry, such as MALDI. The resulting mass spectrum of peptides, the peptide mass fingerprint or PMF, is searched against a sequence database. The PMF is compared to the masses of all theoretical tryptic peptides generated in silico by the search program. Programs such as Prospector, Sequest, and MasCot (Matrix Science, Ltd., London, UK) can be used for the database searching. For example, MasCot produces a statistically-based Mowse score indicates if any matches are significant or not. MS/MS can be used to increase the likelihood of getting a database match. CID-MS/MS (collision induced dissociation of tandem MS) of peptides can be used to give a spectrum of fragment ions that contain information about the amino acid sequence. Adding this information to a peptide mass fingerprint allows Mascot to increase the statistical significance of a match. It is also possible in some cases to identify a protein by submitting only a raw MS/MS spectrum of a single peptide.

A recent improvement in comparisons of protein expression profiles involves the use of a mixture of two or more protein samples, each labeled with a different, spectrally-resolvable, charge- and mass-matched dye, such as Cy3 and Cy5. This improvement, called fluorescent 2-dimensional differential in-gel electrophoresis (DIGE), has the advantage that the test and control protein samples are run in the same gel, facilitating the matching of proteins between the two samples and avoiding complications involving non-identical electrophoresis conditions in different gels. The gels are imaged separately and the resulting images can be overlaid directly without further modification. A third spectrally-resolvable dye, such as Cy2, can be used to label a pool of protein samples to serve as an internal control among different gels run in an experiment. Thus, all detectable proteins are included as an internal standard, facilitating comparisons across different gels.

Exemplary genes and proteins identified using differential expression analysis are described in U.S. application Ser. No. 11/788,872 and PCT/US2007/10002, both filed on Apr. 21, 2007, and U.S. application Ser. No. 12/139,294 and PCT/US2008/066845, both filed on Jun. 13, 2008, the contents of all of which are incorporated by reference herein.

### Pathway Analysis

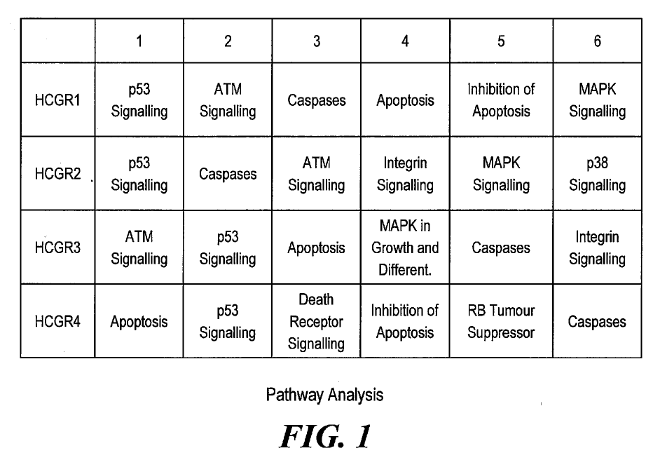

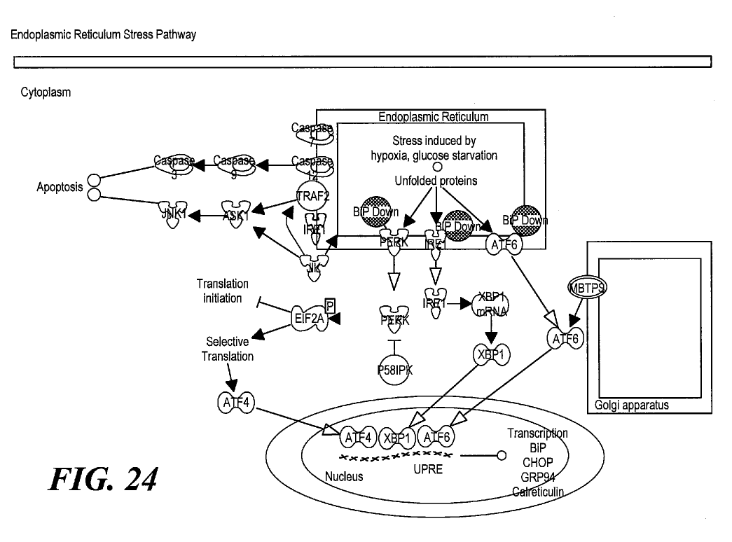

Additional genes and proteins that may influence cell culture phenotypes may be identified through pathway analysis. For example, pathway analysis can be employed to identify regulatory or signaling pathways that may contribute to the regulation of cell phenotypes of interest. For example, identified genes or proteins can be submitted to literature-mining tools such as, for example, Ingenuity Pathway Analysis (v6.5 Ingenuity Systems, www.ingenuity.com), PATHWAY STUDIO (v.5.0; www.ariadnegenomics.com) and PANTHER (v2.2; http://www.pantherdb.org/) to identify links between submitted genes or proteins. Exemplary pathway analysis is described in the Example section. Other methods and tools for pathway analysis are well known and available in the art. For example, additional exemplary pathway analysis tools suitable for the invention include, but are not limited to, MetaMine™ (Agilent Technologies), ePath3D (Protein Lounge), VisANT, PATHWAY ARCHITECT (www.stratagene.com), MetaCore (GeneGo, Inc.), Map Editor (GeneGo, Inc.), MetaLink (GeneGo, Inc.), GENMAPP (http://www.genmapp.org/), and GENEGO (http://www.genego.com/). FIGS. 1-31 illustrate exemplary pathways identified according to the present invention that may contribute to relevant cell phenotypes.

Pathway analysis facilitates prioritizing suitable targets and expands knowledge bases of genes or protiens. For example, if a pathway is identified to regulate a cell phenotype of interest. Genes involved in the pathway or regulating the pathway are likely to be regulators or biomakers of the cell phenotype of interest and can be used as potential targets for engineering cell lines or as biomarkers for evaluating or selecting cell lines with desirable phenotypes. Pathway analysis may identify genes or proteins that would otherwise not be identified using differential expression profiling analysis because those genes are not represented on microarrays, or are not detected as differentially expressed for any number of reasons (e.g., expression too low to detect, expression level too high to detect a difference, or not actually not differentially expressed). Exemplary genes and/or proteins identified using pathway analysis are shown in Tables 1-35. The names of the genes and proteins identified herein are commonly recognized by those skilled in the art and the sequences of the genes and proteins identified herein are readily available in several public databases (e.g., GenBank, SWISS-PROT). The sequences associated with each of the genes and proteins identified herein that are available in public databases (e.g., GenBank, SWISS-PROT) as of the filing date of the present application are incorporate by reference herein.

Pathway analysis may also identify genes and/or proteins that work in concert in regulating relevant cell phenotypes. In addition, metabolic or biosynthesis pathways identified according to the invention may be used to identify overarching limitations or bottlenecks in any particular culture condition, such as fed batch culture, and to determine desirable levels of relevant metabolites for cell culture. Thus, the present invention also provides methods for optimizing cell culture conditions by providing or adjusting the levels of relevant metabolites in cell media or evaluating cell culture conditions by monitoring levels of the metabolites controlled by the pathways of the invention in cells or cell culture media.

### Engineering Cell Lines to Improve Cell Phenotypes

Genes, proteins, and associated cellular and molecular pathways that regulate or are indicative of relevant cell phenotypes of interest according to the present invention can be used to engineer cell lines and to improve cell phenotypes. The genes, proteins, and associated pathways identified herein may be modulated (e.g., up-regulated or down-regulated) to effect a desirable cell phenotype, for example, a phenotype characterized by increased and efficient production of a recombinant transgene or proteins, increased cell growth rate, high peak cell density, sustained high cell viability, high maximum cellular productivity, sustained high cellular productivity, low ammonium production, and low lactate production, etc. For example, the genes, proteins or pathways can be used to improve CHO manufacturing platform to a new level of capability. The current capability of a typical CHO cell line is about 1-3 g Mabs/L or less than 5 g Mabs/L. An engineered CHO cell line of the present invention can have significantly increased capability, for example, >5 g Mabs/L, >10 g Mabs/L, >15 g Mabs/L, >20 g Mabs/L, >25 g Mabs/L, >30 g Mabs/L. The capability increase is not limited to the antibody production (e.g., monoclonal antibodies or fragments thereof). It is applicable to the production of other proteins, such as, for example, growth factors, clotting factors, cytokines, vaccines, enzymes, or Small Modular ImmunoPharmaceuticals™ (SMIPs). In addition, similar capability increases are contemplated for other cell lines. Thus, the present invention provides methods and compositions to better meet capacity demand for successful biopharma products.

The present invention contemplates methods and compositions that may be used to alter (i.e., regulate or modulate (e.g., enhance, reduce, or modify)) the expression and/or the activity of the genes, proteins or pathways according to the invention. Altered expression of the genes, proteins or pathways encompassed by the present invention in a cell or organism may be achieved through down-regulating or up-regulating of relevant genes or proteins. For example, genes and proteins identified herein may be down-regulated by the use of various inhibitory polynucleotides, such as antisense polynucleotides, ribozymes that bind and/or cleave the mRNA transcribed from the genes of the invention, triplex-forming oligonucleotides that target regulatory regions of the genes, and short interfering RNA that causes sequence-specific degradation of target mRNA (e.g., Galderisi et al. (1999) J. Cell. Physiol. 181:251-57; Sioud (2001) Curr. Mol. Med. 1:575-88; Knauert and Glazer (2001) Hum. Mol. Genet. 10:2243-51; Bass (2001) Nature 411:428-29).

The inhibitory antisense or ribozyme polynucleotides suitable for the invention can be complementary to an entire coding strand of a gene of the invention, or to only a portion thereof. Alternatively, inhibitory polynucleotides can be complementary to a noncoding region of the coding strand of a gene of the invention. The inhibitory polynucleotides of the invention can be constructed using chemical synthesis and/or enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Mickelfield (2001) Curr. Med. Chem. 8:1157-70). Alternatively, antisense molecules can be produced biologically using an expression vector into which a polynucleotide of the present invention has been subcloned in an antisense (i.e., reverse) orientation.

In yet another embodiment, the antisense polynucleotide molecule suitable for the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue, according to techniques that are known in the art.

The inhibitory triplex-forming oligonucleotides (TFOs) suitable for the present invention bind in the major groove of duplex DNA with high specificity and affinity (Knauert and Glazer, supra). Expression of the genes of the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nat. Med. 9:347-51). In addition, intrathecally delivered siRNA has recently been reported to block pain responses in two models (agonist-induced pain model and neuropathic pain model) in the rat (Dom et al. (2004) Nucleic Acids Res. 32(5):e49).

The siRNA molecules suitable for the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to genes, proteins or pathways of the present invention can be designed based on criteria well known in the art (e.g., Elbashir et al. (2001) EMBO J. 20:6877-88). For example, the target segment of the target mRNA should begin with AA (preferred), TA, GA, or CA; the GC ratio of the siRNA molecule should be 45-55%; the siRNA molecule should not contain three of the same nucleotides in a row; the siRNA molecule should not contain seven mixed G/Cs in a row; and the target segment should be in the ORF region of the target mRNA and should be at least 75 bp after the initiation ATG and at least 75 bp before the stop codon. siRNA molecules targeted to the polynucleotides of the present invention can be designed by one of ordinary skill in the art using the aforementioned criteria or other known criteria.

In another embodiment of the invention, the inhibitory polynucleotides are microRNA (miRNA) molecules. miRNA are endogenously expressed molecules (typically single-stranded RNA molecules of about 21-23 nucleotides in length), which regulate gene expression at the level of translation. Typically, miRNAs are encoded by genes that are transcribed from DNA but not translated into protein (non-coding RNA). Instead, they are processed from primary transcripts known as pri-miRNA to short stem-loop structures called pre-mIRNA and finally to functional miRNA. Mature miRNA molecules are partially complementary to one or more messenger RNA (mRNA) molecules, and their main function is to downregulate gene expression. miRNA are highly conserved and predicted to be responsible for regulating at least about 30% of the genes in the genome. Thus, CHO miRNA can be identified by relying on high human-mouse homology. For example, human miRNA sequences can be used to screen CHO specific miRNA. CHO specific miRNAs have been cloned. For example, the sequence of an exemplary CHO miRNA, Cgr-mir-21, is described in U.S. application Ser. No. 12/139,294 and PCT/US2008/066845, both filed on Jun. 13, 2008, the contents of both of which are incorporated by reference herein.

Down-regulation of the genes or proteins of the present invention in a cell or organism may also be achieved through the creation of cells or organisms whose endogenous genes corresponding to the differential CHO sequences of the present invention have been disrupted through insertion of extraneous polynucleotides sequences (i.e., a knockout cell or organism). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout cells include homologous recombination and are well known in the art (e.g., Wolfer et al. (2002) Trends Neurosci. 25:336-40).

The expression or activity of the genes, proteins or pathways of the invention may also up-regulated. Up-regulation includes providing an exogenous nucleic acid (e.g., an over-expression construct) encoding a protein or gene of interest or a variant retaining its activity or providing a factor or a molecule indirectly enhancing the protein activity. The variant generally shares common structural features with the protein or gene of interest and should retain the activity permitting the improved cellular phenotype. The variant may correspond to a homolog from another species (e.g. a rodent homolog; a primate homolog, such as a human homolog; another mammalian homolog; or a more distant homolog retaining sequence conservation sufficient to convey the desired effect on cellular phenotype). In some cases, the variant may retain at least 70%, at least 80%, at least 90%, or at least 95% sequence identity with the CHO sequence or with a known homolog. In certain embodiments, the variant is a nucleic acid molecule that hybridizes under stringent conditions to the CHO nucleic acid sequence or to the nucleic acid sequence of a known homolog.

For example, the isolated polynucleotides corresponding to the gene or proteins of the present invention may be operably linked to an expression control sequence such as the pMT2 and pED expression vectors for recombinant production. General methods of expressing recombinant proteins are well known in the art.

The expression or activity of the genes, proteins or pathways of the present invention may also be altered by exogenous agents, small molecules, pharmaceutical compounds, or other factors that may be directly or indirectly modulating the activity of the genes, proteins or pathwyas of the present invention. As a result, these agents, small molecules, pharmaceutical compounds, or other factors may be used to regulate the phenotype of CHO cells, e.g., increased production of a recombinant transgene, increased cell growth rate, high peak cell density, sustained high cell viability, high maximum cellular productivity, sustained high cellular productivity, low ammonium production, and low lactate production, etc.

Any combinations of the methods of altering gene or protein expression described above are within the scope of the invention. Any combination of genes or proteins affecting different cell phenotypes can be modulated based on the methods described herein and are within the scope of the invention.

It should be understood that the above-described embodiments and the following examples are given by way of illustration, not limitation. Various changes and modifications within the scope of the present invention will become apparent to those skilled in the art from the present description.

### EXAMPLES

### Example 1

### Exemplary Pathways Associated with High Cell Viability

Global pathway analysis was performed using, for example, Panther, which allows the identification of overrepresented pathways in a dataset using the entire array as a reference set. This is an unbiased and non-hypothesis driven method to identify key regulatory molecules and pathways that are important regulators for a cell phenotype, such as, enhanced survival. This type of analysis eliminates the bias in a typical custom array because a custom array can be a bias towards specific pathways based purely on the (limited) gene representation on the chip. Such pathway analysis was employed to gain insight into the main regulatory pathways that may contribute to survival in suspension batch culture. As the WyeHamster2a array is a custom oligo array and is predicted to cover approximately 15% of the detectable hamster transcripts there is a possibility of bias in pathway analysis of genelists derived from this array. Using Panther (www.pantherdb.org), a bioinformatics tool for the analysis of genelists and the detection of over-represented pathways and biological processes within a set of data, it is possible to identify potential bias via the use of all the transcripts on the WyeHamster2a array as a reference list, hence the statistical scores are based on the overall array and the size of the input list. For this analysis, each list is compared to the reference list using the binomial test described in Cho & Campbell (2000) “Transcription, genomes, function,” Trends Genet. 16, 409-415.

Based on this type of analysis, one exemplary pathway identified for both early and late culture during time course analysis was the cholesterol biosynthesis pathway. In both early and late culture, the important components of the cholesterol biosynthetic pathway were increased in the high viability B19 cells compared to the parental parent cells. Of the 15 enzymes in the cholesterol biosynthetic pathway, 5 are available on the WyeHamster2a array (HMGCS1, HMGCR, FDPS, MVD and FDFT1) of which 4 are significantly upregulated by more than 1.5-fold in late culture and the other, MVD (mevalonate (diphospho) decarboxylase) is upregulated by 1.4-fold in late batch culture (Table 1). This data is partly substantiated by the 2D DIGE data where HMGCS1 was identified as being almost 3-fold upregulated in B19 (Table 1).

TABLE 1

| | |
|---|---|
| Early | Late |

| | FC^{a} | P Value | FC | P Value |
|---|---|---|---|---|
| HMGCS1 | +2.5 | 9.8E−03 | +2.8 | 5.2E−03 |
| HMGCR | +1.8 | 3.8E−02 | +2.8 | 6.4E−03 |
| FDPS | — | — | +1.5 | 4.7E−03 |
| FDFT1 | — | — | +1.5 | 3.1E−02 |
| MVD^{b} | — | — | +1.4 | 1.2E−03 |

The components of the cholesterol biosynthetic pathway identified from the transcriptional profiling study are presented.
HMGCS1 (3-hydroxy-3-methylglutaryl-Coenzyme A synthase 1);
HMGCR (HMG Coenzyme A reductase);
FDPS (farnesyl diphosphate synthase);
FDFT1 (farnesyl-diphosphate farnesyltransferase 1);
MVD (mevalonate (diphospho) decarboxylase)
^{a}(+) Upregulation in B19, ratio is B19/parent
^{b}MVD did not pass the 1.5F filter applied during original data analysis

Additional softwares for pathway analysis (Ingenuity Pathway Analysis (v6.5 Ingenuity Systems, www.ingenuity.com), PATHWAY STUDIO (v.5.0; www.ariadnegenomics.com) were also used to perform global pathway analysis based on previously identified differentially expressed genes and/or proteins associated with various cell phenotypes of interest (see, U.S. application Ser. No. 11/788,872 and PCT/US2007/10002, both filed on Apr. 21, 2007, and U.S. application Ser. No. 12/139,294 and PCT/US2008/066845, both filed on Jun. 13, 2008, the contents of all of which are incorporated by reference herein).

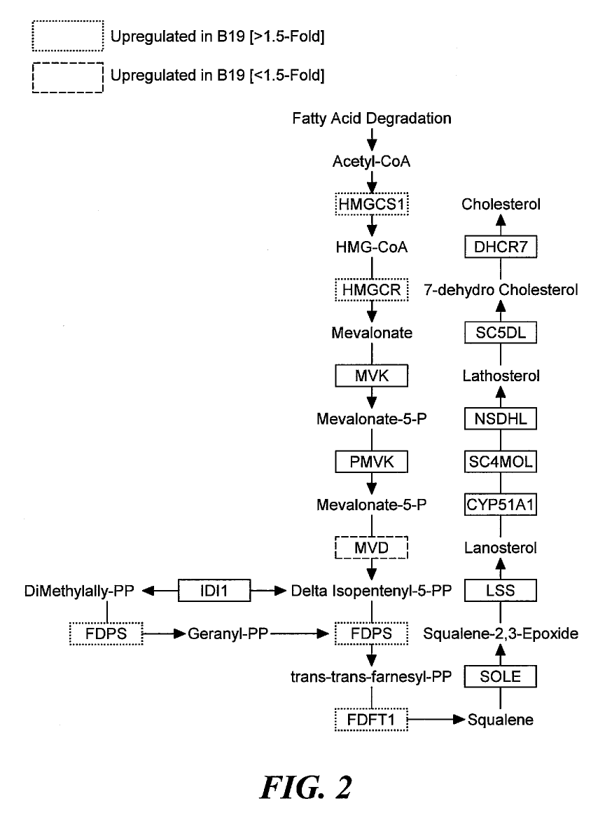

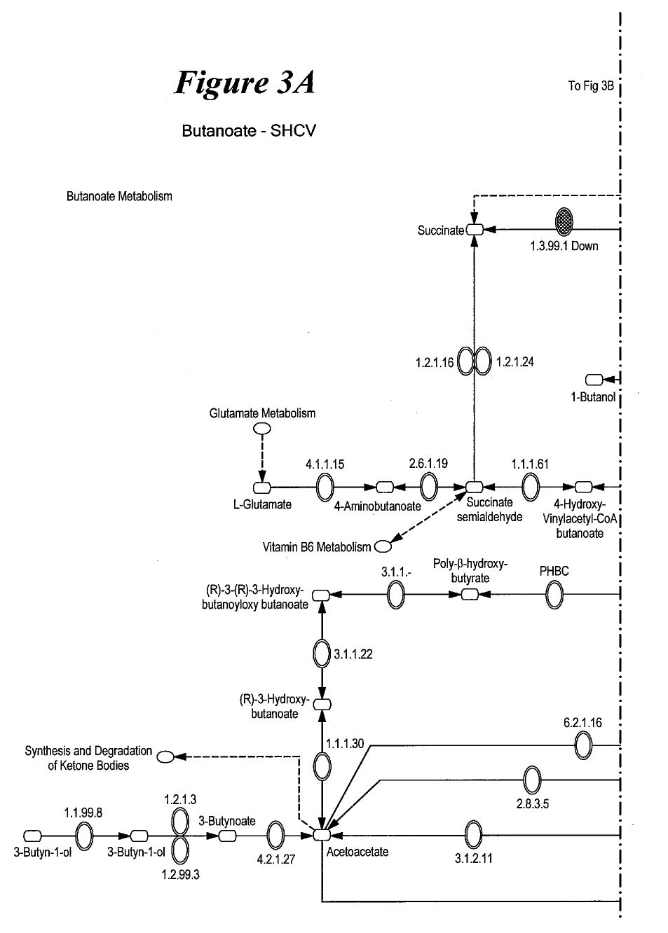

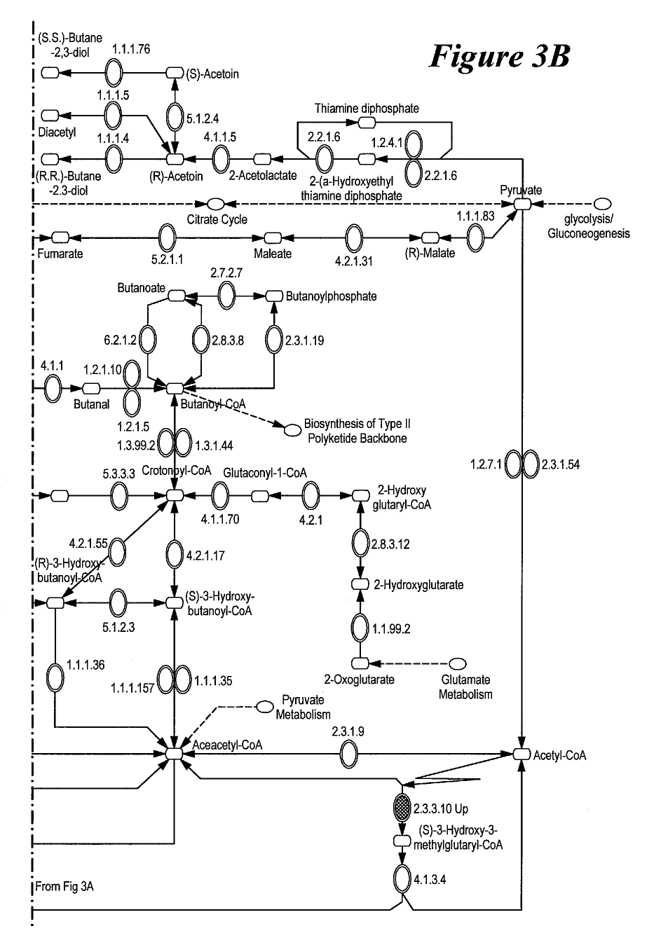

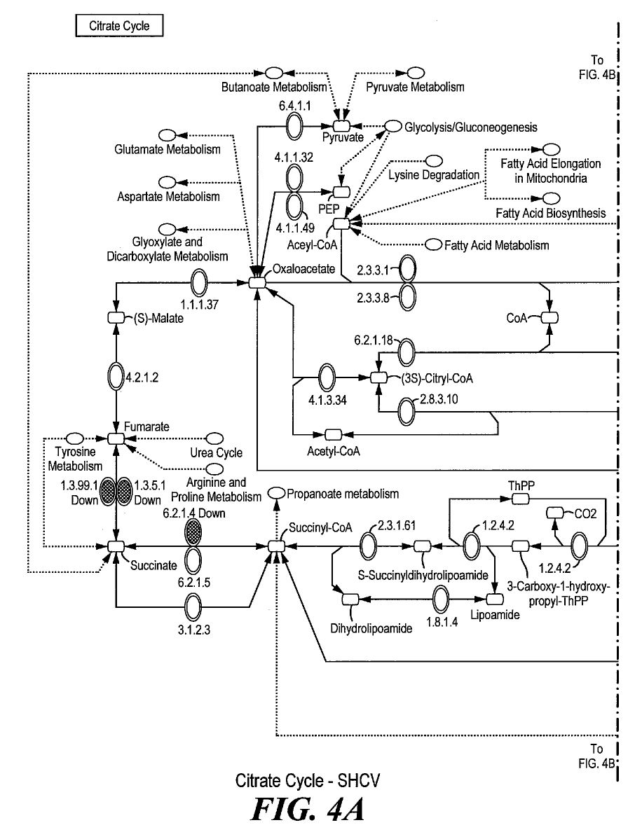

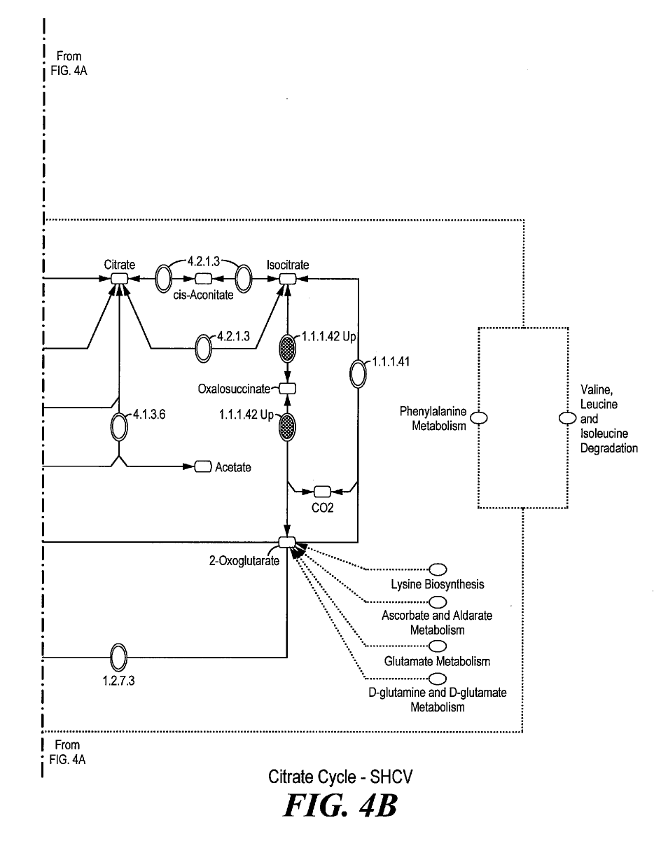

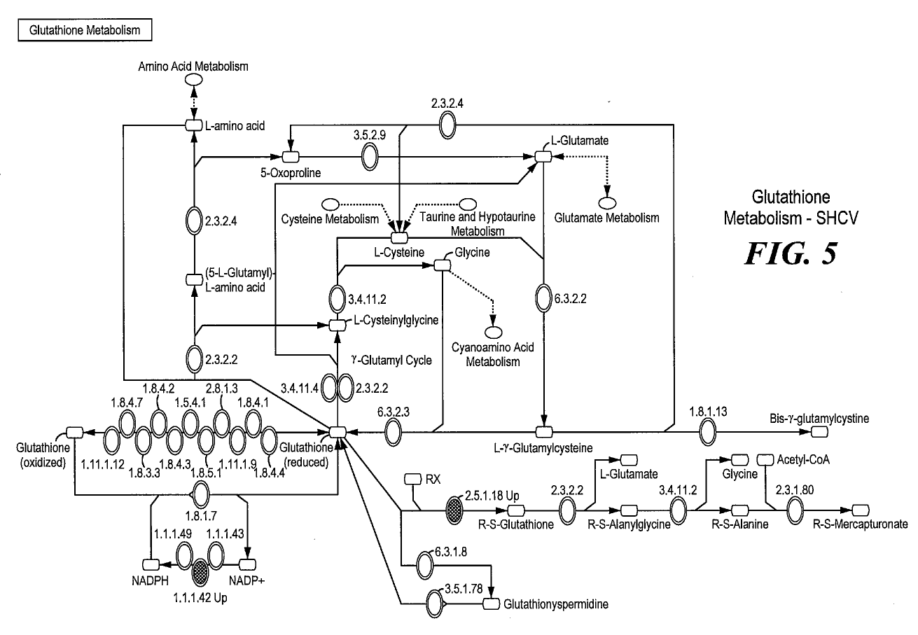

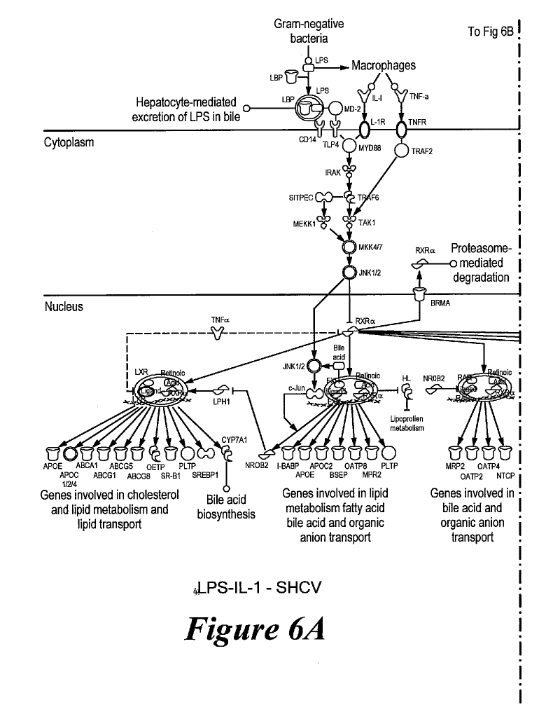

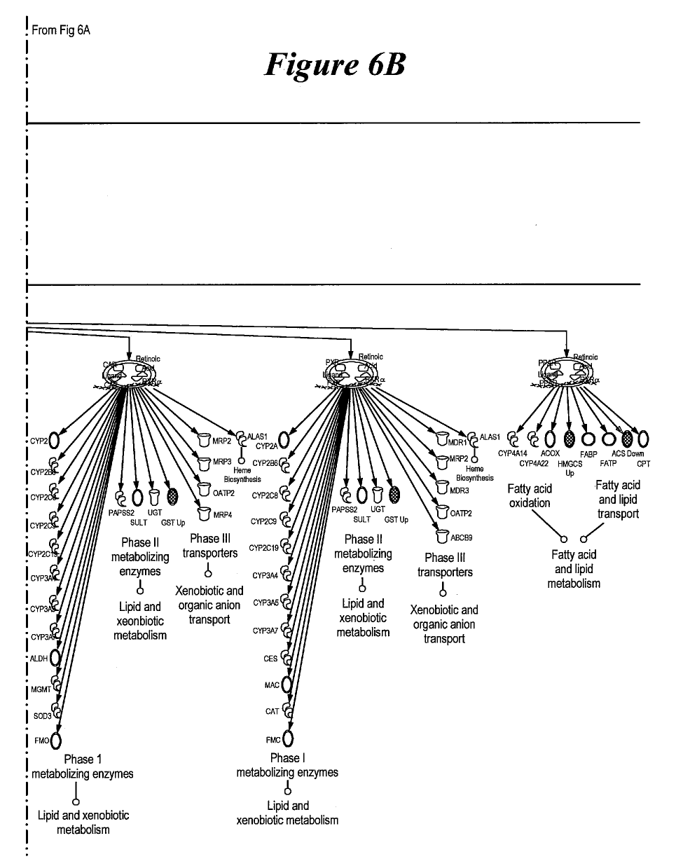

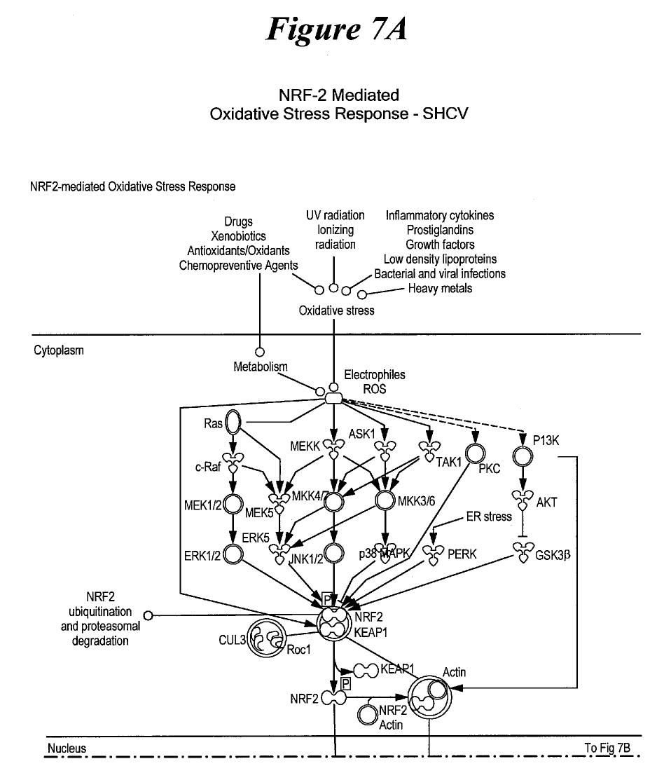

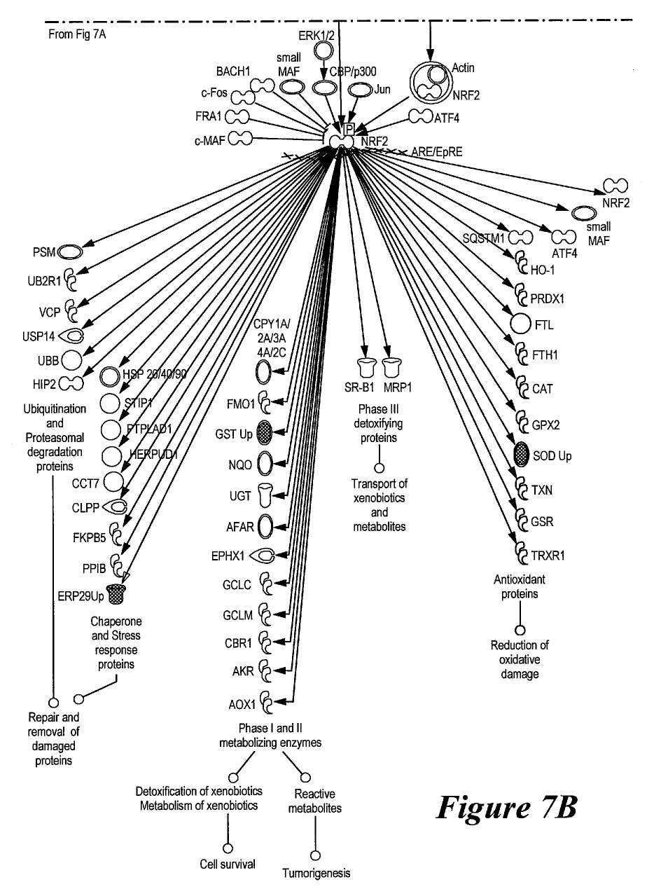

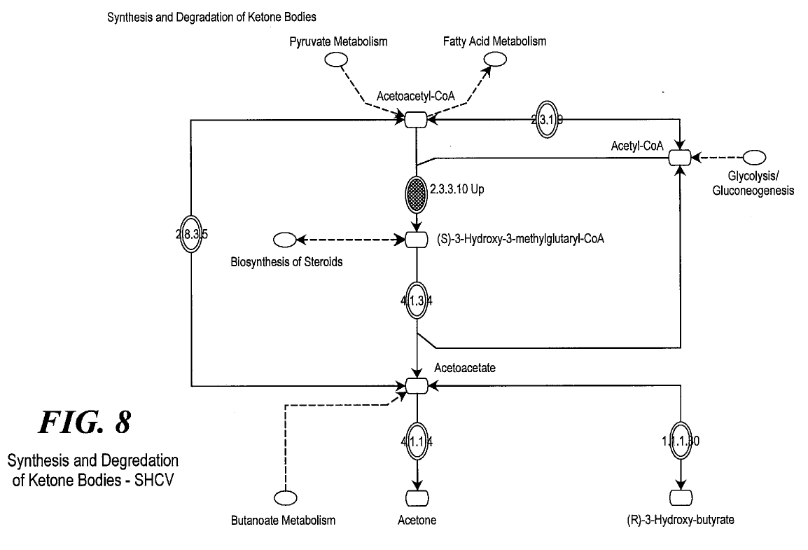

For example, pathway analysis using Ingenuity software based on previously identified differentially expressed genes and/or proteins associated with high cell viability led to the identification of the butanoate metabolism pathway (FIG. 3), the citrate cycle pathway (FIG. 4), the glutathione metabolism pathway (FIG. 5), the LPS-IL-1 Mediated Inhibition of RXR Function pathway (FIG. 6), the NRF-2 mediated oxidative stress response pathway (FIG. 7), and the synthesis and degradation of ketone bodies pathway (FIG. 8). Genes and/or proteins that were used to identify relevant pathways are indicated in FIGS. 2-8. In addition, additional exemplary genes or proteins involved in the above-identified pathways and that may be involved in regulating or indicative of high cell viability are summarized in Table 2 (the butanoate metabolism pathway), Table 3 (the citrate cycle pathway), Table 4 (the glutathione metabolism pathway), Table 5 (the LPS-IL-1 Mediated Inhibition of RXR Function pathway), Table 6 (the NRF-2 mediated oxidative stress response pathway), and Table 7 (the synthesis and degradation of ketone bodies pathway).

TABLE 2
Genes and Proteins Involved in the Butanoate Metabolism Pathway

| Name | Synonyms |
|---|---|
| (R)-3-((R)-3-Hydroxy- | (3R)-3-[(3R)-3-hydroxybutanoyl]oxybutanoic acid, (R)-3-((R)-3- |
| butanoyloxy)butanoate | hydroxybutanoyloxy)-butanoate, C8H14O5 |
| (R)-3-Hydroxy-butanoate | (3R)-3-hydroxybutanoic acid, (R)-(−)-3-hydroxybutyric acid sodium salt, (R)- |
| | 3-hydroxybutanoic acid, (R)-3-hydroxybutyric acid, 13613-65-5, 625-72-9, |
| | C4H8O3, D-beta-hydroxybutyrate, R-3-hydroxybutanoate, sodium (R)-3- |
| | hydroxybutyrate |
| (R)-3-Hydroxy-butanoyl-CoA | (R)-3-hydroxybutanoyl-CoA, (R)-3-hydroxybutyryl-coenzyme A, 21804-29-5, |
| | C25H42N7O18P3S, [(2R,3R,4R,5R)-5-(6-aminopurin-9-yl)-4-hydroxy-2- |
| | [[hydroxy-[hydroxy-[3-hydroxy-3-[2-[2-[(3R)-3- |
| | hydroxybutanoyl]sulfanylethylcarbamoyl]ethylcarbamoyl]-2,2-dimethyl- |
| | propoxy]phosphoryl]oxy-phosphoryl]oxymethyl]oxolan-3-yl]oxyphosphonic |
| | acid |
| (R)-Acetoin | (3R)-3-hydroxybutan-2-one, (R)-2-acetoin, (R)-3-hydroxy-2-butanone, (R)-3- |
| | hydroxybutan-2-one, (R)-dimethylketol, C4H8O2 |
| (R)-Malate | (2R)-2-hydroxybutanedioic acid, (R)-malate, 636-61-3, C4H6O5, D-malate, |
| | malic acid, L(+)- |
| (R,R)-Butane-2,3-diol | (2R,3R)-butane-2,3-diol, (R,R)-(−)-butane-2,3-diol, (R,R)-2,3-butanediol, |
| | (R,R)-butane-2,3-diol, 24347-58-8, C4H10O2, r,r-butane-2,3-diol |
| (S)-3-Hydroxy-butanoyl-CoA | (S)-3-hydroxybutanoyl-CoA, (S)-3-hydroxybutyryl-CoA, (S)-3-hydroxybutyryl- |
| | coenzyme A, 22138-45-0, C25H42N7O18P3S, [(2R,3R,4R,5R)-5-(6- |
| | aminopurin-9-yl)-4-hydroxy-2-[[hydroxy-[hydroxy-[3-hydroxy-3-[2-[2-[(3S)-3- |
| | hydroxybutanoyl]sulfanylethylcarbamoyl]ethylcarbamoyl]-2,2-dimethyl- |
| | propoxy]phosphoryl]oxy-phosphoryl]oxymethyl]oxolan-3-yl]oxyphosphonic |
| | acid |
| (S)-3-Hydroxy-3-methylglutaryl-CoA | (3S)-4-[2-[3-[[4-[[[(2R,3R,4R,5R)-5-(6-aminopurin-9-yl)-4-hydroxy-3- |
| | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-hydroxy-3-methyl- |
| | butanoic acid, (S)-3-hydroxy-3-methylglutaryl-CoA, 1553-55-5, |
| | C27H44N7O20P3S, hydroxymethylglutaryl-CoA, S-(hydrogen 3-hydroxy-3- |
| | methylglutaryl)coenzyme A, S-(hydrogen 3-hydroxy-3-methylpentanedioate) |
| | coenzyme A |
| (S)-Acetoin | (3S)-3-hydroxybutan-2-one, C4H8O2 |
| (S,S)-Butane-2,3-diol | (2S,3S)-butane-2,3-diol, (S,S)-butane-2,3-diol, 19132-06-0, 2,3-butanediol, |
| | (S-(R*,R*))-, C4H10O2 |
| 1-Butanol | 1-butanol, 1-hydroxybutane, 71-36-3, butan-1-ol, butanol, butyl alcohol, |
| | C4H10O, n-butanol |
| 1.1.1.— | |
| 1.1.1.157 | (S)-3-hydroxybutanoyl-CoA:NADP oxidoreductase, beta-hydroxybutyryl |
| | coenzyme A dehydrogenase, beta-hydroxybutyryl-CoA dehydrogenase, |
| | BHBD, dehydrogenase, L-3-hydroxybutyryl coenzyme A (nicotinamide |
| | adenine dinucleotide phosphate), L(+)-3-hydroxybutyryl-CoA dehydrogenase |
| 1.1.1.30 | (R)-3-hydroxybutanoate:NAD oxidoreductase, 3-D-hydroxybutyrate |
| | dehydrogenase, beta-hydroxybutyrate dehydrogenase, beta-hydroxybutyric |
| | acid dehydrogenase, beta-hydroxybutyric dehydrogenase, D-(−)-3- |
| | hydroxybutyrate dehydrogenase, D-3-hydroxybutyrate dehydrogenase, D- |
| | beta-hydroxybutyrate dehydrogenase, hydroxybutyrate oxidoreductase, |
| | NAD-beta-hydroxybutyrate dehydrogenase |
| 1.1.1.35 | (S)-3-hydroxyacyl-CoA:NAD oxidoreductase, 1-specific DPN-linked beta- |
| | hydroxybutyric dehydrogenase, 3-hydroxyacetyl-coenzyme A |
| | dehydrogenase, 3-hydroxyacyl coenzyme A dehydrogenase, 3- |
| | hydroxybutyryl-CoA dehydrogenase, 3-hydroxyisobutyryl-CoA |
| | dehydrogenase, 3-keto reductase, 3-L-hydroxyacyl-CoA dehydrogenase, |
| | 3beta-hydroxyacyl coenzyme A dehydrogenase, beta-hydroxy acid |
| | dehydrogenase, beta-hydroxyacyl CoA dehydrogenase, beta-hydroxyacyl |
| | dehydrogenase, beta-hydroxyacyl-coenzyme A synthetase, beta- |
| | hydroxyacylcoenzyme A dehydrogenase, beta-hydroxybutyrylcoenzyme A |
| | dehydrogenase, beta-keto-reductase, beta-ketoacyl-CoA reductase, L-3- |
| | hydroxyacyl CoA dehydrogenase, L-3-hydroxyacyl coenzyme A |
| | dehydrogenase |
| 1.1.1.36 | (R)-3-hydroxyacyl-CoA dehydrogenase, (R)-3-hydroxyacyl-CoA:NADP |
| | oxidoreductase, acetoacetyl coenzyme A reductase, beta-ketoacyl-CoA |
| | reductase, D(−)-beta-hydroxybutyryl CoA-NADP oxidoreductase, D-3- |
| | hydroxyacyl-CoA reductase, hydroxyacyl coenzyme-A dehydrogenase, |
| | NADP-linked acetoacetyl CoA reductase, NADPH:acetoacetyl-CoA |
| | reductase, short chain beta-ketoacetyl(acetoacetyl)-CoA reductase |
| 1.1.1.4 | (R)-2,3-butanediol dehydrogenase, (R)-diacetyl reductase, (R,R)-butane-2,3- |
| | diol:NAD oxidoreductase, 1-amino-2-propanol dehydrogenase, 1-amino-2- |
| | propanol oxidoreductase, 2,3-butanediol dehydrogenase, aminopropanol |
| | oxidoreductase, butylene glycol dehydrogenase, D-(−)-butanediol |
| | dehydrogenase, D-1-amino-2-propanol dehydrogenase, D-1-amino-2- |
| | propanol:NAD+ oxidoreductase, D-aminopropanol dehydrogenase, D- |
| | butanediol dehydrogenase, diacetyl (acetoin) reductase |
| 1.1.1.5 | acetoin:NAD oxidoreductase, diacetyl reductase |
| 1.1.1.61 | 4-hydroxybutanoate:NAD oxidoreductase, g-hydroxybutyrate dehydrogenase |
| 1.1.1.76 | (S,S)-butane-2,3-diol:NAD oxidoreductase, L(+)-2,3-butanediol |
| | dehydrogenase (L-acetoin forming), L-BDH, L-butanediol dehydrogenase |
| 1.1.1.83 | (R)-malate:NAD oxidoreductase (decarboxylating), bifunctional L(+)-tartrate |
| | dehydrogenase-D(+)-malate (decarboxylating), D-malate dehydrogenase, D- |
| | malic enzyme |
| 1.1.99.2 | (S)-2-hydroxyglutarate:(acceptor) 2-oxidoreductase, alpha-hydroxyglutarate |
| | dehydrogenase, alpha-hydroxyglutarate dehydrogenase (NAD+ specific), |
| | alpha-hydroxyglutarate oxidoreductase, alpha-ketoglutarate reductase, |
| | hydroxyglutaric dehydrogenase, L-alpha-hydroxyglutarate dehydrogenase, |
| | L-alpha-hydroxyglutarate:NAD+ 2-oxidoreductase |
| 1.1.99.8 | alcohol:(acceptor) oxidoreductase, MDH, primary alcohol dehydrogenase, |
| | quinohemoprotein alcohol dehydrogenase, quinoprotein alcohol |
| | dehydrogenase, quinoprotein ethanol dehydrogenase |
| 1.2.1.10 | acetaldehyde:NAD oxidoreductase (CoA-acetylating), aldehyde |
| | dehydrogenase (acylating) |
| 1.2.1.16 | succinate semialdehyde dehydrogenase (nicotinamide adenine dinucleotide |
| | (phosphate)), succinate-semialdehyde:NAD(P) oxidoreductase |
| 1.2.1.24 | succinate semialdehyde: NAD+ oxidoreductase, succinate- |
| | semialdehyde:NAD oxidoreductase, succinic semialdehyde dehydrogenase, |
| | succinyl semialdehyde dehydrogenase |
| 1.2.1.3 | aldehyde:NAD oxidoreductase, CoA-independent aldehyde dehydrogenase, |
| | m-methylbenzaldehyde dehydrogenase, NAD-aldehyde dehydrogenase, |
| | NAD-dependent 4-hydroxynonenal dehydrogenase, NAD-dependent |
| | aldehyde dehydrogenase, NAD-linked aldehyde dehydrogenase, |
| | propionaldehyde dehydrogenase |
| 1.2.1.57 | butanal:NAD(P) oxidoreductase (CoA-acylating) |
| 1.2.4.1 | MtPDC (mitochondrial pyruvate dehydogenase complex), PDH, pyruvate |
| | decarboxylase, pyruvate dehydrogenase, pyruvate dehydrogenase complex, |
| | pyruvate:lipoamide 2-oxidoreductase (decarboxylating and acceptor- |
| | acetylating), pyruvic acid dehydrogenase, pyruvic dehydrogenase |
| 1.2.7.1 | pyruvate oxidoreductase, pyruvate synthetase, pyruvate:ferredoxin 2- |
| | oxidoreductase (CoA-acetylating), pyruvate:ferredoxin oxidoreductase, |
| | pyruvic-ferredoxin oxidoreductase |
| 1.2.99.3 | aldehyde dehydrogenase (acceptor), aldehyde:(pyrroloquinoline-quinone) |
| | oxidoreductase |
| 1.3.1.44 | acyl-CoA:NAD trans-2-oxidoreductase |
| 1.3.99.2 | 3-hydroxyacyl CoA reductase, butanoyl-CoA:(acceptor) 2,3-oxidoreductase, |
| | butyryl coenzyme A dehydrogenase, butyryl dehydrogenase, enoyl- |
| | coenzyme A reductase, ethylene reductase, short-chain acyl CoA |
| | dehydrogenase, short-chain acyl-coenzyme A dehydrogenase, unsaturated |
| | acyl coenzyme A reductase, unsaturated acyl-CoA reductase |
| 2-(&alpha;-Hydroxyethyl)-thiamine | 2-(1-hydroxyethyl)thiamine pyrophosphate, C14H23N4O8P2S+, [2-[3-[(4- |
| diphosphate | amino-2-methyl-pyrimidin-5-yl)methyl]-2-(1-hydroxyethyl)-4-methyl-1-thia-3- |
| | azoniacyclopenta-2,4-dien-5-yl]ethoxy-hydroxy-phosphoryl]oxyphosphonic |
| | acid |
| 2-Acetolactate | 2-acetoxypropanoic acid, 2-acetyloxypropanoic acid, 535-17-1, acetyllactic |
| | acid, alpha-acetolactate, alpha-acetoxypropionic acid, C5H8O4, propanoic |
| | acid, 2-(acetyloxy)- |
| 2-Hydroxy-glutaryl-CoA | 2-hydroxyglutaryl-1-coa, 4-[2-[3-[[4-[[[(2R,3R,4R,5R)-5-(6-aminopurin-9-yl)-4- |
| | hydroxy-3-phosphonooxy-oxolan-2-yl]methoxy-hydroxy-phosphoryl]oxy- |
| | hydroxy-phosphoryl]oxy-2-hydroxy-3,3-dimethyl- |
| | butanoyl]amino]propanoylamino]ethylsulfanylcarbonyl]-4-hydroxy-butanoic |
| | acid, C26H42N7O20P3S, coenzyme A, S-(5-hydrogen 2- |
| | hydroxypentanedioate), (R)- |
| 2-Hydroxyglutarate | 2-hydroxyglutarate, 2-hydroxyglutaric acid, 2-hydroxypentanedioic acid, |
| | 2889-31-8, C5H8O5, pentanedioic acid, 2-hydroxy- |
| 2-Oxoglutarate | 2-ketoglutarate, 2-oxoglutarate, 2-oxopentanedioic acid, 328-50-7, alpha- |
| | ketoglutarate, alpha-ketoglutaric acid, alphaKG, C5H6O5, glutaric acid, 2- |
| | oxo-, glutaric acid, 2-oxo-(8Cl), pentanedioic acid, 2-oxo- |
| 2.2.1.6 | acetohydroxy acid synthetase, acetohydroxyacid synthase, acetolactate |
| | pyruvate-lyase (carboxylating), acetolactic synthetase, alpha-acetohydroxy |
| | acid synthetase, alpha-acetohydroxyacid synthase, alpha-acetolactate |
| | synthase, alpha-acetolactate synthetase |
| 2.3.1.19 | butanoyl-CoA:phosphate butanoyltransferase, phosphotransbutyrylase |
| 2.3.1.54 | acetyl-CoA:formate C-acetyltransferase, formate acetyltransferase, pyruvate |
| | formate-lyase, pyruvic formate-lyase |
| 2.3.1.9 | 2-methylacetoacetyl-CoA thiolase, 3-oxothiolase, acetoacetyl-CoA thiolase, |
| | acetyl coenzyme A thiolase, acetyl-CoA acetyltransferase, acetyl- |
| | CoA:acetyl-CoA C-acetyltransferase, acetyl-CoA:N-acetyltransferase, beta- |
| | acetoacetyl coenzyme A thiolase, thiolase II |
| 2.6.1.19 | 4-aminobutanoate:2-oxoglutarate aminotransferase, 4-aminobutyrate |
| | aminotransferase, 4-aminobutyrate-2-ketoglutarate aminotransferase, 4- |
| | aminobutyrate-2-oxoglutarate aminotransferase, 4-aminobutyrate-2- |
| | oxoglutarate transaminase, 4-aminobutyric acid 2-ketoglutaric acid |
| | aminotransferase, 4-aminobutyric acid aminotransferase, aminobutyrate |
| | aminotransferase, aminobutyrate transaminase, beta-alanine |
| | aminotransferase, beta-alanine-oxoglutarate aminotransferase, beta-alanine- |
| | oxoglutarate transaminase, g-aminobutyrate aminotransaminase, g- |
| | aminobutyrate transaminase, g-aminobutyrate-alpha-ketoglutarate |
| | aminotransferase, g-aminobutyrate-alpha-ketoglutarate transaminase, g- |
| | aminobutyrate:alpha-oxoglutarate aminotransferase, g-aminobutyric acid |
| | aminotransferase, g-aminobutyric acid pyruvate transaminase, g- |
| | aminobutyric acid transaminase, g-aminobutyric acid-2-oxoglutarate |
| | transaminase, g-aminobutyric acid-alpha-ketoglutarate transaminase, g- |
| | aminobutyric acid-alpha-ketoglutaric acid aminotransferase, g-aminobutyric |
| | transaminase, GABA aminotransferase, GABA transaminase, GABA |
| | transferase, GABA-2-oxoglutarate aminotransferase, GABA-2-oxoglutarate |
| | transaminase, GABA-alpha-ketoglutarate aminotransferase, GABA-alpha- |
| | ketoglutarate transaminase, GABA-alpha-ketoglutaric acid transaminase, |
| | GABA-alpha-oxoglutarate aminotransferase, GABA-oxoglutarate |
| | aminotransferase, GABA-oxoglutarate transaminase, glutamate-succinic |
| | semialdehyde transaminase |
| 2.7.2.7 | ATP:butanoate 1-phosphotransferase |
| 2.8.3.12 | (E)-glutaconate CoA-transferase |
| 2.8.3.5 | 3-ketoacid CoA-transferase, 3-ketoacid coenzyme A transferase, 3-oxo-CoA |
| | transferase, 3-oxoacid CoA dehydrogenase, 3-oxoacid coenzyme A- |
| | transferase, acetoacetate succinyl-CoA transferase, acetoacetyl coenzyme |
| | A-succinic thiophorase, succinyl coenzyme A-acetoacetyl coenzyme A- |
| | transferase, succinyl-CoA transferase, succinyl-CoA:3-oxo-acid CoA- |
| | transferase |
| 2.8.3.8 | acetate coenzyme A-transferase, acyl-CoA:acetate CoA-transferase, butyryl |
| | CoA:acetate CoA transferase, butyryl coenzyme A transferase, succinyl- |
| | CoA:acetate CoA transferase |
| 3-Butyn-1-al | 52844-23-2, but-3-ynal, C4H4O |
| 3-Butyn-1-ol | 1-butyn-4-ol, 2-hydroxyethylacetylene, 3-butyne-1-ol, 3-butynol, 3-butynyl |
| | alcohol, 4-hydroxy-1-butyne, 927-74-2, but-3-yn-1-ol, C4H6O |
| 3-Butynoate | 2345-51-9, 3-butynoate, 3-butynoic acid, but-3-ynoic acid, C4H4O2 |
| 3.1.1.— | |
| 3.1.1.22 | (R)-3-((R)-3-hydroxybutanoyloxy)butanoate hydroxybutanoylhydrolase, D-(−)- |
| | 3-hydroxybutyrate-dimer hydrolase |
| 3.1.2.11 | acetoacetyl CoA deacylase, acetoacetyl coenzyme A deacylase, acetoacetyl |
| | coenzyme A hydrolase |
| 4-Aminobutanoate | 4-aminobutanoic acid, 4-aminobutyrate, 4-aminobutyric acid, 56-12-2, |
| | butanoic acid, 4-amino-, C4H9NO2, gamma-amino-N-butyric acid, gamma- |
| | aminobutyric acid |
| 4-Hydroxy-butanoate | 4-hydroxybutanoate, 4-hydroxybutanoic acid, 4-hydroxybutyrate, 4- |
| | hydroxybutyric acid, 591-81-1, butanoic acid, 4-hydroxy-, C4H8O3, gamma- |
| | hydroxybutyrate, gamma-hydroxybutyric acid |
| 4.1.1.15 | aspartate 1-decarboxylase, aspartic alpha-decarboxylase, cysteic acid |
| | decarboxylase, g-glutamate decarboxylase, Glutamate decarboxylase, L- |
| | aspartate-alpha-decarboxylase, L-glutamate 1-carboxy-lyase, L-glutamate |
| | alpha-decarboxylase, L-glutamic acid decarboxylase, L-glutamic |
| | decarboxylase |
| 4.1.1.5 | (S)-2-hydroxy-2-methyl-3-oxobutanoate carboxy-lyase, alpha-acetolactate |
| | decarboxylase |
| 4.1.1.70 | glutaconyl coenzyme A decarboxylase, pent-2-enoyl-CoA carboxy-lyase |
| 4.1.3.4 | (S)-3-hydroxy-3-methylglutaryl-CoA acetoacetate-lyase, 3-hydroxy-3- |
| | methylglutaryl CoA cleaving enzyme, 3-hydroxy-3-methylglutaryl coenzyme |
| | A lyase, 3-hydroxy-3-methylglutaryl-CoA lyase, hydroxymethylglutaryl |
| | coenzyme A lyase, hydroxymethylglutaryl coenzyme A-cleaving enzyme |
| 4.2.1.— | EctC, HPAH, hydratase |
| 4.2.1.17 | (3S)-3-hydroxyacyl-CoA hydro-lyase, 2-enoyl-CoA hydratase, 2-octenoyl |
| | coenzyme A hydrase, acyl coenzyme A hydrase, beta-hydroxyacid |
| | dehydrase, beta-hydroxyacyl-CoA dehydrase, crotonase, crotonyl hydrase, |
| | D-3-hydroxyacyl-CoA dehydratase, ECH, enol-CoA hydratase, enoyl |
| | coenzyme A hydrase (D), enoyl coenzyme A hydrase (L), enoyl coenzyme A |
| | hydratase, enoyl hydrase, hydratase, enoyl coenzyme A, short chain enoyl |
| | coenzyme A hydratase, short-chain enoyl-CoA hydratase, trans-2-enoyl-CoA |
| | hydratase, unsaturated acyl-CoA hydratase |
| 4.2.1.27 | 3-oxopropanoate hydro-lyase, acetylmonocarboxylic acid hydrase |
| 4.2.1.31 | (R)-malate hydro-lyase, D-malate hydro-lyase, malease |
| 4.2.1.55 | (3R)-3-hydroxybutanoyl-CoA hydro-lyase, D-3-hydroxybutyryl coenzyme A |
| | dehydratase, D-3-hydroxybutyryl-CoA dehydratase, enoyl coenzyme A |
| | hydrase (D) |
| 5.1.2.3 | 3-hydroxyacyl-CoA epimerase, 3-hydroxybutanoyl-CoA 3-epimerase, 3- |
| | hydroxybutyryl coenzyme A epimerase |
| 5.1.2.4 | acetylmethylcarbinol racemase |
| 5.2.1.1 | maleate cis-trans-isomerase |
| 5.3.3.3 | D3-cis-D2-trans-enoyl-CoA isomerase, vinylacetyl coenzyme A D-isomerase, |
| | vinylacetyl coenzyme A isomerase, vinylacetyl-CoA D3-D2-isomerase |
| 6.2.1.16 | acetoacetate:CoA ligase (AMP-forming), acetoacetyl-CoA synthetase |
| 6.2.1.2 | acyl-activating enzyme, butanoate:CoA ligase (AMP-forming), butyryl-CoA |
| | synthetase, fatty acid thiokinase (medium chain) |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme |
| | A, [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |
| Butanal | 1-butanal, 123-72-8, aldehyde C4, butal, butalyde, butanal, butyraldehyde, |
| | butyric aldehyde, C4H8O, n-butyraldehyde |
| Butanoate | 107-92-6, 156-54-7, 461-55-2, butanoic acid, butyrate, C4 SCFA, C4H8O2, |
| | n-butyrate, sodium butyrate |
| Butanoyl-CoA | 2140-48-9, butanoyl-coenzyme A, butyryl-CoA, C25H42N7O17P3S, |
| | [(2R,3R,4R,5R)-5-(6-aminopurin-9-yl)-2-[[[[3-[2-(2- |
| | butanoylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-4-hydroxy- |
| | oxolan-3-yl]oxyphosphonic acid |
| Butanoylphosphate | butanoyloxyphosphonic acid, C4H9O5P |
| Crotonoyl-CoA | 102680-35-3, 2-butenoyl-CoA, but-2-enoyl-CoA, C25H40N7O17P3S, |
| | crotonoyl-CoA, crotonyl-CoA, [(2R,3R,4R,5R)-5-(6-aminopurin-9-yl)-2-[[[[3- |
| | [2-(2-but-2-enoylsulfanylethylcarbamoyl)ethylcarbamoyl]-3-hydroxy-2,2- |
| | dimethyl-propoxy]-hydroxy-phosphoryl]oxy-hydroxy-phosphoryl]oxymethyl]- |
| | 4-hydroxy-oxolan-3-yl]oxyphosphonic acid |
| Diacetyl | 2,3-butanedione, 431-03-8, butane-2,3-dione, C4H6O2 |
| Fumarate | (E)-but-2-enedioic acid, 110-17-8, 2-butenedioic acid (2E)-, C4H4O4, |
| | fumarate |
| Glutaconyl-1-CoA | 4-[2-[3-[[4-[[[5-(6-aminopurin-9-yl)-4-hydroxy-3-phosphonooxy-oxolan-2- |
| | yl]methoxy-hydroxy-phosphoryl]oxy-hydroxy-phosphoryl]oxy-2-hydroxy-3,3- |
| | dimethyl-butanoyl]amino]propanoylamino]ethylsulfanylcarbonyl]but-3-enoic |
| | acid, 6712-05-6, C26H40N7O19P3S, coenzyme A, glutaconyl-, coenzyme A, |
| | S-(5-hydrogen 2-pentenedioate), glutaconyl-1-CoA, glutaconyl-1-coenzyme |
| | A, glutaconyl-coa |
| L-Glutamate | (2S)-2-aminopentanedioic acid, 142-47-2, 19473-49-5, 56-86-0, C5H9NO4, |
| | glutamate, glutamic acid, L-Glu, L-glutamate, L-glutamic acid, monosodium |
| | glutamate, potassium glutamate, potassium L-glutamate, sodium glutamate |
| Maleate | (Z)-but-2-enedioic acid, 110-16-7, 2-butenedioic acid, 2-butenedioic acid |
| | (2Z)-, 2-butenedioic acid (Z)-, 2-butenedioic acid (Z)-(9Cl), C4H4O4, cis- |
| | butenedioic acid, toxilic acid |
| PHBC | Acatn, EctA, LAC1, LAG1, PHBC |
| Poly-&beta;-hydroxy-butyrate | ((R)-3-hydroxybutanoyl)(n-2), (C4H6O2)n, 29435-48-1, butanoic acid, 3- |
| | hydroxy-, (R)-, homopolymer, poly(D-beta-hydroxybutyrate), poly-beta- |
| | hydroxybutyrate, (R)-isomer |
| Pyruvate | 127-17-3, 2-oxopropanoate, 2-oxopropanoic acid, 57-60-3, C3H4O3, |
| | propanoic acid, 2-oxo-, propanoic acid, 2-oxo-, ion(1-), propanoic acid, 2- |
| | oxo-, sodium salt, pyruvate, pyruvic acid, sodium salt, sodium pyruvate |
| Succinate | 1,2-ethanedicarboxylic acid, 1,4-butanedioic acid, 110-15-6, 56-14-4, amber |
| | acid, asuccin, butanedioate, butanedioic acid, C4H6O4, ethylenesuccinic |
| | acid, katasuccin, potassium succinate, succinate, wormwood acid |
| Succinate semialdehyde | 3-formylpropanoic acid, 4-oxobutanoic acid, 692-29-5, beta-formylpropionic |
| | acid, butanoic acid, 4-oxo-, butanoic acid, 4-oxo-(9Cl), butryaldehydic acid, |
| | C4H6O3, gamma-oxybutyric acid, succinaldehydic acid, succinate |
| | semialdehyde |
| Thiamine diphosphate | 136-09-4, 154-87-0, 23883-45-6, C12H19N4O7P2S+, cocarboxylase, |
| | thiamin diphosphate, thiamine diphosphate hydrochloride, thiazolium, 3-((4- |
| | amino-2-methyl-5-pyrimidinyl)methyl)-4-methyl-5-(4,6,6-trihydroxy-3,5-dioxa- |
| | 4,6-diphosphahex-1-yl)-, chloride, P,P′-dioxide, [2-[3-[(4-amino-2-methyl- |
| | pyrimidin-5-yl)methyl]-4-methyl-1-thia-3-azoniacyclopenta-2,4-dien-5- |
| | yl]ethoxy-hydroxy-phosphoryl]oxyphosphonic acid |
| Vinylacetyl-CoA | 3-butenoyl-CoA, C25H40N7O17P3S, vinylacetyl-CoA, [(2R,3R,4R,5R)-5-(6- |
| | aminopurin-9-yl)-2-[[[[3-[2-(2-but-3- |
| | enoylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-4-hydroxy- |
| | oxolan-3-yl]oxyphosphonic acid |

*Genes/proteins that were used to identify the pathway:
1.3.99.1: Complex II, Succinate INT Dehydrogenase
2.3.3.10: (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b- methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase

TABLE 3
Genes and Proteins Involved in the Citrate Cycle Pathway

| Name | Synonyms |
|---|---|
| (3S)-Citryl-CoA | (3S)-citryl-coa, 2-[2-[3-[[4-[[[5-(6-aminopurin-9-yl)-4-hydroxy-3-phosphonooxy-oxolan- |
| | 2-yl]methoxy-hydroxy-phosphoryl]oxy-hydroxy-phosphoryl]oxy-2-hydroxy-3,3-dimethyl- |
| | butanoyl]amino]propanoylamino]ethylsulfanylcarbonylmethyl]-2-hydroxy-butanedioic |
| | acid, 3131-26-8, C27H42N7O22P3S, citryl-coa, coenzyme A, S-(2,3-dihydrogen 2- |
| | hydroxy-1,2,3-propanetricarboxylate) |
| (S)-Malate | (−)-malic acid, (2S)-2-hydroxybutanedioic acid, (S)-malate, 97-67-6, butanedioic acid, |
| | hydroxy-, (2S)-, butanedioic acid, hydroxy-, (S)-, butanedioic acid, hydroxy-, (S)-(9Cl), |
| | C4H6O5, L-2-hydroxybutanedioic acid, L-apple acid, S-2-hydroxybutanedioic acid |
| 1.1.1.37 | (S)-malate:NAD oxidoreductase, L-malate dehydrogenase, L-malate-NAD+ |
| | oxidoreductase, malate (NAD) dehydrogenase, malic acid dehydrogenase, malic |
| | dehydrogenase, MDH, NAD-dependent malate dehydrogenase, NAD-dependent malic |
| | dehydrogenase, NAD-L-malate dehydrogenase, NAD-linked malate dehydrogenase, |
| | NAD-malate dehydrogenase, NAD-malic dehydrogenase, NAD-specific malate |
| | dehydrogenase |
| 1.1.1.41 | beta-ketoglutaric-isocitric carboxylase, isocitrate:NAD oxidoreductase |
| | (decarboxylating), isocitric acid dehydrogenase, isocitric dehydrogenase, NAD |
| | dependent isocitrate dehydrogenase, NAD isocitrate dehydrogenase, NAD isocitric |
| | dehydrogenase, NAD-linked isocitrate dehydrogenase, NAD-specific isocitrate |
| | dehydrogenase |
| 1.2.4.2 | 2-ketoglutarate dehydrogenase, 2-oxoglutarate dehydrogenase, 2- |
| | oxoglutarate:lipoamide 2-oxidoreductase (decarboxylating and acceptor-succinylating), |
| | 2-oxoglutarate:lipoate oxidoreductase, AKGDH, alpha-ketoglutarate dehydrogenase, |
| | alpha-ketoglutaric acid dehydrogenase, alpha-ketoglutaric dehydrogenase, alpha- |
| | oxoglutarate dehydrogenase, ketoglutaric dehydrogenase, OGDC, oxoglutarate |
| | decarboxylase, oxoglutarate dehydrogenase |
| 1.2.7.3 | 2-oxoglutarate-ferredoxin oxidoreductase, 2-oxoglutarate:ferredoxin 2-oxidoreductase |
| | (CoA-succinylating), alpha-ketoglutarate synthase, alpha-ketoglutarate-ferredoxin |
| | oxidoreductase, oxoglutarate synthase |
| 1.8.1.4 | dehydrolipoate dehydrogenase, diaphorase, dihydrolipoamide:NAD oxidoreductase, |
| | dihydrolipoic dehydrogenase, dihydrolipoyl dehydrogenase, dihydrothioctic |
| | dehydrogenase, LDP-Glc, LDP-Val, lipoamide dehydrogenase (NADH), lipoamide |
| | oxidoreductase (NADH), lipoamide reductase, lipoamide reductase (NADH2), lipoate |
| | dehydrogenase, lipoic acid dehydrogenase, lipoyl dehydrogenase |
| 2-Oxoglutarate | 2-ketoglutarate, 2-oxoglutarate, 2-oxopentanedioic acid, 328-50-7, alpha-ketoglutarate, |
| | alpha-ketoglutaric acid, alphaKG, C5H6O5, glutaric acid, 2-oxo-, glutaric acid, 2-oxo- |
| | (8Cl), pentanedioic acid, 2-oxo- |
| 2.3.1.61 | dihydrolipoamide succinyltransferase, dihydrolipoic transsuccinylase, dihydrolipolyl |
| | transsuccinylase, dihydrolipoyl transsuccinylase, lipoate succinyltransferase |
| | (Escherichia coli), lipoic transsuccinylase, lipoyl transsuccinylase, succinyl- |
| | CoA:dihydrolipoamide S-succinyltransferase, succinyl-CoA:dihydrolipoate S- |
| | succinyltransferase |
| 2.3.3.1 | (R)-citric synthase, acetyl-CoA:oxaloacetate C-acetyltransferase [thioester-hydrolysing, |
| | (pro-S)-carboxymethyl forming], citrate condensing enzyme, citrate oxaloacetate-lyase |
| | [(pro-3S)-CH2COO-acetyl-CoA], citrate oxaloacetate-lyase, CoA-acetylating, citrate |
| | synthase, citrate synthetase, citric synthase, citric-condensing enzyme, citrogenase, |
| | condensing enzyme, oxalacetic transacetase, oxaloacetate transacetase |
| 2.3.3.8 | acetyl-CoA:oxaloacetate acetyltransferase (isomerizing, ADP-phosphorylating), acetyl- |
| | CoA:oxaloacetate C-acetyltransferase [(pro-S)-carboxymethyl-forming, ADP- |
| | phosphorylating], adenosine triphosphate citrate lyase, ATP citrate (pro-S)-lyase, ATP- |
| | citric lyase, ATP:citrate oxaloacetate-lyase [(pro-S)-CH2COO-&gt;acetyl-CoA] (ATP- |
| | dephosphorylating), ATP:citrate oxaloacetate-lyase [(pro-S)-CH2COO-acetyl-CoA] |
| | (ATP-dephosphorylating), citrate cleavage enzyme, citrate-ATP lyase, citric cleavage |
| | enzyme |
| 2.8.3.10 | acetyl-CoA:citrate CoA-transferase |
| 3-Carboxy-1-hydroxy-propyl-ThPP | 3-carboxy-1-hydroxypropyl-ThPP, 4-[3-[(4-amino-2-methyl-pyrimidin-5-yl)methyl]-5-[2- |
| | (hydroxy-phosphonooxy-phosphoryl)oxyethyl]-4-methyl-1-thia-3-azoniacyclopenta-2,4- |
| | dien-2-yl]-4-hydroxy-butanoic acid, C16H25N4O10P2S+ |
| 3.1.2.3 | succinyl coenzyme A deacylase, succinyl coenzyme A hydrolase, succinyl-CoA |
| | acylase |
| 4.1.1.32 | GTP:oxaloacetate carboxy-lyase (transphosphorylating), PEP carboxylase, |
| | phosphoenolpyruvate carboxykinase, phosphoenolpyruvate carboxylase, |
| | phosphoenolpyruvic carboxykinase, phosphoenolpyruvic carboxykinase (GTP), |
| | phosphoenolpyruvic carboxylase (GTP), phosphopyruvate (guanosine triphosphate) |
| | carboxykinase, phosphopyruvate carboxylase, phosphopyruvate carboxylase (GTP) |
| 4.1.1.49 | ATP:oxaloacetate carboxy-lyase (transphosphorylating), PEP carboxykinase, PEP |
| | carboxylase, PEPCK, PEPCK (ATP), PEPK, phosphoenolpyruvate carboxykinase, |
| | phosphoenolpyruvate carboxylase, phosphoenolpyruvate carboxylase (ATP), |
| | phosphoenolpyruvic carboxykinase, phosphoenolpyruvic carboxylase, |
| | phosphopyruvate carboxykinase, phosphopyruvate carboxykinase (adenosine |
| | triphosphate), phosphopyruvate carboxylase (ATP) |
| 4.1.3.34 | (3S)-citryl-CoA oxaloacetate-lyase |
| 4.1.3.6 | citrase, citratase, citrate aldolase, citrate lyase, citrate oxaloacetate-lyase, citrate |
| | oxaloacetate-lyase [(pro-3S)-CH2COO-acetate], citric aldolase, citridesmolase, |
| | citritase |
| 4.2.1.2 | (S)-malate hydro-lyase, fumarase, L-malate hydro-lyase |
| 4.2.1.3 | Acon, Aconitate hydratase, cis-aconitase, citrate(isocitrate) hydro-lyase |
| 6.2.1.18 | citrate:CoA ligase (ADP-forming) |
| 6.2.1.5 | succinate:CoA ligase (ADP-forming), Succinic Thiokinase, succinyl-CoA synthetase |
| | (ADP-forming) |
| 6.4.1.1 | pyruvate:carbon-dioxide ligase (ADP-forming), pyruvic carboxylase |
| Acetate | 64-19-7, Acetasol, acetic acid, C2 short-chain fatty acid, C2H4O2, ethanoic acid, |
| | glacial acetic acid, Vasotate, Vosol |
| Acetyl-CoA | 72-89-9, acetyl-CoA, C23H38N7O17P3S, coenzyme A, S-acetate, S-acetyl coenzyme |
| | A, [(2R,3R,4R,5R)-2-[[[[3-[2-(2-acetylsulfanylethylcarbamoyl)ethylcarbamoyl]-3- |
| | hydroxy-2,2-dimethyl-propoxy]-hydroxy-phosphoryl]oxy-hydroxy- |
| | phosphoryl]oxymethyl]-5-(6-aminopurin-9-yl)-4-hydroxy-oxolan-3-yl]oxyphosphonic |
| | acid |
| cis-Aconitate | (Z)-1-propene-1,2,3-tricarboxylic acid, (Z)-prop-1-ene-1,2,3-tricarboxylic acid, 1- |
| | propene-1,2,3-tricarboxylic acid, (1Z)-, 1-propene-1,2,3-tricarboxylic acid, (Z)-, 1- |
| | propene-1,2,3-tricarboxylic acid, (Z)-(8Cl)(9Cl), 585-84-2, C6H6O6, cis-1-propene- |
| | 1,2,3-tricarboxylic acid, cis-aconic acid, cis-aconitate, cis-oxaloacetic acid |
| Citrate | 1,2,3-propanetricarboxylic acid, 2-hydroxy-, 126-44-3, 2-hydroxypropane-1,2,3- |
| | tricarboxylic acid, 77-92-9, ammounium citrate, C6H8O7, citrate, sodium citrate |
| CO2 | 124-38-9, carbon dioxide, carbonic anhydride, CO2, dry ice |
| CoA | 85-61-0, C21H36N7O16P3S, CoA, CoASH, coenzyme A, [(2R,3R,4R,5R)-5-(6- |
| | aminopurin-9-yl)-4-hydroxy-2-[[hydroxy-[hydroxy-[3-hydroxy-2,2-dimethyl-3-[2-(2- |
| | sulfanylethylcarbamoyl)ethylcarbamoyl]propoxy]phosphoryl]oxy- |
| | phosphoryl]oxymethyl]oxolan-3-yl]oxyphosphonic acid |
| Dihydrolipoamide | 3884-47-7, 6,8-bis-sulfanyloctanamide, 6,8-dimercapto-octanamide, C8H17NOS2, |
| | dihydrothioctamide, octanamide, 6,8-dimercapto- |
| Fumarate | (E)-but-2-enedioic acid, 110-17-8, 2-butenedioic acid (2E)-, C4H4O4, fumarate |
| Isocitrate | 1-hydroxypropane-1,2,3-tricarboxylic acid, 1637-73-6, 3-carboxy-2,3-dideoxy-1- |
| | hydroxypropan-1,2,3-tricarboxylic acid, 320-77-4, C6H8O7, isocitrate |
| Lipoamide | 1,2-dithiolane-3-pentanamide, 1,2-dithiolane-3-pentanamide (9Cl), 1,2-dithiolane-3- |
| | valeramide, 5-(1,2-dithiolan-3-yl)valeramide, 5-(dithiolan-3-yl)pentanamide, 6,8-thioctic |
| | amide, 940-69-2, alpha-lipoic acid amide, alpha-lipoic amide, C8H15NOS2, vitamin N |
| Oxaloacetate | 2-ketosuccinic acid, 2-oxobutanedioic acid, 328-42-7, butanedioic acid, oxo-, C4H4O5, |
| | OAA, oxaloacetate, oxaloacetic acid |
| Oxalosuccinate | 1-oxopropane-1,2,3-tricarboxylic acid, 1948-82-9, C6H6O7, oxalosuccinic acid |
| PEP | 138-08-9, 2-(phosphonooxy)acrylate, 2-dihydroxyphosphinoyloxyacrylic acid, 2- |
| | phosphonooxyprop-2-enoic acid, 2-propenoic acid, 2-(phosphonooxy)-, 2-propenoic |
| | acid, 2-(phosphonooxy)-, ion(1-), 2-propenoic acid, 2-(phosphonooxy)-, |
| | monopotassium salt, 4265-07-0, 73-89-2, C3H5O6P, phosphopyruvic acid |
| Pyruvate | 127-17-3, 2-oxopropanoate, 2-oxopropanoic acid, 57-60-3, C3H4O3, propanoic acid, |
| | 2-oxo-, propanoic acid, 2-oxo-, ion(1-), propanoic acid, 2-oxo-, sodium salt, pyruvate, |
| | pyruvic acid, sodium salt, sodium pyruvate |
| S-Succinyldihydrolipoamide | 3-[(3R)-7-carbamoyl-1-sulfanyl-heptan-3-yl]sulfanylcarbonylpropanoic acid, |
| | C12H21NO4S2 |
| Succinate | 1,2-ethanedicarboxylic acid, 1,4-butanedioic acid, 110-15-6, 56-14-4, amber acid, |
| | asuccin, butanedioate, butanedioic acid, C4H6O4, ethylenesuccinic acid, katasuccin, |
| | potassium succinate, succinate, wormwood acid |
| Succinyl-CoA | 3-[2-[3-[[4-[[[(2R,3R,4R,5R)-5-(6-aminopurin-9-yl)-4-hydroxy-3-phosphonooxy-oxolan- |
| | 2-yl]methoxy-hydroxy-phosphoryl]oxy-hydroxy-phosphoryl]oxy-2-hydroxy-3,3-dimethyl- |
| | butanoyl]amino]propanoylamino]ethylsulfanylcarbonyl]propanoic acid, 604-98-8, |
| | C25H40N7O19P3S, coenzyme A, S-(hydrogen butanedioate), succinyl-CoA |
| ThPP | 136-09-4, 154-87-0, 23883-45-6, C12H19N4O7P2S+, cocarboxylase, thiamin |
| | diphosphate, thiamine diphosphate hydrochloride, thiazolium, 3-((4-amino-2-methyl-5- |
| | pyrimidinyl)methyl)-4-methyl-5-(4,6,6-trihydroxy-3,5-dioxa-4,6-diphosphahex-1-yl)-, |
| | chloride, P,P′-dioxide, [2-[3-[(4-amino-2-methyl-pyrimidin-5-yl)methyl]-4-methyl-1-thia- |
| | 3-azoniacyclopenta-2,4-dien-5-yl]ethoxy-hydroxy-phosphoryl]oxyphosphonic acid |

*Genes/proteins that were used to identify the pathway:
1.1.1.42: isocitrate (NADP) dehydrogenase, isocitrate (nicotinamide adenine dinucleotide phosphate) dehydrogenase, Isocitrate dehydrogenase (NADP), isocitrate dehydrogenase (NADP-dependent), isocitrate: NADP oxidoreductase (decarboxylating), NADP isocitric dehydrogenase, NADP+-linked isocitrate dehydrogenase, NADP-dependent isocitrate dehydrogenase, NADP-dependent isocitric dehydrogenase, NADP-linked isocitrate dehydrogenase, NADP-specific isocitrate dehydrogenase, oxalosuccinate decarboxylase, oxalsuccinic decarboxylase
1.3.5.1: complex II, fumarate reductase complex, menaquinol:fumarate oxidoreductase, succinate dehydrogenase complex, succinate:ubiquinone oxidoreductase, succinic dehydrogenase
1.3.99.1: Complex II, Succinate INT Dehydrogenase
6.2.1.4: succinate:CoA ligase (GDP-forming), succinyl-CoA synthetase (GDP-forming)

TABLE 4
Genes and Proteins Involved in the Glutathione Metabolism Pathway

| Name | Synonyms |
|---|---|
| (5-L-Glutamyl)-L-amino acid | L-gamma-glutamyl-L-amino acid |
| 1.1.1.43 | 2-keto-6-phosphogluconate reductase, 6-phospho-D-gluconate:NAD(P) 2-oxidoreductase, |
| | 6-phosphogluconate dehydrogenase (NAD), 6-phosphogluconic dehydrogenase, |
| | gluconate 6-phosphate dehydrogenase, phosphogluconate dehydrogenase |
| 1.1.1.49 | 6-phosphoglucose dehydrogenase, D-glucose 6-phosphate dehydrogenase, D-glucose-6- |
| | phosphate:NADP 1-oxidoreductase, Entner-Doudoroff enzyme, glucose 6-phosphate |
| | dehydrogenase (NADP), NADP-dependent glucose 6-phosphate dehydrogenase, NADP- |
| | glucose-6-phosphate dehydrogenase, Zwischenferment |
| 1.11.1.12 | glutathione:lipid-hydroperoxide oxidoreductase, hydroperoxide glutathione peroxidase, |
| | peroxidation-inhibiting protein, peroxidation-inhibiting protein:peroxidase, glutathione |
| | (phospholipid hydroperoxide-reducing), PHGPX |
| 1.11.1.9 | Glutathioine peroxidase, glutathione:hydrogen-peroxide oxidoreductase, Gpx, GSH |
| | peroxidase, Gsh-px, reduced glutathione peroxidase, selenium-glutathione peroxidase |
| 1.5.4.1 | PDA synthase, pyrimidodiazepine:oxidized-glutathione oxidoreductase (ring-opening, |
| | cyclizing) |
| 1.8.1.13 | g-glutamylcysteine:NADP+ oxidoreductase, NADPH2:bis-g-glutamylcysteine |
| | oxidoreductase |
| 1.8.1.7 | glutathione reductase, glutathione reductase (NADPH), glutathione S-reductase, |
| | glutathione:NADP+ oxidoreductase, GSH reductase, GSSG reductase, NADPH- |
| | glutathione reductase, NADPH-GSSG reductase, NADPH:oxidized-glutathione |
| | oxidoreductase |
| 1.8.3.3 | glutathione:oxygen oxidoreductase |
| 1.8.4.1 | glutathione:homocystine oxidoreductase |
| 1.8.4.2 | glutathione-insulin transhydrogenase, glutathione-protein disulfide oxidoreductase, |
| | glutathione:protein-disulfide oxidoreductase, GSH-insulin transhydrogenase, insulin |
| | reductase, protein disulfide transhydrogenase, protein-disulfide interchange enzyme, |
| | protein-disulfide isomerase/oxidoreductase, reductase, protein disulfide (glutathione), thiol- |
| | protein disulphide oxidoreductase, thiol:protein-disulfide oxidoreductase |
| 1.8.4.3 | coenzyme A:oxidized-glutathione oxidoreductase, glutathione coenzyme A-glutathione |
| | transhydrogenase, glutathione-coenzyme A glutathione disulfide transhydrogenase, |
| | glutathione:coenzyme A-glutathione transhydrogenase |
| 1.8.4.4 | glutathione:cystine oxidoreductase, GSH-cystine transhydrogenase, NADPH-dependent |
| | GSH-cystine transhydrogenase |
| 1.8.4.7 | glutathione-dependent thiol:disulfide oxidoreductase, thiol:disulphide oxidoreductase, |
| | [xanthine-dehydrogenase]:oxidized-glutathione S-oxidoreductase |
| 1.8.5.1 | dehydroascorbate reductase, dehydroascorbic acid reductase, dehydroascorbic |
| | reductase, DHA reductase, GDOR, glutathione dehydroascorbate reductase, |
| | glutathione:dehydroascorbate oxidoreductase, glutathione:dehydroascorbic acid |
| | oxidoreductase |
| 2.3.1.80 | acetyl-CoA:S-substituted L-cysteine N-acetyltransferase |
| 2.3.2.2 | (5-L-glutamyl)-peptide:amino-acid 5-glutamyltransferase, alpha-glutamyl transpeptidase, |
| | g-glutamyl peptidyltransferase, g-glutamyl transpeptidase, g-GPT, g-GTP, Gamma Gt, |
| | Gamma-glutamyltransferase, Gamma-Gtp, glutamyl transpeptidase, L-g-glutamyl |
| | transpeptidase, L-g-glutamyltransferase, L-glutamyltransferase |
| 2.3.2.4 | (5-L-glutamyl)-L-amino-acid 5-glutamyltransferase (cyclizing), g-glutamyl-amino acid |
| | cyclotransferase, g-L-glutamylcyclotransferase, L-glutamic cyclase |
| 2.8.1.3 | glutathione-dependent thiosulfate reductase, sulfane reductase, sulfane sulfurtransferase, |
| | thiosulfate:thiol sulfurtransferase |
| 3.4.11.2 | alanine aminopeptidase, alanine-specific aminopeptidase, alanyl aminopeptidase, amino- |
| | oligopeptidase, aminopeptidase M, aminopeptidase N, CD13, cysteinylglycinase, |
| | cysteinylglycine dipeptidase, L-alanine aminopeptidase, membrane aminopeptidase I, |
| | microsomal aminopeptidase, particle-bound aminopeptidase, pseudo leucine |
| | aminopeptidase |
| 3.4.11.4 | alanine-phenylalanine-proline arylamidase, aminoexotripeptidase, aminotripeptidase, |
| | imidoendopeptidase, lymphopeptidase, peptidase B, peptidase T, tripeptidase |
| 3.5.1.78 | g-L-glutamyl-L-cysteinyl-glycine:spermidine amidase, glutathionylspermidine |
| | amidohydrolase (spermidine-forming) |
| 3.5.2.9 | 5-oxo-L-prolinase, 5-oxo-L-proline amidohydrolase (ATP-hydrolysing), 5-oxoprolinase, L- |
| | pyroglutamate hydrolase, oxoprolinase, pyroglutamase, pyroglutamase (ATP-hydrolysing), |
| | pyroglutamate hydrolase, pyroglutamic hydrolase |
| 5-Oxoproline | (2S)-5-oxopyrrolidine-2-carboxylic acid, 5-oxo-L-proline, 5-oxoproline, 98-79-3, C5H7NO3, |
| | L-Proline, 5-oxo-, pidolic acid, pyroglutamic acid |
| 6.3.1.8 | g-L-Glutamyl-L-cysteinyl-glycine:spermidine ligase (ADP-forming), glutathione:spermidine |
| | ligase (ADP-forming) |
| 6.3.2.2 | g-glutamylcysteine synthetase, L-glutamate:L-cysteine g-ligase (ADP-forming) |
| 6.3.2.3 | g-L-glutamyl-L-cysteine:glycine ligase (ADP-forming), glutathione synthetase |
| Acetyl-CoA | 72-89-9, acetyl-CoA, C23H38N7O17P3S, coenzyme A, S-acetate, S-acetyl coenzyme A, |
| | [(2R,3R,4R,5R)-2-[[[[3-[2-(2-acetylsulfanylethylcarbamoyl)ethylcarbamoyl]-3-hydroxy-2,2- |
| | dimethyl-propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |
| Bis-&gamma;-glutamylcystine | (2S)-2-amino-4-[[2-[(2R)-2-[[(4S)-4-amino-4-carboxy-butanoyl]amino]-2-carboxy- |
| | ethyl]disulfanyl-1-carboxy-ethyl]carbamoyl]butanoic acid, 23052-19-9, C16H26N4O10S2, |
| | gamma-Glu-Cys disulfide, L-cysteine, L-gamma-glutamyl-, (2-2′)-disulfide |
| Glutathione(oxidized) | (2S)-2-amino-4-[[(1R)-2-[(2R)-2-[[(4S)-4-amino-4-carboxy-butanoyl]amino]-2- |
| | (carboxymethylcarbamoyl)ethyl]disulfanyl-1- |
| | (carboxymethylcarbamoyl)ethyl]carbamoyl]butanoic acid, 27025-41-8, bis(gamma- |
| | glutamyl-L-cysteinylglycine) disulfide, C20H32N6O12S2, glutathione, oxidized, GSSG, |
| | oxiglutatione |
| Glutathione(reduced) | (2S)-2-amino-4-[[(1R)-1-(carboxymethylcarbamoyl)-2-sulfanyl-ethyl]carbamoyl]butanoic |
| | acid, 70-18-8, C10H17N3O6S, gamma-Glu-Cys-Gly, gamma-L-glutamylcysteinylglycine, |
| | glutathione-reduced, glycine, N-(N-L-gamma-glutamyl-L-cysteinyl)-, GSH |
| Glutathionylspermidine | (2S)-2-amino-4-[[(1R)-1-[3-(4-aminobutylamino)propylcarbamoylmethylcarbamoyl]-2- |
| | sulfanyl-ethyl]carbamoyl]butanoic acid, 33932-35-3, C17H34N6O5S, glycinamide, L- |
| | gamma-glutamyl-L-cysteinyl-N-(3-((4-aminobutyl)amino)propyl)-, N′-glutathionylspermidine |
| | disulfide |
| Glycine | 2-aminoacetic acid, 56-40-6, C2H5NO2, glycine, zirconium aluminum glycine |
| L-Amino acid | C2H4NO2R, L-2-amino acid |
| L-Cysteine | (2S)-2-amino-3-sulfanyl-propanoic acid, 52-90-4, C3H7NO2S, cysteine, L-cysteine |
| L-Cysteinylglycine | 19246-18-5, 2-[[(2R)-2-amino-3-sulfanyl-propanoyl]amino]acetic acid, C5H10N2O3S, Cys- |
| | Gly, glycine, N-cysteinyl, glycine, N-L-cysteinyl-, L-cysteinyl-glycine |
| L-Glutamate | (2S)-2-aminopentanedioic acid, 142-47-2, 19473-49-5, 56-86-0, C5H9NO4, glutamate, |
| | glutamic acid, L-Glu, L-glutamate, L-glutamic acid, monosodium glutamate, potassium |
| | glutamate, potassium L-glutamate, sodium glutamate |
| L-&gamma;-Glutamylcysteine | (2S)-2-amino-4-[[(1R)-1-carboxy-2-sulfanyl-ethyl]carbamoyl]butanoic acid, 636-58-8, |
| | C8H14N2O5S, gamma-Glu-Cys, gamma-L-glutamyl-L-cysteine, L-Cysteine, N-L-gamma- |
| | glutamyl- |
| NADP+ | 1184-16-3, 53-59-8, adenosine 5′-(trihydrogen diphosphate), 2′-(dihydrogen phosphate), |
| | P′-5′-ester with 3-(aminocarbonyl)-1-beta-D-ribofuranosylpyridinium, inner salt, beta- |
| | NADP, C21H29N7O17P3+, NAD phosphate, nicotinamide adenine dinucleotide |
| | phosphate, [(2R,3R,4R,5R)-2-(6-aminopurin-9-yl)-5-[[[[(2R,3R,4R,5R)-5-(5- |
| | carbamoylpyridin-1-yl)-3,4-dihydroxy-oxolan-2-yl]methoxy-hydroxy-phosphoryl]oxy- |
| | hydroxy-phosphoryl]oxymethyl]-4-hydroxy-oxolan-3-yl]oxyphosphonic acid |
| NADPH | 2646-71-1, 53-57-6, adenosine 5′-(trihydrogen diphosphate), 2′-(dihydrogen phosphate), |
| | P′-5′-ester with 1,4-dihydro-1-beta-D-ribofuranosyl-3-pyridinecarboxamide, |
| | C21H30N7O17P3, dihydronicotinamide-adenine dinucleotide phosphate, NADPH |
| | tetrasodium salt, [(2R,3R,4R,5R)-2-(6-aminopurin-9-yl)-5-[[[[(2R,3R,4R,5R)-5-(3- |
| | carbamoyl-4H-pyridin-1-yl)-3,4-dihydroxy-oxolan-2-yl]methoxy-hydroxy-phosphoryl]oxy- |
| | hydroxy-phosphoryl]oxymethyl]-4-hydroxy-oxolan-3-yl]oxyphosphonic acid |
| R-S-Alanine | |
| R-S-Alanylglycine | |
| R-S-Glutathione | |
| R-S-Mercapturonate | R-S-mercapturonate |
| RX | halogenated hydrocarbon, RX |

*Genes/proteins that were used to identify the pathway:
1.1.1.42 isocitrate (NADP) dehydrogenase, isocitrate (nicotinamide adenine dinucleotide phosphate) dehydrogenase, Isocitrate dehydrogenase (NADP), isocitrate dehydrogenase (NADP-dependent), isocitrate:NADP oxidoreductase (decarboxylating), NADP isocitric dehydrogenase, NADP+-linked isocitrate dehydrogenase, NADP-dependent isocitrate dehydrogenase, NADP-dependent isocitric dehydrogenase, NADP-linked isocitrate dehydrogenase, NADP-specific isocitrate dehydrogenase, oxalosuccinate decarboxylase, oxalsuccinic decarboxylase
2.5.1.18 glutathione S-alkyl transferase, glutathione S-aralkyltransferase, glutathione S-aryltransferase, Glutathione S-transferase, RX: glutathione R-transferase, S-(hydroxyalkyl)glutathione lyase

TABLE 5
Gene and Proteins Involved in the LPS-IL-1 Mediated Inhibition of RXR
Function pathway.

| Name | Synonyms |
|---|---|
| ABCA1 | ABC-1, CERP, FLJ14958, HDLDT1, MGC164864, MGC165011, |
| | TGD |
| ABCB9 | EST122234, KIAA1520, mKIAA1520, TAPL |
| ABCG1 | ABC8, AW413978, MGC141022, MGC34313, White, WHITE1 |
| ABCG5 | AW112016, MGC123400, sterolin 1, STSL |
| ABCG8 | 1300003C16Rik, AI114946, GBD4, MGC142217, sterolin 2, STSL |
| ACOX | acyl coenzyme A oxidase, Acyl-CoA, Acyl-CoA oxidase, acyl- |
| | CoA:oxygen 2-oxidoreductase, fatty acyl-CoA oxidase, fatty acyl- |
| | coenzyme A oxidase |
| ALAS1 | 5-AMINOLEVULINATE SYNTHASE, ALAS, ALAS-N, ALAS3, |
| | ALASH, Delta Alas, Delta Aminolevulinate Synthase, MIG4 |
| ALDH | ALDEHYDE DEHYDROGENASE |
| APOC1/2/4 | |
| APOC2 | MGC117889, MGC75082, RGD1560725 |
| APOE | AD2, AI255918, APOEA, APOLIPOPROTEIN E, apoprotein, LPG, |
| | MGC1571 |
| Bile acid | |
| BSEP | ABC16, Bile Salt Export Pump, BSEP, Lith1, PFIC-2, PGY4, SPGP |
| c-Jun | Activator protein 1, AP-1, C-JUN, JUNC, v-Jun |
| CAR | AA209988, AI551208, CAR, CAR-BETA, CAR1, CAR2, Care2, |
| | Constitutive androstane receptor, ESTM32, MB67, MGC107281, |
| | MGC108525, MGC150433, MGC97144, MGC97209 |
| CAR Ligand | |
| CAR ligand-CAR-Retinoic acid-RXR&alpha; | CAR ligand-CAR-Retinoic acid-RXRalpha |
| CAT | 2210418N07, Cas-1, CATALASE, Catalase1, Cs-1, MGC128112, |
| | MGC138422, MGC138424, RATCAT01, RATCATL |
| CD14 | CD14 ANTIGEN |
| CES | CE-2, CES2, CES2A1, EG436059, ICE, LOC498940, PCE-2 |
| CETP | HDLCQ10 |
| CPT | acylcarnitine transferase, Carnitine O-palmitoyltransferase, carnitine |
| | palmitoyltransferase, carnitine palmitoyltransferase I, carnitine |
| | palmitoyltransferase II, carnitine palmitoyltransferase-A, CPT I (outer |
| | membrane carnitine palmitoyl transferase), CPT-A, CPT-B, CPTi, |
| | CPTo, L-carnitine palmitoyltransferase, outer malonyl-CoA |
| | inhibitable carnitine palmitoyltransferase, palmitoyl-CoA:L-carnitine |
| | O-palmitoyltransferase, palmitoylcarnitine transferase |
| CRM-1 | AA420417, CRM1, CRMI, DKFZp686B1823 |
| CYP2A | CYP2 |
| CYP2B6 | CPB6, CYP2B, CYP2B10, CYP2B2, Cyp2b20, Cyp2b20/10, |
| | CYP2B6, Cyp2b6/7, Cyp450e, Cype, CYPIIB6, IIB1, LOC361523, |
| | p16, P450 |
| CYP2C19 | Ah-2, Ahh-1, AHOH, AHOHase, AI159681, AI196010, CPCJ, |
| | CYP2C, Cyp2c29, Cyp2c39, Cyp2c7, CYP450-2C, CYPIIC17, |
| | CYPIIC19, Cytochrome p450, MGC156667, P450-11A, P450-2C, |
| | P450C2C, P450IIC19 |
| CYP2C8 | 2010301M18Rik, 2210009K14Rik, CPC8, CYP2C65, Cyp2c66, |
| | Cyp2c79, MGC144816, MGC144817, MP-12/MP-20 |
| CYP2C9 | CPC9, CYP2C, CYP2C10, CYP2C9-ARG, CYP2CII, MGC149605, |
| | MGC88320, p450 2c29, P450IIC9 |
| CYP3A4 | AI256190, cDEX, CP33, CP34, CYP, CYP3A, CYP3A1, Cyp3a11, |
| | CYP3A23, Cyp3a23/3a1, CYP3A3, CYP3A41, Cyp3a41a, Cyp3a44, |
| | CYTOCHROME P450 3A3, HLP, IIIAm1, MGC108757, |
| | MGC126680, NF-25, p450 3A4, P450C3, P450PCN1, Pcn, RL33 |
| CYP3A5 | CP35, Cyp3a13, Cyp3a9, Cytochrome p450 3a9, IIIAm2, |
| | MGC93139, P450olf3, P450PCN3, PCN3 |
| CYP3A7 | CP37, Cyp3a11, Cyp3a16, Cyp3a2, CYP3A6, MGC108545, |
| | MGC130513, P-450ut-a, P450-HFLA |
| CYP4A14 | AI314743, Cyp4a3, CYPIVA3, MGC107660, Omega hydroxylase, |
| | P450-4A14 |
| CYP4A22 | AI647584, CP4Y, Cyp4a, Cyp4a1, Cyp4a10, CYP4A2, CYP4A22, |
| | Cyp4a32, CYP4AII, CYPIVA11, CYTOCHROME p450 4A10, |
| | D4Rp1, LOC100044540, P-450 HK omega, RP1, RP23-118K16.4 |
| CYP7A1 | 7 ALPHA-HYDROXYLASE, C7 ALPHA H, CHAP, Cholesterol 7 |
| | alpha hydroxylase, Cholesterol hydroxylase 7 alpha, CP7A, CYP7, |
| | CYP7A, CYP7S1, MGC126826, MGC138389 |
| FABP | FATTY ACID-BINDING PROTEIN |
| FATP | Fatp, Fatty Acid Transporter, SOLUTE CARRIER FAMILY 27 |
| FMO | Dimethylaniline monooxygenase (N-oxide-forming), dimethylaniline |
| | N-oxidase, dimethylaniline oxidase, DMA oxidase, FAD-containing |
| | monooxygenase, flavin monooxygenase, flavin-containing |
| | monooxygenase, FMO-I, FMO-II, mixed-function amine oxidase, |
| | N,N-dimethylaniline monooxygenase, N,N- |
| | dimethylaniline, NADPH2:oxygen oxidoreductase (N-oxide-forming) |
| FXR | AI957360, BAR, Farnesol Receptor, FXR, Fxr alpha, Fxr/bar, HRR- |
| | 1, MGC163445, MGC94878, RIP14, Rxrip14 |
| FXR ligand-FXR-Retinoic acid-RXR&alpha; | FXR ligand-FXR-Retinoic acid-RXRalpha |
| HL | AI256194, HDLCQ12, Hepatic Lipase, Hepatic Triglyceride Lipase, |
| | Hepatolipase, HL, Hpl, HTGL, LIPH, MGC108746, Triacylglycerol |
| | Lipase |
| I-BABP | I-15P, I-BABP, I-BALB, I-BAP, ILBP, ILBP3, ILLBP, RP23-26M1.3 |
| IL-1 | IL-1, IL1-BETA, IL1F2 |
| IL-1R | II1 receptor |
| IRAK | AA408924, II1rak, IRAK, Irak1 predicted, IRAK1-S Irak1_predicted, |
| | mPLK, pelle, Plk, Plpk, RGD1563841 |
| JNK1/2 | JNK1/2 |
| LBP | LIPOPOLYSACCHARIDE-BINDING PROTEIN, Ly88, MGC124626, |
| | MGC22233 |
| Lbp-lipopolysaccharide | |
| LPS | endotoxin, endotoxin protein, LPS |
| LRH-1 | AU020803, B1F, B1F2, CPF, D1Ertd308e, FTF, FTF-2, FTZ-F1, |
| | FTZ-F1beta, hB1F, hB1F-2, LRH-1 |
| LXR | Lxr |
| LXR ligand-LXR-Retinoic acid-RXR&alpha; | LXR ligand-LXR-Retinoic acid-RXRalpha |
| MAO | adrenalin oxidase, adrenaline oxidase, amine oxidase, Amine |
| | oxidase (flavin-containing), amine:oxygen oxidoreductase |
| | (deaminating) (flavin-containing), epinephrine oxidase, monoamine |
| | oxidase, monoamine:O2 oxidoreductase (deaminating), polyamine |
| | oxidase, serotonin deaminase, spermidine oxidase, spermine |
| | oxidase, tyraminase, tyramine oxidase |
| MD-2 | ESOP-1, MD-2, MD-2 protein, MGC151162 |
| MDR1 | ABC20, Abcb1a, Abcb4, CD243, CLCS, EVI32, GP170, MDR, mdr- |
| | 3, MDR1, Mdr1a, MDR1B, MGC163296, P-GLYCOPROTEIN, P-GP, |
| | Pgy-3, PGY1 |
| MEKK1 | MAPK, MAPKKK1, MEK KINASE, MEK KINASE 1, MEKK, MEKK1, |
| | Raf |
| MGMT | AGAT, AGT, AI267024, ATase, MGC107020, O6-ALKYLGUANINE |
| | DNA ALKYLTRANSFERASE |
| MKK4/7 | MKK4/7 |
| MRP2 | ABC30, AI173996, CANALICULAR MULTIDRUG RESISTANCE |
| | PROTEIN, CMOAT, cMRP, DJS, Ebcr, KIAA1010, MRP2 |
| MRP3 | 1700019L09Rik, ABC31, ATP-binding cassette C3, cMOAT2, |
| | EST90757, MLP2, MOAT-D, MRP3, Multidrug Resistant Protein 3 |
| MRP4 | D630049P08Rik, EST170205, MOAT-B, MRP4 |
| MYD88 | |
| NR0B2 | SHP, SHP-1 |
| NTCP | NTCP, NTCP1, SBACT, Sodium dependent taurocholate |
| | cotransporting polypeptide, sodium/bile Acid Cotransporter, |
| | Sodium/Taurocholate Cotransporting Polypeptide1 |
| OATP2 | AI785519, OATP, OATP-A, OATP1A2, Oatp1a4, Oatp2, Organic |
| | anion transporting polypeptide 5, SLC21A3, SLC21A5, SLCO1A3, |
| | Slco1a4 |
| OATP4 | lst-1, LST-3TM13, LST3, mlst-1, OATP-C, OATP1B2, OATP1B3, |
| | OATP2, OATP4, OATP8, rlst-1, SLC21A10, Slc21a6, SLC21A8, |
| | Slco1b2 |
| PAPSS2 | 1810018P12RIK, AI159688, ATP SULFURYLASE, ATPSK2, |
| | AtpsU2, bm, code642, SK2 |
| PLTP | HDLCQ9, OD107 |
| PPAR | 4933429D07Rik, AI118064, AW742785, hPPAR, MGC2237, |
| | MGC2452, NR1C1, PPAR, PPAR-ALPHA |
| PPAR ligand-PPAR-Retinoic acid-RXR&alpha; | PPAR ligand-PPAR-Retinoic acid-RXRalpha |
| PXR | BXR, MGC108643, mPXR, ONR1, PAR, PAR1, PAR2, PARq, PRR, |
| | PXR, PXR.1, PXR.2, PXR1, SAR, SXR, XONR |
| PXR Ligand | pregnanes |
| PXR ligand-PXR-Retinoic acid-RXR&alpha; | PXR ligand-PXR-Retinoic acid-RXRalpha |
| RAR ligand-RAR&alpha;-Retinoic acid-RXR&alpha; | RAR ligand-RARalpha-Retinoic acid-RXRalpha |
| RAR&alpha; | ALPHA RAR, NR1B1, RAR, RAR ALPHA, RAR ALPHA 1, |
| | RETINOIC ACID RECEPTOR ALPHA |
| Retinoic Acid | (2E,4E,6E,8E)-3,7-dimethyl-9-(2,6,6-trimethyl-1-cyclohexenyl)nona- |
| | 2,4,6,8-tetraenoic acid, 13497-05-7, 302-79-4, all-trans retinoic acid, |
| | Altinac, Avita, C20H28O2, Renova, Retin A Micro Gel, Retin-A, |
| | retinoic acid, all-trans-, retinoic acid, sodium salt, Tretinoin Topical, |
| | Vesanoid, vitamin A acid |
| RXR&alpha; | 9530071D11RIK, FLJ16020, FLJ16733, MGC102720, NR2B1, |
| | RETINOID X RECEPTOR ALPHA, RXR ALPHA, RXRalpha1 |
| SITPEC | MGC94704, SITPEC |
| SOD3 | AI314465, EC-SOD, ECSODPT, MGC13799, MGC20077 |
| SR-BI | AI120173, CD36, CD36L1, CLA-1, D5Ertd460e, HDL Receptor, |
| | MGC138242, mSR-BI, Scavenger receptor class b1, SR-B, SR-B1, |
| | SR-BI |
| SREBP1 | ADD-1, D630008H06, S14, SREBP-1, SREBP-1a, STEROL |
| | RESPONSE ELEMENT |
| SULT | CHST1, CHST2, CHST4, CHST6, SULT, UST |
| TAK1 | B430101B05, C87327, Map3k7 predicted, Map3k7_predicted, TAK1, |
| | Tgf beta Activated Kinase1, TGF1A |
| TLR4 | ARMD10, CD284, HTOLL, Ly87, Ran/M1, RasI2-8, TOLL, Toll like |
| | receptor 4, TOLL RECEPTOR |
| TNFR | Tnfr |
| TNF&alpha; | AT-TNF, DIF, Differentiation-induced Factor, MGC124630, |
| | MGC151434, RATTNF, TMTNF, TNF-ALPHA, TNFA, Tnfsf1a, |
| | TNFSF2 |
| TRAF2 | AI325259, MGC: 45012, TNF Receptor-Associated Factor 2, TRAP, |
| | TRAP3 |
| TRAF6 | 2310003F17Rik, AI851288, C630032O20Rik, MGC: 3310, RNF85, |
| | Traf6 predicted, Traf6_predicted |
| UGT | AI327289, Had-1, MGC188623, SfcB, UGALT, UGAT, UGT, UGT1, |
| | UGT2, UGTL |

*Genes/proteins that were used to identify the pathway:
ACS acid:CoA ligase (AMP-forming), acyl-activating enzyme, acyl-CoA synthetase, arachidonyl-CoA synthetase, Facl, fatty acid thiokinase (long chain), Fatty Acyl-CoA Synthetase, Lacs, Lignoceroyl CoA Ligase, lignoceroyl-CoA synthase, long-chain acyl-coa synthetase, Long-chain-fatty-acid-CoA ligase, Palmitoyl CoA Ligase, palmitoyl-CoA synthase
GST Glutathione s-transferase
HMGCS(S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b- methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase

TABLE 6
Genes/proteins Involved in the NRF-2 mediated oxidative stress response pathway.

| Name | Synonyms |
|---|---|
| Actin | G-actin |
| Actin-Nrf2 | |
| AFAR | AFLATOXIN B1 ALDEHYDE REDUCTASE |
| AKR | 2610201A18Rik, Akr1a4, Akra, ALDEHYDE REDUCTASE, ALDR1, ALR, DD3, MGC12529, |
| | MGC1380 |
| AKT | AKT, MGC99656, PKB, PKB-ALPHA, PKB/AKT, PRKBA, Protein kinase B, RAC, RAC-ALPHA, |
| | Thymoma viral proto-oncogene 1 |
| AOX1 | AI196512, AI255253, ALDEHYDE OXIDASE, AO, AOH1, Aox-2, MGC13774, Moro, RO, XD |
| ASK1 | 7420452D20Rik, APOPTOSIS SIGNAL REGULATED KINASE 1, ASK, ASK1, Map3k5, MAPKKK5, |
| | MEKK5, MGC141518, MGC141519, RGD1306565 predicted, RGD1306565_predicted |
| ATF4 | C/ATF, CREB-2, MGC96460, TAXREB67, TXREB |
| BACH1 | 6230421P05RIK, AI323795, C21ORF41 |
| c-Fos | AP-1, C-FOS, D12Rfj1, V-FOS |
| c-MAF | 2810401A20Rik, A230108G15RIK, AW047063, C-MAF, Maf2, MGC71685 |
| c-Raf | 6430402F14Rik, AA990557, BB129353, C-RAF, C-RAF1, D830050J10Rik, MGC102375, MURINE |
| | LEUKEMIA VIRAL ONCOGENE HOMOLOG1, NS5, Raf, RAF KINASE, v-Raf |
| CAT | 2210418N07, Cas-1, CATALASE, Catalase1, Cs-1, MGC128112, MGC138422, MGC138424, |
| | RATCAT01, RATCATL |
| CBP/p300 | CBP |
| CBR1 | AW261796, Carbonyl Reductase, CBR, CR, hCBR1, MGC124927, Ocr |
| CCT7 | AA408524, AL022769, CCT-ETA, Ccth, Cctz, Chaperonin subunit 7, MGC110985, Nip7-1, TCP-1- |
| | eta |
| CLPP | AU019820, D17Wsu160e |
| CUL3 | AI467304, AW146203, mKIAA0617 |
| Cul3-Roc1 | |
| CYP1A/2A/3A/4A/2C | |
| ElectophilesROS | oxygen and reactive oxygen species, reactive oxygen metabolites, ROI, ROS |
| EPHX1 | AI195553, Ehm, Eph-1, EPHX, EPOX, Epoxide Hydrolase, EPXH1, MEH, MEH8, MICROSOMAL |
| | EPOXIDE HYDROLASE |
| ERK1/2 | |
| ERK5 | BMK, BMK1, ERK4, ERK5, Erk5-T, ERK7, PRKM7 |
| FKBP5 | 51 kDa, AIG6, D17Ertd592e, Dit1, FKBP51, Fkbp51/54, FKBP54, MGC111006, P54, PPlase, Ptg-10 |
| FMO1 | Flavin-containing monooxygenase, RFMO1A |
| FRA1 | AW538199, FRA, FRA-1 |
| FTH1 | AL022624, AL033366, APOFERRITIN H CHAIN, FERRITIN H, FERRITIN H CHAIN, Ferritin heavy |
| | chain, Ferritin subunit H, FHC, FTH, FTHL6, H FERRITIN, Hcf, MFH, MGC104426, PIG15, PLIF |
| FTL | FERRITIN LIGHT CHAIN, FTL1, Ftl2, L-FERRITIN, MGC102130, MGC102131, MGC118079, |
| | MGC118080, MGC71996, RGD1560687 predicted, RGD1560687_predicted, RGD1561055 |
| | predicted, RGD1561055_predicted, RGD1566189 predicted, RGD1566189_predicted, YB24D08 |
| GCLC | D9Wsu168e, GAMMA GCS HEAVY CHAIN, Gamma Glutamyl Cysteine Synthetase Light Subunit, |
| | Gamma Glutamylcysteine Synthetase, Gamma glutamylcysteine synthetase heavy subunit, |
| | GAMMA-GCS, GAMMA-GCSH, Gcl, GCS, GCS, Catalytic, GCS-HS, GCSH, Ggcs-hs, GLCL, |
| | GLCL-H, GLCLC, MGC93096 |
| GCLM | AI649393, Gamma gclm, GAMMA GCS LIGHT CHAIN, Gamma glutamylcysteine synthase |
| | (regulatory), GAMMA GLUTAMYLCYSTEINE SYNTHETASE, Gcs Ls, Gcs, Regulatory, GCS-L, |
| | GCS1, Gcslc, GLCLR, glutamat-cystein ligase, regulatory subunit |
| GPX2 | GI-GPx, GPRP, GPX-GI, GSHPx-2, GSHPX-GI |
| GSK3&beta; | 7330414F15Rik, 8430431H08Rik, C86142, GSK-3, GSK-3BETA, Tpk1 |
| GSR | AI325518, D8Ertd238e, GLUTATHIONE REDUCTASE, Gr, Gr-1, Gred, GRX, MGC78522 |
| HERPUD1 | HERP, KIAA0025, Mif1, MifI, SUP |
| HIP2 | AW492011, D5Ertd601e, DKFZp564C1216, DKFZp686J24237, E2-25K, HIP2, HYPG, LIG, |
| | UBIQUITIN CARRIER PROTEIN |
| HO-1 | bK286B10, D8Wsu38e, HEME OXYGENASE (DECYCLIZING) 1, HEME OXYGENASE-1, Hemox, |
| | Heox, HEOXG, Hmox, HO-1, HSP32 |
| HSP22/40/90 | |
| JNK1/2 | JNK1/2 |
| Jun | JUN |
| KEAP1 | INRF2, KIAA0132, KLHL19, MGC10630, MGC1114, MGC20887, MGC4407, MGC9454, |
| | mKIAA0132 |
| Keap1-Nrf2 | |
| MEK1/2 | MEK1/2, Mkk 1/2 |
| MEK5 | AI324775, AI428457, HsT17454, MAP kinase kinase 5, MAPKK5, MEK5, MKK5, PRKMK5 |
| MEKK | MAPK, MAPKKK1, MEK KINASE, MEK KINASE 1, MEKK, MEKK1, Raf |
| MKK3/6 | Mkk3/6 (mitogen activated protein kinase kinase 3/6), MKK3/MKK6 |
| MKK4/7 | MKK4/7 |
| MRP1 | ABC29, ABCC, Abcc1a, Abcc1b, Avcc1a, DKFZp686N04233, DKFZp781G125, GS-X, Mdrap, MRP, |
| | MRP1 |
| NQO | NADPH QUINONE OXIDOREDUCTASE, Nadph-d |
| NRF2 | AI194320, NRF2 |
| p38 MAPK | CRK1, CSBP, CSBP1, CSBP2, CSPB1, EXIP, Hog, MAPK p38, MGC102436, MGC105413, MXI2, |
| | P38, P38 KINASE, P38 Map Kinase, p38 Mapk alpha, P38-ALPHA, p38-RK, p38/Hog1, p38/Mpk2, |
| | P38/RK, p38a, p38Hog, p38MAPK, PRKM14, PRKM15, RK, SAPK2A |
| PERK | AI427929, DKFZp781H1925, HRI, PEK, PERK, WRS |
| PI3K | Pi 3-kinase |
| PKC | Cnpkc, Pkc, PKC protein, Pkm, Protein kinase c |
| PPIB | AA408962, AA553318, AI844835, CPHN2, Cy-Lp, CYCLOPHILIN-B, CyP-20b, CYP-S1, CYPB, |
| | MGC14109, MGC2224, SCYLP |
| PRDX1 | ENHANCER PROTEIN, Hbp23, MGC108617, MSP23, NKEFA, OSF-3, PAG, PAGA, PAGB, |
| | PEROXIREDOXIN 1, Prdx-I, PRX I, PRX1, TDPX2, TDX2, TPx-A, TPX2 |
| PSM | |
| PTPLAD1 | 4930523M17RIK, AW742319, B-IND1, FLJ90376, HSPC121, MGC25483 |
| Ras | p21 Ras, p21 Ras protein, Ras protein |
| Roc1 | 1500002P15Rik, AA517855, BA554C12.1, ENSMUSG00000049832, HRT1, MGC13357, |
| | MGC1481, RBX1, RNF75, ROC1 |
| small MAF | MAF, SMALL MAF |
| SQSTM1 | A170, OSF-6, Osi, OSIL, Oxidative Stress Protein, p60, P62, p62B, PDB3, Pkc zeta interacting |
| | protein, STAP, Ubiquitin-binding protein a, ZIP, ZIP3 |
| SR-BI | AI120173, CD36, CD36L1, CLA-1, D5Ertd460e, HDL Receptor, MGC138242, mSR-BI, Scavenger |
| | receptor class b1, SR-B, SR-B1, SR-BI |
| STIP1 | HOP, IEF-SSP-3521, mSTI1, P60, SIP1, STI1, STI1L, Stress-induced phosphoprotein 1 |
| TAK1 | B430101B05, C87327, Map3k7 predicted, Map3k7_predicted, TAK1, Tgf beta Activated Kinase1, |
| | TGF1A |
| TRXR1 | GRIM-12, KM 102 DERIVED REDUCTASE LIKE FACTOR, MGC9145, MGC93353, Tgr, |
| | Thioredoxin reductase, TR, TR1, TRXR1, TXNR |
| TXN | ADF, AW550880, DKFZp686B1993, EOSINOPHIL CYTOTOXICITY FACTOR, MGC151960, |
| | MGC61975, THIOREDOXIN, TRX, TRX1, Txn1 |
| UB2R1 | AI327276, Cdc34, E2-CDC34, UBC3, UBE2R1, Ubiquitin conjugating enzyme e2-32 |
| UBB | AL033289, FLJ25987, Loc192255, MGC8385, Polyubiquitin ub2, Ubb2, UBC, UBIQUITIN, |
| | UBIQUITIN B |
| UGT | AI327289, Had-1, MGC188623, Sfc8, UGALT, UGAT, UGT, UGT1, UGT2, UGTL |
| USP14 | 2610005K12Rik, AW107924, ax, C78769, MGC95160, TGT, Ubiquitin specific protease 14 |
| VCP | 3110001E05, CDC48, IBMPFD, MGC131997, MGC148092, MGC8560, P97, P97 kinase, p97/VCP, |
| | Ter atpase, TERA, TRANSITIONAL ENDOPLASMIC RETICULUM ATPASE, XSG7 |

*Genes/proteins that were used to identify the pathway:
ERP29 1200015M03Rik, 2810446M09Rik, AW209030, C12ORF8, ERp28, ERp31, PDI-DB
GST Glutathione s-transferase
SOD copper-zinc superoxide dismutase, Cu,Zn-SOD, Cu—Zn superoxide dismutase, cuprein, cytocuprein, erythrocuprein, Fe-SOD, ferrisuperoxide dismutase, hemocuprein, hepatocuprein, Mn-SOD, Sod protein, SOD-1, SOD-2, SOD-3, SOD-4, SODF, SODS, superoxidase dismutase, Superoxide dismutase, superoxide dismutase I, superoxide dismutase II, superoxide:superoxide oxidoreductase

TABLE 7
Genes and Proteins Involved in the Synthesis and Degradation of Ketone Bodies Pathway

| Name | Synonyms |
|---|---|
| (R)-3-Hydroxy-butyrate | (3R)-3-hydroxybutanoic acid, (R)-(−)-3-hydroxybutyric acid sodium salt, (R)- |
| | 3-hydroxybutanoic acid, (R)-3-hydroxybutyric acid, 13613-65-5, 625-72-9, |
| | C4H8O3, D-beta-hydroxybutyrate, R-3-hydroxybutanoate, sodium (R)-3- |
| | hydroxybutyrate |
| (S)-3-Hydroxy-3- | (3S)-4-[2-[3-[[4-[[[(2R,3R,4R,5R)-5-(6-aminopurin-9-yl)-4-hydroxy-3- |
| methylglutaryl-CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-hydroxy-3-methyl- |
| | butanoic acid, (S)-3-hydroxy-3-methylglutaryl-CoA, 1553-55-5, |
| | C27H44N7O20P3S, hydroxymethylglutaryl-CoA, S-(hydrogen 3-hydroxy-3- |
| | methylglutaryl)coenzyme A, S-(hydrogen 3-hydroxy-3-methylpentanedioate)coenzyme A |
| 1.1.1.30 | (R)-3-hydroxybutanoate:NAD oxidoreductase, 3-D-hydroxybutyrate |
| | dehydrogenase, beta-hydroxybutyrate dehydrogenase, beta-hydroxybutyric |
| | acid dehydrogenase, beta-hydroxybutyric dehydrogenase, D-(−)-3- |
| | hydroxybutyrate dehydrogenase, D-3-hydroxybutyrate dehydrogenase, D- |
| | beta-hydroxybutyrate dehydrogenase, hydroxybutyrate oxidoreductase, |
| | NAD-beta-hydroxybutyrate dehydrogenase |
| 2.3.1.9 | 2-methylacetoacetyl-CoA thiolase, 3-oxothiolase, acetoacetyl-CoA thiolase, |
| | acetyl coenzyme A thiolase, acetyl-CoA acetyltransferase, acetyl- |
| | CoA:acetyl-CoA C-acetyltransferase, acetyl-CoA:N-acetyltransferase, beta- |
| | acetoacetyl coenzyme A thiolase, thiolase II |
| 2.8.3.5 | 3-ketoacid CoA-transferase, 3-ketoacid coenzyme A transferase, 3-oxo-CoA |
| | transferase, 3-oxoacid CoA dehydrogenase, 3-oxoacid coenzyme A- |
| | transferase, acetoacetate succinyl-CoA transferase, acetoacetyl coenzyme |
| | A-succinic thiophorase, succinyl coenzyme A-acetoacetyl coenzyme A- |
| | transferase, succinyl-CoA transferase, succinyl-CoA:3-oxo-acid CoA- |
| | transferase |
| 4.1.1.4 | acetoacetate carboxy-lyase, acetoacetic acid decarboxylase |
| 4.1.3.4 | (S)-3-hydroxy-3-methylglutaryl-CoA acetoacetate-lyase, 3-hydroxy-3- |
| | methylglutaryl CoA cleaving enzyme, 3-hydroxy-3-methylglutaryl coenzyme |
| | A lyase, 3-hydroxy-3-methylglutaryl-CoA lyase, hydroxymethylglutaryl |
| | coenzyme A lyase, hydroxymethylglutaryl coenzyme A-cleaving enzyme |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme |
| | A, [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetone | 2-Propanone, 67-64-1, acetone, C3H6O, dimethyl ketone, |
| | dimethylformaldehyde, dimethylketal, propanone |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |

*Genes/proteins that were used to identify the pathway:
2.3.3.10 (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b-methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase

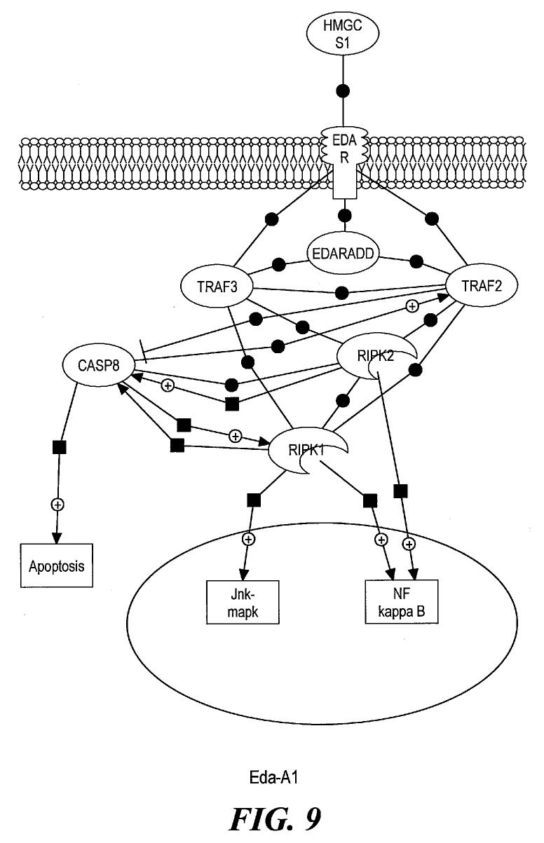

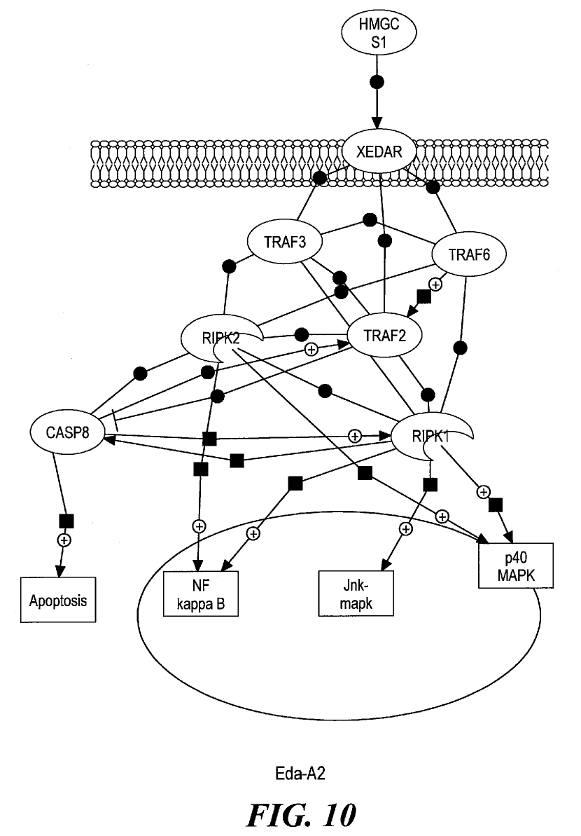

In addition, pathway analysis using Pathway Studio software based on previously identified differentially expressed genes or proteins associated with high cell viability led to the identification of the Eda A1 pathway (FIG. 9), Eda-A2 pathway (FIG. 10). Genes and/or proteins that were used to identify relevant pathways are indicated in FIGS. 9 and 10. In addition, additional exemplary genes or proteins involved in the above-identified pathways and that may be involved in regulating or indicative of high cell viability are summarized in Table 8 (Eda-A1 pathway) and Table 9 (Eda-A2 pathway).

TABLE 8
Genes and Proteins Involved in Eda-A1 pathway

| Name | Type | Description |
|---|---|---|
| Apoptosis | Cell Process | |
| CASP8 | Protein | caspase 8, apoptosis-related cysteine |
| | | peptidase |
| EDAR | Protein | ectodysplasin A receptor |
| EDARADD | Protein | EDAR-associated death domain |
| Jnk-mapk | Pathway | |
| NF kappa B | Pathway | |
| RIPK1 | Protein | receptor (TNFRSF)-interacting |
| | | serine-threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |

*Genes/proteins that were used to identify the pathway:
HMGCS1 3-hydroxy-3-methylglutaryl-Coenzyme A synthase 1 (soluble)

TABLE 9
Eda-A2 pathway

| Name | Type | Description |
|---|---|---|
| Apoptosis | Cell Process | |
| CASP8 | Protein | caspase 8, apoptosis-related |
| | | cysteine peptidase |
| Jnk-mapk | Pathway | |
| NF kappa B | Pathway | |
| p40 MAPK | Pathway | |
| RIPK1 | Protein | receptor (TNFRSF)-interacting |
| | | serine-threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |
| TRAF6 | Protein | TNF receptor-associated factor 6 |
| XEDAR | Protein | microtubule-associated protein 2 |

*Genes/proteins that were used to identify the pathway:
HMGCS1 3-hydroxy-3-methylglutaryl-Coenzyme A synthase 1 (soluble)

### Example 2

### Exemplary Pathways Associated with High Cell Density

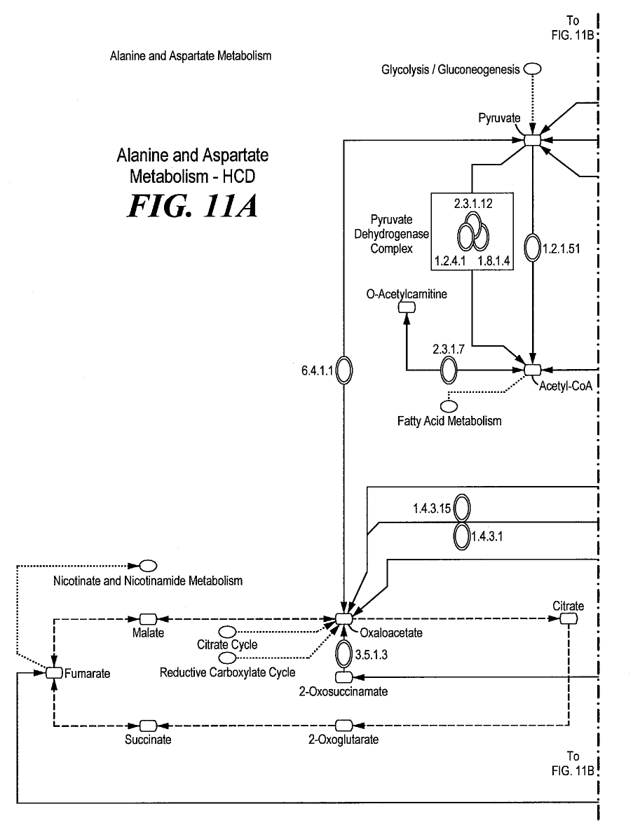

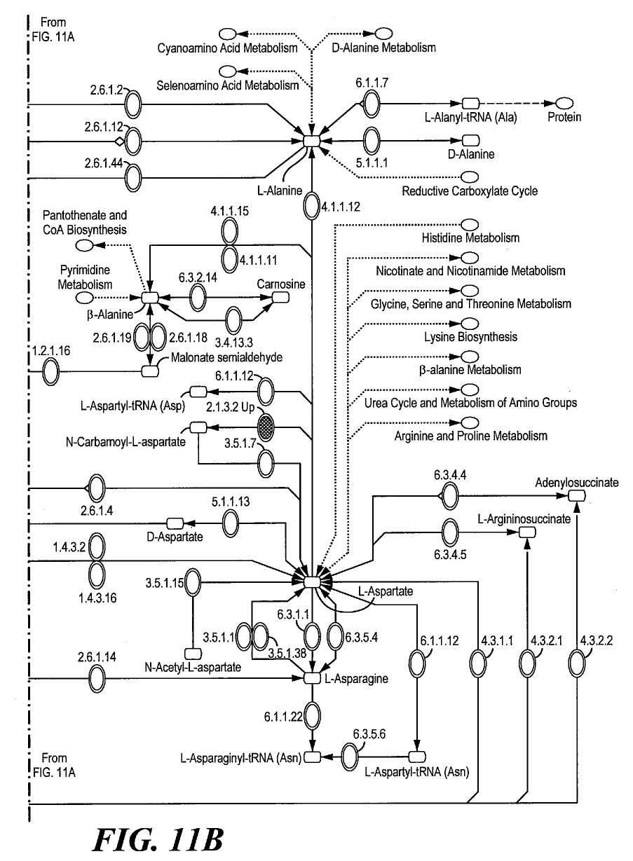

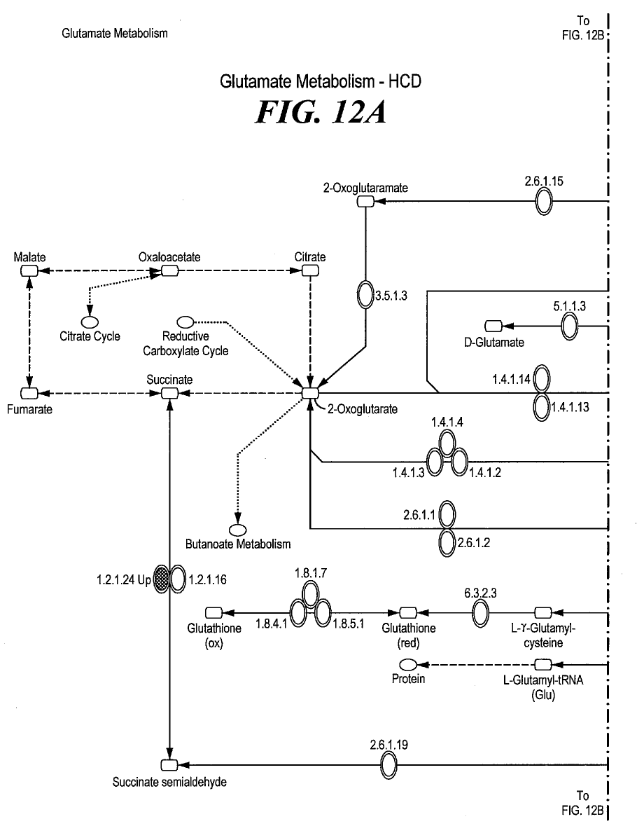

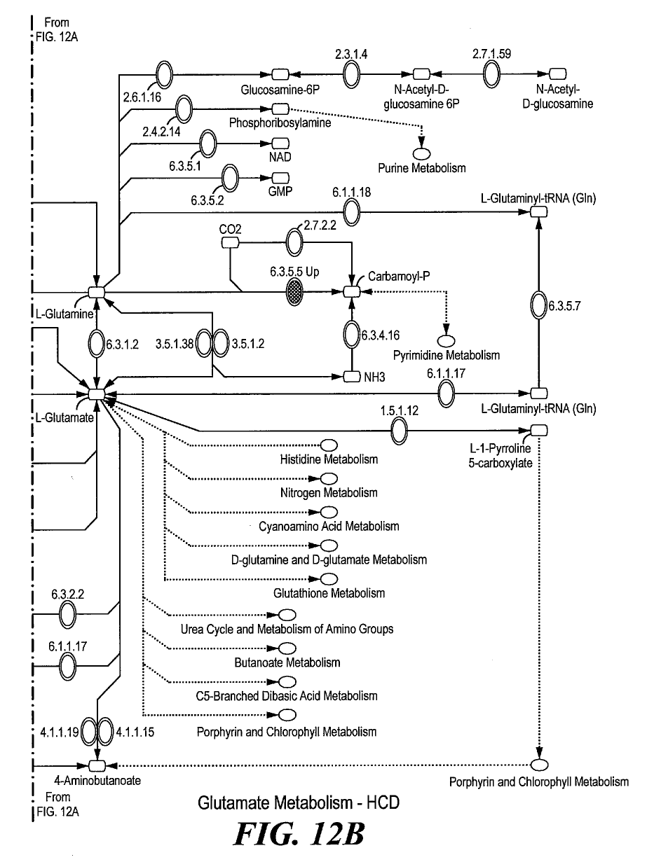

Pathway analysis using Ingenuity software based on previously identified differently expressed genes or proteins associated with high cell density led to the identification of the alanine and aspartate metabolism pathway (FIG. 11) and the glutamate metabolism pathway (FIG. 12). Genes and/or proteins that were used to identify relevant pathways are indicated in FIGS. 11 and 12. In addition, additional exemplary genes or proteins involved in the above-identified pathways and that may be involved in regulating or indicative of high cell density are summarized in Table 10 (the alanine and aspartate metabolism pathway) and Table 11 (the glutamate metabolism pathway).

TABLE 10
Genes/Proteins Involved in the Alanine and aspartate metabolism pathway

| Name | Synonyms |
|---|---|
| 1.2.1.18 | 3-oxopropanoate:NAD(P) oxidoreductase (decarboxylating, CoA-acetylating), malonic |
| | semialdehyde oxidative decarboxylase |
| 1.2.1.51 | pyruvate:NADP 2-oxidoreductase (CoA-acetylating) |
| 1.2.4.1 | MtPDC (mitochondrial pyruvate dehydogenase complex), PDH, pyruvate decarboxylase, |
| | pyruvate dehydrogenase, pyruvate dehydrogenase complex, pyruvate:lipoamide 2- |
| | oxidoreductase (decarboxylating and acceptor-acetylating), pyruvic acid dehydrogenase, |
| | pyruvic dehydrogenase |
| 1.4.3.1 | aspartic oxidase, D-aspartate:oxygen oxidoreductase (deaminating), D-aspartic oxidase |
| 1.4.3.15 | D-glutamate(D-aspartate):oxygen oxidoreductase (deaminating), D-glutamic-aspartic oxidase, |
| | D-monoaminodicarboxylic acid oxidase |
| 1.4.3.16 | L-aspartate:oxygen oxidoreductase (deaminating) |
| 1.4.3.2 | L-amino-acid:oxygen oxidoreductase (deaminating), ophio-amino-acid oxidase |
| 1.8.1.4 | dehydrolipoate dehydrogenase, diaphorase, dihydrolipoamide:NAD oxidoreductase, |
| | dihydrolipoic dehydrogenase, dihydrolipoyl dehydrogenase, dihydrothioctic dehydrogenase, |
| | LDP-Glc, LDP-Val, lipoamide dehydrogenase (NADH), lipoamide oxidoreductase (NADH), |
| | lipoamide reductase, lipoamide reductase (NADH2), lipoate dehydrogenase, lipoic acid |
| | dehydrogenase, lipoyl dehydrogenase |
| 2-Oxoglutarate | 2-ketoglutarate, 2-oxoglutarate, 2-oxopentanedioic acid, 328-50-7, alpha-ketoglutarate, alpha- |
| | ketoglutaric acid, alphaKG, C5H6O5, glutaric acid, 2-oxo-, glutaric acid, 2-oxo-(8Cl), |
| | pentanedioic acid, 2-oxo- |
| 2-Oxosuccinamate | 2-oxosuccinamate, 3-carbamoyl-2-oxo-propanoic acid, 33239-40-6, 4-amino-2,4-dioxo- |
| | butanoic acid, butanoic acid, 4-amino-2,4-dioxo-, C4H5NO4 |
| 2.3.1.12 | acetyl-CoA:dihydrolipoamide S-acetyltransferase, dihydrolipoate acetyltransferase, |
| | dihydrolipoic transacetylase, dihydrolipoyl acetyltransferase, lipoate acetyltransferase, lipoate |
| | transacetylase, lipoic acetyltransferase, lipoic acid acetyltransferase, lipoic transacetylase, |
| | lipoylacetyltransferase, thioltransacetylase A, transacetylase X |
| 2.3.1.7 | acetyl-CoA-carnitine O-acetyltransferase, acetyl-CoA:carnitine O-acetyltransferase, |
| | acetylcarnitine transferase, carnitine acetyl coenzyme A transferase, carnitine acetylase, |
| | carnitine acetyltransferase, carnitine-acetyl-CoA transferase, CATC |
| 2.6.1.1 | 2-oxoglutarate-glutamate aminotransferase, AAT, aspartate alpha-ketoglutarate transaminase, |
| | aspartate aminotransferase, Aspartate transaminase, aspartate-2-oxoglutarate transaminase, |
| | aspartate:2-oxoglutarate aminotransferase, aspartic acid aminotransferase, aspartic |
| | aminotransferase, aspartyl aminotransferase, AspT, AST, glutamate oxaloacetate |
| | transaminase, glutamate-oxalacetate aminotransferase, glutamate-oxalate transaminase, |
| | glutamic oxalic transaminase, glutamic-aspartic aminotransferase, glutamic-aspartic |
| | transaminase, glutamic-oxalacetic transaminase, glutamic-oxaloacetic transaminase, GOT |
| | (enzyme), L-aspartate transaminase, L-aspartate-2-ketoglutarate aminotransferase, L- |
| | aspartate-2-oxoglutarate aminotransferase, L-aspartate-2-oxoglutarate-transaminase, L- |
| | aspartate-alpha-ketoglutarate transaminase, L-aspartate:2-oxoglutarate aminotransferase, L- |
| | aspartic aminotransferase, oxaloacetate transferase, oxaloacetate-aspartate aminotransferase, |
| | Sgot, transaminase A |
| 2.6.1.12 | alanine-keto acid aminotransferase, alanine-oxo acid aminotransferase, L-alanine-alpha-keto |
| | acid aminotransferase, L-alanine:2-oxo-acid aminotransferase, leucine-alanine transaminase |
| 2.6.1.14 | asparagine-keto acid aminotransferase, L-asparagine:2-oxo-acid aminotransferase |
| 2.6.1.18 | beta-alanine-alpha-alanine transaminase, beta-alanine-pyruvate aminotransferase, L-alanine:3- |
| | oxopropanoate aminotransferase |
| 2.6.1.19 | 4-aminobutanoate:2-oxoglutarate aminotransferase, 4-aminobutyrate aminotransferase, 4- |
| | aminobutyrate-2-ketoglutarate aminotransferase, 4-aminobutyrate-2-oxoglutarate |
| | aminotransferase, 4-aminobutyrate-2-oxoglutarate transaminase, 4-aminobutyric acid 2- |
| | ketoglutaric acid aminotransferase, 4-aminobutyric acid aminotransferase, aminobutyrate |
| | aminotransferase, aminobutyrate transaminase, beta-alanine aminotransferase, beta-alanine- |
| | oxoglutarate aminotransferase, beta-alanine-oxoglutarate transaminase, g-aminobutyrate |
| | aminotransaminase, g-aminobutyrate transaminase, g-aminobutyrate-alpha-ketoglutarate |
| | aminotransferase, g-aminobutyrate-alpha-ketoglutarate transaminase, g-aminobutyrate:alpha- |
| | oxoglutarate aminotransferase, g-aminobutyric acid aminotransferase, g-aminobutyric acid |
| | pyruvate transaminase, g-aminobutyric acid transaminase, g-aminobutyric acid-2-oxoglutarate |
| | transaminase, g-aminobutyric acid-alpha-ketoglutarate transaminase, g-aminobutyric acid- |
| | alpha-ketoglutaric acid aminotransferase, g-aminobutyric transami |
| 2.6.1.2 | alanine aminotransferase, Alanine transaminase, alanine-alpha-ketoglutarate |
| | aminotransferase, alanine-pyruvate aminotransferase, beta-alanine aminotransferase, glutamic |
| | acid-pyruvic acid transaminase, glutamic-alanine transaminase, glutamic-pyruvic |
| | aminotransferase, glutamic-pyruvic transaminase, GPT, L-alanine aminotransferase, L-alanine |
| | transaminase, L-alanine-alpha-ketoglutarate aminotransferase, L-alanine:2-oxoglutarate |
| | aminotransferase, pyruvate transaminase, pyruvate-alanine aminotransferase, pyruvate- |
| | glutamate transaminase |
| 2.6.1.44 | AGT, alanine-glyoxylate aminotransferase, alanine-glyoxylic aminotransferase, L-alanine- |
| | glycine transaminase, L-alanine:glyoxylate aminotransferase |
| 3.4.13.3 | aminoacylhistidine dipeptidase, carnosinase, dipeptidase M, homocarnosinase |
| 3.5.1.1 | alpha-asparaginase, asparaginase II, colaspase, crasnitin, elspar, kidrolase, L-asparaginase, |
| | L-asparagine amidohydrolase, leunase |
| 3.5.1.15 | acetyl-aspartic deaminase, acylase II, aminoacylase II, N-acetylaspartate amidohydrolase, N- |
| | acyl-L-aspartate amidohydrolase |
| 3.5.1.3 | alpha-keto acid-omega-amidase, omega-amidodicarboxylate amidohydrolase |
| 3.5.1.38 | L-glutamine(L-asparagine) amidohydrolase |
| 3.5.1.7 | N-carbamoyl-L-aspartate amidohydrolase |
| 4.1.1.11 | aspartate alpha-decarboxylase, aspartic alpha-decarboxylase, L-aspartate 1-carboxy-lyase, L- |
| | aspartate alpha-decarboxylase |
| 4.1.1.12 | aminomalonic decarboxylase, aspartate beta-decarboxylase, aspartate omega-decarboxylase, |
| | aspartic beta-decarboxylase, aspartic omega-decarboxylase, cysteine sulfinic desulfinase, |
| | desulfinase, L-aspartate 4-carboxy-lyase, L-aspartate beta-decarboxylase, L-cysteine sulfinate |
| | acid desulfinase |
| 4.1.1.15 | aspartate 1-decarboxylase, aspartic alpha-decarboxylase, cysteic acid decarboxylase, g- |
| | glutamate decarboxylase, Glutamate decarboxylase, L-aspartate-alpha-decarboxylase, L- |
| | glutamate 1-carboxy-lyase, L-glutamate alpha-decarboxylase, L-glutamic acid decarboxylase, |
| | L-glutamic decarboxylase |
| 4.3.1.1 | aspartase, fumaric aminase, L-aspartase, L-aspartate ammonia-lyase |
| 4.3.2.1 | arginine-succinate lyase, argininosuccinic acid lyase, arginosuccinase, N-(L-argininosuccinate) |
| | arginine-lyase |
| 4.3.2.2 | adenylosuccinase, N6-(1,2-dicarboxyethyl)AMP AMP-lyase, succino AMP-lyase |
| 5.1.1.1 | L-alanine racemase |
| 5.1.1.13 | D-aspartate racemase |
| 6.1.1.12 | aspartyl-tRNA synthetase, L-aspartate:tRNAAsp ligase (AMP-forming) |
| 6.1.1.22 | asparaginyl-tRNA synthetase, L-asparagine:tRNAAsn ligase (AMP-forming) |
| 6.1.1.7 | alanyl-tRNA synthetase, L-alanine:tRNAAla ligase (AMP-forming) |
| 6.3.1.1 | asparagine synthetase, L-aspartate:ammonia ligase (AMP-forming) |
| 6.3.2.11 | carnosine synthetase, L-histidine:beta-alanine ligase (AMP-forming) |
| 6.3.4.4 | IMP-aspartate ligase, IMP:L-aspartate ligase (GDP-forming) |
| 6.3.4.5 | citrulline-aspartate ligase, L-citrulline:L-aspartate ligase (AMP-forming) |
| 6.3.5.4 | asparagine synthetase (glutamine-hydrolysing), L-aspartate:L-glutamine amido-ligase (AMP- |
| | forming) |
| 6.3.5.6 | |
| 6.4.1.1 | pyruvate:carbon-dioxide ligase (ADP-forming), pyruvic carboxylase |
| Acetyl-CoA | 72-89-9, acetyl-CoA, C23H38N7O17P3S, coenzyme A, S-acetate, S-acetyl coenzyme A, |
| | [(2R,3R,4R,5R)-2-[[[[3-[2-(2-acetylsulfanylethylcarbamoyl)ethylcarbamoyl]-3-hydroxy-2,2- |
| | dimethyl-propoxy]-hydroxy-phosphoryl]oxy-hydroxy-phosphoryl]oxymethyl]-5-(6-aminopurin-9- |
| | yl)-4-hydroxy-oxolan-3-yl]oxyphosphonic acid |
| Adenylosuccinate | 19046-78-7, 2-[[9-[(2R,3R,4R,5R)-3,4-dihydroxy-5-(phosphonooxymethyl)oxolan-2-yl]purin-6- |
| | yl]amino]butanedioic acid, adenylosuccinate, adenylosuccinic acid, C14H18N5O11P, L- |
| | Aspartic acid, N-(9-(5-O-phosphono-beta-D-ribofuranosyl)-9H-purin-6-yl)-, N6-(1,2- |
| | dicarboxyethyl)-AMP |
| Carnosine | (2S)-2-(3-aminopropanoylamino)-3-(3H-imidazol-4-yl)propanoic acid, 305-84-0, C9H14N4O3, |
| | ignotine, L-carnosine, L-histidine, N-beta-alanyl-, Nalpha-(beta-alanyl)-L-histidine |
| Citrate | 1,2,3-propanetricarboxylic acid, 2-hydroxy-, 126-44-3, 2-hydroxypropane-1,2,3-tricarboxylic |
| | acid, 77-92-9, ammounium citrate, C6H8O7, citrate, sodium citrate |
| D-Alanine | (2R)-2-aminopropanoic acid, (R)-alanine, 338-69-2, alanine D-form, C3H7NO2, D-alanine |
| D-Aspartate | (2R)-2-aminobutanedioic acid, 1783-96-6, C4H7NO4, D-aspartate, D-aspartic acid |
| Fumarate | (E)-but-2-enedioic acid, 110-17-8, 2-butenedioic acid (2E)-, C4H4O4, fumarate |
| L-Alanine | (2S)-2-aminopropanoic acid, 56-41-7, C3H7NO2, L-2-aminopropionic acid, L-Ala, L-alanine |
| L-Alanyl-tRNA (Ala) | alanyl-tRNA, L-alanyl-tRNA |
| L-Argininosuccinate | (2S)-2-[[N′-[(4S)-4-amino-4-carboxy-butyl]carbamimidoyl]amino]butanedioic acid, 2387-71-5, |
| | argininosuccinic acid, C10H18N4O6, L-aspartic acid, N-(((4-amino-4- |
| | carboxybutyl)amino)iminomethyl)-, (S)-, N(omega)-(L-arginino)succinate, N-(L- |
| | arginino)succinate |
| L-Asparagine | (2S)-2-amino-3-carbamoyl-propanoic acid, (S)-2,4-diamino-4-oxobutanoic acid, 70-47-3, |
| | asparagine, aspartic acid beta-amide, C4H8N2O3, L-asparagine |
| L-Asparaginyl-tRNA (Asn) | |
| L-Aspartate | (2S)-2-aminobutanedioic acid, (S)-aminobutanedioic acid, 56-84-8, alpha-aminosuccinic acid, |
| | Asp, aspartate, aspartic acid, C4H7NO4, L-aspartate, L-aspartic acid |
| L-Aspartyl-tRNA (Asn) | |
| L-Aspartyl-tRNA (Asp) | aspartyl-tRNA |
| Malate | (−)-malic acid, (2S)-2-hydroxybutanedioic acid, (S)-malate, 97-67-6, butanedioic acid, hydroxy-, |
| | (2S)-, butanedioic acid, hydroxy-, (S)-, butanedioic acid, hydroxy-, (S)-(9Cl), C4H6O5, L-2- |
| | hydroxybutanedioic acid, L-apple acid, S-2-hydroxybutanedioic acid |
| Malonate semialdehyde | 3-oxopropanoate, 3-oxopropanoic acid, 926-61-4, C3H4O3, malonate semialdehyde, propanoic |
| | acid, 3-oxo- |
| N-Acetyl-L-aspartate | (2S)-2-acetamidobutanedioic acid, 997-55-7, C6H9NO5, N-acetyl-L-aspartate, n- |
| | acetylaspartate |
| N-Carbamoyl-L-aspartate | (2S)-2-(carbamoylamino)butanedioic acid, 13184-27-5, 16649-79-9, 2-ureidobutanedioic acid, |
| | C5H8N2O5, calcium N-carbamoylaspartate, L-Aspartic acid, N-(aminocarbonyl)-, L- |
| | ureidosuccinate, N-carbamoyl-L-aspartate |
| O-Acetylcarnitine | (3S)-3-acetyloxy-4-trimethylammonio-butanoate, 1-propanaminium, 2-(acetyloxy)-3-carboxy- |
| | N,N,N-trimethyl-, hydroxide, inner salt, (R)-, 1-propanaminium, 2-(acetyloxy)-3-carboxy-N,N,N- |
| | trimethyl-, hydroxide, inner salt, (R)-(9Cl), 3040-38-8, 5080-50-2, C9H17NO4, L-carnitine |
| | acetyl ester, L-O-acetylcarnitine, O-acetyl-L-carnitine, O-acetylcarnitine, R-acetylcarnitine |
| Oxaloacetate | 2-ketosuccinic acid, 2-oxobutanedioic acid, 328-42-7, butanedioic acid, oxo-, C4H4O5, OAA, |
| | oxaloacetate, oxaloacetic acid |
| Pyruvate | 127-17-3, 2-oxopropanoate, 2-oxopropanoic acid, 57-60-3, C3H4O3, propanoic acid, 2-oxo-, |
| | propanoic acid, 2-oxo-, ion(1-), propanoic acid, 2-oxo-, sodium salt, pyruvate, pyruvic acid, |
| | sodium salt, sodium pyruvate |
| Succinate | 1,2-ethanedicarboxylic acid, 1,4-butanedioic acid, 110-15-6, 56-14-4, amber acid, asuccin, |
| | butanedioate, butanedioic acid, C4H6O4, ethylenesuccinic acid, katasuccin, potassium |
| | succinate, succinate, wormwood acid |
| &beta;-Alanine | 107-95-9, 2-carboxyethylamine, 28854-76-4, 3-aminopropanoic acid, abufene, beta-alanine, |
| | beta-aminopropionic acid, C3H7NO2 |

*Genes and/or protein that were used to identify the pathway:
2.1.3.2 aspartate carbamyltransferase, aspartate transcarbamoylase, aspartate transcarbamylase, aspartic acid transcarbamoylase, aspartic carbamyltransferase, aspartic transcarbamylase, ATCase, carbamoyl-phosphate:L-aspartate carbamoyltransferase, carbamoylaspartotranskinase, carbamylaspartotranskinase, L-aspartate transcarbamoylase, L-aspartate transcarbamylase

TABLE 11
Genes/Proteins Involved in the Glutamate metabolism pathway

| Name | Synonyms |
|---|---|
| 1.2.1.16 | succinate semialdehyde dehydrogenase (nicotinamide adenine dinucleotide (phosphate)), |
| | succinate-semialdehyde:NAD(P) oxidoreductase |
| 1.4.1.13 | glutamate (reduced nicotinamide adenine dinucleotide phosphate) synthase, glutamate |
| | synthetase (NADP), glutamine amide-2-oxoglutarate aminotransferase (oxidoreductase, |
| | NADP), glutamine-ketoglutaric aminotransferase, L-glutamate synthase, L-glutamate |
| | synthetase, L-glutamate:NADP+ oxidoreductase (transaminating), L-glutamine:2- |
| | oxoglutarate aminotransferase, NADPH oxidizing, NADPH-dependent glutamate |
| | synthase, NADPH-glutamate synthase, NADPH-linked glutamate synthase |
| 1.4.1.14 | glutamate (reduced nicotinamide adenine dinucleotide) synthase, L-glutamate synthase |
| | (NADH), L-glutamate synthetase, L-glutamate:NAD oxidoreductase (transaminating), |
| | NADH-dependent glutamate synthase, NADH-glutamate synthase, NADH:GOGAT |
| 1.4.1.2 | glutamate dehydrogenase (NAD), glutamate oxidoreductase, glutamic acid |
| | dehydrogenase, glutamic dehydrogenase, L-glutamate dehydrogenase, L-glutamate:NAD |
| | oxidoreductase (deaminating), NAD-dependent glutamate dehydrogenase, NAD- |
| | dependent glutamic dehydrogenase, NAD-glutamate dehydrogenase, NAD-linked |
| | glutamate dehydrogenase, NAD-linked glutamic dehydrogenase, NAD-specific glutamate |
| | dehydrogenase, NAD-specific glutamic dehydrogenase, NAD:glutamate oxidoreductase, |
| | NADH-linked glutamate dehydrogenase |
| 1.4.1.3 | glutamic dehydrogenase, L-glutamate:NAD(P) oxidoreductase (deaminating) |
| 1.4.1.4 | dehydrogenase, glutamate (nicotinamide adenine dinucleotide (phosphate)), glutamic acid |
| | dehydrogenase, glutamic dehydrogenase, L-glutamate dehydrogenase, L- |
| | glutamate:NADP oxidoreductase (deaminating), L-glutamic acid dehydrogenase, NAD(P)- |
| | glutamate dehydrogenase, NAD(P)H-dependent glutamate dehydrogenase |
| 1.5.1.12 | 1-pyrroline dehydrogenase, 1-pyrroline-5-carboxylate:NAD oxidoreductase, D1-pyrroline- |
| | 5-carboxylate dehydrogenase, L-pyrroline-5-carboxylate-NAD+ oxidoreductase, pyrroline- |
| | 5-carboxylate dehydrogenase, pyrroline-5-carboxylic acid dehydrogenase |
| 1.8.1.7 | glutathione reductase, glutathione reductase (NADPH), glutathione S-reductase |
| | glutathione:NADP+ oxidoreductase, GSH reductase, GSSG reductase, NADPH- |
| | glutathione reductase, NADPH-GSSG reductase, NADPH:oxidized-glutathione |
| | oxidoreductase |
| 1.8.4.— | |
| 1.8.5.1 | dehydroascorbate reductase, dehydroascorbic acid reductase, dehydroascorbic |
| | reductase, DHA reductase, GDOR, glutathione dehydroascorbate reductase, |
| | glutathione:dehydroascorbate oxidoreductase, glutathione:dehydroascorbic acid |
| | oxidoreductase |
| 2-Oxoglutaramate | 18465-19-5, 2-oxoglutaramate, 2-oxoglutaramic acid, 4-carbamoyl-2-oxo-butanoic acid, |
| | alpha-ketoglutaramic acid, C5H7NO4, pentanoic acid, 5-amino-2,5-dioxo- |
| 2-Oxoglutarate | 2-ketoglutarate, 2-oxoglutarate, 2-oxopentanedioic acid, 328-50-7, alpha-ketoglutarate, |
| | alpha-ketoglutaric acid, alphaKG, C5H6O5, glutaric acid, 2-oxo-, glutaric acid, 2-oxo- |
| | (8Cl), pentanedioic acid, 2-oxo- |
| 2.3.1.4 | acetyl-CoA:D-glucosamine-6-phosphate N-acetyltransferase, |
| | aminodeoxyglucosephosphate acetyltransferase, D-glucosamine-6-P N-acetyltransferase, |
| | glucosamine 6-phosphate acetylase, glucosamine-phosphate N-acetyltransferase, N- |
| | acetylglucosamine-6-phosphate synthase, phosphoglucosamine acetylase, |
| | phosphoglucosamine N-acetylase, phosphoglucosamine transacetylase |
| 2.4.2.14 | 5′-phosphoribosylpyrophosphate amidotransferase, 5-phosphoribosyl-1-pyrophosphate |
| | amidotransferase, 5-phosphoribosylamine:diphosphate phospho-alpha-D- |
| | ribosyltransferase (glutamate-amidating), 5-phosphororibosyl-1-pyrophosphate |
| | amidotransferase, alpha-5-phosphoribosyl-1-pyrophosphate amidotransferase, glutamine |
| | 5-phosphoribosylpyrophosphate amidotransferase, glutamine phosphoribosyldiphosphate |
| | amidotransferase, glutamine ribosylpyrophosphate 5-phosphate amidotransferase, |
| | phosphoribose pyrophosphate amidotransferase, phosphoribosyl pyrophosphate |
| | amidotransferase, phosphoribosyldiphosphate 5-amidotransferase, |
| | phosphoribosylpyrophosphate glutamyl amidotransferase |
| 2.6.1.1 | 2-oxoglutarate-glutamate aminotransferase, AAT, aspartate alpha-ketoglutarate |
| | transaminase, aspartate aminotransferase, Aspartate transaminase, aspartate-2- |
| | oxoglutarate transaminase, aspartate:2-oxoglutarate aminotransferase, aspartic acid |
| | aminotransferase, aspartic aminotransferase, aspartyl aminotransferase, AspT, AST, |
| | glutamate oxaloacetate transaminase, glutamate-oxalacetate aminotransferase |
| | glutamate-oxalate transaminase, glutamic oxalic transaminase, glutamic-aspartic |
| | aminotransferase, glutamic-aspartic transaminase, glutamic-oxalacetic transaminase, |
| | glutamic-oxaloacetic transaminase, GOT (enzyme), L-aspartate transaminase, L- |
| | aspartate-2-ketoglutarate aminotransferase, L-aspartate-2-oxoglutarate aminotransferase, |
| | L-aspartate-2-oxoglutarate-transaminase, L-aspartate-alpha-ketoglutarate transaminase, |
| | L-aspartate:2-oxoglutarate aminotransferase, L-aspartic aminotransferase, oxaloacetate |
| | transferase, oxaloacetate-aspartate aminotransferase, Sgot, transaminase A |
| 2.6.1.15 | g-glutaminyltransferase, glutaminase II, glutamine transaminase, glutamine-alpha-keto |
| | acid transamidase, glutamine-alpha-keto acid transaminase, glutamine-keto acid |
| | aminotransferase, glutamine-oxo acid aminotransferase, glutamine-oxo-acid transaminase |
| | glutamine transaminase L, L-glutamine transaminase L, L-glutamine:pyruvate |
| | aminotransferase |
| 2.6.1.16 | D-fructose-6-phosphate amidotransferase, GlcN6P synthase, glucosamine 6-phosphate |
| | synthase, glucosamine-6-phosphate isomerase (glutamine-forming), |
| | glucosaminephosphate isomerase, hexosephosphate aminotransferase, L-glutamine:D- |
| | fructose-6-phosphate isomerase (deaminating) |
| 2.6.1.19 | 4-aminobutanoate:2-oxoglutarate aminotransferase, 4-aminobutyrate aminotransferase, 4- |
| | aminobutyrate-2-ketoglutarate aminotransferase, 4-aminobutyrate-2-oxoglutarate |
| | aminotransferase, 4-aminobutyrate-2-oxoglutarate transaminase, 4-aminobutyric acid 2- |
| | ketoglutaric acid aminotransferase, 4-aminobutyric acid aminotransferase, aminobutyrate |
| | aminotransferase, aminobutyrate transaminase, beta-alanine aminotransferase, beta- |
| | alanine-oxoglutarate aminotransferase, beta-alanine-oxoglutarate transaminase, g- |
| | aminobutyrate aminotransaminase, g-aminobutyrate transaminase, g-aminobutyrate- |
| | alpha-ketoglutarate aminotransferase, g-aminobutyrate-alpha-ketoglutarate transaminase, |
| | g-aminobutyrate:alpha-oxoglutarate aminotransferase, g-aminobutyric acid |
| | aminotransferase, g-aminobutyric acid pyruvate transaminase, g-aminobutyric acid |
| | transaminase, g-aminobutyric acid-2-oxoglutarate transaminase, g-aminobutyric acid- |
| | alpha-ketoglutarate transaminase, g-aminobutyric acid-alpha-ketoglutaric acid |
| | aminotransferase, g-aminobutyric transami |
| 2.6.1.2 | alanine aminotransferase, Alanine transaminase, alanine-alpha-ketoglutarate |
| | aminotransferase, alanine-pyruvate aminotransferase, beta-alanine aminotransferase, |
| | glutamic acid-pyruvic acid transaminase, glutamic-alanine transaminase, glutamic-pyruvic |
| | aminotransferase, glutamic-pyruvic transaminase, GPT, L-alanine aminotransferase, L- |
| | alanine transaminase, L-alanine-alpha-ketoglutarate aminotransferase, L-alanine:2- |
| | oxoglutarate aminotransferase, pyruvate transaminase, pyruvate-alanine |
| | aminotransferase, pyruvate-glutamate transaminase |
| 2.7.1.59 | 2-acetylamino-2-deoxy-D-glucose kinase, acetylaminodeoxyglucokinase, |
| | acetylglucosamine kinase (phosphorylating), ATP:2-acetylamino-2-deoxy-D-glucose 6- |
| | phosphotransferase, ATP:N-acetyl-D-glucosamine 6-phosphotransferase |
| 2.7.2.2 | ATP:carbamate phosphotransferase, carbamoyl phosphokinase, carbamyl |
| | phosphokinase, CKase |
| 3.5.1.2 | glutaminase I, glutamine aminohydrolase, L-glutaminase, L-glutamine amidohydrolase |
| 3.5.1.3 | alpha-keto acid-omega-amidase, omega-amidodicarboxylate amidohydrolase |
| 3.5.1.38 | L-glutamine(L-asparagine) amidohydrolase |
| 4-Aminobutanoate | 4-aminobutanoic acid, 4-aminobutyrate, 4-aminobutyric acid, 56-12-2, butanoic acid, 4- |
| | amino-, C4H9NO2, gamma-amino-N-butyric acid, gamma-aminobutyric acid |
| 4.1.1.15 | aspartate 1-decarboxylase, aspartic alpha-decarboxylase, cysteic acid decarboxylase, g- |
| | glutamate decarboxylase, Glutamate decarboxylase, L-aspartate-alpha-decarboxylase, L- |
| | glutamate 1-carboxy-lyase, L-glutamate alpha-decarboxylase, L-glutamic acid |
| | decarboxylase, L-glutamic decarboxylase |
| 4.1.1.19 | L-arginine carboxy-lyase |
| 5-Phosphoribosylamine | 14050-66-9, 5-phospho-beta-D-ribosylamine, 5-phospho-D-ribosylamine, 5- |
| | phosphoribosyl-1-amine, C5H12NO7P, D-Ribofuranosylamine, 5-(dihydrogen phosphate), |
| | phosphoribosylamine, [(2R,3R,4R)-5-amino-3,4-dihydroxy-oxolan-2- |
| | yl]methoxyphosphonic acid |
| 5.1.1.3 | |
| 6.1.1.17 | glutamyl-tRNA synthetase, L-glutamate:tRNAGlu ligase (AMP-forming) |
| 6.1.1.18 | glutaminyl-tRNA synthetase, L-glutamine:tRNAGln ligase (AMP-forming) |
| 6.3.1.2 | glutamine synthetase, L-glutamate:ammonia ligase (ADP-forming) |
| 6.3.2.2 | g-glutamylcysteine synthetase, L-glutamate:L-cysteine g-ligase (ADP-forming) |
| 6.3.2.3 | g-L-glutamyl-L-cysteine:glycine ligase (ADP-forming), glutathione synthetase |
| 6.3.4.16 | carbon-dioxide-ammonia ligase, carbon-dioxide:ammonia ligase (ADP-forming, |
| | carbamate-phosphorylating) |
| 6.3.5.1 | deamido-NAD:L-glutamine amido-ligase (AMP-forming), NAD synthetase (glutamine- |
| | hydrolysing) |
| 6.3.5.2 | GMP synthetase (glutamine-hydrolysing), xanthosine-5′-phosphate:L-glutamine amido- |
| | ligase (AMP-forming) |
| 6.3.5.7 | |
| Carbamoyl-P | 590-55-6, carbamic acid, monoanhydride with phosphoric acid, carbamoyloxyphosphonic |
| | acid, CH4NO5P |
| Citrate | 1,2,3-propanetricarboxylic acid, 2-hydroxy-, 126-44-3, 2-hydroxypropane-1,2,3- |
| | tricarboxylic acid, 77-92-9, ammounium citrate, C6H8O7, citrate, sodium citrate |
| CO2 | 124-38-9, carbon dioxide, carbonic anhydride, CO2, dry ice |
| D-Glutamate | (2R)-2-aminopentanedioic acid, 6893-26-1, C5H9NO4, D-2-aminoglutaric acid, D-2- |
| | aminopentanedioic acid, D-glutamate, D-glutamic acid, D-glutaminic acid, R-(−)-glutamic |
| | acid |
| Fumarate | (E)-but-2-enedioic acid, 110-17-8, 2-butenedioic acid (2E)-, C4H4O4, fumarate |
| Glucosamine-6P | 3616-42-0, C6H14NO8P, D-glucosamine-6-phosphate, D-Glucose, 2-amino-2-deoxy-, 6- |
| | (dihydrogen phosphate), [(2R,3S,4R,5R,6S)-5-amino-3,4,6-trihydroxy-oxan-2- |
| | yl]methoxyphosphonic acid |
| Glutathione (ox) | (2S)-2-amino-4-[[(1R)-2-[(2R)-2-[[(4S)-4-amino-4-carboxy-butanoyl]amino]-2- |
| | (carboxymethylcarbamoyl)ethyl]disulfanyl-1- |
| | (carboxymethylcarbamoyl)ethyl]carbamoyl]butanoic acid, 27025-41-8, bis(gamma- |
| | glutamyl-L-cysteinylglycine) disulfide, C20H32N6O12S2, glutathione, oxidized, GSSG, |
| | oxiglutatione |
| Glutathione (red) | (2S)-2-amino-4-[[(1R)-1-(carboxymethylcarbamoyl)-2-sulfanyl-ethyl]carbamoyl]butanoic |
| | acid, 70-18-8, C10H17N3O6S, gamma-Glu-Cys-Gly, gamma-L-glutamylcysteinylglycine, |
| | glutathione-reduced, glycine, N-(N-L-gamma-glutamyl-L-cysteinyl)-, GSH |
| GMP | 5′-GMP, 5′-guanylic acid, 85-32-5, C10H14N5O8P, GMP5′, guanosine monophosphate, |
| | [(2R,3R,4R,5R)-5-(2-amino-6-oxo-3H-purin-9-yl)-3,4-dihydroxy-oxolan-2- |
| | yl]methoxyphosphonic acid |
| L-1-Pyrroline 5-carboxylate | (2S)-3,4-dihydro-2H-pyrrole-2-carboxylic acid, (S)-1-pyrroline-5-carboxylate, C5H7NO2, L- |
| | 1-pyrroline-5-carboxylate, L-pyrroline-5-carboxylic acid |
| L-Glutamate | (2S)-2-aminopentanedioic acid, 142-47-2, 19473-49-5, 56-86-0, C5H9NO4, glutamate, |
| | glutamic acid, L-Glu, L-glutamate, L-glutamic acid, monosodium glutamate, potassium |
| | glutamate, potassium L-glutamate, sodium glutamate |
| L-Glutamine | (2S)-2-amino-4-carbamoyl-butanoic acid, (S)-2,5-diamino-5-oxopentanoic acid, 56-85-9, |
| | C5H10N2O3, L-2-aminoglutaramidic acid, L-glutamine, levoglutamide |
| L-Glutaminyl-tRNA (Gln) | glutaminyl tRNA |
| L-Glutamyl-tRNA(Glu) | |
| L-Glutamyl-tRNA (Gln) | |
| L-&gamma;-Glutamyl-cysteine | (2S)-2-amino-4-[[(1R)-1-carboxy-2-sulfanyl-ethyl]carbamoyl]butanoic acid, 636-58-8, |
| | C8H14N2O5S, gamma-Glu-Cys, gamma-L-glutamyl-L-cysteine, L-Cysteine, N-L-gamma- |
| | glutamyl- |
| Malate | (−)-malic acid, (2S)-2-hydroxybutanedioic acid, (S)-malate, 97-67-6, butanedioic acid, |
| | hydroxy-, (2S)-, butanedioic acid, hydroxy-, (S)-, butanedioic acid, hydroxy-, (S)-(9Cl), |
| | C4H6O5, L-2-hydroxybutanedioic acid, L-apple acid, S-2-hydroxybutanedioic acid |
| N-Acetyl-D-glucosamine | 2-acetamido-2-deoxy-D-glucose, 2-acetamido-2-deoxyglucose, 7512-17-6, C8H15NO6, |
| | D-GlcNAc, D-Glucose, 2-(acetylamino)-2-deoxy-, GlcNAc, N-acetyl-D-glucosamine, N- |
| | [(3R,4R,5S,6R)-2,4,5-trihydroxy-6-(hydroxymethyl)oxan-3-yl]acetamide, O-GlcNAc |
| N-Acetyl-D-glucosamine 6P | C8H16NO9P, [(2R,3S,4R,5R,6S)-5-acetamido-3,4,6-trihydroxy-oxan-2- |
| | yl]methoxyphosphonic acid |
| NAD | 53-84-9, adenosine 5′-(trihydrogen diphosphate), P′-5′-ester with 3-(aminocarbonyl)-1- |
| | beta-D-ribofuranosylpyridinium, inner salt, beta-NAD+, beta-nicotinamide adenine |
| | dinucleotide+, C21H28N7O14P2+, NAD, [[(2R,3R,4R,5R)-5-(6-aminopurin-9-yl)-3,4- |
| | dihydroxy-oxolan-2-yl]methoxy-hydroxy-phosphoryl]oxy-[[(2R,3R,4R,5R)-5-(5- |
| | carbamoylpyridin-1-yl)-3,4-dihydroxy-oxolan-2-yl]methoxy]phosphinic acid |
| NH3 | 7664-41-7, ammonia, anhydrous, anhydrous ammonia, azane, H3N |
| Oxaloacetate | 2-ketosuccinic acid, 2-oxobutanedioic acid, 328-42-7, butanedioic acid, oxo-, C4H4O5, |
| | OAA, oxaloacetate, oxaloacetic acid |
| Succinate | 1,2-ethanedicarboxylic acid, 1,4-butanedioic acid, 110-15-6, 56-14-4, amber acid, asuccin, |
| | butanedioate, butanedioic acid, C4H6O4, ethylenesuccinic acid, katasuccin, potassium |
| | succinate, succinate, wormwood acid |
| Succinate semialdehyde | 3-formylpropanoic acid, 4-oxobutanoic acid, 692-29-5, beta-formylpropionic acid, butanoic |
| | acid, 4-oxo-, butanoic acid, 4-oxo-(9Cl), butryaldehydic acid, C4H6O3, gamma-oxybutyric |
| | acid, succinaldehydic acid, succinate semialdehyde |

*Genes and/or proteins that were used to identify the pathway:
1.2.1.24 succinate semialdehyde:NAD+ oxidoreductase, succinate-semialdehyde:NAD oxidoreductase, succinic semialdehyde dehydrogenase, succinyl semialdehyde dehydrogenase
6.3.5.5 carbamoyl-phosphate synthetase (glutamine-hydrolysing), carbon-dioxide:L-glutamine amido-ligase (ADP-forming, carbamate-phosphorylating)

### Example 3

### Exemplary Pathways Relating to High Cell Growth Rate

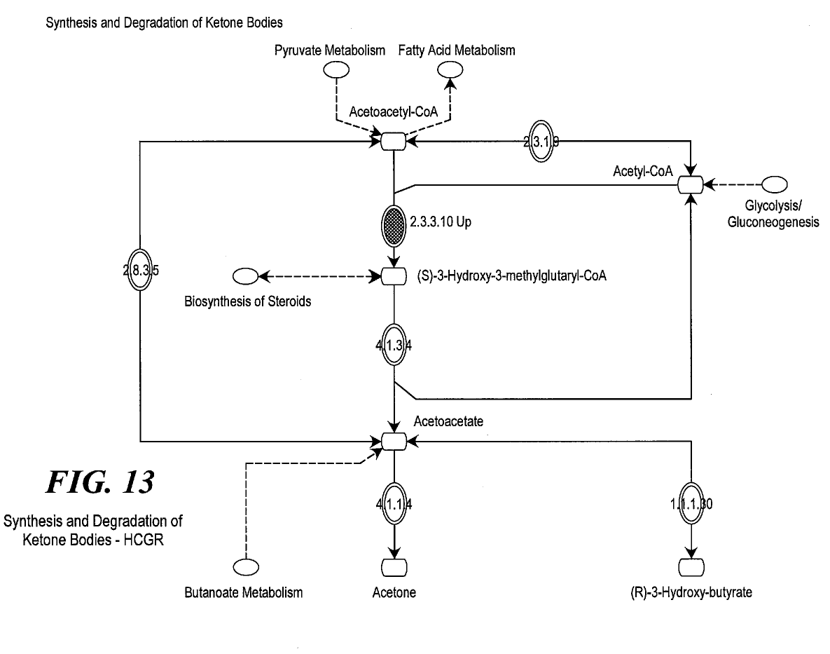

Pathway analysis using Ingenuity software based on previously identified differently expressed genes or proteins associated with high cell growth rate led to the identification of the synthesis and degradation of ketone bodies pathway (FIG. 13). Genes and/or proteins that were used to identify the pathway are indicated in FIG. 13. In addition, additional exemplary genes or proteins involved in the above-identified pathway and that may be involved in regulating or indicative of high cell growth rate are summarized in Table 12.

TABLE 12
Genes/Proteins Involved in the Synthesis and degradation of ketone bodies pathway

| Name | Synonyms |
|---|---|
| (R)-3-Hydroxy-butyrate | (3R)-3-hydroxybutanoic acid, (R)-(−)-3-hydroxybutyric acid sodium salt, (R)- |
| | 3-hydroxybutanoic acid, (R)-3-hydroxybutyric acid, 13613-65-5, 625-72-9, |
| | C4H8O3, D-beta-hydroxybutyrate, R-3-hydroxybutanoate, sodium (R)-3- |
| | hydroxybutyrate |
| (S)-3-Hydroxy-3- | (3S)-4-[2-[3-[[4-[[[(2R,3R,4R,5R)-5-(6-aminopurin-9-yl)-4-hydroxy-3- |
| methylglutaryl-CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-hydroxy-3-methyl- |
| | butanoic acid, (S)-3-hydroxy-3-methylglutaryl-CoA, 1553-55-5, |
| | C27H44N7O20P3S, hydroxymethylglutaryl-CoA, S-(hydrogen 3-hydroxy-3- |
| | methylglutaryl)coenzyme A, S-(hydrogen 3-hydroxy-3-methylpentanedioate)coenzyme A |
| 1.1.1.30 | (R)-3-hydroxybutanoate:NAD oxidoreductase, 3-D-hydroxybutyrate |
| | dehydrogenase, beta-hydroxybutyrate dehydrogenase, beta-hydroxybutyric |
| | acid dehydrogenase, beta-hydroxybutyric dehydrogenase, D-(−)-3- |
| | hydroxybutyrate dehydrogenase, D-3-hydroxybutyrate dehydrogenase, D- |
| | beta-hydroxybutyrate dehydrogenase, hydroxybutyrate oxidoreductase, |
| | NAD-beta-hydroxybutyrate dehydrogenase |
| 2.3.1.9 | 2-methylacetoacetyl-CoA thiolase, 3-oxothiolase, acetoacetyl-CoA thiolase, |
| | acetyl coenzyme A thiolase, acetyl-CoA acetyltransferase, acetyl- |
| | CoA:acetyl-CoA C-acetyltransferase, acetyl-CoA:N-acetyltransferase, beta- |
| | acetoacetyl coenzyme A thiolase, thiolase II |
| 2.8.3.5 | 3-ketoacid CoA-transferase, 3-ketoacid coenzyme A transferase, 3-oxo-CoA |
| | transferase, 3-oxoacid CoA dehydrogenase, 3-oxoacid coenzyme A- |
| | transferase, acetoacetate succinyl-CoA transferase, acetoacetyl coenzyme |
| | A-succinic thiophorase, succinyl coenzyme A-acetoacetyl coenzyme A- |
| | transferase, succinyl-CoA transferase, succinyl-CoA:3-oxo-acid CoA- |
| | transferase |
| 4.1.1.4 | acetoacetate carboxy-lyase, acetoacetic acid decarboxylase |
| 4.1.3.4 | (S)-3-hydroxy-3-methylglutaryl-CoA acetoacetate-lyase, 3-hydroxy-3- |
| | methylglutaryl CoA cleaving enzyme, 3-hydroxy-3-methylglutaryl coenzyme |
| | A lyase, 3-hydroxy-3-methylglutaryl-CoA lyase, hydroxymethylglutaryl |
| | coenzyme A lyase, hydroxymethylglutaryl coenzyme A-cleaving enzyme |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme |
| | A, [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetone | 2-Propanone, 67-64-1, acetone, C3H6O, dimethyl ketone, |
| | dimethylformaldehyde, dimethylketal, propanone |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |

*Genes and/or proteins that was used to identify the pathway:
2.3.3.10 (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b-methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase

### Example 4

### Exemplary Pathways Associated with High Maximum Cellular Productivity

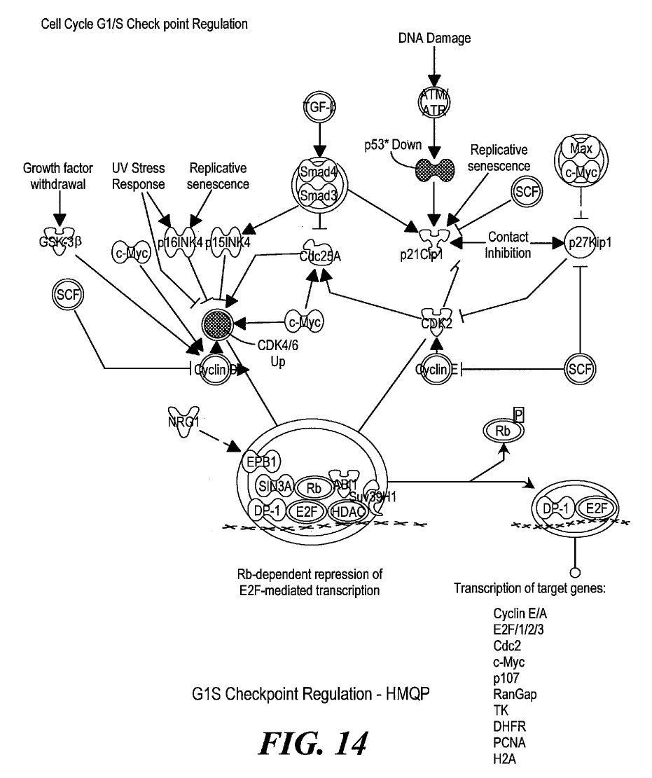

Pathway analysis using Ingenuity software based on previously identified differently expressed genes or proteins associated with high maximum cellular productivity led to the identification of the G1/S checkpoint regulation pathway (FIG. 14). Genes and/or proteins that were used to identify the pathway are indicated in FIG. 14. In addition, additional exemplary genes or proteins involved in the above-identified pathway and that may be involved in regulating or indicative of high maximum cellular productivity are summarized in Table 13.

TABLE 13
Genes/Proteins Involved in the G1/S checkpoint regulation pathway

| Name | Synonyms |
|---|---|
| Abl1 | ABL, AI325092, bcr/abl, C-ABL, C-ABL 1B, CABL1, E430008G22Rik, JTK7, |
| | MGC117749, p145Abl, p150, v-abl |
| ATM/ATR | |
| c-Myc | AU016757, C-MYC, C-MYC-P64, MGC105490, MGC138120, mMyc, Myc2, |
| | Niard, Nird, RNCMYC |
| Cdc25A | CDC25A2, D9Ertd393e |
| CDK2 | A630093N05Rik, Cyclin A associated kinase, CYCLIN E ASSOCIATED |
| | KINASE, CYCLIN E-DEPENDENT KINASE, p33(CDK2), p33CDK2 |
| Cyclin D | CycD |
| Cyclin E | |
| DP-1 | DP-1, DRTF1, TB2/DP1 |
| E2F | |
| E2f-Tfdp1 | |
| EBP1 | 38 kDa, AA672939, EBP1, HG4-1, Itaf45, MGC94070, p38-2G4, PIfap, |
| | PROLIFERATION ASSOCIATED 2G4, Proliveration-associated protein 1 |
| GSK-3&beta; | 7330414F15Rik, 8430431H08Rik, C86142, GSK-3, GSK-3BETA, Tpk1 |
| HDAC | Hdac protein |
| Max | AA960152, AI875693, MGC10775, MGC11225, MGC124611, MGC18164, |
| | MGC34679, MGC36767, orf1 |
| Max-Myc | |
| NRG1 | 6030402G23RIK, ARIA, D230005F13Rik, Doc4, GGF, GGF2, GGFII, GP30, |
| | HEREGULIN, HGL, HRG, HRG1, HRGA, HRGalpha, NAF, NDF, |
| | NEUREGULIN, Nrg alpha, Nrg beta, NRG1 SECRETED, NRG1B1, SMDF, |
| | Ten-m4, Type I Nrg1, Type III Nrg1 |
| p15INK4 | AV083695, CDK4I, INK4B, MTS2, P15, p15(INK4b), P15INK4B, TP15 |
| p16INK4 | ARF, ARF-INK4a, CDK4I, CDKN2, CMM2, CYCLIN-DEPENDENT KINASE |
| | INHIBITOR 2A, INK4, INK4A, INK4a-ARF, MLM, MTS1, p14, p14/ARF, |
| | p14ARF, P16, p16(INK4a), p16Cdkn2a, p16INK4, P16INK4A, p19, p19<ARF>, |
| | p19arf, PCTR1, TP16 |
| p21Cip1 | CAP20, CDKI, CDKN1, CDKNA1, CIP1, MDA-6, P21, p21/WAF1, P21CIP1, |
| | P21WAF, p21waf1, SDI1, WAF1 |
| p27Kip1 | AA408329, AI843786, Cdki1b, CDKN4, CYCLIN-DEPENDENT KINASE |
| | INHIBITOR P27, KIP1, MEN1B, MEN4, P27, P27kip, P27KIP1, P28-ICK |
| Rb | pRb, Rb Tumor Suppressor |
| Rb-E2F transcription | |
| repression complex | |
| SCF | Scf protein |
| SIN3A | AW553200, DKFZP434K2235, FLJ90319, KIAA0700, KIAA4126, mKIAA4126, |
| | MSIN3A, SIN3, Sin3a predicted, Sin3a_predicted |
| Smad3 | AU022421, DKFZP586N0721, DKFZp686J10186, hMAD-3, HSPC193, |
| | HsT17436, JV15-2, MAD3, MADH3, MGC60396 |
| Smad3-Smad4 | |
| Smad4 | AW743858, D18Wsu70e, DPC4, JIP, MADH4, Smaug1 |
| Suv39H1 | AI852103, AL022883, DXHXS7466e, KMT1A, MG44, mIS6, RGD1565028, |
| | SUV39H, Suv39h1 predicted, Suv39h1_predicted |
| TGF-&beta; | LAP, Tgfb |

*Genes and/or proteins that were used to identify the pathway:
CDK4/6
p53 bbl, bfy, bhy, FLJ92943, LFS1, MGC112612, P53, Trp53

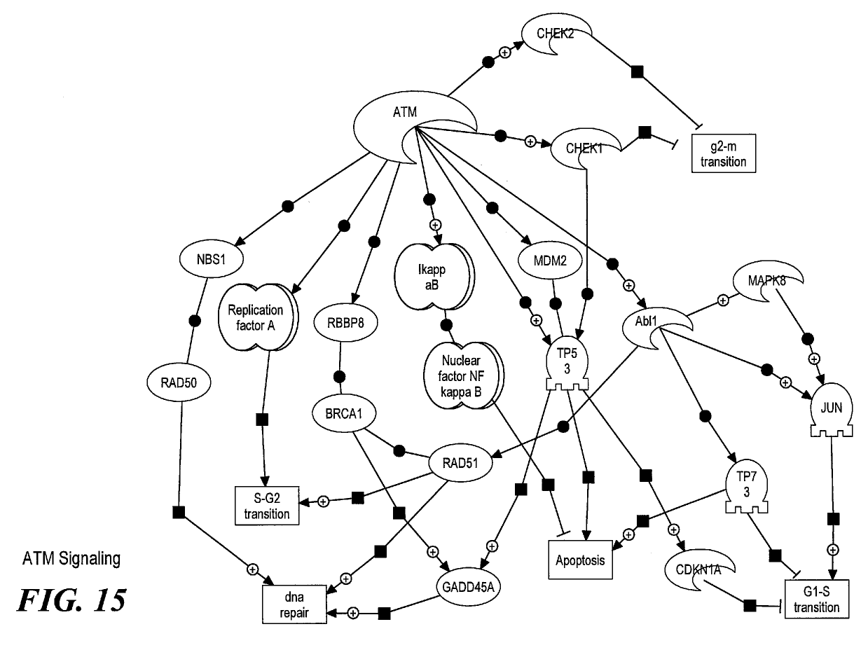

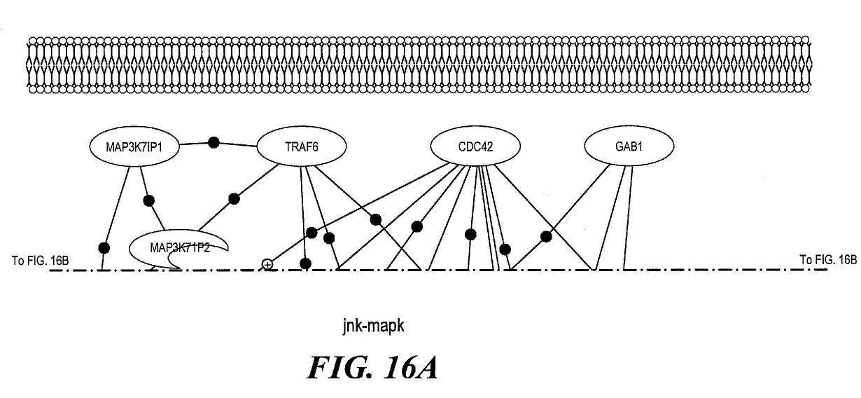

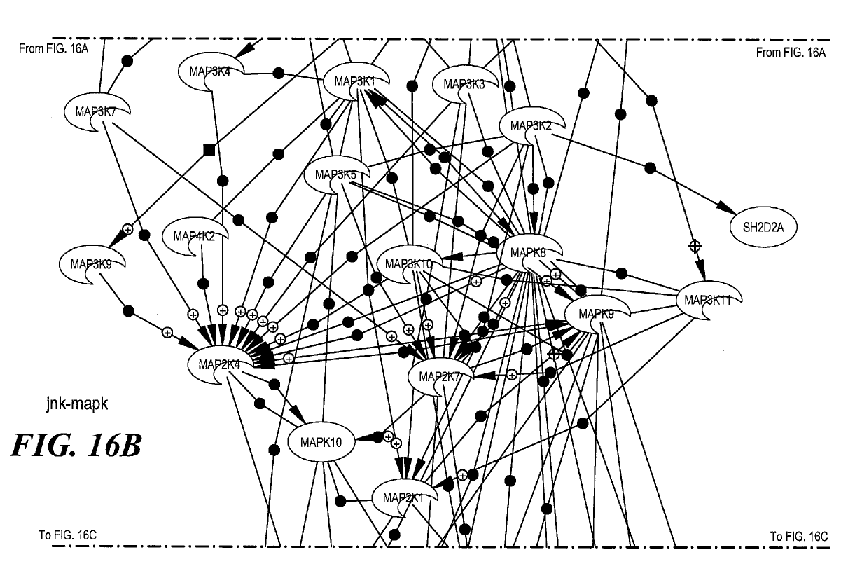

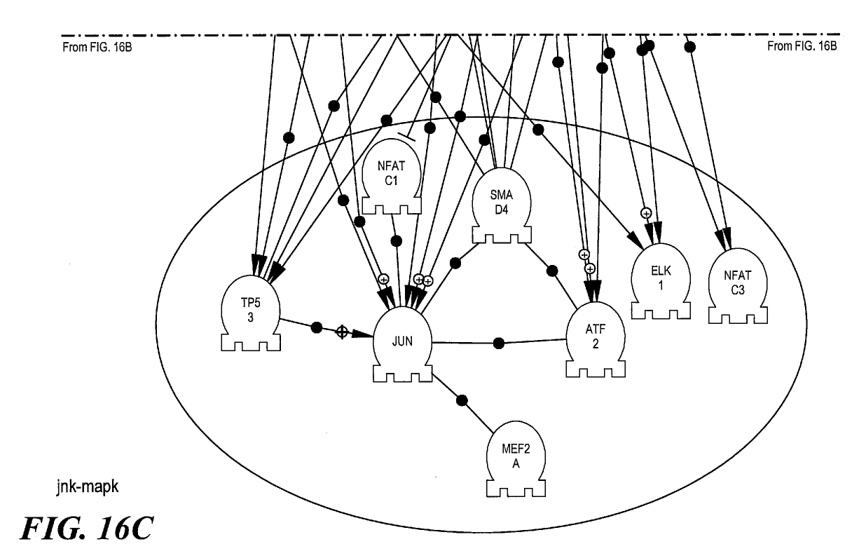

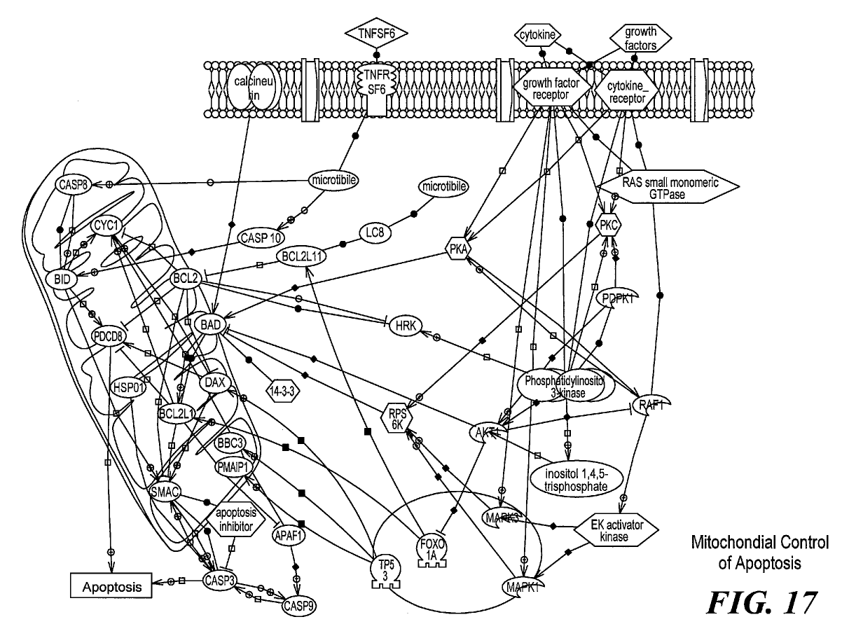

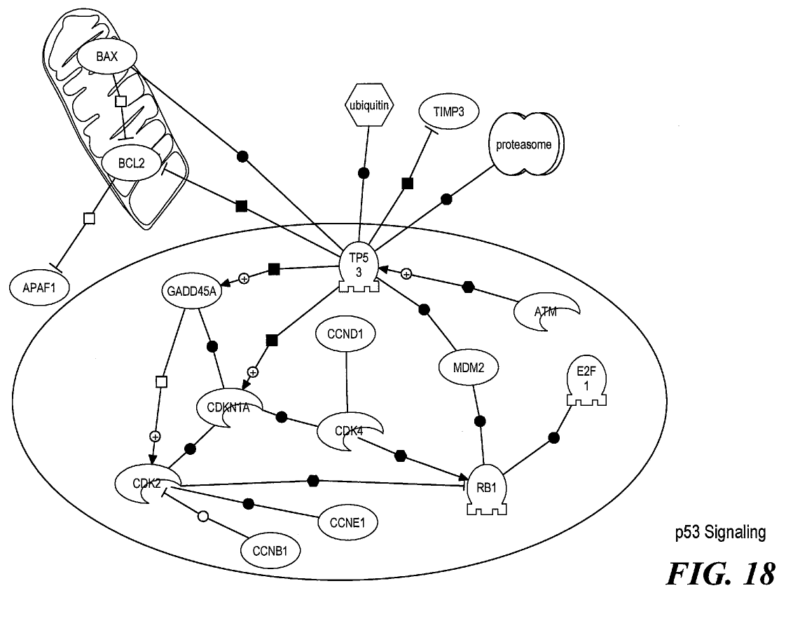

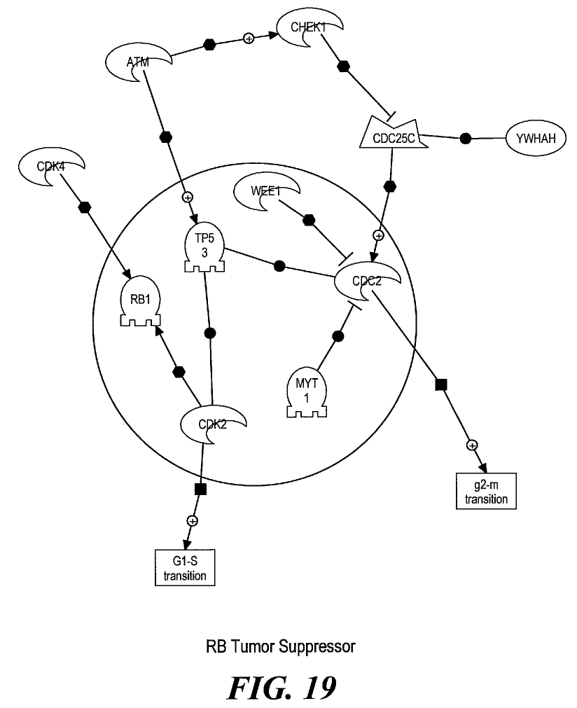

Pathway analysis using Pathway Studio software based on previously identified differently expressed genes or proteins associated with high maximum cellular productivity led to the identification of the ATM signaling pathway (FIG. 15), the Eda-A1 pathway (FIG. 9), the Eda-A2 pathway (FIG. 10), the Jnk-mapk pathway (FIG. 16), and the mitochondrial control of apoptosis pathway (FIG. 17), the p53 signaling pathway (FIG. 18), the RB tumor suppressor pathway (FIG. 19). Previously identified genes and/or proteins that were used to identify relevant pathways are indicated in FIGS. 15-19. In addition, additional exemplary genes or proteins involved in the above-identified pathways and that may be involved in regulating or indicative of high maximum cellular productivity are summarized in Table 14 (ATM signaling pathway), Table 15 (the Eda-A1 pathway), Table 16 (the Eda-A2 pathway), Table 17 (the Jnk-mapk pathway), Table 18 (the mitochondrial control of apoptosis pathway), Table 19 (the p53 signaling pathway), and Table 20 (the RB tumor suppressor pathway).

TABLE 14
ATM signaling pathway

| Name | Type | Description |
|---|---|---|
| Abl1 | Protein | v-abl Abelson murine leukemia viral |
| | | oncogene homolog 1 |
| Apoptosis | Cell Process | |
| ATM | Protein | ataxia telangiectasia mutated (includes |
| | | complementation groups A, C and D) |
| BRCA1 | Protein | breast cancer 1, early onset |
| CDKN1A | Protein | cyclin-dependent kinase |
| | | inhibitor 1A (p21, Cip1) |
| CHEK1 | Protein | CHK1 checkpoint homolog (S. pombe) |
| CHEK2 | Protein | CHK2 checkpoint homolog (S. pombe) |
| dna repair | Cell Process | |
| G1-S transition | Cell Process | |
| g2-m transition | Cell Process | |
| GADD45A | Protein | growth arrest and DNA-damage- |
| | | inducible, alpha |
| IkappaB | Complex | |
| JUN | Protein | v-jun sarcoma virus 17 oncogene |
| | | homolog (avian) |
| MAPK8 | Protein | mitogen-activated protein kinase 8 |
| MDM2 | Protein | Mdm2, transformed 3T3 cell double |
| | | minute 2, p53 binding protein (mouse) |
| NBS1 | Protein | nibrin |
| Nuclear | Complex | |
| factor NF | | |
| kappa B | | |
| RAD50 | Protein | RAD50 homolog (S. cerevisiae) |
| RAD51 | Protein | RAD51 homolog (RecA homolog, |
| | | E. coli) (S. cerevisiae) |
| RBBP8 | Protein | retinoblastoma binding protein 8 |
| Replication | Complex | |
| factor A | | |
| S-G2 transition | Cell Process | |
| TP73 | Protein | tumor protein p73 |

*Genes and/or proteins that were used to identify the pathway:
TP53 tumor protein p53 (Li-Fraumeni syndrome)

TABLE 15
Genes/Proteins Involved in the Eda-A1 pathway

| Name | Type | Description |
|---|---|---|
| Apoptosis | Cell Process | |
| CASP8 | Protein | caspase 8, apoptosis-related cysteine |
| | | peptidase |
| EDAR | Protein | ectodysplasin A receptor |
| EDARADD | Protein | EDAR-associated death domain |
| Jnk-mapk | Pathway | |
| NF kappa B | Pathway | |
| RIPK1 | Protein | receptor (TNFRSF)-interacting |
| | | serine-threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |

*Genes and/or proteins that were used to identify the pathway:
HMGCS1 3-hydroxy-3-methylglutaryl-Coenzyme A synthase 1 (soluble)

TABLE 16
Genes/Proteins Involved in the Eda-A2 pathway

| Name | Type | Description |
|---|---|---|
| Apoptosis | Cell Process | |
| CASP8 | Protein | caspase 8, apoptosis-related cysteine |
| | | peptidase |
| Jnk-mapk | Pathway | |
| NE kappa B | Pathway | |
| p40 MAPK | Pathway | |
| RIPK1 | Protein | receptor (TNFRSF)-interacting |
| | | serine-threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |
| TRAF6 | Protein | TNF receptor-associated factor 6 |
| XEDAR | Protein | microtubule-associated protein 2 |

*Genes and/or proteins that were used to identify the pathway:
HMGCS1 3-hydroxy-3-methylglutaryl-Coenzyme A synthase 1 (soluble)

TABLE 17
Genes/Proteins Involved in the Jnk-mapk pathway

| Name | Type | Description |
|---|---|---|
| Abl1 | Protein | v-abl Abelson murine leukemia viral |
| | | oncogene homolog 1 |
| Apoptosis | Cell Process | |
| ATM | Protein | ataxia telangiectasia mutated (includes |
| | | complementation groups A, C and D) |
| BRCA1 | Protein | breast cancer 1, early onset |
| CDKN1A | Protein | cyclin-dependent kinase inhibitor 1A |
| | | (p21, Cip1) |
| CHEK1 | Protein | CHK1 checkpoint homolog (S. pombe) |
| CHEK2 | Protein | CHK2 checkpoint homolog (S. pombe) |
| dna repair | Cell Process | |
| G1-S transition | Cell Process | |
| g2-m transition | Cell Process | |
| GADD45A | Protein | growth arrest and DNA-damage- |
| | | inducible, alpha |
| IkappaB | Complex | |
| JUN | Protein | v-jun sarcoma virus 17 oncogene homolog |
| | | (avian) |
| MAPK8 | Protein | mitogen-activated protein kinase 8 |
| MDM2 | Protein | Mdm2, transformed 3T3 cell double |
| | | minute 2, p53 binding protein (mouse) |
| NBS1 | Protein | nibrin |
| Nuclear | Complex | |
| factor NF | | |
| kappa B | | |
| RAD50 | Protein | RAD50 homolog (S. cerevisiae) |
| RAD51 | Protein | RAD51 homolog (RecA homolog, E. coli) |
| | | (S. cerevisiae) |
| RBBP8 | Protein | retinoblastoma binding protein 8 |
| Replication | Complex | |
| factor A | | |
| S-G2 transition | Cell Process | |
| TP73 | Protein | tumor protein p73 |

*Genes and/or proteins that were used to identify the pathway:
TP53 tumor protein p53 (Li-Fraumeni syndrome)

TABLE 18
Genes/Proteins Involved in the Mitochondrial control of apoptosis pathway

| Name | Type | Description |
|---|---|---|
| 14-3-3 | Functional | |
| | Class | |
| AKT1 | Protein | v-akt murine thymoma viral oncogene homolog 1 |
| APAF1 | Protein | apoptotic peptidase activating factor |
| Apoptosis | Cell Process | |
| apoptosis inhibitor | Functional | |
| | Class | |
| BAD | Protein | BCL2-antagonist of cell death |
| BAX | Protein | BCL2-associated X protein |
| BBC3 | Protein | BCL2 binding component 3 |
| BCL2 | Protein | B-cell CLL/lymphoma 2 |
| BCL2L1 | Protein | BCL2-like 1 |
| BCL2L11 | Protein | BCL2-like 11 (apoptosis facilitator) |
| BID | Protein | BH3 interacting domain death agonist |
| calcineurin | Complex | |
| CASP10 | Protein | caspase 10, apoptosis-related cysteine peptidase |
| CASP3 | Protein | caspase 3, apoptosis-related cysteine peptidase |
| CASP8 | Protein | caspase 8, apoptosis-related cysteine peptidase |
| CASP9 | Protein | caspase 9, apoptosis-related cysteine peptidase |
| CYC1 | Protein | cytochrome c-1 |
| cytokine | Functional | |
| | Class | |
| cytokine_receptor | Functional | |
| | Class | |
| ERK activator | Functional | |
| kinase | Class | |
| FADD | Protein | Fas (TNFRSF6)-associated via death domain |
| FOXO1A | Protein | forkhead box O1A (rhabdomyosarcoma) |
| growth factor | Functional | |
| receptor | Class | |
| growth factors | Functional | |
| | Class | |
| HRK | Protein | harakiri, BCL2 interacting protein (contains only BH3 domain) |
| HSPD1 | Protein | heat shock 60 kDa protein 1 (chaperonin) |
| inositol 1,4,5- | Small | |
| trisphosphate | Molecule | |
| LC8 | Protein | |
| MAPK1 | Protein | mitogen-activated protein kinase 1 |
| MAPK3 | Protein | mitogen-activated protein kinase 3 |
| microtubule | Cell Object | |
| PDCD8 | Protein | programmed cell death 8 (apoptosis-inducing factor) |
| PDPK1 | Protein | 3-phosphoinositide dependent protein kinase-1 |
| Phosphatidylinositol | Complex | |
| 3-kinase | | |
| PKA | Functional | |
| | Class | |
| PKC | Functional | |
| | Class | |
| PMAIP1 | Protein | phorbol-12-myristate-13-acetate-induced protein 1 |
| RAF1 | Protein | v-raf-1 murine leukemia viral oncogene homolog 1 |
| RAS small | Functional | |
| monomeric | Class | |
| GTPase | | |
| RPS6K | Functional | |
| | Class | |
| SMAC | Protein | diablo homolog (Drosophila) |
| TNFRSF6 | Protein | Fas (TNF receptor superfamily, member 6) |
| TNFSF6 | Protein | Fas ligand (TNF superfamily, member 6) |

*Genes and/or proteins that were used to identify the pathway:
TP53 tumor protein p53 (Li-Fraumeni syndrome)

TABLE 19
Genes/Proteins Involved in the p53 signaling pathway

| Name | Type | Description |
|---|---|---|
| APAF1 | Protein | apoptotic peptidase activating factor |
| ATM | Protein | ataxia telangiectasia mutated (includes |
| | | complementation groups A, C and D) |
| BAX | Protein | BCL2-associated X protein |
| BCL2 | Protein | B-cell CLL/lymphoma 2 |
| CCNB1 | Protein | cyclin B1 |
| CCND1 | Protein | cyclin D1 |
| CCNE1 | Protein | cyclin E1 |
| CDK2 | Protein | cyclin-dependent kinase 2 |
| CDK4 | Protein | cyclin-dependent kinase 4 |
| CDKN1A | Protein | cyclin-dependent kinase inhibitor 1A |
| | | (p21, Cip1) |
| E2F1 | Protein | E2F transcription factor 1 |
| GADD45A | Protein | growth arrest and DNA-damage-inducible, |
| | | alpha |
| MDM2 | Protein | Mdm2, transformed 3T3 cell double minute 2, |
| | | p53 binding protein (mouse) |
| proteasome | Complex | |
| RB1 | Protein | retinoblastoma 1 (including osteosarcoma) |
| TIMP3 | Protein | TIMP metallopeptidase inhibitor 3 (Sorsby |
| | | fundus dystrophy, pseudoinflammatory) |
| ubiquitin | Functional | |
| | Class | |

*Genes and/or proteins that were used to identify the pathway:
TP53 tumor protein p53 (Li-Fraumeni syndrome)

TABLE 20
Genes/Proteins Involved in the RB tumor suppressor pathway

| Name | Type | Description |
|---|---|---|
| ATM | Protein | ataxia telangiectasia mutated (includes |
| | | complementation groups A, C and D) |
| CDC2 | Protein | cell division cycle 2, G1 to S and G2 to M |
| CDC25C | Protein | cell division cycle 25C |
| CDK2 | Protein | cyclin-dependent kinase 2 |
| CDK4 | Protein | cyclin-dependent kinase 4 |
| CHEK1 | Protein | CHK1 checkpoint homolog (S. pombe) |
| G1-S transition | Cell Process | |
| g2-m transition | Cell Process | |
| MYT1 | Protein | myelin transcription factor 1 |
| RB1 | Protein | retinoblastoma 1 (including osteosarcoma) |
| WEE1 | Protein | WEE1 homolog (S. pombe) |
| YWHAH | Protein | tyrosine 3-monooxygenase/tryptophan |
| | | 5-monooxygenase activation protein, |
| | | eta polypeptide |

*Genes and/or proteins that were used to identify the pathway:
TP53 tumor protein p53 (Li-Fraumeni syndrome)

### Example 5

### Exemplary Pathways Relating to Sustained High Cellular Productivity

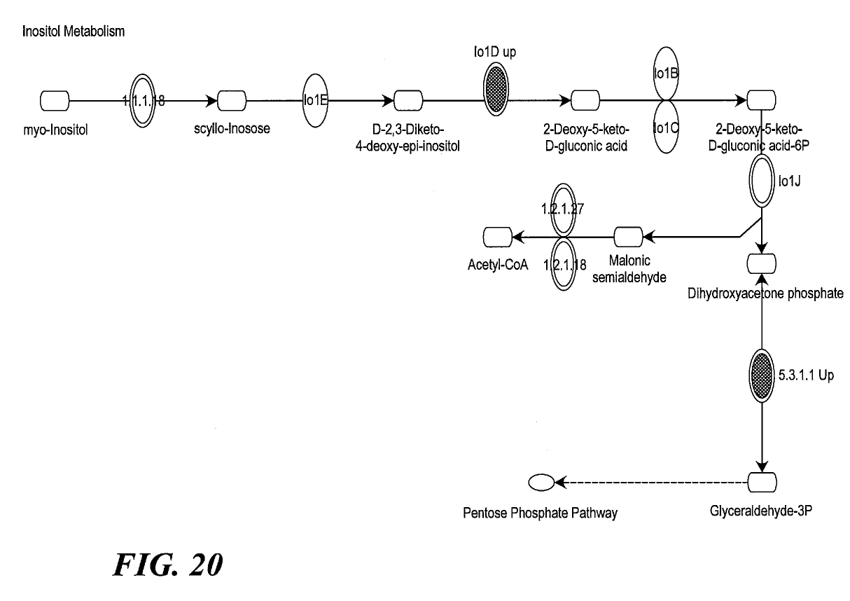

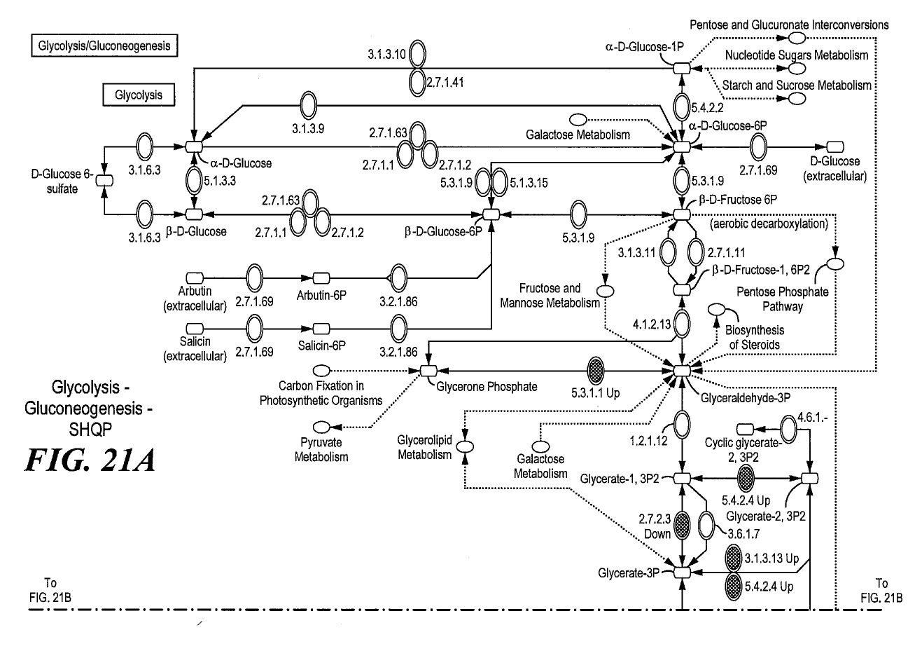

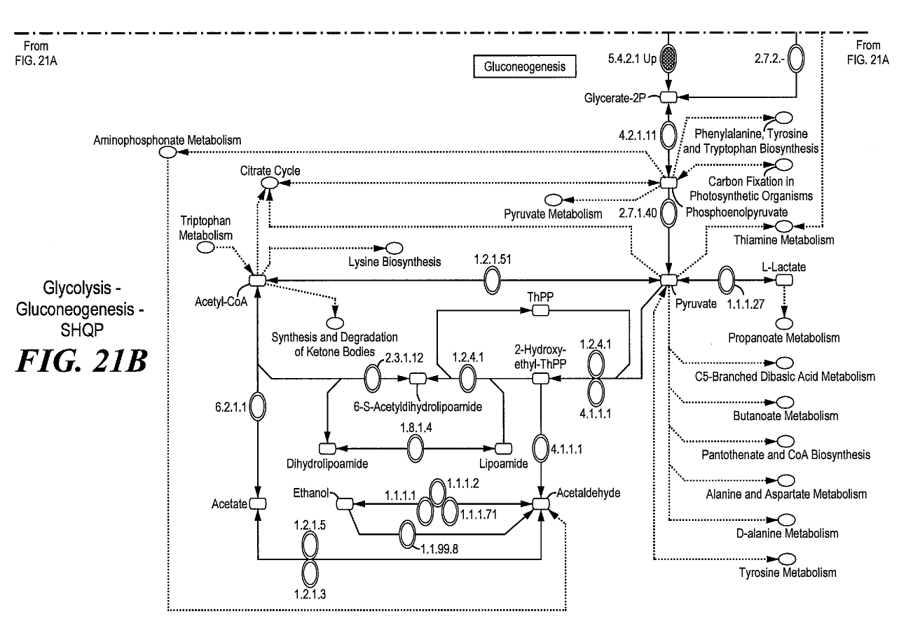

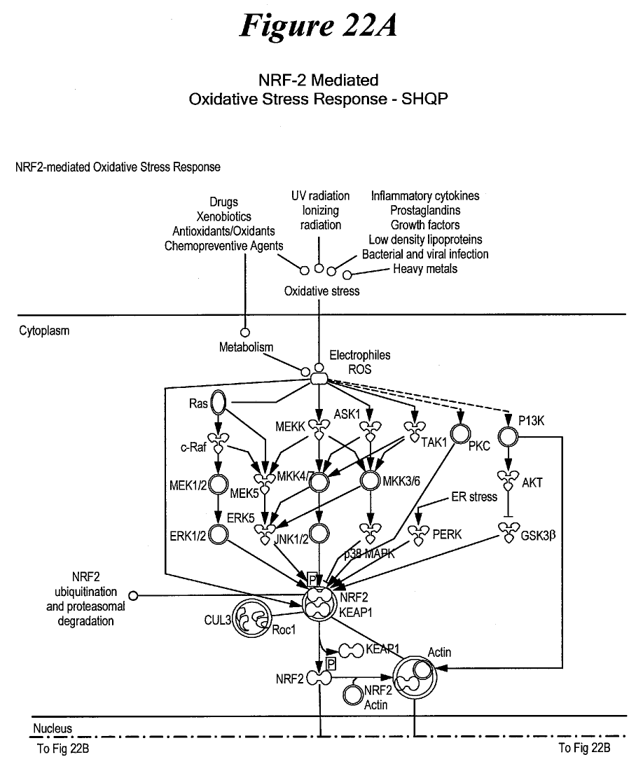

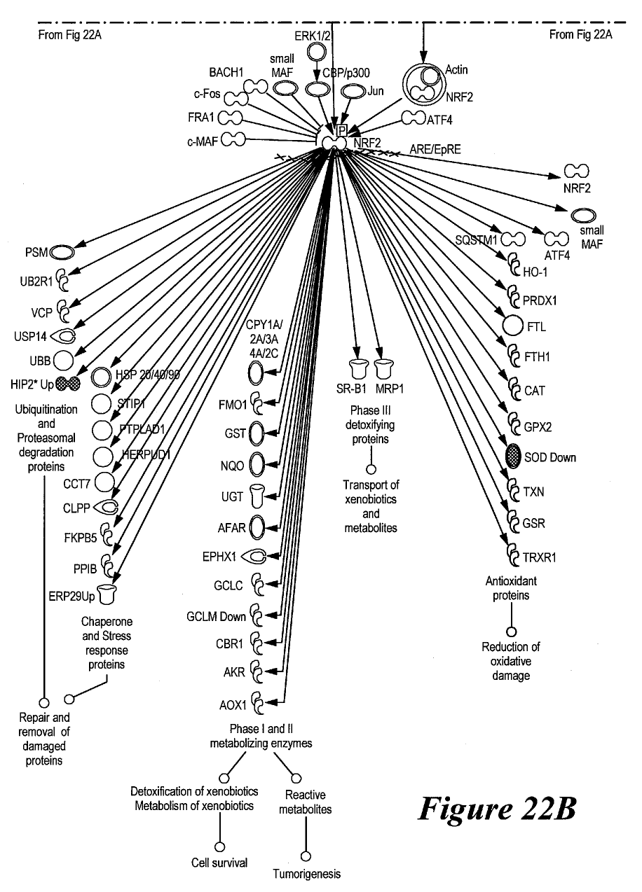

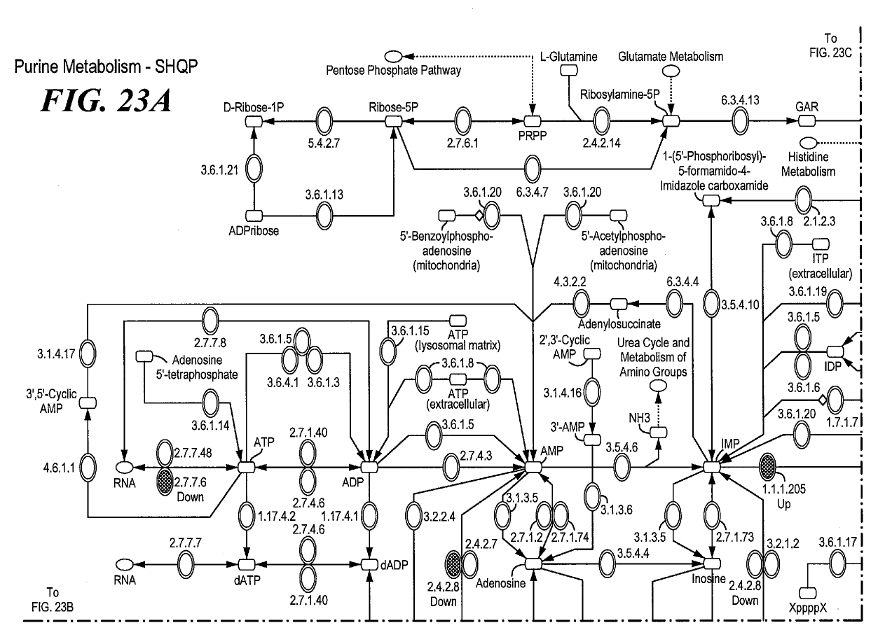

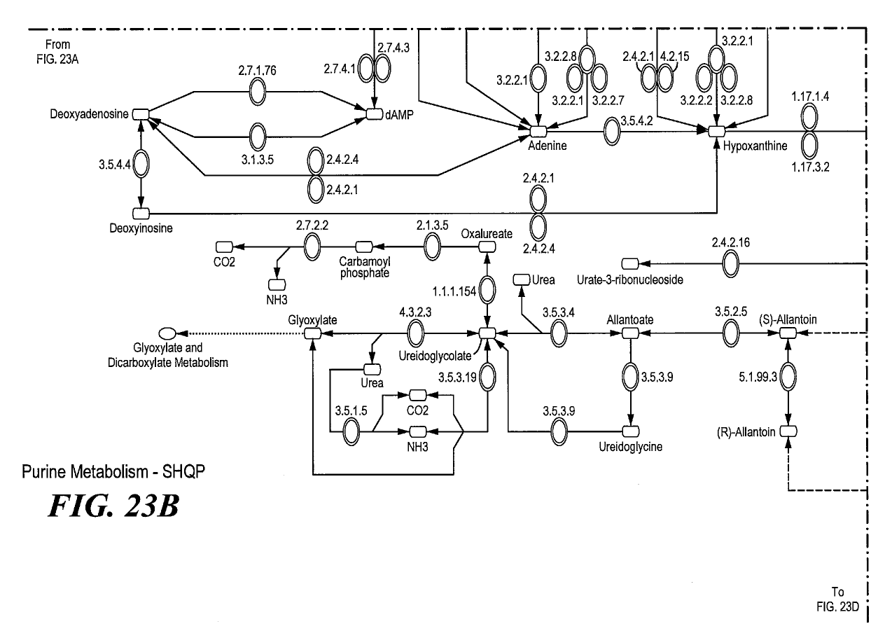

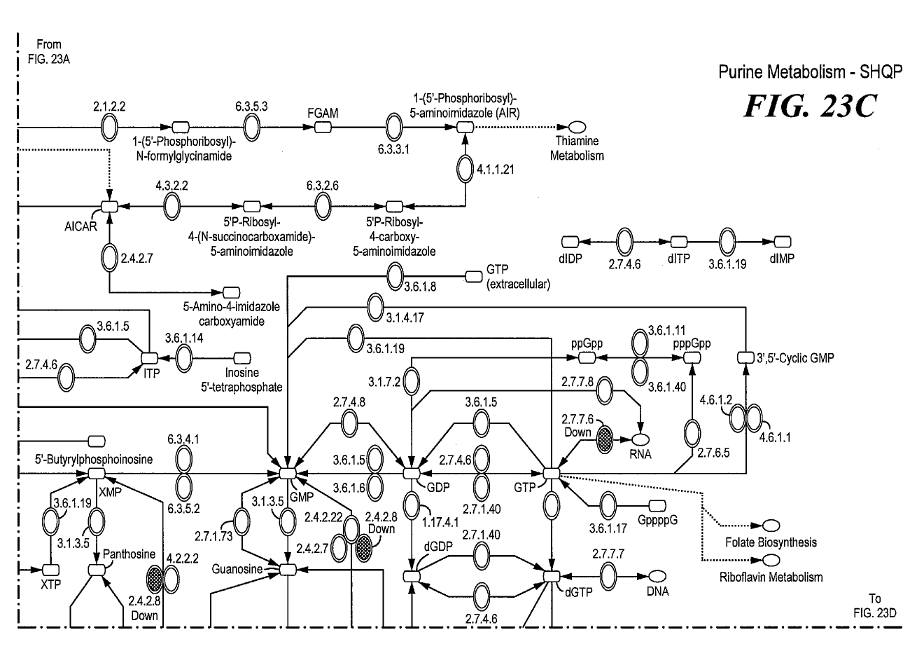

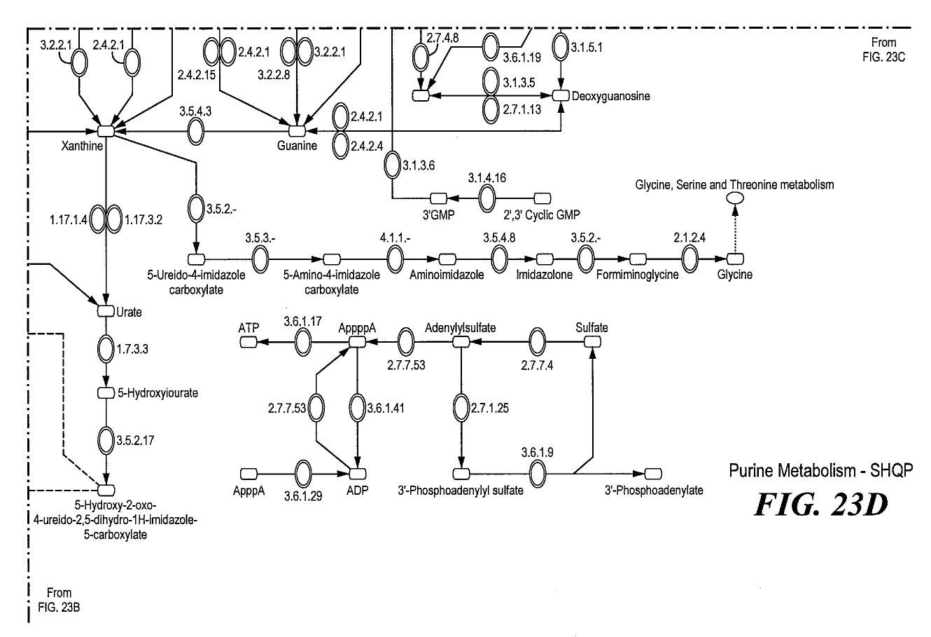

Pathway analysis using Ingenuity software based on previously identified differently expressed genes or proteins associated with high cellular productivity led to the identification of the inositol metabolism pathway (FIG. 20), the glycolysis/gluconeogenesis pathway (FIG. 21), the NRF-mediated oxidative stress response pathway (FIG. 22), and the purine metabolism pathway (FIG. 23). Genes/proteins that were used to identify relevant pathways are indicated in FIGS. 20-23. In addition, additional exemplary genes or proteins involved in the above-identified pathways and that may be involved in regulating or indicative of high cell density are summarized in Table 21 (the inositol metabolism pathway), Table 22 (the glycolysis/gluconeogenesis pathway), Table 23 (the NRF-mediated oxidative stress response pathway), and Table 24 (the purine metabolism pathway).

TABLE 21
Genes/Proteins Involved in the Inositol metabolism pathway

| Name | Synonyms |
|---|---|
| 1.1.1.18 | inositol dehydrogenase, myo-inositol 2-dehydrogenase, myo-inositol |
| | dehydrogenase, myo-inositol:NAD 2-oxidoreductase, myo-inositol:NAD+ |
| | oxidoreductase |
| 1.2.1.18 | 3-oxopropanoate:NAD(P) oxidoreductase (decarboxylating, CoA- |
| | acetylating), malonic semialdehyde oxidative decarboxylase |
| 1.2.1.27 | 2-methyl-3-oxopropanoate:NAD 3-oxidoreductase (CoA-propanoylating) |
| 2-Deoxy-5-keto-D-gluconic acid | (3R,4S)-3,4,6-trihydroxy-5-oxo-hexanoic acid, C6H10O6, DKH |
| 2-Deoxy-5-keto-D-gluconic | (3R,4S)-3,4-dihydroxy-5-oxo-6-phosphonooxy-hexanoic acid, |
| acid-6P | C6H11O9P, DKHP |
| Acetyl-CoA | 72-89-9, acetyl-CoA, C23H38N7O17P3S, coenzyme A, S-acetate, S- |
| | acetyl coenzyme A, [(2R,3R,4R,5R)-2-[[[[3-[2-(2- |
| | acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |
| D-2,3-Diketo-4-deoxy-epi- | (4R,5S,6R)-2,4,5,6-tetrahydroxycyclohex-2-en-1-one, C6H8O5, DKDI |
| inositol | |
| Dihydroxyacetone phosphate | (3-hydroxy-2-oxo-propoxy)phosphonic acid, 1-hydroxy-3- |
| | (phosphonooxy)acetone, 2-propanone, 1-hydroxy-3-(phosphonooxy)-, |
| | 57-04-5, C3H7O6P, DHAP, dihydroxyacetone 3-phosphate, glycerone- |
| | phosphate |
| Glyceraldehyde-3P | 591-57-1, C3H7O6P, D-glyceraldehyde 3-phosphate, [(2R)-2-hydroxy-3- |
| | oxo-propoxy]phosphonic acid |
| Io1J | 1,6-Diphosphofructose aldolase, aldolase, D-fructose-1,6-bisphosphate |
| | D-glyceraldehyde-3-phosphate-lyase, diphosphofructose aldolase, |
| | fructoaldolase, fructose 1,6-diphosphate aldolase, fructose 1- |
| | monophosphate aldolase, fructose 1-phosphate aldolase, fructose |
| | diphosphate aldolase, fructose-1,6-bisphosphate triosephosphate-lyase, |
| | Io1J, ketose 1-phosphate aldolase, phosphofructoaldolase, SMALDO, |
| | zymohexase |
| Malonicsemialdehyde | 3-oxopropanoate, 3-oxopropanoic acid, 926-61-4, C3H4O3, malonate |
| | semialdehyde, propanoic acid, 3-oxo- |
| myo-Inositol | 87-89-8, cis-1,2,3,5-trans-4,6-cyclohexanehexol, i-inositol, inositol, myo-, |
| | inositol, myo-(8Cl), meat sugar |
| scyllo-Inosose | (2S,3R,5S,6R)-2,3,4,5,6-pentahydroxycyclohexan-1-one, 2,4,6/3,5- |
| | pentahydroxycyclohexanone, 2-inosose, C6H10O6 |

*Genes and/or proteins that were used to identify the pathway:
5.3.1.1 D-glyceraldehyde-3-phosphate ketol-isomerase, phosphotriose isomerase, triose phosphate mutase, triose phosphoisomerase
Io1D ALOX12B, ALOX15B, CrtR, CYP4F, DEGS, Io1D, LcyB, LcyE, LysY, SUR2

TABLE 22
Genes/Proteins Involved in the Glycolysis/gluconeogenesis pathway

| Name | Synonyms |
|---|---|
| 1.1.1.1 | ADH, alcohol dehydrogenase (NAD), alcohol:NAD oxidoreductase, aldehyde |
| | reductase, aliphatic alcohol dehydrogenase, ethanol dehydrogenase, NAD- |
| | dependent alcohol dehydrogenase, NAD-specific aromatic alcohol |
| | dehydrogenase, NADH-alcohol dehydrogenase, NADH-aldehyde |
| | dehydrogenase, primary alcohol dehydrogenase, yeast alcohol dehydrogenase |
| 1.1.1.2 | alcohol:NADP oxidoreductase, aldehyde reductase (NADPH2), ALR 1, high-Km |
| | aldehyde reductase, low-Km aldehyde reductase, NADP-alcohol |
| | dehydrogenase, NADP-aldehyde reductase, NADP-dependent aldehyde |
| | reductase, NADPH-aldehyde reductase, NADPH-dependent aldehyde |
| | reductase, nonspecific succinic semialdehyde reductase |
| 1.1.1.27 | (S)-lactate:NAD oxidoreductase, L(+)-nLDH, L-(+)-lactate dehydrogenase, L- |
| | lactic acid dehydrogenase, L-lactic dehydrogenase, lactate dehydrogenase, |
| | lactate dehydrogenase NAD-dependent, lactic acid dehydrogenase, lactic |
| | dehydrogenase, NAD-lactate dehydrogenase |
| 1.1.1.71 | alcohol:NAD(P) oxidoreductase, aldehyde reductase (NADPH/NADH), retinal |
| | reductase |
| 1.1.99.8 | alcohol:(acceptor) oxidoreductase, MDH, primary alcohol dehydrogenase, |
| | quinohemoprotein alcohol dehydrogenase, quinoprotein alcohol |
| | dehydrogenase, quinoprotein ethanol dehydrogenase |
| 1.2.1.12 | 3-phosphoglyceraldehyde dehydrogenase, D-glyceraldehyde-3-phosphate:NAD |
| | oxidoreductase (phosphorylating), dehydrogenase, glyceraldehyde phosphate, |
| | glyceraldehyde phosphate dehydrogenase (NAD), glyceraldehyde-3-P- |
| | dehydrogenase, glyceraldehyde-3-phosphate dehydrogenase (NAD), NAD- |
| | dependent glyceraldehyde phosphate dehydrogenase, NADH-glyceraldehyde |
| | phosphate dehydrogenase, phosphoglyceraldehyde dehydrogenase, |
| | triosephosphate dehydrogenase |
| 1.2.1.3 | aldehyde:NAD oxidoreductase, CoA-independent aldehyde dehydrogenase, m- |
| | methylbenzaldehyde dehydrogenase, NAD-aldehyde dehydrogenase, NAD- |
| | dependent 4-hydroxynonenal dehydrogenase, NAD-dependent aldehyde |
| | dehydrogenase, NAD-linked aldehyde dehydrogenase, propionaldehyde |
| | dehydrogenase |
| 1.2.1.5 | aldehyde:NAD(P) oxidoreductase, ALDH |
| 1.2.1.51 | pyruvate:NADP 2-oxidoreductase (CoA-acetylating) |
| 1.2.4.1 | MtPDC (mitochondrial pyruvate dehydogenase complex), PDH, pyruvate |
| | decarboxylase, pyruvate dehydrogenase, pyruvate dehydrogenase complex, |
| | pyruvate:lipoamide 2-oxidoreductase (decarboxylating and acceptor- |
| | acetylating), pyruvic acid dehydrogenase, pyruvic dehydrogenase |
| 1.8.1.4 | dehydrolipoate dehydrogenase, diaphorase, dihydrolipoamide:NAD |
| | oxidoreductase, dihydrolipoic dehydrogenase, dihydrolipoyl dehydrogenase, |
| | dihydrothioctic dehydrogenase, LDP-Glc, LDP-Val, lipoamide dehydrogenase |
| | (NADH), lipoamide oxidoreductase (NADH), lipoamide reductase, lipoamide |
| | reductase (NADH2), lipoate dehydrogenase, lipoic acid dehydrogenase, lipoyl |
| | dehydrogenase |
| 2-Hydroxy-ethyl-ThPP | 2-(1-hydroxyethyl)thiamine pyrophosphate, C14H23N4O8P2S+, [2-[3-[(4- |
| | amino-2-methyl-pyrimidin-5-yl)methyl]-2-(1-hydroxyethyl)-4-methyl-1-thia-3- |
| | azoniacyclopenta-2,4-dien-5-yl]ethoxy-hydroxy-phosphoryl]oxyphosphonic acid |
| 2.3.1.12 | acetyl-CoA:dihydrolipoamide S-acetyltransferase, dihydrolipoate |
| | acetyltransferase, dihydrolipoic transacetylase, dihydrolipoyl acetyltransferase, |
| | lipoate acetyltransferase, lipoate transacetylase, lipoic acetyltransferase, lipoic |
| | acid acetyltransferase, lipoic transacetylase, lipoylacetyltransferase, |
| | thioltransacetylase A, transacetylase X |
| 2.7.1.1 | ATP-dependent hexokinase, ATP:D-hexose 6-phosphotransferase, glucose |
| | ATP phosphotransferase, hexokinase (phosphorylating), hexokinase D, |
| | hexokinase type IV, hexokinase type IV glucokinase |
| 2.7.1.11 | 6-phosphofructose 1-kinase, ATP-dependent phosphofructokinase, ATP:D- |
| | fructose-6-phosphate 1-phosphotransferase, D-fructose-6-phosphate 1- |
| | phosphotransferase, fructose 6-phosphate kinase, fructose 6-phosphokinase, |
| | nucleotide triphosphate-dependent phosphofructokinase, PFK, phospho-1,6- |
| | fructokinase, phosphofructokinase (phosphorylating), phosphofructokinase I, |
| | phosphohexokinase |
| 2.7.1.2 | ATP:D-glucose 6-phosphotransferase, glucokinase (phosphorylating) |
| 2.7.1.40 | ATP:pyruvate 2-O-phosphotransferase, fluorokinase, fluorokinase |
| | (phosphorylating), phosphoenol transphosphorylase pyruvate kinase |
| | (phosphorylating), phosphoenolpyruvate kinase, Pk, pyruvate |
| | phosphotransferase, pyruvic kinase |
| 2.7.1.41 | D-glucose-1-phosphate:D-glucose-1-phosphate 6-phosphotransferase, glucose |
| | 1-phosphate transphosphorylase, phosphodismutase |
| 2.7.1.63 | polyphosphate glucokinase, polyphosphate-D-(+)-glucose-6- |
| | phosphotransferase, polyphosphate-glucose 6-phosphotransferase, |
| | polyphosphate:D-glucose 6-phosphotransferase |
| 2.7.1.69 | enzyme III4ac, gene bgIC RNA formation factors, gene gIC proteins, glucose |
| | permease, PEP-dependent phosphotransferase enzyme II, PEP-sugar |
| | phosphotransferase enzyme II, phosphoenolpyruvate-sugar |
| | phosphotransferase enzyme II, phosphohistidinoprotein-hexose |
| | phosphoribosyltransferase, phosphohistidinoprotein-hexose |
| | phosphotransferase, phosphoprotein factor-hexose phosophotransferase, |
| | phosphotransferase, phosphohistidinoprotein-hexose, protein, specific or class, |
| | gene bgIC, protein-Np-phosphohistidine:sugar N-pros-phosphotransferase, PTS |
| | permease, ribonucleic acid formation factor, gene gIC, sucrose |
| | phosphotransferase system II |
| 2.7.2.— | LysZ |
| 3.1.3.10 | D-glucose-1-phosphate phosphohydrolase |
| 3.1.3.11 | D-fructose 1,6-diphosphatase, D-fructose-1,6-bisphosphate 1- |
| | phosphohydrolase, D-fructose-1,6-bisphosphate phosphatase, F1,6pase, |
| | FBPase, fructose 1,6-bisphosphatase, fructose 1,6-bisphosphate 1- |
| | phosphatase, fructose 1,6-bisphosphate phosphatase, fructose 1,6- |
| | diphosphatase, fructose 1,6-diphosphate phosphatase, fructose bisphosphate |
| | phosphatase, fructose diphosphatase, fructose diphosphate phosphatase, |
| | Fructose-bisphosphatase, hexose bisphosphatase, hexose diphosphatase |
| 3.1.3.9 | D-glucose-6-phosphate phosphohydrolase, glucose 6-phosphate phosphatase |
| 3.1.6.3 | glucosulfatase, sugar-sulfate sulfohydrolase |
| 3.2.1.86 | 6-phospho-beta-D-glucosyl-(1,4)-D-glucose glucohydrolase, phospho-beta- |
| | glucosidase, phospho-beta-glucosidase A, phosphocellobiase |
| 3.6.1.7 | 1,3-diphosphoglycerate phosphatase, acetic phosphatase, acetylphosphatase, |
| | acylphosphate phosphohydrolase, GP 1-3, Ho 1-3 |
| 4.1.1.1 | 2-oxo-acid carboxy-lyase, alpha-carboxylase, alpha-ketoacid carboxylase, |
| | pyruvic decarboxylase |
| 4.1.2.13 | 1,6-Diphosphofructose aldolase, aldolase, D-fructose-1,6-bisphosphate D- |
| | glyceraldehyde-3-phosphate-lyase, diphosphofructose aldolase, fructoaldolase, |
| | fructose 1,6-diphosphate aldolase, fructose 1-monophosphate aldolase, |
| | fructose 1-phosphate aldolase, fructose diphosphate aldolase, fructose-1,6- |
| | bisphosphate triosephosphate-lyase, lo1J, ketose 1-phosphate aldolase, |
| | phosphofructoaldolase, SMALDO, zymohexase |
| 4.2.1.11 | 14-3-2-protein, 2-phospho-D-glycerate hydro-lyase, 2-phosphoglycerate |
| | dehydratase, 2-phosphoglycerate enolase, 2-phosphoglyceric dehydratase, g- |
| | enolase, nervous-system specific enolase, phosphoenolpyruvate hydratase, |
| | Phosphopyruvate hydratase |
| 4.6.1.— | |
| 5.1.3.15 | D-glucose-6-phosphate 1-epimerase |
| 5.1.3.3 | aldose mutarotase, mutarotase |
| 5.3.1.9 | D-glucose-6-phosphate ketol-isomerase, glucose phosphate isomerase, hexose |
| | phosphate, hexosephosphate isomerase, oxoisomerase, |
| | phosphoglucoisomerase, phosphoglucose isomerase, phosphohexoisomerase, |
| | phosphohexomutase, phosphohexose isomerase, phosphosaccharomutase |
| 5.4.2.2 | alpha-D-glucose 1,6-phosphomutase, glucose phosphomutase, |
| | Phosphoglucomutase, phosphoglucose mutase |
| 6-S- | 6-acetylsulfanyl-8-sulfanyl-octanamide, 6-S-acetyldihydrolipoamide, |
| Acetyldihydrolipoamide | C10H19NO2S2 |
| 6.2.1.1 | acetate thiokinase, acetate:CoA ligase (AMP-forming), acetyl activating |
| | enzyme, acetyl-CoA synthetase, acyl-activating enzyme |
| Acetaldehyde | 75-07-0, acetaldehyde, C2H4O, ethyl aldehyde |
| Acetate | 64-19-7, Acetasol, acetic acid, C2 short-chain fatty acid, C2H4O2, ethanoic |
| | acid, glacial acetic acid, Vasotate, Vosol |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl-propoxy]- |
| | hydroxy-phosphoryl]oxy-hydroxy-phosphoryl]oxymethyl]-5-(6-aminopurin-9-yl)- |
| | 4-hydroxy-oxolan-3-yl]oxyphosphonic acid |
| Arbutin (extracellular) | (2R,3R,4S,5R,6S)-2-(hydroxymethyl)-6-(4-hydroxyphenoxy)oxane-3,4,5-triol, 4- |
| | hydroxyphenyl-beta-D-glucopyranoside, 497-76-7, arbutoside, beta-D- |
| | glucopyranoside, 4-hydroxyphenyl-, beta-D-glucopyranoside, 4-hydroxyphenyl- |
| | (9Cl), C12H16O7, p-hydroxyphenyl beta-D-glucopyranoside, p-hydroxyphenyl |
| | beta-D-glucoside, ursin, uvasol |
| Arbutin-6P | C12H17O10P, [(2R,3R,4S,5R,6S)-3,4,5-trihydroxy-6-(4-hydroxyphenoxy)oxan- |
| | 2-yl]methoxyphosphonic acid |
| Cyclic glycerate-2,3P2 | (6R)-2,4-dihydroxy-2,4-dioxo-1,3,5-trioxa- |
| | 2&lambda;<sup>5</sup>,4&lambda;<sup>5</sup>-diphosphacycloheptane-6- |
| | carboxylic acid, 1,3,5,2,4-Trioxadiphosphepane-6-carboxylic acid, 2,4- |
| | dihydroxy-, 2,4-dioxide, (R)-, 88280-54-0, C3H6O9P2, cDPG, cyclic 2,3- |
| | diphospho-D-glycerate, cyclic glycerate-2,3P2 |
| D-Glucose 6-sulfate | (2R,3R,4S,5R,6R)-2,3,4,5-tetrahydroxy-6-(sulfooxymethyl)oxane, C6H12O9S |
| Dihydrolipoamide | 3884-47-7, 6,8-bis-sulfanyloctanamide, 6,8-dimercapto-octanamide, |
| | C8H17NOS2, dihydrothioctamide, octanamide, 6,8-dimercapto- |
| Ethanol | 1-hydroxyethane, 64-17-5, C2H6O, ethanol, ethyl alcohol |
| Glyceraldehyde-3P | 591-57-1, C3H7O6P, D-glyceraldehyde 3-phosphate, [(2R)-2-hydroxy-3-oxo- |
| | propoxy]phosphonic acid |
| Glycerate-1,3P2 | 3-phospho-D-glyceroyl phosphate, 38168-82-0, C3H8O10P2, [(2R)-2-hydroxy- |
| | 2-phosphonooxycarbonyl-ethoxy]phosphonic acid |
| Glycerate-2,3P2 | (2R)-2,3-diphosphonooxypropanoic acid, (2S)-2,3-diphosphonooxypropanoic |
| | acid, 14438-19-8, 2,3-bisphospho-D-glycerate, C3H8O10P2 |
| Glycerate-2P | (2R)-3-hydroxy-2-phosphonooxy-propanoic acid, 3443-57-0, C3H7O7P, D- |
| | glycerate 2-phosphate, PGA |
| Glycerate-3P | (2R)-2-hydroxy-3-phosphonooxy-propanoic acid, C3H7O7P, D-glycerate 3- |
| | phosphate |
| Glycerone phosphate | (3-hydroxy-2-oxo-propoxy)phosphonic acid, 1-hydroxy-3- |
| | (phosphonooxy)acetone, 2-propanone, 1-hydroxy-3-(phosphonooxy)-, 57-04-5, |
| | C3H7O6P, DHAP, dihydroxyacetone 3-phosphate, glycerone-phosphate |
| L-Lactate | (2S)-2-hydroxypropanoic acid, (S)-lactate, 79-33-4, C3H6O3, L-(+)-lactic acid, |
| | L-2-hydroxypropionic acid, L-lactate, propanoic acid, 2-hydroxy-, (2S)-, S-lactic |
| | acid |
| Lipoamide | 1,2-dithiolane-3-pentanamide, 1,2-dithiolane-3-pentanamide (9Cl), 1,2- |
| | dithiolane-3-valeramide, 5-(1,2-dithiolan-3-yl)valeramide, 5-(dithiolan-3- |
| | yl)pentanamide, 6,8-thioctic amide, 940-69-2, alpha-lipoic acid amide, alpha- |
| | lipoic amide, C8H15NOS2, vitamin N |
| Phosphoenolpyruvate | 138-08-9, 2-(phosphonooxy)acrylate, 2-dihydroxyphosphinoyloxyacrylic acid, 2- |
| | phosphonooxyprop-2-enoic acid, 2-propenoic acid, 2-(phosphonooxy)-, 2- |
| | propenoic acid, 2-(phosphonooxy)-, ion(1-), 2-propenoic acid, 2- |
| | (phosphonooxy)-, monopotassium salt, 4265-07-0, 73-89-2, C3H5O6P, |
| | phosphopyruvic acid |
| Pyruvate | 127-17-3, 2-oxopropanoate, 2-oxopropanoic acid, 57-60-3, C3H4O3, propanoic |
| | acid, 2-oxo-, propanoic acid, 2-oxo-, ion(1-), propanoic acid, 2-oxo-, sodium |
| | salt, pyruvate, pyruvic acid, sodium salt, sodium pyruvate |
| Salicin (extracellular) | (2R,3R,4S,5R,6S)-2-(hydroxymethyl)-6-[2-(hydroxymethyl)phenoxy]oxane- |
| | 3,4,5-triol, 138-52-3, 2-(hydroxymethyl)phenyl beta-D-glucopyranoside, alpha- |
| | hydroxy-o-tolyl beta-D-glucopyranoside, beta-D-glucopyranoside, 2- |
| | (hydroxymethyl)phenyl, C13H18O7, D-(−)-salicin, o-(hydroxymethyl)phenyl beta- |
| | D-glucopyranoside, salicin (6Cl,8Cl), salicoside, salicyl alcohol glucoside, |
| | saligenin-beta-D-glucopyranoside |
| Salicin-6P | C13H19O10P, salicin-6P, [(2R,3R,4S,5R,6S)-3,4,5-trihydroxy-6-[2- |
| | (hydroxymethyl)phenoxy]oxan-2-yl]methoxyphosphonic acid |
| ThPP | 136-09-4, 154-87-0, 23883-45-6, C12H19N4O7P2S+, cocarboxylase, thiamin |
| | diphosphate, thiamine diphosphate hydrochloride, thiazolium, 3-((4-amino-2- |
| | methyl-5-pyrimidinyl)methyl)-4-methyl-5-(4,6,6-trihydroxy-3,5-dioxa-4,6- |
| | diphosphahex-1-yl)-, chloride, P,P′-dioxide, [2-[3-[(4-amino-2-methyl-pyrimidin- |
| | 5-yl)methyl]-4-methyl-1-thia-3-azoniacyclopenta-2,4-dien-5-yl]ethoxy-hydroxy- |
| | phosphoryl]oxyphosphonic acid |
| &alpha;-D-Glucose | (2S,3R,4S,5R,6R)-6-(hydroxymethyl)oxane-2,3,4,5-tetrol, 26655-34-5, |
| | C6H12O6 |
| &alpha;-D-Glucose-1P | 59-56-3, C6H13O9P, glucose 1-(dihydrogen phosphate), [(3R,4S,5S,6R)-3,4,5- |
| | trihydroxy-6-(hydroxymethyl)oxan-2-yl]oxyphosphonic acid |
| &alpha;-D-Glucose-6P | C6H13O9P, [(2R,3R,4S,5R,6S)-3,4,5,6-tetrahydroxyoxan-2- |
| | yl]methoxyphosphonic acid |
| &beta;-D-Fructose 6P | 41452-29-3, C6H13O9P, [(2R,3R,4S,5R)-3,4,5-trihydroxy-5- |
| | (hydroxymethyl)oxolan-2-yl]methoxyphosphonic acid |
| &beta;-D-Fructose- | 34693-15-7, beta-D-fructose 1,6-bisphosphate, C6H14O12P2, [(2R,3S,4R,5R)- |
| 1,6P2 | 2,3,4-trihydroxy-5-(phosphonooxymethyl)oxolan-2-yl]methoxyphosphonic acid |
| &beta-D-Glucose | (3R,4S,5S,6R)-6-(hydroxymethyl)oxane-2,3,4,5-tetrol, 50-99-7, C6H12O6, D- |
| | glucose, dextrose, glucose, sugar |
| &beta;-D-Glucose-6P | C6H13O9P, [(2R,3R,4S,5R,6R)-3,4,5,6-tetrahydroxyoxan-2- |
| | yl]methoxyphosphonic acid |

*Genes and/or proteins that were used to identify the pathway:
2.7.2.3 3-PGK, 3-phosphoglycerate kinase, 3-phosphoglycerate phosphokinase, 3-phosphoglyceric acid kinase, 3-phosphoglyceric acid phosphokinase, 3-phosphoglyceric kinase, ATP-3-phospho-D-glycerate-1-phosphotransferase, ATP:3-phospho-D-glycerate 1-phosphotransferase, ATP:D-3-phosphoglycerate 1-phosphotransferase, glycerate 3-phosphate kinase, glycerophosphate kinase, Phosphoglycerate kinase, phosphoglyceric acid kinase, phosphoglyceric kinase, phosphoglycerokinase
3.1.3.13 2,3-bisphospho-D-glycerate 2-phosphohydrolase, 2,3-bisphosphoglycerate phosphatase, 2,3-diphosphoglycerate phosphatase, 2,3-diphosphoglyceric acid phosphatase, diphosphoglycerate phosphatase, glycerate-2,3-diphosphate phosphatase
5.3.1.1 D-glyceraldehyde-3-phosphate ketol-isomerase, phosphotriose isomerase, triose phosphate mutase, triose phosphoisomerase
5.4.2.1 bisphosphoglyceromutase, D-phosphoglycerate 2,3-phosphomutase, diphosphoglycerate mutase, diphosphoglycomutase, glycerate phosphomutase (diphosphoglycerate cofactor), GriP mutase, monophosphoglycerate mutase, monophosphoglyceromutase, MPGM, PGA mutase, PGAM, PGAM-d, PGAM-i, PGM, Phosphoglycerate mutase, phosphoglycerate phosphomutase, phosphoglyceromutase
5.4.2.4 2,3-bisphosphoglycerate mutase, 2,3-diphosphoglycerate mutase, 2,3-diphosphoglycerate synthase, 2,3-diphosphoglyceromutase, 3-phospho-D-glycerate 1,2-phosphomutase, biphosphoglycerate synthase, bisphosphoglycerate synthase, bisphosphoglyceromutase, BPGM, diphosphoglycerate mutase, diphosphoglyceric mutase, diphosphoglyceromutase, DPGM, glycerate phosphomutase, phosphoglyceromutase

TABLE 23
Genes/Proteins Involved in the NRF-2 mediated oxidative stress response pathway

| Name | Synonyms |
|---|---|
| Actin | G-actin |
| Actin-Nrf2 | |
| AFAR | AFLATOXIN B1 ALDEHYDE REDUCTASE |
| AKR | 2610201A18Rik, Akr1a4, Akra, ALDEHYDE REDUCTASE, ALDR1, ALR, DD3, |
| | MGC12529, MGC1380 |
| AKT | AKT, MGC99656, PKB, PKB-ALPHA, PKB/AKT, PRKBA, Protein kinase B, RAC, |
| | RAC-ALPHA, Thymoma viral proto-oncogene 1 |
| AOX1 | AI196512, AI255253, ALDEHYDE OXIDASE, AO, AOH1, Aox-2, MGC13774, Moro, |
| | RO, XD |
| ASK1 | 7420452D20Rik, APOPTOSIS SIGNAL REGULATED KINASE 1, ASK, ASK1, |
| | Map3k5, MAPKKK5, MEKK5, MGC141518, MGC141519, RGD1306565 predicted, |
| | RGD1306565_predicted |
| ATF4 | C/ATF, CREB-2, MGC96460, TAXREB67, TXREB |
| BACH1 | 6230421P05RIK, AI323795, C21ORF41 |
| c-Fos | AP-1, C-FOS, D12Rfj1, V-FOS |
| c-MAF | 2810401A20Rik, A230108G15RIK, AW047063, C-MAF, Maf2, MGC71685 |
| c-Raf | 6430402F14Rik, AA990557, BB129353, C-RAF, C-RAF1, D830050J10Rik, |
| | MGC102375, MURINE LEUKEMIA VIRAL ONCOGENE HOMOLOG1, NS5, Raf, |
| | RAF KINASE, v-Raf |
| CAT | 2210418N07, Cas-1, CATALASE, Catalase1, Cs-1, MGC128112, MGC138422, |
| | MGC138424, RATCAT01, RATCATL |
| CBP/p300 | CBP |
| CBR1 | AW261796, Carbonyl Reductase, CBR, CR, hCBR1, MGC124927, Ocr |
| CCT7 | AA408524, AL022769, CCT-ETA, Ccth, Cctz, Chaperonin subunit 7, MGC110985, |
| | Nip7-1, TCP-1-eta |
| CLPP | AU019820, D17Wsu160e |
| CUL3 | AI467304, AW146203, mKIAA0617 |
| Cul3-Roc1 | |
| CYP1A/2A/3A/4A/2C | |
| ElectophilesROS | oxygen and reactive oxygen species, reactive oxygen metabolites, ROI, ROS |
| EPHX1 | AI195553, Ehm, Eph-1, EPHX, EPOX, Epoxide Hydrolase, EPXH1, MEH, MEH8, |
| | MICROSOMAL EPOXIDE HYDROLASE |
| ERK1/2 | |
| ERK5 | BMK, BMK1, ERK4, ERK5, Erk5-T, ERK7, PRKM7 |
| ERP29 | 1200015M03Rik, 2810446M09Rik, AW209030, C12ORF8, ERp28, ERp31, PDI-DB |
| FKBP5 | 51kDa, AIG6, D17Ertd592e, Dit1, FKBP51, Fkbp51/54, FKBP54, MGC111006, |
| | P54, PPlase, Ptg-10 |
| FMO1 | Flavin-containing monooxygenase, RFMO1A |
| FRA1 | AW538199, FRA, FRA-1 |
| FTH1 | AL022624, AL033366, APOFERRITIN H CHAIN, FERRITIN H, FERRITIN H |
| | CHAIN, Ferritin heavy chain, Ferritin subunit H, FHC, FTH, FTHL6, H FERRITIN, |
| | Hcf, MFH, MGC104426, PIG15, PLIF |
| FTL | FERRITIN LIGHT CHAIN, FTL1, Ftl2, L-FERRITIN, MGC102130, MGC102131, |
| | MGC118079, MGC118080, MGC71996, RGD1560687 predicted, |
| | RGD1560687_predicted, RGD1561055 predicted, RGD1561055_predicted, |
| | RGD1566189 predicted, RGD1566189_predicted, YB24D08 |
| GCLC | D9Wsu168e, GAMMA GCS HEAVY CHAIN, Gamma Glutamyl Cysteine Synthetase |
| | Light Subunit, Gamma Glutamylcysteine Synthetase, Gamma glutamylcysteine |
| | synthetase heavy subunit, GAMMA-GCS, GAMMA-GCSH, Gcl, GCS, GCS, |
| | Catalytic, GCS-HS, GCSH, Ggcs-hs, GLCL, GLCL-H, GLCLC, MGC93096 |
| GCLM | AI649393, Gamma gclm, GAMMA GCS LIGHT CHAIN, Gamma glutamylcysteine |
| | synthase (regulatory), GAMMA GLUTAMYLCYSTEINE SYNTHETASE, Gcs Ls, |
| | Gcs, Regulatory, GCS-L, GCS1, Gcslc, GLCLR, glutamat-cystein ligase, regulatory |
| | subunit |
| GPX2 | GI-GPx, GPRP, GPX-GI, GSHPx-2, GSHPX-GI |
| GSK3&beta; | 7330414F15Rik, 8430431H08Rik, C86142, GSK-3, GSK-3BETA, Tpk1 |
| GSR | AI325518, D8Ertd238e, GLUTATHIONE REDUCTASE, Gr, Gr-1, Gred, GRX, |
| | MGC78522 |
| GST | Glutathione s-transferase |
| HERPUD1 | HERP, KIAA0025, Mif1, MifI, SUP |
| HO-1 | bK286B10, D8Wsu38e, HEME OXYGENASE (DECYCLIZING) 1, HEME |
| | OXYGENASE-1, Hemox, Heox, HEOXG, Hmox, HO-1, HSP32 |
| HSP22/40/90 | |
| JNK1/2 | JNK1/2 |
| Jun | JUN |
| KEAP1 | INRF2, KIAA0132, KLHL19, MGC10630, MGC1114, MGC20887, MGC4407, |
| | MGC9454, mKIAA0132 |
| Keap1-Nrf2 | |
| MEK1/2 | MEK1/2, Mkk 1/2 |
| MEK5 | AI324775, AI428457, HsT17454, MAP kinase kinase 5, MAPKK5, MEK5, MKK5, |
| | PRKMK5 |
| MEKK | MAPK, MAPKKK1, MEK KINASE, MEK KINASE 1, MEKK, MEKK1, Raf |
| MKK3/6 | Mkk3/6 (mitogen activated protein kinase kinase 3/6), MKK3/MKK6 |
| MKK4/7 | MKK4/7 |
| MRP1 | ABC29, ABCC, Abcc1a, Abcc1b, Avcc1a, DKFZp686N04233, DKFZp781G125, |
| | GS-X, Mdrap, MRP, MRP1 |
| NQO | NADPH QUINONE OXIDOREDUCTASE, Nadph-d |
| NRF2 | AI194320, NRF2 |
| p38 MAPK | CRK1, CSBP, CSBP1, CSBP2, CSPB1, EXIP, Hog, MAPK p38, MGC102436, |
| | MGC105413, MXI2, P38, P38 KINASE, P38 Map Kinase, p38 Mapk alpha, P38- |
| | ALPHA, p38-RK, p38/Hog1, p38/Mpk2, P38/RK, p38a, p38Hog, p38MAPK, |
| | PRKM14, PRKM15, RK, SAPK2A |
| PERK | AI427929, DKFZp781H1925, HRI, PEK, PERK, WRS |
| PI3K | Pi 3-kinase |
| PKC | Cnpkc, Pkc, PKC protein, Pkm, Protein kinase c |
| PPIB | AA408962, AA553318, AI844835, CPHN2, Cy-Lp, CYCLOPHILIN-B, CyP-20b, |
| | CYP-S1, CYPB, MGC14109, MGC2224, SCYLP |
| PRDX1 | ENHANCER PROTEIN, Hbp23, MGC108617, MSP23, NKEFA, OSF-3, PAG, |
| | PAGA, PAGB, PEROXIREDOXIN 1, Prdx-I, PRX I, PRX1, TDPX2, TDX2, TPx-A, |
| | TPX2 |
| PSM | |
| PTPLAD1 | 4930523M17RIK, AW742319, B-IND1, FLJ90376, HSPC121, MGC25483 |
| Ras | p21 Ras, p21 Ras protein, Ras protein |
| Roc1 | 1500002P15Rik, AA517855, BA554C12.1, ENSMUSG00000049832, HRT1, |
| | MGC13357, MGC1481, RBX1, RNF75, ROC1 |
| small MAF | MAF, SMALL MAF |
| SQSTM1 | A170, OSF-6, Osi, OSIL, Oxidative Stress Protein, p60, P62, p62B, PDB3, Pkc zeta |
| | interacting protein, STAP, Ubiquitin-binding protein a, ZIP, ZIP3 |
| SR-BI | AI120173, CD36, CD36L1, CLA-1, D5Ertd460e, HDL Receptor, MGC138242, mSR- |
| | BI, Scavenger receptor class b1, SR-B, SR-B1, SR-BI |
| STIP1 | HOP, IEF-SSP-3521, mSTI1, P60, SIP1, STI1, STI1L, Stress-induced |
| | phosphoprotein 1 |
| TAK1 | B430101B05, C87327, Map3k7 predicted, Map3k7_predicted, TAK1, Tgf beta |
| | Activated Kinase1, TGF1A |
| TRXR1 | GRIM-12, KM 102 DERIVED REDUCTASE LIKE FACTOR, MGC9145, MGC93353, |
| | Tgr, Thioredoxin reductase, TR, TR1, TRXR1, TXNR |
| TXN | ADF, AW550880, DKFZp686B1993, EOSINOPHIL CYTOTOXICITY FACTOR, |
| | MGC151960, MGC61975, THIOREDOXIN, TRX, TRX1, Txn1 |
| UB2R1 | AI327276, Cdc34, E2-CDC34, UBC3, UBE2R1, Ubiquitin conjugating enzyme e2- |
| | 32 |
| UBB | AL033289, FLJ25987, Loc192255, MGC8385, Polyubiquitin ub2, Ubb2, UBC, |
| | UBIQUITIN, UBIQUITIN B |
| UGT | AI327289, Had-1, MGC188623, Sfc8, UGALT, UGAT, UGT, UGT1, UGT2, UGTL |
| USP14 | 2610005K12Rik, AW107924, ax, C78769, MGC95160, TGT, Ubiquitin specific |
| | protease 14 |
| VCP | 3110001E05, CDC48, IBMPFD, MGC131997, MGC148092, MGC8560, P97, P97 |
| | kinase, p97/VCP, Ter atpase, TERA, TRANSITIONAL ENDOPLASMIC |
| | RETICULUM ATPASE, XSG7 |

*Genes and/or proteins that were used to identify the pathway:
HIP2 AW492011, D5Ertd601e, DKFZp564C1216, DKFZp686J24237, E2-25K, HIP2, HYPG, LIG, UBIQUITIN CARRIER PROTETN
SOD copper-zinc superoxide dismutase, Cu,Zn-SOD, Cu—Zn superoxide dismutase, cuprein, cytocuprein, erythrocuprein, Fe-SOD, ferrisuperoxide dismutase, hemocuprein, hepatocuprein, Mn-SOD, Sod protein, SOD-1, SOD-2, SOD-3, SOD-4, SODF, SODS, superoxidase dismutase, Superoxide dismutase, superoxide dismutase I, superoxide dismutase II, superoxide:superoxide oxidoreductase

TABLE 24
Genes/Proteins Involved in the Purine metabolism pathway

| Gene Name | Synonyms |
|---|---|
| (R)-Allantoin | C4H6N4O3, [(4R)-2,5-dioxoimidazolidin-4-yl]urea |
| (S)-Allantoin | C4H6N4O3, [(4S)-2,5-dioxoimidazolidin-4-yl]urea |
| 1-(5′-Phosphoribosyl)-5- | 1-(5′-phosphoribosyl)-5-aminoimidazole, 1-(5-phospho-D-ribosyl)-5- |
| aminoimidazole (AIR) | aminoimidazole, 1H-Imidazol-5-amine, 1-(5-O-phosphono-beta-D- |
| | ribofuranosyl), 25635-88-5, 5′-phosphoribosyl-5-aminoimidazole, 5-amino-1- |
| | ribofuranosylimidazole 5′-phosphate, 5-aminoimidazole ribotide, AIR, |
| | aminoimidazole ribotide, (alpha-D-ribofuranosyl)-isomer, aminoimidazole |
| | ribotide, (beta-D-ribofuranosyl)-isomer, aminoimidazole ribotide, |
| | phosphonoribofuranosyl-isomer, C8H14N3O7P, [(2R,3R,4R,5R)-5-(5- |
| | aminoimidazol-1-yl)-3,4-dihydroxy-oxolan-2-yl]methoxyphosphonic acid |
| 1-(5′-Phosphoribosyl)-5- | 1-(5′-phosphoribosyl)-5-formamido-4-imidazolecarboxamide, 13018-54-7, |
| formamido-4-imidazole | 1H-imidazole-4-carboxamide, 5-(formylamino)-1-(5-O-phosphono-beta-D- |
| carboxamide | ribofuranosyl)-, 5-(formylamino)-1-(5-O-phosphono-beta-D-1H-imidazole-4- |
| | carboxamide, 5-formamidoimidazole-4-carboxamide ribotide, 5-formyl-5- |
| | aminoimidazole-4-carboxamide ribonucleotide, C10H15N4O9P, |
| | [(2R,3R,4R,5R)-5-(4-carbamoyl-5-formamido-imidazol-1-yl)-3,4-dihydroxy- |
| | oxolan-2-yl]methoxyphosphonic acid |
| 1-(5′-Phosphoribosyl)-N- | 349-34-8, C8H15N2O9P, N-formyl-GAR, N-formylglycinamide ribonucleotide, |
| formylglycinamide | [(2R,3R,4R,5R)-5-[(2-formamidoacetyl)amino]-3,4-dihydroxy-oxolan-2- |
| | yl]methoxyphosphonic acid |
| 1.1.1.154 | (S)-ureidoglycolate:NAD(P) oxidoreductase |
| 1.1.1.205 | IMP oxidoreductase, IMP:NAD oxidoreductase, inosinate dehydrogenase, |
| | inosine 5′-monophosphate dehydrogenase, inosine monophosphate |
| | dehydrogenase, inosine monophosphate oxidoreductase, inosine-5′- |
| | phosphate dehydrogenase, inosinic acid dehydrogenase |
| 1.17.1.4 | NAD-xanthine dehydrogenase, xanthine oxidoreductase, xanthine-NAD |
| | oxidoreductase, xanthine/NAD+ oxidoreductase, xanthine:NAD |
| | oxidoreductase |
| 1.17.3.2 | hypoxanthine oxidase, hypoxanthine-xanthine oxidase, hypoxanthine:oxygen |
| | oxidoreductase, Schardinger enzyme, xanthine oxidoreductase, xanthine:O2 |
| | oxidoreductase, xanthine:oxygen oxidoreductase, xanthine:xanthine oxidase |
| 1.17.4.1 | 2′-deoxyribonucleoside-diphosphate:oxidized-thioredoxin 2′-oxidoreductase, |
| | ADP reductase, CDP reductase, nucleoside diphosphate reductase, |
| | ribonucleoside 5′-diphosphate reductase, ribonucleotide diphosphate |
| | reductase, ribonucleotide reductase, UDP reductase |
| 1.17.4.2 | 2′-deoxyribonucleoside-triphosphate:oxidized-thioredoxin 2′-oxidoreductase, |
| | ribonucleotide reductase |
| 1.7.1.7 | guanosine 5′-monophosphate reductase, guanosine 5′-phosphate reductase, |
| | guanosine monophosphate reductase, guanylate reductase, inosine-5′- |
| | phosphate:NADP+ oxidoreductase (aminating), NADPH2:guanosine-5′- |
| | phosphate oxidoreductase (deaminating), NADPH:GMP oxidoreductase |
| | (deaminating) |
| 1.7.3.3 | urate:oxygen oxidoreductase, uric acid oxidase, uricase, uricase II |
| 2′,3′-Cyclic AMP | 634-01-5, adenosine cyclic 2′,3′-(hydrogen phosphate), C10H12N5O6P, |
| | [(1R,2R,4R,5R)-2-(6-aminopurin-9-yl)-7-hydroxy-7-oxo-3,6,8-trioxa- |
| | 7&lambda;<sup>5</sup>-phosphabicyclo[3.3.0]oct-4-yl]methanol |
| 2′,3′-Cyclic GMP | 2-amino-9-[(1R,2R,4R,5R)-7-hydroxy-4-(hydroxymethyl)-7-oxo-3,6,8-trioxa- |
| | 7&lambda;<sup>5</sup>-phosphabicyclo[3.3.0]oct-2-yl]-3H-purin-6-one, |
| | 634-02-6, C10H12N5O7P, guanosine cyclic 2′,3′-(hydrogen phosphate) |
| 2.1.2.2 | 10-formyltetrahydrofolate:5′-phosphoribosylglycinamide N-formyltransferase, |
| | 2-amino-N-ribosylacetamide 5′-phosphate transformylase, 5,10- |
| | methenyltetrahydrofolate:2-amino-N-ribosylacetamide ribonucleotide |
| | transformylase, GAR formyltransferase, GAR TFase, GAR transformylase, |
| | glycinamide ribonucleotide transformylase |
| 2.1.2.3 | 10-formyltetrahydrofolate:5′-phosphoribosyl-5-amino-4-imidazole- |
| | carboxamide N-formyltransferase, 10-formyltetrahydrofolate:5′- |
| | phosphoribosyl-5-amino-4-imidazolecarboxamide formyltransferase, 5′- |
| | phosphoribosyl-5-amino-4-imidazolecarboxamide formyltransferase, 5- |
| | amino-1-ribosyl-4-imidazolecarboxamide 5′-phosphate transformylase, 5- |
| | amino-4-imidazolecarboxamide ribonucleotide transformylase, 5-amino-4- |
| | imidazolecarboxamide ribotide transformylase, AICAR formyltransferase, |
| | AICAR transformylase, aminoimidazolecarboxamide ribonucleotide |
| | transformylase |
| 2.1.2.4 | 5-formimidoyltetrahydrofolate:glycine N-formimidoyltransferase, FIG |
| | formiminotransferase, formiminoglycine formiminotransferase |
| 2.1.3.5 | carbamoyl-phosphate:oxamate carbamoyltransferase, oxamic |
| | transcarbamylase |
| 2.4.2.1 | inosine phosphorylase, inosine-guanosine phosphorylase, nucleotide |
| | phosphatase, PNPase, PUNPI, PUNPII, purine deoxynucleoside |
| | phosphorylase, purine deoxyribonucleoside phosphorylase, purine |
| | ribonucleoside phosphorylase, purine-nucleoside:phosphate |
| | ribosyltransferase |
| 2.4.2.14 | 5′-phosphoribosylpyrophosphate amidotransferase, 5-phosphoribosyl-1- |
| | pyrophosphate amidotransferase, 5-phosphoribosylamine:diphosphate |
| | phospho-alpha-D-ribosyltransferase (glutamate-amidating), 5- |
| | phosphororibosyl-1-pyrophosphate amidotransferase, alpha-5- |
| | phosphoribosyl-1-pyrophosphate amidotransferase, glutamine 5- |
| | phosphoribosylpyrophosphate amidotransferase, glutamine |
| | phosphoribosyldiphosphate amidotransferase, glutamine |
| | ribosylpyrophosphate 5-phosphate amidotransferase, phosphoribose |
| | pyrophosphate amidotransferase, phosphoribosyl pyrophosphate |
| | amidotransferase, phosphoribosyldiphosphate 5-amidotransferase, |
| | phosphoribosylpyrophosphate glutamyl amidotransferase |
| 2.4.2.15 | guanosine:phosphate D-ribosyltransferase |
| 2.4.2.16 | UAR phosphorylase, urate-ribonucleotide:phosphate D-ribosyltransferase |
| 2.4.2.22 | 5-phospho-alpha-D-ribose-1-diphosphate:xanthine phospho-D- |
| | ribosyltransferase, Xan phosphoribosyltransferase, xanthosine 5′-phosphate |
| | pyrophosphorylase, xanthylate pyrophosphorylase, xanthylic |
| | pyrophosphorylase, XMP pyrophosphorylase |
| 2.4.2.4 | animal growth regulators, blood platelet-derived endothelial cell growth |
| | factors, blood platelet-derived endothelial cell growth factor, deoxythymidine |
| | phosphorylase, gliostatins, pyrimidine deoxynucleoside phosphorylase, |
| | pyrimidine phosphorylase, thymidine-orthophosphate |
| | deoxyribosyltransferase, thymidine:phosphate deoxy-D-ribosyltransferase |
| 2.4.2.7 | adenine phosphoribosylpyrophosphate transferase, adenosine |
| | phosphoribosyltransferase, adenylate pyrophosphorylase, adenylic |
| | pyrophosphorylase, AMP pyrophosphorylase, AMP-pyrophosphate |
| | phosphoribosyltransferase, AMP:diphosphate phospho-D-ribosyltransferase, |
| | APRT, transphosphoribosidase |
| 2.7.1.113 | (dihydroxypropoxymethyl)guanine kinase, 2′-deoxyguanosine kinase, |
| | ATP:deoxyguanosine 5′-phosphotransferase, deoxyguanosine kinase |
| | (phosphorylating), NTP-deoxyguanosine 5′-phosphotransferase |
| 2.7.1.20 | adenosine kinase (phosphorylating), ATP:adenosine 5′-phosphotransferase |
| 2.7.1.25 | 5′-phosphoadenosine sulfate kinase, adenosine 5′-phosphosulfate kinase, |
| | adenosine phosphosulfate kinase, adenosine phosphosulfokinase, |
| | adenosine-5′-phosphosulfate-3′-phosphokinase, Adenylyl-sulfate kinase, |
| | adenylylsulfate kinase (phosphorylating), ATP:adenylyl-sulfate 3′- |
| | phosphotransferase |
| 2.7.1.40 | ATP:pyruvate 2-O-phosphotransferase, fluorokinase, fluorokinase |
| | (phosphorylating), phosphoenol transphosphorylase pyruvate kinase |
| | (phosphorylating), phosphoenolpyruvate kinase, Pk, pyruvate |
| | phosphotransferase, pyruvic kinase |
| 2.7.1.73 | ATP:inosine 5′-phosphotransferase, inosine kinase (phosphorylating), |
| | inosine-guanosine kinase |
| 2.7.1.74 | 2′-deoxycytidine kinase, Ara-C kinase, arabinofuranosylcytosine kinase, |
| | deoxycytidine kinase (phosphorylating), deoxycytidine-cytidine kinase, |
| | NTP:deoxycytidine 5′-phosphotransferase |
| 2.7.1.76 | ATP:deoxyadenosine 5′-phosphotransferase, purine-deoxyribonucleoside |
| | kinase, purine-deoxyribonucleoside kinase deoxyadenosine kinase |
| | (phosphorylating) |
| 2.7.2.2 | ATP:carbamate phosphotransferase, carbamoyl phosphokinase, carbamyl |
| | phosphokinase, CKase |
| 2.7.4.11 | ATP:(d)AMP phosphotransferase |
| 2.7.4.3 | 5′-AMP-kinase, adenylic kinase, adenylokinase, AK, ATP:AMP |
| | phosphotransferase, myokinase |
| 2.7.4.6 | ATP:nucleoside-diphosphate phosphotransferase, NDP kinase, nucleoside |
| | 5′-diphosphate kinase, nucleoside diphosphate (UDP) kinase, nucleoside |
| | diphosphokinase, Nucleoside-diphosphate kinase, nucleotide phosphate |
| | kinase, UDP kinase, uridine diphosphate kinase |
| 2.7.4.8 | 5′-GMP kinase, ATP:(d)GMP phosphotransferase, ATP:GMP |
| | phosphotransferase, deoxyguanylate kinase, GMP kinase, guanosine |
| | monophosphate kinase, Guanylate kinase |
| 2.7.6.1 | 5-phosphoribose pyrophosphorylase, 5-phosphoribosyl-1-pyrophosphate |
| | synthetase, 5-phosphoribosyl-alpha-1-pyrophosphate synthetase, ATP:D- |
| | ribose-5-phosphate diphosphotransferase, phosphoribosyl-diphosphate |
| | synthetase, phosphoribosylpyrophosphate synthase, |
| | phosphoribosylpyrophosphate synthetase, PP-ribose P synthetase, PPRibP |
| | synthetase, PRPP synthetase, pyrophosphoribosylphosphate synthetase, |
| | ribophosphate pyrophosphokinase, ribose-5-phosphate pyrophosphokinase, |
| | ribose-phosphate pyrophosphokinase |
| 2.7.6.5 | (p)ppGpp synthetase I, (p)ppGpp synthetase II, ATP-GTP 3′- |
| | diphosphotransferase, ATP:GTP 3′-diphosphotransferase, GPSI, GPSII, GTP |
| | pyrophosphokinase, guanosine 3′,5′-polyphosphate synthase, guanosine |
| | 5′,3′-polyphosphate synthetase, guanosine pentaphosphate synthetase, |
| | stringent factor |
| 2.7.7.4 | adenosine-5′-triphosphate sulfurylase, adenosinetriphosphate sulfurylase, |
| | adenylylsulfate pyrophosphorylase, ATP sulfurylase, ATP:sulfate |
| | adenylyltransferase, sulfurylase |
| 2.7.7.48 | 3D polymerase, nucleoside-triphosphate:RNA nucleotidyltransferase (RNA- |
| | directed), PB1 proteins, PB2 proteins, phage f2 replicase, polymerase L, Q- |
| | beta replicase, RDRP, ribonucleic acid replicase, ribonucleic acid-dependent |
| | ribonucleate nucleotidyltransferase, ribonucleic acid-dependent ribonucleic |
| | acid polymerase, ribonucleic replicase, ribonucleic synthetase, RNA |
| | nucleotidyltransferase (RNA-directed), RNA replicase, RNA synthetase, RNA |
| | transcriptase, RNA-dependent ribonucleate nucleotidyltransferase, RNA- |
| | dependent RNA polymerase, RNA-dependent RNA replicase, transcriptase |
| 2.7.7.53 | adenine triphosphate adenylyltransferase, ADP:ATP adenylyltransferase, |
| | bis(5′-nucleosyl)-tetraphosphate phosphorylase (NDP-forming), diadenosine |
| | 5′,5′″-P1,P4-tetraphosphate alphabeta-phosphorylase, |
| | diadenosinetetraphosphate ab-phosphorylase, dinucleoside oligophosphate |
| | ab-phosphorylase |
| 2.7.7.7 | deoxynucleate polymerase, deoxynucleoside-triphosphate:DNA |
| | deoxynucleotidyltransferase (DNA-directed), deoxyribonucleate |
| | nucleotidyltransferase, deoxyribonucleic acid duplicase, deoxyribonucleic |
| | acid polymerase, deoxyribonucleic duplicase, deoxyribonucleic polymerase, |
| | deoxyribonucleic polymerase I, DNA duplicase, DNA nucleotidyltransferase, |
| | DNA nucleotidyltransferase (DNA-directed), DNA polymerase, DNA |
| | polymerase alpha, DNA polymerase beta, DNA polymerase g, DNA |
| | polymerase I, DNA polymerase II, DNA polymerase III, DNA replicase, DNA- |
| | dependent DNA polymerase, duplicase, Klenow fragment, sequenase, Taq |
| | DNA polymerase, Taq Pol I, Tca DNA polymerase |
| 2.7.7.8 | nucleoside diphosphate:polynucleotidyl transferase, PNPase, polynucleotide |
| | phosphorylase, polyribonucleotide phosphorylase, |
| | polyribonucleotide:phosphate nucleotidyltransferase |
| 3′,5′-Cyclic AMP | (1S,6R,8R,9R)-8-(6-aminopurin-9-yl)-3-hydroxy-3-oxo-2,4,7-trioxa- |
| | 3&lambda;<sup>5</sup>-phosphabicyclo[4.3.0]nonan-9-ol, 3′,5′-cyclic AMP, |
| | 60-92-4, adenosine 3′,5′-phosphate, adenosine cyclic 3′,5′-monophosphate, |
| | C10H12N5O6P, cAMP, cyclic adenosine monophosphate, cyclic adenylic |
| | acid |
| 3′,5′-Cyclic GMP | 2-amino-9-[(1R,6R,8R,9R)-3,9-dihydroxy-3-oxo-2,4,7-trioxa- |
| | 3&lambda;<sup>5</sup>-phosphabicyclo[4.3.0]non-8-yl]-3H-purin-6-one, |
| | 3′,5′-cyclic GMP, 7665-99-8, C10H12N5O7P, cGMP, guanosine 3′,5′-cyclic |
| | phosphate, guanosine cyclic 3′,5′-(hydrogen phosphate) |
| 3′-AMP | 3′-adenylic acid, 3′-AMP, 84-21-9, adenosine 3′-monophosphate, adenosine |
| | 3′-phosphate, adenosine-3′-monophosphoric acid, C10H14N5O7P, |
| | [(2R,3R,4R,5R)-5-(6-aminopurin-9-yl)-4-hydroxy-2-(hydroxymethyl)oxolan-3- |
| | yl]oxyphosphonic acid |
| 3′-Phosphoadenylate | 1053-73-2, 3′,5′-adenosine 5′-diphosphate, 3′-phosphoadenosine 5′- |
| | phosphate, 3′-phosphoadenylate, A3P5P, adenosine 3′,5′-bisphosphate, |
| | adenosine 3′,5′-diphosphate, adenosine 3′-phosphate-5′-phosphate, |
| | C10H15N5O10P2, PAP, phosphoadenosine phosphate, [(2R,3R,4R,5R)-5- |
| | (6-aminopurin-9-yl)-4-hydroxy-3-phosphonooxy-oxolan-2- |
| | yl]methoxyphosphonic acid |
| 3′-Phosphoadenylyl | 3′-adenylic acid, 5′-(dihydrogen phosphate), 5′-anhydride with sulfuric acid, |
| sulfate | 3′-phosphoadenosine 5′-phosphosulfate, 3′-phosphoadenylyl sulfate, 482-67- |
| | 7, 6-amino-9-[(2R,3R,4R,5R)-3-hydroxy-5-[(hydroxy-sulfooxy- |
| | phosphoryl)oxymethyl]-4-phosphonooxy-oxolan-2-yl]purine, adenosine, 3′,5′- |
| | bis(dihydrogen phosphate), 5′-monoanhydride with sulfuric acid, adenosine, |
| | 3′,5′-bis(dihydrogen phosphate), 5′-monoanhydride with sulfuric acid (8Cl), |
| | adenosine, 3′,5′-diphosphate, 5′-anhydride with sulfuric acid, |
| | C10H15N5O13P2S, PAPS |
| 3′GMP | 117-68-0, 3′-guanylic acid, C10H14N5O8P, GMP3′, guanosine 3′- |
| | (dihydrogen phosphate), [(2R,3R,4R,5R)-5-(2-amino-6-oxo-3H-purin-9-yl)-4- |
| | hydroxy-2-(hydroxymethyl)oxolan-3-yl]oxyphosphonic acid |
| 3.1.3.5 | 5′-adenylic phosphatase, 5′-AMP nucleotidase, 5′-AMPase, 5′- |
| | mononucleotidase, 5′-Nt, 5′-ribonucleotide phosphohydrolase, adenosine 5′- |
| | phosphatase, adenosine monophosphatase, AMP phosphatase, AMP |
| | phosphohydrolase, AMPase, IMP 5′-nucleotidase, snake venom 5′- |
| | nucleotidase, thimidine monophosphate nucleotidase, UMPase, uridine 5′- |
| | nucleotidase |
| 3.1.3.6 | 3′-mononucleotidase, 3′-phosphatase, 3′-ribonucleotidase, 3′-ribonucleotide |
| | phosphohydrolase |
| 3.1.4.16 | 2′,3′-cyclic AMP phosphodiesterase, 2′,3′-cyclic AMP 2′-phosphohydrolase, |
| | 2′,3′-cyclic nucleoside monophosphate phosphodiesterase, 2′,3′-cyclic |
| | nucleotidase, 2′,3′-cyclic nucleotide phosphohydrolase, 2′:3′-cyclic nucleotide |
| | phosphodiesterase:3′-nucleotidase, 2′:3′-cyclic phosphodiesterase, cyclic |
| | 2′,3′-nucleotide 2′-phosphodiesterase, cyclic 2′,3′-nucleotide |
| | phosphodiesterase, cyclic phosphodiesterase:3′-nucleotidase, nucleoside- |
| | 2′,3′-cyclic-phosphate 3′-nucleotidohydrolase, ribonucleoside 2′,3′-cyclic |
| | phosphate diesterase |
| 3.1.4.17 | 3′,5′-cyclic nucleoside monophosphate phosphodiesterase, 3′,5′-cyclic- |
| | nucleotide 5′-nucleotidohydrolase, 3′,5′-cyclonucleotide phosphodiesterase, |
| | 3′,5′-nucleotide phosphodiesterase, 3′:5′-cyclic nucleotide 5′- |
| | nucleotidohydrolase, 3′:5′-monophosphate phosphodiesterase (cyclic CMP), |
| | cyclic 3′,5′-mononucleotide phosphodiesterase, cyclic 3′,5′-nucleotide |
| | phosphodiesterase, cyclic 3′,5′-phosphodiesterase, cyclic 3′,5-nucleotide |
| | monophosphate phosphodiesterase, cyclic nucleotide phosphodiesterase, |
| | cytidine 3′:5′-monophosphate phosphodiesterase (cyclic CMP), nucleoside |
| | 3′,5′-cyclic phosphate diesterase, nucleoside-3′,5-monophosphate |
| | phosphodiesterase, PDE |
| 3.1.5.1 | deoxy-GTPase, deoxyguanosine 5-triphosphate triphosphohydrolase, |
| | deoxyguanosine triphosphatase, deoxyguanosine triphosphate |
| | triphosphohydrolase, dGTP triphosphohydrolase |
| 3.1.7.2 | guanosine-3′,5′-bis(diphosphate) 3′-diphosphohydrolase, guanosine-3′,5′- |
| | bis(diphosphate) 3′-pyrophosphatase, PpGpp phosphohydrolase, PpGpp-3′- |
| | pyrophosphohydrolase |
| 3.2.2.1 | N-D-ribosylpurine ribohydrolase, N-ribosyl purine ribohydrolase, |
| | nucleosidase, nucleosidase g, nucleoside hydrolase, purine beta-ribosidase, |
| | purine nucleoside hydrolase, purine ribonucleosidase, ribonucleoside |
| | hydrolase |
| 3.2.2.12 | 5′-inosinate phosphoribohydrolase |
| 3.2.2.2 | inosinase, inosine ribohydrolase, inosine-guanosine nucleosidase |
| 3.2.2.4 | adenosine monophosphate nucleosidase, adenylate nucleosidase, AMP |
| | phosphoribohydrolase |
| 3.2.2.7 | adenosinase, adenosine hydrolase, adenosine ribohydrolase, ANase, N- |
| | ribosyladenine ribohydrolase |
| 3.2.2.8 | N-ribosylpyrimidine nucleosidase, N-ribosylpyrimidine ribohydrolase, |
| | nucleoside ribohydrolase, pyrimidine nucleosidase |
| 3.5.1.5 | urea amidohydrolase |
| 3.5.2.— | |
| 3.5.2.17 | |
| 3.5.2.5 | allantoin amidohydrolase |
| 3.5.3.— | |
| 3.5.3.19 | ureidoglycolate amidohydrolase (decarboxylating) |
| 3.5.3.4 | allantoate amidinohydrolase |
| 3.5.3.9 | allantoate amidinohydrolase (decarboxylating), allantoate amidohydrolase |
| 3.5.4.10 | IMP 1,2-hydrolase (decyclizing), inosinate cyclohydrolase, inosinicase |
| 3.5.4.2 | ADase, adenase, adenine aminase, adenine aminohydrolase |
| 3.5.4.3 | GAH, guanase, guanine aminase, guanine aminohydrolase |
| 3.5.4.4 | adenosine aminohydrolase, deoxyadenosine deaminase |
| 3.5.4.6 | 5-adenylate deaminase, 5-adenylic acid deaminase, 5-AMP deaminase, |
| | adenosine 5-monophosphate deaminase, adenosine 5-phosphate |
| | aminohydrolase, adenosine monophosphate deaminase, adenyl deaminase, |
| | adenylate aminohydrolase, adenylate deaminase, adenylate desaminase, |
| | adenylic acid deaminase, adenylic deaminase, AMP aminase, AMP |
| | aminohydrolase, AMP deaminase |
| 3.5.4.8 | 4-aminoimidazole aminohydrolase, 4-aminoimidazole hydrolase |
| 3.6.1.11 | acid phosphoanhydride phosphohydrolase, Gra-Pase, metaphosphatase, |
| | polyphosphate phosphohydrolase |
| 3.6.1.13 | adenosine diphosphoribose pyrophosphatase, ADPR-PPase, ADPribose |
| | pyrophosphatase, ADPribose ribophosphohydrolase |
| 3.6.1.14 | adenosine-tetraphosphate phosphohydrolase |
| 3.6.1.15 | nucleoside 5-triphosphatase, nucleoside triphosphate phosphohydrolase, |
| | nucleoside-5-triphosphate phosphohydrolase, unspecific diphosphate |
| | phosphohydrolase |
| 3.6.1.17 | bis(5′-adenosyl)-tetraphosphatase, bis(5′-guanosyl)-tetraphosphatase, |
| | diadenosine P1,P4-tetraphosphatase, diguanosinetetraphosphatase |
| | (asymmetrical), dinucleoside tetraphosphatase, |
| | dinucleosidetetraphosphatase (asymmetrical), P1,P4-bis(5′-nucleosyl)-tetraphosphate |
| | nucleotidohydrolase |
| 3.6.1.19 | nucleoside-triphosphate diphosphohydrolase, nucleoside-triphosphate |
| | pyrophosphatase |
| 3.6.1.20 | 5′-acylphosphoadenosine acylhyrolase, 5-phosphoadenosine hydrolase |
| 3.6.1.21 | adenosine diphosphosugar pyrophosphatase, ADP-sugar pyrophosphatase, |
| | ADP-sugar sugarphosphohydrolase |
| 3.6.1.29 | diadenosine 5,5-P1,P3-triphosphatase, dinucleosidetriphosphatase, P1,P3- |
| | bis(5′-adenosyl)-triphosphate adenylohydrolase |
| 3.6.1.3 | (Ca2+ + Mg2+)-ATPase, adenosine 5′-triphosphatase, adenosine |
| | triphosphatase, adenylpyrophosphatase, ATP hydrolase, ATP |
| | monophosphatase, ATP phosphohydrolase, complex V (mitochondrial |
| | electron transport), HCO3−-ATPase, SV40 T-antigen, triphosphatase |
| 3.6.1.40 | guanosine 5′-triphosphate 3′-diphosphate 5′-phosphatase, guanosine 5′- |
| | triphosphate-3′-diphosphate 5′-phosphohydrolase, guanosine |
| | pentaphosphatase, guanosine pentaphosphate phosphatase, guanosine |
| | pentaphosphate phosphohydrolase, guanosine-5′-triphosphate,3′- |
| | diphosphate 5′-phosphohydrolase, guanosine-5′-triphosphate,3′-diphosphate |
| | pyrophosphatase, pppGpp 5′-phosphohydrolase |
| 3.6.1.41 | adenosine tetraphosphate phosphodiesterase, Ap4A hydrolase, bis(5′- |
| | adenosyl) tetraphosphatase, diadenosine 5′,5′″-P1,P4-tetraphosphatase, |
| | diadenosine polyphosphate hydrolase, diadenosine tetraphosphate |
| | hydrolase, diadenosinetetraphosphatase (symmetrical), |
| | dinucleosidetetraphosphate (symmetrical), P1,P4-bis(5′-nucleosyl)-tetraphosphate |
| | nucleosidebisphosphohydrolase, symmetrical diadenosine |
| | tetraphosphate hydrolase |
| 3.6.1.5 | adenosine diphosphatase, ADPase, ATP diphosphohydrolase, ATP- |
| | diphosphatase |
| 3.6.1.6 | adenosine diphosphatase, adenosinepyrophosphatase, ADPase, CDPase, |
| | GDPase, guanosine 5′-diphosphatase, guanosine diphosphatase, IDPase, |
| | inosine 5′-diphosphatase, inosine diphosphatase, NDPase, nucleoside 5′- |
| | diphosphatase, nucleoside diphosphate phosphatase, nucleoside |
| | diphosphate phosphohydrolase, thiaminpyrophosphatase, type B nucleoside |
| | diphosphatase, type L nucleoside diphosphatase, UDPase, uridine 5′- |
| | diphosphatase, uridine diphosphatase |
| 3.6.1.8 | adenosine triphosphate pyrophosphatase, ATP diphosphohydrolase, ATP |
| | pyrophosphatase, ATPase |
| 3.6.1.9 | dinucleotide nucleotidohydrolase, nucleotide pyrophosphatase, nucleotide- |
| | sugar pyrophosphatase |
| 3.6.4.1 | actomyosin, ATP phosphohydrolase (actin-translocating) |
| 4.1.1.— | |
| 4.1.1.21 | 1-(5-phosphoribosyl)-5-amino-4-imidazolecarboxylate carboxy-lyase, 5- |
| | amino-1-ribosylimidazole 5-phosphate carboxylase, 5-phosphoribosyl-5- |
| | aminoimidazole carboxylase, AIR carboxylase |
| 4.3.2.2 | adenylosuccinase, N6-(1,2-dicarboxyethyl)AMP AMP-lyase, succino AMP- |
| | lyase |
| 4.3.2.3 | (−)-ureidoglycolate urea-lyase, ureidoglycolase, ureidoglycolatase, |
| | ureidoglycolate hydrolase |
| 4.6.1.1 | 3′,5′-cyclic AMP synthetase, ADENYL CYCLASE, Adenylate Cyclase, |
| | Adenylyl Cyclase, Adenylyl Cyclase protein, ATP diphosphate-lyase |
| | (cyclizing) |
| 4.6.1.2 | GTP diphosphate-lyase (cyclizing), guanyl cyclase, Guanylate cyclase, |
| | guanylyl cyclase |
| 5′-Acetylphospho- | 13015-87-7, 5′-acetylphosphoadenosine, 5′-adenylic acid, monoanhydride |
| adenosine (mitochondria) | with acetic acid, acetyl adenylate, acetyl AMP, acetyloxy-[[(2R,3R,4R,5R)-5- |
| | (6-aminopurin-9-yl)-3,4-dihydroxy-oxolan-2-yl]methoxy]phosphinic acid, |
| | C12H16N5O8P |
| 5′-Benzoylphospho- | C17H18N5O8P, [(2R,3R,4R,5R)-5-(6-aminopurin-9-yl)-3,4-dihydroxy-oxolan- |
| adenosine (mitochondria) | 2-yl]methoxy-benzoyloxy-phosphinic acid |
| 5′-Butyrylphosphoinosine | butanoyloxy-[[(2R,3R,4R,5R)-3,4-dihydroxy-5-(6-oxo-3H-purin-9-yl)oxolan-2- |
| | yl]methoxy]phosphinic acid, C14H19N4O9P |
| 5′P-Ribosyl-4-(N- | (2S)-2-[[5-amino-1-[(2R,3R,4R,5R)-3,4-dihydroxy-5- |
| succinocarboxamide)-5- | (phosphonooxymethyl)oxolan-2-yl]imidazole-4-carbonyl]amino]butanedioic |
| aminoimidazole | acid, 1-(5′-phosphoribosyl)-5-amino-4-(N-succinocarboxamide)-imidazole, |
| | 3031-95-6, C13H19N4O12P, N-((5-amino-1-(5-O-phosphono-beta-D- |
| | ribofuranosyl)-1H-imidazol-4-yl)carbonyl)-L-aspartic acid |
| 5′P-Ribosyl-4-carboxy-5- | 1-(5′-phosphoribosyl)-5-amino-4-carboxyimidazole, 1-(5-phospho-D-ribosyl)- |
| aminoimidazole | 5-amino-4-imidazolecarboxylate, 1H-imidazole-4-carboxylic acid, 5-amino-1- |
| | (5-O-phosphono-beta-D-ribofuranosyl)-, 5-amino-1-[(2R,3R,4R,5R)-3,4- |
| | dihydroxy-5-(phosphonooxymethyl)oxolan-2-yl]imidazole-4-carboxylic acid, |
| | 6001-14-5, AICOR, C9H14N3O9P |
| 5-Amino-4-imidazole | 5-amino-3H-imidazole-4-carboxylic acid, C4H5N3O2 |
| carboxylate | |
| 5-Amino-4- | 360-97-4, 5-amino-3H-imidazole-4-carboxamide, aminoimidazole |
| imidazolecarboxyamide | carboxamide, C4H6N4O |
| 5-Hydroxy-2-oxo-4- | 5-(carbamoylamino)-4-hydroxy-2-oxo-3H-imidazole-4-carboxylic acid, 5- |
| ureido-2,5-dihydro-1H- | hydroxy-2-oxo-4-ureido-2,5-dihydro-1H-imidazole-5-carboxylate, C5H6N4O5 |
| imidazole-5-carboxylate | |
| 5-Hydroxyiourate | 5-hydroxy-3,7-dihydropurine-2,6,8-trione, 5-hydroxyisourate, 6960-30-1, |
| | C5H4N4O4 |
| 5-Ureido-4-imidazole | 5-(carbamoylamino)-3H-imidazole-4-carboxylic acid, C5H6N4O3 |
| carboxylate | |
| 5.1.99.3 | |
| 5.4.2.7 | alpha-D-glucose-1,6-bisphosphate:deoxy-D-ribose-1-phosphate |
| | phosphotransferase, D-ribose 1,5-phosphomutase, deoxyribomutase, |
| | deoxyribose phosphomutase, phosphodeoxyribomutase, phosphoribomutase |
| 6.3.2.6 | 1-(5-phosphoribosyl)-5-amino-4-carboxyimidazole:L-aspartate ligase (ADP- |
| | forming) |
| 6.3.3.1 | 2-(formamido)-N1-(5-phosphoribosyl)acetamidine cyclo-ligase (ADP- |
| | forming), phosphoribosylaminoimidazole synthetase |
| 6.3.4.1 | xanthosine-5′-phosphate-ammonia ligase, xanthosine-5′-phosphate:ammonia |
| | ligase (AMP-forming) |
| 6.3.4.13 | 5-phospho-D-ribosylamine:glycine ligase (ADP-forming), glycinamide |
| | ribonucleotide synthetase, phosphoribosylglycinamide synthetase |
| 6.3.4.4 | IMP-aspartate ligase, IMP:L-aspartate ligase (GDP-forming) |
| 6.3.4.7 | 5-phosphoribosylamine synthetase, ribose-5-phosphate:ammonia ligase |
| | (ADP-forming) |
| 6.3.5.2 | GMP synthetase (glutamine-hydrolysing), xanthosine-5′-phosphate:L- |
| | glutamine amido-ligase (AMP-forming) |
| 6.3.5.3 | 5′-phosphoribosylformylglycinamide:L-glutamine amido-ligase (ADP-forming), |
| | phosphoribosylformylglycinamidine synthetase |
| Adenine | 1H-purin-6-amine, 73-24-5, 7H-purin-6-amine, C5H5N5 |
| Adenosine | (2R,3R,4R,5R)-2-(6-aminopurin-9-yl)-5-(hydroxymethyl)oxolane-3,4-diol, 58- |
| | 61-7, 9-beta-D-ribofuranosyl-9H-purin-6-amine, adenine riboside, adenine-9- |
| | b-D-ribofuranoside, Adeno-jec, Adenocard, Adenoscan, Adenosine-5, |
| | Adenosine-5-Triphosphate Disodium, beta-adenosine, beta-D-adenosine, |
| | C10H13N5O4, My-O-Den |
| Adenosine 5′- | 1062-98-2, adenosine 5′-(pentahydrogen tetraphosphate), adenosine 5′- |
| tetraphosphate | tetraphosphate, C10H17N5O16P4, [[[[(2R,3R,4R,5R)-5-(6-aminopurin-9-yl)- |
| | 3,4-dihydroxy-oxolan-2-yl]methoxy-hydroxy-phosphoryl]oxy-hydroxy- |
| | phosphoryl]oxy-hydroxy-phosphoryl]oxyphosphonic acid |
| Adenylosuccinate | 19046-78-7, 2-[[9-[(2R,3R,4R,5R)-3,4-dihydroxy-5- |
| | (phosphonooxymethyl)oxolan-2-yl]purin-6-yl]amino]butanedioic acid, |
| | adenylosuccinate, adenylosuccinic acid, C14H18N5O11P, L-Aspartic acid, |
| | N-(9-(5-O-phosphono-beta-D-ribofuranosyl)-9H-purin-6-yl)-, N6-(1,2- |
| | dicarboxyethyl)-AMP |
| Adenylylsulfate | 485-84-7, 5′-Aaenylic acid, monoanhydride with sulfuric acid, 5′-adenylyl |
| | sulfate, 6-amino-9-[(2R,3R,4R,5R)-3,4-dihydroxy-5-[(hydroxy-sulfooxy- |
| | phosphoryl)oxymethyl]oxolan-2-yl]purine, adenosine 5′-phosphosulfate, |
| | adenylylsulfate, APS, C10H14N5O10PS |
| ADP | 20398-34-9, 58-64-0, 9-beta-D-arabinofuranosyladenine 5′-diphosphate, |
| | adenosine 5′-(trihydrogen diphosphate), adenosine diphosphate, |
| | C10H15N5O10P2, [[(2R,3R,4R,5R)-5-(6-aminopurin-9-yl)-3,4-dihydroxy- |
| | oxolan-2-yl]methoxy-hydroxy-phosphoryl]oxyphosphonic acid |
| ADPribose | 20762-30-5, 68414-18-6, adenosine 5′-diphosphoribose, adenosine |
| | diphosphate ribose, adenosine diphosphoribose, ADP-ribose, |
| | C15H24N5O14P2+, ribose adenosinediphosphate, [(2R,3R,4R,5R)-5-(6- |
| | amino-7H-purin-9-yl)-3,4-dihydroxy-oxolan-2-yl]methoxy-[[(2R,3S,4S,5S)-3,4- |
| | dihydroxy-5-(hydroxymethyl)oxolan-2-yl]oxy-hydroxy-phosphoryl]oxy- |
| | phosphinic acid |
| AICAR | 1H-imidazole-4-carboxamide, 5-amino-1-(5-O-phosphono-beta-D- |
| | ribofuranosyl)-, 1H-imidazole-4-carboxamide, 5-amino-1-(5-O-phosphono- |
| | beta-D-ribofuranosyl)-(9Cl), 3031-94-5, 5-amino-1-(5-O-phosphono-beta-D- |
| | ribofuranosyl)-1H-imidazole-4-carboxamide, 5-amino-4-imidazole |
| | carboxamide ribonucleotide, 5-amino-4-imidazolecarboxamide |
| | ribonucleoside 5′-monophosphate, 5-amino-4-imidazolecarboxamide ribotide, |
| | 5-aminoimidazole-4-carboxamide-1-beta-d-ribonucleotide, AICA |
| | ribonucleotide, C9H15N4O8P, imidazole-4-carboxamide, 5-amino-1-beta-D- |
| | ribofuranosyl-, 5′-(dihydrogen phosphate), imidazole-4-carboxamide, 5- |
| | amino-1-beta-D-ribofuranosyl-, 5′-(dihydrogen phosphate) (8Cl), Z- |
| | nucleotide, [(2R,3R,4R,5R)-5-(5-amino-4-carbamoyl-imidazol-1-yl)-3,4- |
| | dihydroxy-oxolan-2-yl]methoxyphosphonic acid |
| Allantoate | 2,2-bis(carbamoylamino)acetic acid, 99-16-1, acetic acid, |
| | bis((aminocarbonyl)amino)-, allantoate, bis((aminocarbonyl)amino)acetic |
| | acid, C4H8N4O4, diureidoacetic acid |
| Aminoimidazole | 1H-imidazol-4-amine, 3H-imidazol-4-amine, 4919-03-3, 5-aminoimidazole, |
| | C3H5N3 |
| AMP | 5′-adenylic acid, 5′-AMP, 61-19-8, adenosine monophosphate, |
| | C10H14N5O7P, [(2R,3R,4R,5R)-5-(6-aminopurin-9-yl)-3,4-dihydroxy-oxolan- |
| | 2-yl]methoxyphosphonic acid |
| ApppA | 5′Ap3A, 56432-02-1, adenosine 3′-(tetrahydrogen triphosphate), 3′-5′-ester |
| | with adenosine, adenosine(3)triphosphate adenosine, Ap3A, ApppA, |
| | bis[[[(2R,3R,4R,5R)-5-(6-aminopurin-9-yl)-3,4-dihydroxy-oxolan-2- |
| | yl]methoxy-hydroxy-phosphoryl]oxy]phosphinic acid, C20H27N10O16P3, |
| | P1,P3-bis(5′-adenosyl) triphosphate |
| AppppA | 5542-28-9, Ap4A, C20H28N10O19P4, [[(2R,3R,4R,5R)-5-(6-aminopurin-9- |
| | yl)-3,4-dihydroxy-oxolan-2-yl]methoxy-hydroxy-phosphoryl]oxy- |
| | [[[(2R,3R,4R,5R)-5-(6-aminopurin-9-yl)-3,4-dihydroxy-oxolan-2-yl]methoxy- |
| | hydroxy-phosphoryl]oxy-hydroxy-phosphoryl]oxy-phosphinic acid |
| ATP | 56-65-5, 9-beta-D-arabinofuranosyladenine 5′-triphosphate, adenosine 5′- |
| | (tetrahydrogen triphosphate), adenosine 5′-triphosphate, adenosine |
| | triphosphate, ATP4−, C10H16N5O13P3, [[[(2R,3R,4R,5R)-5-(6-aminopurin-9- |
| | yl)-3,4-dihydroxy-oxolan-2-yl]methoxy-hydroxy-phosphoryl]oxy-hydroxy- |
| | phosphoryl]oxyphosphonic acid |
| Carbamoyl phosphate | 590-55-6, carbamic acid, monoanhydride with phosphoric acid, |
| | carbamoyloxyphosphonic acid, CH4NO5P |
| CO2 | 124-38-9, carbon dioxide, carbonic anhydride, CO2, dry ice |
| D-Ribose-1P | 18646-11-2, alpha-D-ribofuranose 1-(dihydrogen phosphate), C5H11O8P, |
| | [(2R,3R,4S,5R)-3,4-dihydroxy-5-(hydroxymethyl)oxolan-2-yl]oxyphosphonic |
| | acid |
| dADP | 2′-deoxyadenosine 5′-(trihydrogen diphosphate), 2′-deoxyadenosine 5′- |
| | diphosphate, 2793-06-8, 72003-83-9, adenosine 5′-(trihydrogen |
| | diphosphate), 2′-deoxy-, disodium salt, C10H15N5O9P2, [[(2R,3S,5R)-5-(6- |
| | aminopurin-9-yl)-3-hydroxy-oxolan-2-yl]methoxy-hydroxy- |
| | phosphoryl]oxyphosphonic acid |
| dAMP | 2′-deoxy-5′-adenosine monophosphate, 2′-deoxy-5′-adenylic acid, 2′-deoxy- |
| | AMP, 2′-deoxyadenosine 5′-(dihydrogen phosphate), 5′-adenylic acid, 2′- |
| | deoxy-, 5′-adenylic acid, 2′-deoxy-(9Cl), 653-63-4, adenosine, 2′-deoxy-, 5′- |
| | (dihydrogen phosphate), adenosine, 2′-deoxy-, 5′-(dihydrogen phosphate) |
| | (8Cl), C10H14N5O6P, deoxy-AMP, deoxyadenosine monophosphate, |
| | [(2R,3S,5R)-5-(6-aminopurin-9-yl)-3-hydroxy-oxolan-2-yl]methoxyphosphonic |
| | acid |
| dATP | 1927-31-7, 2′-deoxyadenosine 5′-(tetrahydrogen triphosphate), 2′- |
| | deoxyadenosine triphosphate, C10H16N5O12P3, [[[(2R,3S,5R)-5-(6- |
| | aminopurin-9-yl)-3-hydroxy-oxolan-2-yl]methoxy-hydroxy-phosphoryl]oxy- |
| | hydroxy-phosphoryl]oxyphosphonic acid |
| Deoxyadenosine | (2R,3S,5R)-5-(6-aminopurin-9-yl)-2-(hydroxymethyl)oxolan-3-ol, 2′-d- |
| | adenosine, 958-09-8, adenine deoxy nucleoside, adenosine, 2′-deoxy-, |
| | adenyldeoxyriboside, C10H13N5O3, deoxyadenosine |
| Deoxyguanosine | 2′-deoxyguanosine, 2-amino-9-[(2R,4S,5R)-4-hydroxy-5- |
| | (hydroxymethyl)oxolan-2-yl]-3H-purin-6-one, 961-07-9, 9H-purin-6-ol, 2- |
| | amino-9-(2-deoxy-9-beta-D-ribofuranosyl)-, C10H13N5O4, guanine |
| | deoxyriboside |
| Deoxyinosine | 2′-deoxyinosine, 890-38-0, 9-[(2R,4S,5R)-4-hydroxy-5- |
| | (hydroxymethyl)oxolan-2-yl]-3H-purin-6-one, C10H12N4O4, inosine, 2′- |
| | deoxy- |
| dGDP | 102783-74-4, 2′-deoxyguanosine 5′-diphosphate, 2′-deoxyguanosine 5′- |
| | diphosphate sodium salt, C10H15N5O10P2, deoxyguanine diphosphate, [[5- |
| | (2-amino-6-oxo-3H-purin-9-yl)-3-hydroxy-oxolan-2-yl]methoxy-hydroxy- |
| | phosphoryl]oxyphosphonic acid |
| dGMP | 2′-deoxy-5′-guanylic acid, 2′-deoxyguanosine 5′-phosphate, 5′-dGMP, 5′- |
| | guanylic acid, 2′-deoxy-, 5′-guanylic acid, 2′-deoxy-(9Cl), 902-04-5, |
| | C10H14N5O7P, deoxy-GMP, guanosine, 2′-deoxy-, 5′-(dihydrogen |
| | phosphate), guanosine, 2′-deoxy-, 5′-(dihydrogen phosphate) (8Cl), |
| | [(2R,3S,5R)-5-(2-amino-6-oxo-3H-purin-9-yl)-3-hydroxy-oxolan-2- |
| | yl]methoxyphosphonic acid |
| dGTP | 2′-deoxyguanosine 5′-(tetrahydrogen triphosphate), 2564-35-4, 5′-dGTP, |
| | C10H16N5O13P3, deoxy-GTP, deoxyguanosine triphosphate, guanosine 5′- |
| | (tetrahydrogen triphosphate), 2′-deoxy-, guanosine 5′-(tetrahydrogen |
| | triphosphate), 2′-deoxy-(9Cl), guanosine, 2′-deoxy-, 5′-(tetrahydrogen |
| | triphosphate), [[[(2R,3S,5R)-5-(2-amino-6-oxo-3H-purin-9-yl)-3-hydroxy- |
| | oxolan-2-yl]methoxy-hydroxy-phosphoryl]oxy-hydroxy- |
| | phosphoryl]oxyphosphonic acid |
| dIDP | 2′-deoxyinosine 5′-diphosphate, C10H14N4O10P2, [hydroxy-[[(2R,3S,5R)-3- |
| | hydroxy-5-(6-oxo-3H-purin-9-yl)oxolan-2- |
| | yl]methoxy]phosphoryl]oxyphosphonic acid |
| dIMP | 2′-deoxyinosine 5′-monophosphate, 2′-deoxyinosine 5′-phosphate, 3393-18- |
| | 8, C10H13N4O7P, hypoxanthine deoxyriboside, [(2R,3S,5R)-3-hydroxy-5-(6- |
| | oxo-3H-purin-9-yl)oxolan-2-yl]methoxyphosphonic acid |
| dITP | 16595-02-1, 2′-deoxyinosine 5′-triphosphate, 2′-dITP, C10H15N4O13P3, |
| | inosine 5′-(tetrahydrogen triphosphate), 2′-deoxy-, [hydroxy-[hydroxy- |
| | [[(2R,3S,5R)-3-hydroxy-5-(6-oxo-3H-purin-9-yl)oxolan-2- |
| | yl]methoxy]phosphoryl]oxy-phosphoryl]oxyphosphonic acid |
| FGAM | 1-(5′-phosphoribosyl)-N-formylglycinamidine, 2-(formamido)-N1-(5-phospho- |
| | D-ribosyl)acetamidine, 5′-phosphoribosyl-N-formylglycinamidine, 5′- |
| | phosphoribosylformylglycinamidine, C8H16N3O8P, [(2R,3R,4R,5R)-5-[(1- |
| | amino-2-formamido-ethylidene)amino]-3,4-dihydroxy-oxolan-2- |
| | yl]methoxyphosphonic acid |
| Formiminoglycine | 2-(aminomethylideneamino)acetic acid, 2140-03-6, C3H6N2O2, |
| | formimidoylglycine, glycine, N-(iminomethyl)-, N-formiminoglycine |
| GAR | 10074-18-7, 2-amino-(N-D-ribofuranosyl)acetamide 5′-phosphate, 5′- |
| | phosphoribosylglycinamide, 5′-phosphoribosylglycineamide, C7H15N2O8P, |
| | GAR, glycinamide ribonucleotide, [(2R,3R,4R,5R)-5-[(2-aminoacetyl)amino]- |
| | 3,4-dihydroxy-oxolan-2-yl]methoxyphosphonic acid |
| GDP | 146-91-8, C10H15N5O11P2, guanosine 5′-(trihydrogen diphosphate), |
| | guanosine diphosphate, [[(2R,3R,4R,5R)-5-(2-amino-6-oxo-3H-purin-9-yl)- |
| | 3,4-dihydroxy-oxolan-2-yl]methoxy-hydroxy-phosphoryl]oxyphosphonic acid |
| Glycine | 2-aminoacetic acid, 56-40-6, C2H5NO2, glycine, zirconium aluminum glycine |
| Glyoxylate | 298-12-4, alpha-ketoacetic acid, C2H2O3, formylformic acid, glyoxylate, |
| | oxaldehydic acid, oxoacetic acid |
| GMP | 5′-GMP, 5′-guanylic acid, 85-32-5, C10H14N5O8P, GMP5′, guanosine |
| | monophosphate, [(2R,3R,4R,5R)-5-(2-amino-6-oxo-3H-purin-9-yl)-3,4- |
| | dihydroxy-oxolan-2-yl]methoxyphosphonic acid |
| GppppG | 4130-19-2, bis(5′-guanosyl) tetraphosphate, C20H28N10O21P4, GP4G, |
| | GppppG, P1,P4-bis(5′-guanosyl) tetraphosphate, [[(2R,3R,4R,5R)-5-(2- |
| | amino-6-oxo-3H-purin-9-yl)-3,4-dihydroxy-oxolan-2-yl]methoxy-hydroxy- |
| | phosphoryl]oxy-[[[(2R,3R,4R,5R)-5-(2-amino-6-oxo-3H-purin-9-yl)-3,4- |
| | dihydroxy-oxolan-2-yl]methoxy-hydroxy-phosphoryl]oxy-hydroxy- |
| | phosphoryl]oxy-phosphinic acid |
| GTP | 86-01-1, C10H16N5O14P3, guanosine 5′-(tetrahydrogen triphosphate), |
| | guanosine triphosphate, Mg-GTP, [[[(2R,3R,4R,5R)-5-(2-amino-6-oxo-3H- |
| | purin-9-yl)-3,4-dihydroxy-oxolan-2-yl]methoxy-hydroxy-phosphoryl]oxy- |
| | hydroxy-phosphoryl]oxyphosphonic acid |
| Guanine | 2-amino-3,7-dihydropurin-6-one, 2-aminohypoxanthine, 6H-purin-6-one, 2- |
| | amino-1,7-dihydro, 73-40-5, C5H5N5O |
| Guanosine | 118-00-3, 2(3H)-imino-9-beta-D-ribofuranosyl-9H-purin-6(1H)-one, 2-amino- |
| | 9-[(2R,3R,4R,5R)-3,4-dihydroxy-5-(hydroxymethyl)oxolan-2-yl]-3H-purin-6- |
| | one, 6H-purin-6-one, 2-amino-1,9-dihydro-9-beta-D-ribofuranosyl-, 9-beta-D- |
| | ribofuranosylguanine, C10H13N5O5, guanine riboside, guanine, 9-beta-D- |
| | ribofuranosyl-(VAN), inosine, 2-amino- |
| Hypoxanthine | 3,7-dihydropurin-6-one, 3H-purin-6-ol, 6(1H)-purinone, 6-hydroxy-1H-purine, |
| | 6-hydroxypurine, 6-oxopurine, 68-94-0, 6H-purin-6-one, 1,7-dihydro-, |
| | C5H4N4O, hypoxanthine (VAN) (8Cl), purin-6(1H)-one |
| IDP | 86-04-4, C10H14N4O11P2, idp, inosine 5′-(trihydrogen diphosphate), inosine |
| | 5′-diphosphate, [[(2R,3R,4R,5R)-3,4-dihydroxy-5-(6-oxo-3H-purin-9- |
| | yl)oxolan-2-yl]methoxy-hydroxy-phosphoryl]oxyphosphonic acid |
| Imidazolone | 1968-28-1, 3,5-dihydroimidazol-4-one, C3H4N2O |
| IMP | 131-99-7, 5′-IMP, 5′-inosinic acid, C10H13N4O8P, inosine monophosphate, |
| | inosine-5′-monophosphoric acid, inosinic acid, [(2R,3R,4R,5R)-3,4-dihydroxy- |
| | 5-(6-oxo-3H-purin-9-yl)oxolan-2-yl]methoxyphosphonic acid |
| Inosine | 58-63-9, 9-[(2R,3R,4R,5R)-3,4-dihydroxy-5-(hydroxymethyl)oxolan-2-yl]-3H- |
| | purin-6-one, beta-inosine, C10H12N4O5, hypoxanthine nucleoside, |
| | hypoxanthine riboside, hypoxanthine, 9-beta-D-ribofuranosyl-, oxiamin |
| Inosine 5′-tetraphosphate | C10H16N4O17P4, [[[[(2R,3R,4R,5R)-3,4-dihydroxy-5-(6-oxo-3H-purin-9- |
| | yl)oxolan-2-yl]methoxy-hydroxy-phosphoryl]oxy-hydroxy-phosphoryl]oxy- |
| | hydroxy-phosphoryl]oxyphosphonic acid |
| ITP(extracellular) | 132-06-9, C10H15N4O14P3, inosine 5′-(tetrahydrogen triphosphate), inosine |
| | triphosphate, [[[(2R,3R,4R,5R)-3,4-dihydroxy-5-(6-oxo-3H-purin-9-yl)oxolan- |
| | 2-yl]methoxy-hydroxy-phosphoryl]oxy-hydroxy-phosphoryl]oxyphosphonic |
| | acid |
| L-Glutamine | (2S)-2-amino-4-carbamoyl-butanoic acid, (S)-2,5-diamino-5-oxopentanoic |
| | acid, 56-85-9, C5H10N2O3, L-2-aminoglutaramidic acid, L-glutamine, |
| | levoglutamide |
| NH3 | 7664-41-7, ammonia, anhydrous, anhydrous ammonia, azane, H3N |
| Oxalureate | 585-05-7, acetic acid, ((aminocarbonyl)amino)oxo-, C3H4N2O4, |
| | carbamoylcarbamoylformic acid, carbamoyloxamic acid, monooxalylurea, |
| | oxalureate |
| ppGpp | C10H17N5O17P4, guanosine 3′,5′-bis(diphosphate), [[(2R,3R,4R,5R)-5-(2- |
| | amino-6-oxo-3H-purin-9-yl)-4-hydroxy-3-(hydroxy-phosphonooxy- |
| | phosphoryl)oxy-oxolan-2-yl]methoxy-hydroxy-phosphoryl]oxyphosphonic acid |
| pppGpp | 38918-96-6, C10H18N5O20P5, guanosine pentaphosphate, magic spot II, |
| | [[[(2R,3R,4R,5R)-5-(2-amino-6-oxo-3H-purin-9-yl)-4-hydroxy-3-(hydroxy- |
| | phosphonooxy-phosphoryl)oxy-oxolan-2-yl]methoxy-hydroxy-phosphoryl]oxy- |
| | hydroxy-phosphoryl]oxyphosphonic acid |
| PRPP | 7540-64-9, alpha-D-ribofuranose 5-(dihydrogen phosphate) 1-(trihydrogen |
| | diphosphate), C5H13O14P3, PRPP, [[(2R,3R,4R,5R)-3,4-dihydroxy-5- |
| | (phosphonooxymethyl)oxolan-2-yl]oxy-hydroxy-phosphoryl]oxyphosphonic |
| | acid |
| Ribose-5P | 4300-28-1, C5H11O8P, D-ribose 5-(dihydrogen phosphate), D-ribose-5- |
| | phosphoric acid, R-5-P, ribose 5-monophosphate, ribose 5-phosphate, ribose |
| | phosphate, [(2R,3S,4R)-3,4,5-trihydroxyoxolan-2-yl]methoxyphosphonic acid |
| Ribosylamine-5P | 14050-66-9, 5-phospho-beta-D-ribosylamine, 5-phospho-D-ribosylamine, 5- |
| | phosphoribosyl-1-amine, C5H12NO7P, D-Ribofuranosylamine, 5- |
| | (dihydrogen phosphate), phosphoribosylamine, [(2R,3R,4R)-5-amino-3,4- |
| | dihydroxy-oxolan-2-yl]methoxyphosphonic acid |
| Sulfate | 14808-79-8, O4S-2, sulfate, sulfate ion, sulfate(2-) |
| Urate | 1198-77-2, 1H-purine-2,6,8(3H)-trione, 7,9-dihydro-, 1H-purine-2,6,8(3H)- |
| | trione, 7,9-dihydro-, monosodium salt, 69-93-2, 7,9-dihydro-3H-purine-2,6,8- |
| | trione, C5H4N4O3, lithic acid, monosodium urate, monosodium urate |
| | microcrystals, MSU, urate |
| Urate-3-ribonucleoside | 2124-54-1, 3-ribosyluric acid, 3-[(2R,3R,4R,5R)-3,4-dihydroxy-5- |
| | (hydroxymethyl)oxolan-2-yl]-7,9-dihydropurine-2,6,8-trione, 7,9-dihydro-3- |
| | beta-D-ribofuranosyl-1H-purine-2,6,8(3H)-trione, C10H12N4O7, urate-3- |
| | ribonucleoside, uric acid ribonucleoside |
| Urea | 57-13-6, carbonyl diamide, CH4N2O, urea, urea extract |
| Ureidoglycine | 2-amino-2-(carbamoylamino)acetic acid, C3H7N3O3 |
| Ureidoglycolate | (2S)-2-(carbamoylamino)-2-hydroxy-acetic acid, C3H6N2O4 |
| Xanthine | 1H-purine-2,6-dione, 3,7-dihydro, 2,6-dioxo-1,2,3,6-tetrahydropurine, 2,6- |
| | dioxopurine, 3,7-dihydropurine-2,6-dione, 69-89-6, C5H4N4O2, purine- |
| | 2(3H),6(1H)-dione, xanthic oxide, xanthine (VAN) (8Cl) |
| Xanthosine | 146-80-5, 1H-purine-2,6-dione, 3,9-dihydro-9-beta-D-ribofuranosyl-, 9-beta- |
| | D-ribofuranosylxanthine, 9-[(2R,3R,4R,5R)-3,4-dihydroxy-5- |
| | (hydroxymethyl)oxolan-2-yl]-3H-purine-2,6-dione, C10H12N4O6, xanthine |
| | riboside |
| XMP | (9-D-ribosylxanthine)-5′-phosphate, 5′-xanthylic acid, 523-98-8, |
| | C10H13N4O9P, xanthosine 5′-phosphate, xanthosine monophosphate, |
| | xanthylic acid, [(2R,3R,4R,5R)-5-(2,6-dioxo-3H-purin-9-yl)-3,4-dihydroxy- |
| | oxolan-2-yl]methoxyphosphonic acid |
| XppppX | C20H26N8O23P4, XppppX, [[(2R,3R,4R,5R)-5-(2,6-dioxo-3H-purin-9-yl)-3,4- |
| | dihydroxy-oxolan-2-yl]methoxy-hydroxy-phosphoryl]oxy-[[[(2R,3R,4R,5R)-5- |
| | (2,6-dioxo-3H-purin-9-yl)-3,4-dihydroxy-oxolan-2-yl]methoxy-hydroxy- |
| | phosphoryl]oxy-hydroxy-phosphoryl]oxy-phosphinic acid |
| XTP | 6253-56-1, C10H15N4O15P3, xanthosine 5′-(tetrahydrogen triphosphate), |
| | xanthosine 5′-triphosphate, xanthosine triphosphate, [[[(2R,3R,4R)-5-(2,6- |
| | dioxo-3H-purin-9-yl)-3,4-dihydroxy-oxolan-2-yl]methoxy-hydroxy- |
| | phosphoryl]oxy-hydroxy-phosphoryl]oxyphosphonic acid |

*Genes/proteins that were used to identify the pathway:
2.4.2.8 6-hydroxypurine phosphoribosyltransferase, 6-mercaptopurine phosphoribosyltransferase, GMP pyrophosphorylase, GPRT, guanine phosphoribosyltransferase, guanine-hypoxanthine phosphoribosyltransferase, guanosine 5′-phosphate pyrophosphorylase, guanosine phosphoribosyltransferase, guanylate pyrophosphorylase, guanylic pyrophosphorylase, HGPRTase, HPRT, hypoxanthine-guanine phosphoribosyltransferase, IMP pyrophosphorylase, IMP-GMP pyrophosphorylase, IMP:diphosphate phospho-D-ribosyltransferase, inosinate pyrophosphorylase, inosine 5′-phosphate pyrophosphorylase, inosinic acid pyrophosphorylase, inosinic pyrophosphorylase, purine-6-thiol phosphoribosyltransferase, transphosphoribosidase
2.7.7.6 C ribonucleic acid formation factors, C RNA formation factors, deoxyribonucleic acid-dependent ribonucleic acid polymerase, DNA-dependent ribonucleate nucleotidyltransferase, DNA-dependent RNA nucleotidyltransferase, DNA-dependent RNA polymerase, nucleoside-triphosphate:RNA nucleotidyltransferase (DNA-directed), ribonucleate nucleotidyltransferase, ribonucleate polymerase, ribonucleic acid nucleotidyltransferase, ribonucleic acid polymerase, ribonucleic acid transcriptase, ribonucleic polymerase, ribonucleic transcriptase, RNA nucleotidyltransferase, RNA nucleotidyltransferase (DNA-directed), RNA polymerase, RNA polymerase I, RNA polymerase II, RNA polymerase III, RNA transcriptase, transcriptase

### Example 6

### Exemplary Pathways Relating to Low Ammonium Production

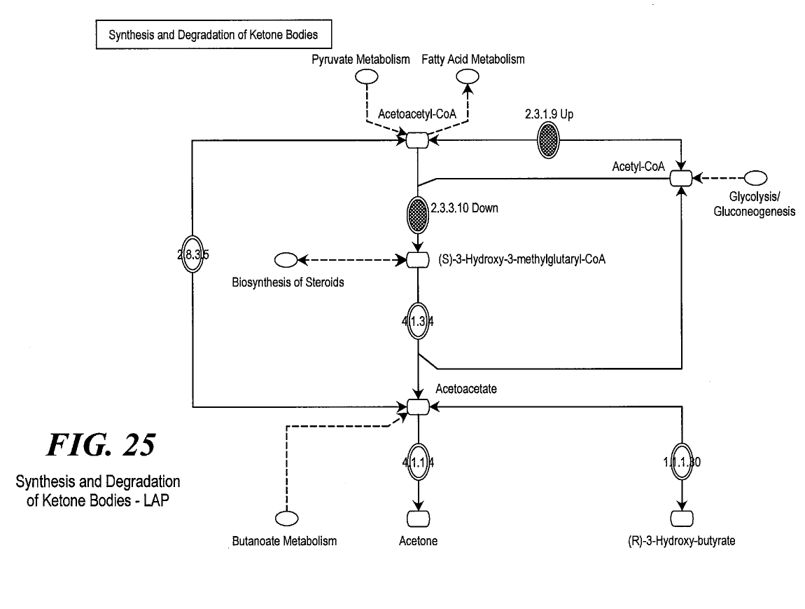

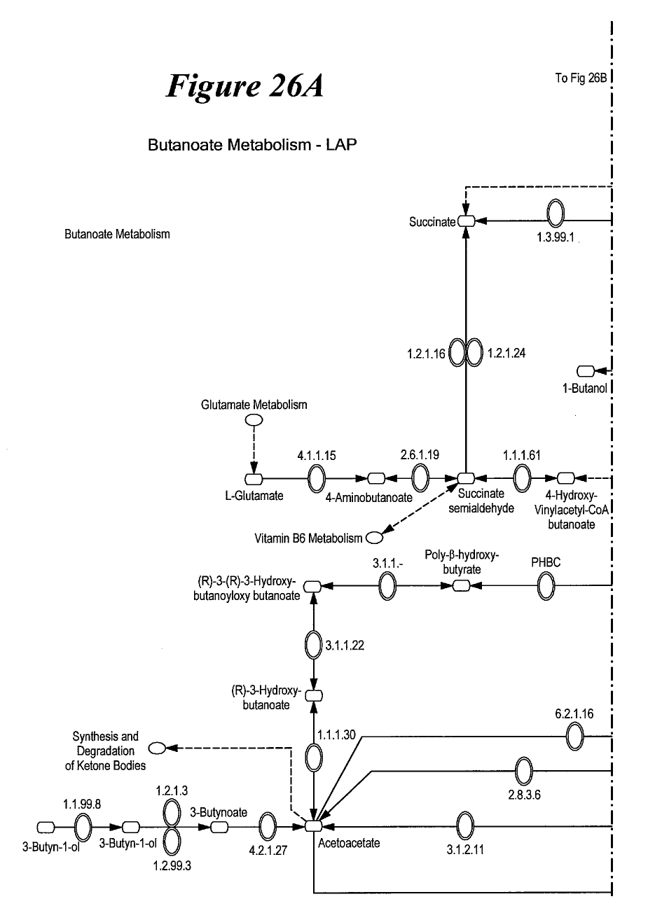

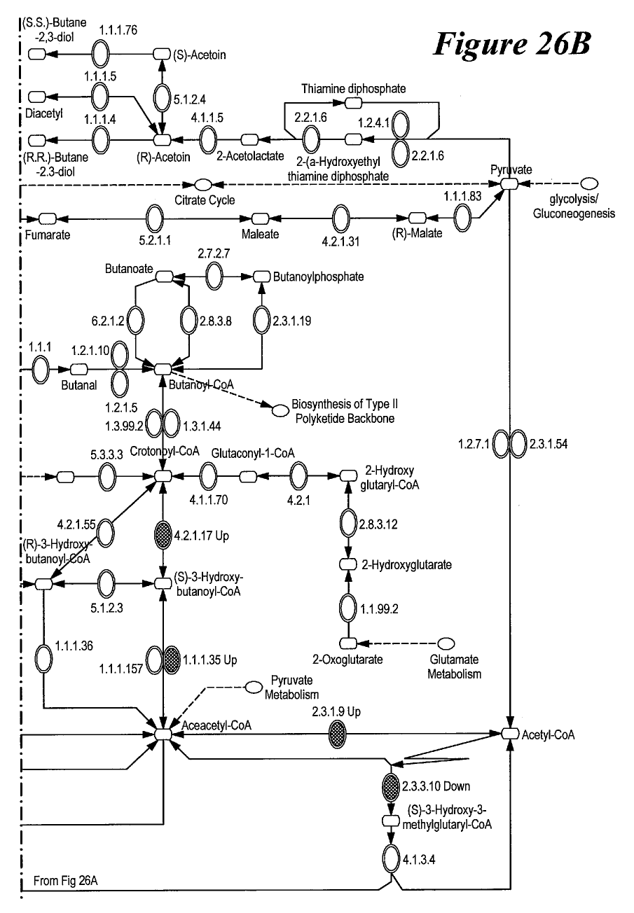

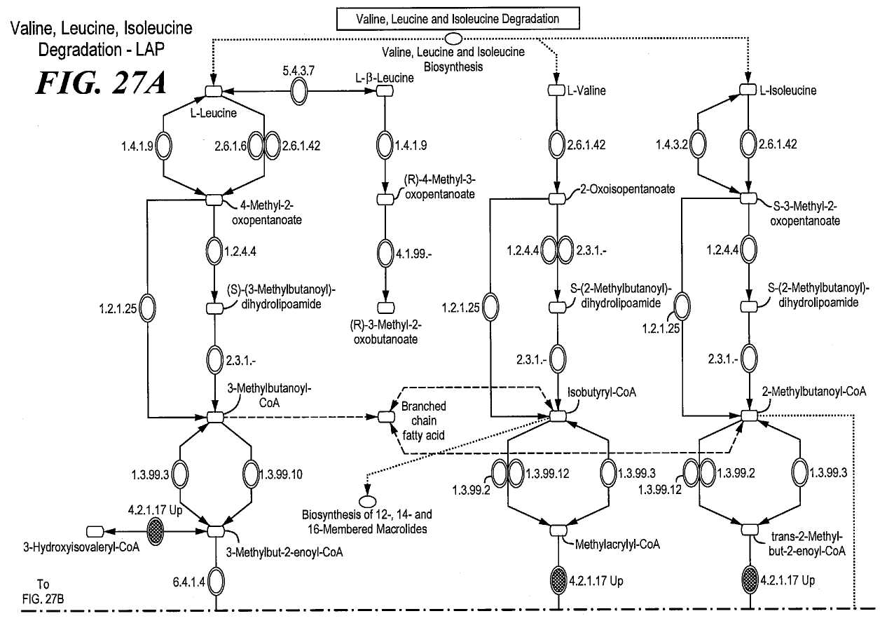

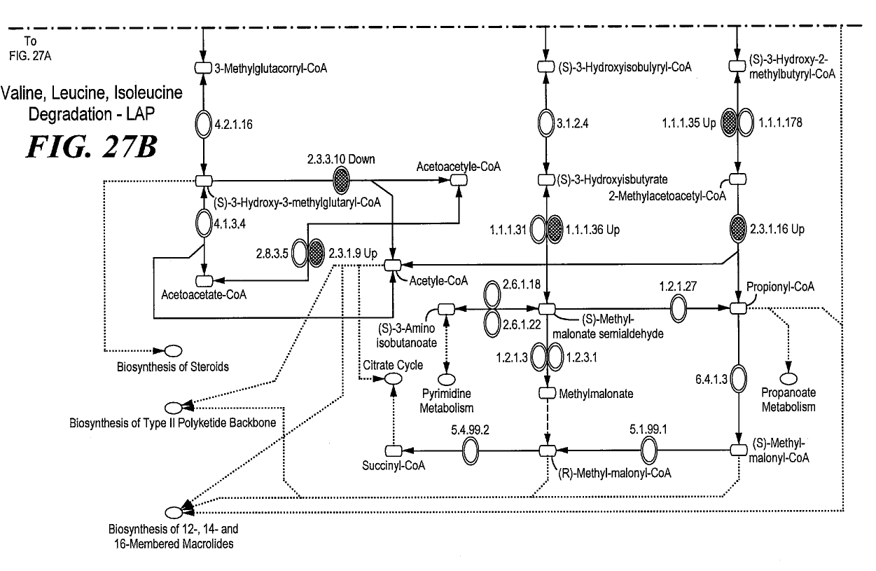

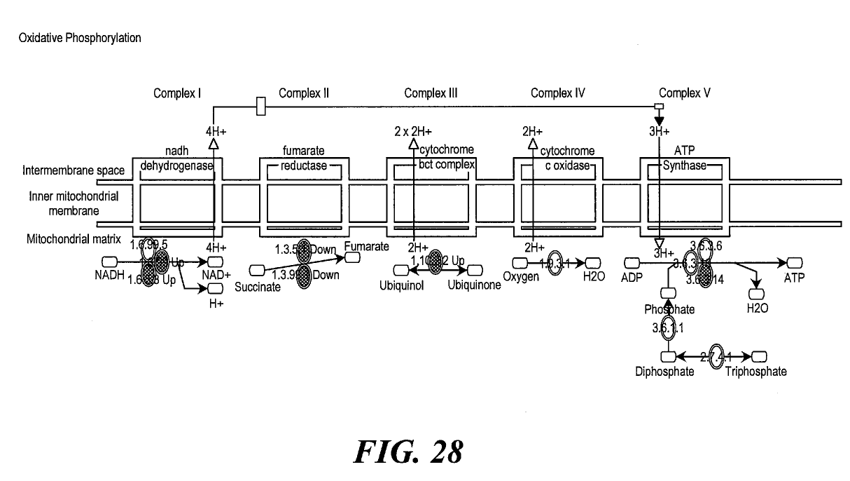

Pathway analysis using Ingenuity software based on previously identified differently expressed genes or proteins associated with low ammonium production led to the identification of the ER stress pathway (FIG. 25), the synthesis and degradation of ketone bodies pathway (FIG. 26), the butanoate metabolism pathway (FIG. 27), and the valine, leucine, isoleucine degradation pathway (FIG. 28). Genes/proteins that were used to identify relevant pathways are indicated in FIGS. 25-28. In addition, additional exemplary genes or proteins involved in the above-identified pathways and that may be involved in regulating or indicative of low ammonium production are summarized in Table 25 (the ER stress pathway), Table 26 (the synthesis and degradation of ketone bodies pathway), Table 27 (the butanoate metabolism pathway), and Table 28 (the valine, leucine, isoleucine degradation pathway).

TABLE 25
Genes/Proteins Involved in the ER stress pathway

| Gene | |
|---|---|
| Name | Synonyms |
| ASK1 | 7420452D20Rik, APOPTOSIS SIGNAL REGULATED KINASE 1, ASK, ASK1, Map3k5, |
| | MAPKKK5, MEKK5, MGC141518, MGC141519, RGD1306565 predicted, |
| | RGD1306565_predicted |
| ATF4 | C/ATF, CREB-2, MGC96460, TAXREB67, TXREB |
| ATF6 | 9130025P16RIK, 9630036G24, AA789574, Atf6 (predicted), ATF6 ALPHA, ESTM49 |
| Caspase12 | Casp12, CASPASE12 |
| Caspase3 | A830040C14Rik, Apopain, CASPASE-3, CPP32, CPP32B, Cpp32beta, Ice-like cysteine |
| | protease, Lice, MGC93645, P17, PROCASPASE 3, SCA-1, YAMA |
| Caspase7 | AI314680, CASPASE7, CMH-1, ICE-IAP3, ICE-LAP3, mCASP-7, MCH3 |
| Caspase9 | AI115399, APAF-3, AW493809, Casp-9-CTD, Casp9 v1, CASPASE-9, CASPASE-9c, ICE- |
| | LAP6, MCH6 |
| EIF2A | 0910001O23Rik, 2410026C18Rik, 35 kDa, EIF-2, EIF-2A, EIF-2ALPHA, Eukaryotic Translation |
| | Initiation Factor 2 Alpha Subunit, MGC93488 |
| IRE1 | 9030414B18Rik, AI225830, C85377, ERN1, FLJ30999, hIRE1p, Inositol-requiring 1, IRE1, |
| | IRE1-ALPHA, IRE1A, IRE1P, MGC163277, MGC163279, RGD1559716, RGD1559716 |
| | predicted, RGD1559716_predicted |
| JIK | A130052D22, A430105I05Rik, DKFZp666H245, DPK, FLJ31808, JIK, MAP3K18 |
| JNK1 | AI849689, C-JUN N-TERMINAL KINASE1, JNK, JNK1, JNK1 PROTEIN KINASE, JNK1A2, |
| | JNK21B1/2, p46JNK1, p46JNK1 ALPHA, PRKM8, Sapk gamma, SAPK P46, SAPK1, |
| | SAPK1/JNK, STRESS-ACTIVATED PROTEIN KINASE-LIKE KINASE |
| MBTPS | |
| P58IPK | 58 kda Inhibitor Of RNA Activated Protein Kinase, AA408985, AU067833, Dnajc3a, Dnajc3b, |
| | HP58, LOC63880, MGC6474, mp58, P58, P58IPK, p58K, Pkip58, PRKRI |
| PERK | AI427929, DKFZp781H1925, HRI, PEK, PERK, WRS |
| TRAF2 | AI325259, MGC: 45012, TNF Receptor-Associated Factor 2, TRAP, TRAP3 |
| XBP1 | D11Ertd39e, Hepatocarcinogenesis-related transcription factor, HTF, Sxbp-1, TREB-5, XBP2 |
| mRNA | |

Genes/proteins that were used to identify the pathway:
BiP 78 kDa, AL022860, AU019543, BIP, D2Wsu141e, D2Wsu17e, FLJ26106, GRP78, HEAT SHOCK 70 KDA PROTEIN5, Hsce70, HSP70-5, Immunoglobulin heavy chain binding protein, mBiP, MIF2, SEZ-7

TABLE 26
Genes/Proteins Involved in the Synthesis and degradation of ketone bodies pathway

| Name | Synonyms |
|---|---|
| (R)-3-Hydroxy-butyrate | (3R)-3-hydroxybutanoic acid, (R)-(−)-3-hydroxybutyric acid sodium salt, (R)-3- |
| | hydroxybutanoic acid, (R)-3-hydroxybutyric acid, 13613-65-5, 625-72-9, |
| | C4H8O3, D-beta-hydroxybutyrate, R-3-hydroxybutanoate, sodium (R)-3- |
| | hydroxybutyrate |
| (S)-3-Hydroxy-3- | (3S)-4-[2-[3-[[4-[[[(2R,3R,4R,5R)-5-(6-aminopurin-9-yl)-4-hydroxy-3- |
| methylglutaryl-CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-hydroxy-3-methyl- |
| | butanoic acid, (S)-3-hydroxy-3-methylglutaryl-CoA, 1553-55-5, |
| | C27H44N7O20P3S, hydroxymethylglutaryl-CoA, S-(hydrogen 3-hydroxy-3- |
| | methylglutaryl)coenzyme A, S-(hydrogen 3-hydroxy-3-methylpentanedioate)coenzyme A |
| 1.1.1.30 | (R)-3-hydroxybutanoate:NAD oxidoreductase, 3-D-hydroxybutyrate |
| | dehydrogenase, beta-hydroxybutyrate dehydrogenase, beta-hydroxybutyric |
| | acid dehydrogenase, beta-hydroxybutyric dehydrogenase, D-(−)-3- |
| | hydroxybutyrate dehydrogenase, D-3-hydroxybutyrate dehydrogenase, D- |
| | beta-hydroxybutyrate dehydrogenase, hydroxybutyrate oxidoreductase, NAD- |
| | beta-hydroxybutyrate dehydrogenase |
| 2.8.3.5 | 3-ketoacid CoA-transferase, 3-ketoacid coenzyme A transferase, 3-oxo-CoA |
| | transferase, 3-oxoacid CoA dehydrogenase, 3-oxoacid coenzyme A- |
| | transferase, acetoacetate succinyl-CoA transferase, acetoacetyl coenzyme A- |
| | succinic thiophorase, succinyl coenzyme A-acetoacetyl coenzyme A- |
| | transferase, succinyl-CoA transferase, succinyl-CoA:3-oxo-acid CoA- |
| | transferase |
| 4.1.1.4 | acetoacetate carboxy-lyase, acetoacetic acid decarboxylase |
| 4.1.3.4 | (S)-3-hydroxy-3-methylglutaryl-CoA acetoacetate-lyase, 3-hydroxy-3- |
| | methylglutaryl CoA cleaving enzyme, 3-hydroxy-3-methylglutaryl coenzyme A |
| | lyase, 3-hydroxy-3-methylglutaryl-CoA lyase, hydroxymethylglutaryl coenzyme |
| | A lyase, hydroxymethylglutaryl coenzyme A-cleaving enzyme |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme A, |
| | [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetone | 2-Propanone, 67-64-1, acetone, C3H6O, dimethyl ketone, |
| | dimethylformaldehyde, dimethylketal, propanone |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |

*Genes/proteins that were used to identify the pathway:
2.3.1.9 2-methylacetoacetyl-CoA thiolase, 3-oxothiolase, acetoacetyl-CoA thiolase, acetyl coenzyme A thiolase, acetyl-CoA acetyltransferase, acetyl-CoA:acetyl-CoA C-acetyltransferase, acetyl-CoA:N-acetyltransferase, beta-acetoacetyl coenzyme A thiolase, thiolase II
2.3.3.10 (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b-methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase

TABLE 27
Genes/Proteins Involved in the Butanoate metabolism pathway

| Name | Synonyms |
|---|---|
| (R)-3-((R)-3- | (3R)-3-[(3R)-3-hydroxybutanoyl]oxybutanoic acid, (R)-3-((R)-3-hydroxybutanoyloxy)-butanoate, |
| Hydroxy- | C8H14O5 |
| butanoyloxy)butanoate | |
| (R)-3-Hydroxy- | (3R)-3-hydroxybutanoic acid, (R)-(−)-3-hydroxybutyric acid sodium salt, (R)-3-hydroxybutanoic acid, |
| butanoate | (R)-3-hydroxybutyric acid, 13613-65-5, 625-72-9, C4H8O3, D-beta-hydroxybutyrate, R-3- |
| | hydroxybutanoate, sodium (R)-3-hydroxybutyrate |
| (R)-3-Hydroxy- | (R)-3-hydroxybutanoyl-CoA, (R)-3-hydroxybutyryl-coenzyme A, 21804-29-5, C25H42N7O18P3S, |
| butanoyl-CoA | [(2R,3R,4R,5R)-5-(6-aminopurin-9-yl)-4-hydroxy-2-[[hydroxy-[hydroxy-[3-hydroxy-3-[2-[2-[(3R)-3- |
| | hydroxybutanoyl]sulfanylethylcarbamoyl]ethylcarbamoyl]-2,2-dimethyl-propoxy]phosphoryl]oxy- |
| | phosphoryl]oxymethyl]oxolan-3-yl]oxyphosphonic acid |
| (R)-Acetoin | (3R)-3-hydroxybutan-2-one, (R)-2-acetoin, (R)-3-hydroxy-2-butanone, (R)-3-hydroxybutan-2-one, |
| | (R)-dimethylketol, C4H8O2 |
| (R)-Malate | (2R)-2-hydroxybutanedioic acid, (R)-malate, 636-61-3, C4H6O5, D-malate, malic acid, L(+)- |
| (R,R)-Butane-2,3- | (2R,3R)-butane-2,3-diol, (R,R)-(−)-butane-2,3-diol, (R,R)-2,3-butanediol, (R,R)-butane-2,3-diol, |
| diol | 24347-58-8, C4H10O2, r,r-butane-2,3-diol |
| (S)-3-Hydroxy- | (S)-3-hydroxybutanoyl-CoA, (S)-3-hydroxybutyryl-CoA, (S)-3-hydroxybutyryl-coenzyme A, 22138- |
| butanoyl-CoA | 45-0, C25H42N7O18P3S, [(2R,3R,4R,5R)-5-(6-aminopurin-9-yl)-4-hydroxy-2-[[hydroxy-[hydroxy-[3- |
| | hydroxy-3-[2-[2-[(3S)-3-hydroxybutanoyl]sulfanylethylcarbamoyl]ethylcarbamoyl]-2,2-dimethyl- |
| | propoxy]phosphoryl]oxy-phosphoryl]oxymethyl]oxolan-3-yl]oxyphosphonic acid |
| (S)-3-Hydroxy-3- | (3S)-4-[2-[3-[[4-[[[(2R,3R,4R,5R)-5-(6-aminopurin-9-yl)-4-hydroxy-3-phosphonooxy-oxolan-2- |
| methylglutaryl-CoA | yl]methoxy-hydroxy-phosphoryl]oxy-hydroxy-phosphoryl]oxy-2-hydroxy-3,3-dimethyl- |
| | butanoyl]amino]propanoylamino]ethylsulfanylcarbonyl]-3-hydroxy-3-methyl-butanoic acid, (S)-3- |
| | hydroxy-3-methylglutaryl-CoA, 1553-55-5, C27H44N7O20P3S, hydroxymethylglutaryl-CoA, S- |
| | (hydrogen 3-hydroxy-3-methylglutaryl)coenzyme A, S-(hydrogen 3-hydroxy-3-methylpentanedioate)coenzyme A |
| (S)-Acetoin | (3S)-3-hydroxybutan-2-one, C4H8O2 |
| (S,S)-Butane-2,3- | (2S,3S)-butane-2,3-diol, (S,S)-butane-2,3-diol, 19132-06-0, 2,3-butanediol, (S-(R*,R*))-, C4H10O2 |
| diol | |
| 1-Butanol | 1-butanol, 1-hydroxybutane, 71-36-3, butan-1-ol, butanol, butyl alcohol, C4H10O, n-butanol |
| 1.1.1.— | |
| 1.1.1.157 | (S)-3-hydroxybutanoyl-CoA:NADP oxidoreductase, beta-hydroxybutyryl coenzyme A |
| | dehydrogenase, beta-hydroxybutyryl-CoA dehydrogenase, BHBD, dehydrogenase, L-3- |
| | hydroxybutyryl coenzyme A (nicotinamide adenine dinucleotide phosphate), L(+)-3-hydroxybutyryl- |
| | CoA dehydrogenase |
| 1.1.1.30 | (R)-3-hydroxybutanoate:NAD oxidoreductase, 3-D-hydroxybutyrate dehydrogenase, beta- |
| | hydroxybutyrate dehydrogenase, beta-hydroxybutyric acid dehydrogenase, beta-hydroxybutyric |
| | dehydrogenase, D-(−)-3-hydroxybutyrate dehydrogenase, D-3-hydroxybutyrate dehydrogenase, D- |
| | beta-hydroxybutyrate dehydrogenase, hydroxybutyrate oxidoreductase, NAD-beta-hydroxybutyrate |
| | dehydrogenase |
| 1.1.1.36 | (R)-3-hydroxyacyl-CoA dehydrogenase, (R)-3-hydroxyacyl-CoA:NADP oxidoreductase, acetoacetyl |
| | coenzyme A reductase, beta-ketoacyl-CoA reductase, D(−)-beta-hydroxybutyryl CoA-NADP |
| | oxidoreductase, D-3-hydroxyacyl-CoA reductase, hydroxyacyl coenzyme-A dehydrogenase, NADP- |
| | linked acetoacetyl CoA reductase, NADPH:acetoacetyl-CoA reductase, short chain beta- |
| | ketoacetyl(acetoacetyl)-CoA reductase |
| 1.1.1.4 | (R)-2,3-butanediol dehydrogenase, (R)-diacetyl reductase, (R,R)-butane-2,3-diol:NAD |
| | oxidoreductase, 1-amino-2-propanol dehydrogenase, 1-amino-2-propanol oxidoreductase, 2,3- |
| | butanediol dehydrogenase, aminopropanol oxidoreductase, butylene glycol dehydrogenase, D-(−)- |
| | butanediol dehydrogenase, D-1-amino-2-propanol dehydrogenase, D-1-amino-2-propanol:NAD+ |
| | oxidoreductase, D-aminopropanol dehydrogenase, D-butanediol dehydrogenase, diacetyl (acetoin)reductase |
| 1.1.1.5 | acetoin:NAD oxidoreductase, diacetyl reductase |
| 1.1.1.61 | 4-hydroxybutanoate:NAD oxidoreductase, g-hydroxybutyrate dehydrogenase |
| 1.1.1.76 | (S,S)-butane-2,3-diol:NAD oxidoreductase, L(+)-2,3-butanediol dehydrogenase (L-acetoin forming), |
| | L-BDH, L-butanediol dehydrogenase |
| 1.1.1.83 | (R)-malate:NAD oxidoreductase (decarboxylating), bifunctional L(+)-tartrate dehydrogenase-D(+)- |
| | malate (decarboxylating), D-malate dehydrogenase, D-malic enzyme |
| 1.1.99.2 | (S)-2-hydroxyglutarate:(acceptor) 2-oxidoreductase, alpha-hydroxyglutarate dehydrogenase, alpha- |
| | hydroxyglutarate dehydrogenase (NAD+ specific), alpha-hydroxyglutarate oxidoreductase, alpha- |
| | ketoglutarate reductase, hydroxyglutaric dehydrogenase, L-alpha-hydroxyglutarate dehydrogenase, |
| | L-alpha-hydroxyglutarate:NAD+ 2-oxidoreductase |
| 1.1.99.8 | alcohol:(acceptor) oxidoreductase, MDH, primary alcohol dehydrogenase, quinohemoprotein |
| | alcohol dehydrogenase, quinoprotein alcohol dehydrogenase, quinoprotein ethanol dehydrogenase |
| 1.2.1.10 | acetaldehyde:NAD oxidoreductase (CoA-acetylating), aldehyde dehydrogenase (acylating) |
| 1.2.1.16 | succinate semialdehyde dehydrogenase (nicotinamide adenine dinucleotide (phosphate)), |
| | succinate-semialdehyde:NAD(P) oxidoreductase |
| 1.2.1.24 | succinate semialdehyde:NAD+ oxidoreductase, succinate-semialdehyde:NAD oxidoreductase, |
| | succinic semialdehyde dehydrogenase, succinyl semialdehyde dehydrogenase |
| 1.2.1.3 | aldehyde:NAD oxidoreductase, CoA-independent aldehyde dehydrogenase, m-methylbenzaldehyde |
| | dehydrogenase, NAD-aldehyde dehydrogenase, NAD-dependent 4-hydroxynonenal |
| | dehydrogenase, NAD-dependent aldehyde dehydrogenase, NAD-linked aldehyde dehydrogenase, |
| | propionaldehyde dehydrogenase |
| 1.2.1.57 | butanal:NAD(P) oxidoreductase (CoA-acylating) |
| 1.2.4.1 | MtPDC (mitochondrial pyruvate dehydogenase complex), PDH, pyruvate decarboxylase, pyruvate |
| | dehydrogenase, pyruvate dehydrogenase complex, pyruvate:lipoamide 2-oxidoreductase |
| | (decarboxylating and acceptor-acetylating), pyruvic acid dehydrogenase, pyruvic dehydrogenase |
| 1.2.7.1 | pyruvate oxidoreductase, pyruvate synthetase, pyruvate:ferredoxin 2-oxidoreductase (CoA- |
| | acetylating), pyruvate:ferredoxin oxidoreductase, pyruvic-ferredoxin oxidoreductase |
| 1.2.99.3 | aldehyde dehydrogenase (acceptor), aldehyde:(pyrroloquinoline-quinone) oxidoreductase |
| 1.3.1.44 | acyl-CoA:NAD trans-2-oxidoreductase |
| 1.3.99.1 | Complex II, Succinate INT Dehydrogenase |
| 1.3.99.2 | 3-hydroxyacyl CoA reductase, butanoyl-CoA:(acceptor) 2,3-oxidoreductase, butyryl coenzyme A |
| | dehydrogenase, butyryl dehydrogenase, enoyl-coenzyme A reductase, ethylene reductase, short- |
| | chain acyl CoA dehydrogenase, short-chain acyl-coenzyme A dehydrogenase, unsaturated acyl |
| | coenzyme A reductase, unsaturated acyl-CoA reductase |
| 2-(&alpha;- | 2-(1-hydroxyethyl)thiamine pyrophosphate, C14H23N4O8P2S+, [2-[3-[(4-amino-2-methyl-pyrimidin- |
| Hydroxyethyl)- | 5-yl)methyl]-2-(1-hydroxyethyl)-4-methyl-1-thia-3-azoniacyclopenta-2,4-dien-5-yl]ethoxy-hydroxy- |
| thiamine | phosphoryl]oxyphosphonic acid |
| diphosphate | |
| 2-Acetolactate | 2-acetoxypropanoic acid, 2-acetyloxypropanoic acid, 535-17-1, acetyllactic acid, alpha-acetolactate, |
| | alpha-acetoxypropionic acid, C5H8O4, propanoic acid, 2-(acetyloxy)- |
| 2-Hydroxy-glutaryl- | 2-hydroxyglutaryl-1-coa, 4-[2-[3-[[4-[[[(2R,3R,4R,5R)-5-(6-aminopurin-9-yl)-4-hydroxy-3- |
| CoA | phosphonooxy-oxolan-2-yl]methoxy-hydroxy-phosphoryl]oxy-hydroxy-phosphoryl]oxy-2-hydroxy- |
| | 3,3-dimethyl-butanoyl]amino]propanoylamino]ethylsulfanylcarbonyl]-4-hydroxy-butanoic acid, |
| | C26H42N7O20P3S, coenzyme A, S-(5-hydrogen 2-hydroxypentanedioate), (R)- |
| 2-Hydroxyglutarate | 2-hydroxyglutarate, 2-hydroxyglutaric acid, 2-hydroxypentanedioic acid, 2889-31-8, C5H8O5, |
| | pentanedioic acid, 2-hydroxy- |
| 2-Oxoglutarate | 2-ketoglutarate, 2-oxoglutarate, 2-oxopentanedioic acid, 328-50-7, alpha-ketoglutarate, alpha- |
| | ketoglutaric acid, alphaKG, C5H6O5, glutaric acid, 2-oxo-, glutaric acid, 2-oxo- (8Cl), pentanedioic |
| | acid, 2-oxo- |
| 2.2.1.6 | acetohydroxy acid synthetase, acetohydroxyacid synthase, acetolactate pyruvate-lyase |
| | (carboxylating), acetolactic synthetase, alpha-acetohydroxy acid synthetase, alpha- |
| | acetohydroxyacid synthase, alpha-acetolactate synthase, alpha-acetolactate synthetase |
| 2.3.1.19 | butanoyl-CoA:phosphate butanoyltransferase, phosphotransbutyrylase |
| 2.3.1.54 | acetyl-CoA:formate C-acetyltransferase, formate acetyltransferase, pyruvate formate-lyase, pyruvic |
| | formate-lyase |
| 2.6.1.19 | 4-aminobutanoate:2-oxoglutarate aminotransferase, 4-aminobutyrate aminotransferase, 4- |
| | aminobutyrate-2-ketoglutarate aminotransferase, 4-aminobutyrate-2-oxoglutarate aminotransferase, |
| | 4-aminobutyrate-2-oxoglutarate transaminase, 4-aminobutyric acid 2-ketoglutaric acid |
| | aminotransferase, 4-aminobutyric acid aminotransferase, aminobutyrate aminotransferase, |
| | aminobutyrate transaminase, beta-alanine aminotransferase, beta-alanine-oxoglutarate |
| | aminotransferase, beta-alanine-oxoglutarate transaminase, g-aminobutyrate aminotransaminase, g- |
| | aminobutyrate transaminase, g-aminobutyrate-alpha-ketoglutarate aminotransferase, g- |
| | aminobutyrate-alpha-ketoglutarate transaminase, g-aminobutyrate:alpha-oxoglutarate |
| | aminotransferase, g-aminobutyric acid aminotransferase, g-aminobutyric acid pyruvate |
| | transaminase, g-aminobutyric acid transaminase, g-aminobutyric acid-2-oxoglutarate transaminase, |
| | g-aminobutyric acid-alpha-ketoglutarate transaminase, g-aminobutyric acid-alpha-ketoglutaric acid |
| | aminotransferase, g-aminobutyric transaminase, GABA aminotransferase, GABA transaminase, |
| | GABA transferase, GABA-2-oxoglutarate aminotransferase, GABA-2-oxoglutarate transaminase, |
| | GABA-alpha-ketoglutarate aminotransferase, GABA-alpha-ketoglutarate transaminase, GABA- |
| | alpha-ketoglutaric acid transaminase, GABA-alpha-oxoglutarate aminotransferase, GABA- |
| | oxoglutarate aminotransferase, GABA-oxoglutarate transaminase, glutamate-succinic semialdehyde |
| | transaminase |
| 2.7.2.7 | ATP:butanoate 1-phosphotransferase |
| 2.8.3.12 | (E)-glutaconate CoA-transferase |
| 2.8.3.5 | 3-ketoacid CoA-transferase, 3-ketoacid coenzyme A transferase, 3-oxo-CoA transferase, 3-oxoacid |
| | CoA dehydrogenase, 3-oxoacid coenzyme A-transferase, acetoacetate succinyl-CoA transferase, |
| | acetoacetyl coenzyme A-succinic thiophorase, succinyl coenzyme A-acetoacetyl coenzyme A- |
| | transferase, succinyl-CoA transferase, succinyl-CoA:3-oxo-acid CoA-transferase |
| 2.8.3.8 | acetate coenzyme A-transferase, acyl-CoA:acetate CoA-transferase, butyryl CoA:acetate CoA |
| | transferase, butyryl coenzyme A transferase, succinyl-CoA:acetate CoA transferase |
| 3-Butyn-1-al | 52844-23-2, but-3-ynal, C4H4O |
| 3-Butyn-1-ol | 1-butyn-4-ol, 2-hydroxyethylacetylene, 3-butyne-1-ol, 3-butynol, 3-butynyl alcohol, 4-hydroxy-1- |
| | butyne, 927-74-2, but-3-yn-1-ol, C4H6O |
| 3-Butynoate | 2345-51-9, 3-butynoate, 3-butynoic acid, but-3-ynoic acid, C4H4O2 |
| 3.1.1.— | |
| 3.1.1.22 | (R)-3-((R)-3-hydroxybutanoyloxy)butanoate hydroxybutanoylhydrolase, D-(−)-3-hydroxybutyrate- |
| | dimer hydrolase |
| 3.1.2.11 | acetoacetyl CoA deacylase, acetoacetyl coenzyme A deacylase, acetoacetyl coenzyme A hydrolase |
| 4-Aminobutanoate | 4-aminobutanoic acid, 4-aminobutyrate, 4-aminobutyric acid, 56-12-2, butanoic acid, 4-amino-, |
| | C4H9NO2, gamma-amino-N-butyric acid, gamma-aminobutyric acid |
| 4-Hydroxy-butanoate | 4-hydroxybutanoate, 4-hydroxybutanoic acid, 4-hydroxybutyrate, 4-hydroxybutyric acid, 591-81-1, |
| | butanoic acid, 4-hydroxy-, C4H8O3, gamma-hydroxybutyrate, gamma-hydroxybutyric acid |
| 4.1.1.15 | aspartate 1-decarboxylase, aspartic alpha-decarboxylase, cysteic acid decarboxylase, g-glutamate |
| | decarboxylase, Glutamate decarboxylase, L-aspartate-alpha-decarboxylase, L-glutamate 1- |
| | carboxy-lyase, L-glutamate alpha-decarboxylase, L-glutamic acid decarboxylase, L-glutamic |
| | decarboxylase |
| 4.1.1.5 | (S)-2-hydroxy-2-methyl-3-oxobutanoate carboxy-lyase, alpha-acetolactate decarboxylase |
| 4.1.1.70 | glutaconyl coenzyme A decarboxylase, pent-2-enoyl-CoA carboxy-lyase |
| 4.1.3.4 | (S)-3-hydroxy-3-methylglutaryl-CoA acetoacetate-lyase, 3-hydroxy-3-methylglutaryl CoA cleaving |
| | enzyme, 3-hydroxy-3-methylglutaryl coenzyme A lyase, 3-hydroxy-3-methylglutaryl-CoA lyase, |
| | hydroxymethylglutaryl coenzyme A lyase, hydroxymethylglutaryl coenzyme A-cleaving enzyme |
| 4.2.1.— | EctC, HPAH, hydratase |
| 4.2.1.27 | 3-oxopropanoate hydro-lyase, acetylmonocarboxylic acid hydrase |
| 4.2.1.31 | (R)-malate hydro-lyase, D-malate hydro-lyase, malease |
| 4.2.1.55 | (3R)-3-hydroxybutanoyl-CoA hydro-lyase, D-3-hydroxybutyryl coenzyme A dehydratase, D-3- |
| | hydroxybutyryl-CoA dehydratase, enoyl coenzyme A hydrase (D) |
| 5.1.2.3 | 3-hydroxyacyl-CoA epimerase, 3-hydroxybutanoyl-CoA 3-epimerase, 3-hydroxybutyryl coenzyme A |
| | epimerase |
| 5.1.2.4 | acetylmethylcarbinol racemase |
| 5.2.1.1 | maleate cis-trans-isomerase |
| 5.3.3.3 | D3-cis-D2-trans-enoyl-CoA isomerase, vinylacetyl coenzyme A D-isomerase, vinylacetyl coenzyme |
| | A isomerase, vinylacetyl-CoA D3-D2-isomerase |
| 6.2.1.16 | acetoacetate:CoA ligase (AMP-forming), acetoacetyl-CoA synthetase |
| 6.2.1.2 | acyl-activating enzyme, butanoate:CoA ligase (AMP-forming), butyryl-CoA synthetase, fatty acid |
| | thiokinase (medium chain) |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme A, [(2R,3R,4R,5R)-5-(6- |
| | aminopurin-9-yl)-4-hydroxy-2-[[hydroxy-[hydroxy-[3-hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetyl-CoA | 72-89-9, acetyl-CoA, C23H38N7O17P3S, coenzyme A, S-acetate, S-acetyl coenzyme A, |
| | [(2R,3R,4R,5R)-2-[[[[3-[2-(2-acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6-aminopurin-9-yl)-4-hydroxy- |
| | oxolan-3-yl]oxyphosphonic acid |
| Butanal | 1-butanal, 123-72-8, aldehyde C4, butal, butalyde, butanal, butyraldehyde, butyric aldehyde, |
| | C4H8O, n-butyraldehyde |
| Butanoate | 107-92-6, 156-54-7, 461-55-2, butanoic acid, butyrate, C4 SCFA, C4H8O2, n-butyrate, sodium |
| | butyrate |
| Butanoyl-CoA | 2140-48-9, butanoyl-coenzyme A, butyryl-CoA, C25H42N7O17P3S, [(2R,3R,4R,5R)-5-(6- |
| | aminopurin-9-yl)-2-[[[[3-[2-(2-butanoylsulfanylethylcarbamoyl)ethylcarbamoyl]-3-hydroxy-2,2- |
| | dimethyl-propoxy]-hydroxy-phosphoryl]oxy-hydroxy-phosphoryl]oxymethyl]-4-hydroxy-oxolan-3- |
| | yl]oxyphosphonic acid |
| Butanoylphosphate | butanoyloxyphosphonic acid, C4H9O5P |
| Crotonoyl-CoA | 102680-35-3, 2-butenoyl-CoA, but-2-enoyl-CoA, C25H40N7O17P3S, crotonoyl-CoA, crotonyl-CoA, |
| | [(2R,3R,4R,5R)-5-(6-aminopurin-9-yl)-2-[[[[3-[2-(2-but-2- |
| | enoylsulfanylethylcarbamoyl)ethylcarbamoyl]-3-hydroxy-2,2-dimethyl-propoxy]-hydroxy- |
| | phosphoryl]oxy-hydroxy-phosphoryl]oxymethyl]-4-hydroxy-oxolan-3-yl]oxyphosphonic acid |
| Diacetyl | 2,3-butanedione, 431-03-8, butane-2,3-dione, C4H6O2 |
| Fumarate | (E)-but-2-enedioic acid, 110-17-8, 2-butenedioic acid (2E)-, C4H4O4, fumarate |
| Glutaconyl-1-CoA | 4-[2-[3-[[4-[[[5-(6-aminopurin-9-yl)-4-hydroxy-3-phosphonooxy-oxolan-2-yl]methoxy-hydroxy- |
| | phosphoryl]oxy-hydroxy-phosphoryl]oxy-2-hydroxy-3,3-dimethyl- |
| | butanoyl]amino]propanoylamino]ethylsulfanylcarbonyl]but-3-enoic acid, 6712-05-6, |
| | C26H40N7O19P3S, coenzyme A, glutaconyl-, coenzyme A, S-(5-hydrogen 2-pentenedioate), |
| | glutaconyl-1-CoA, glutaconyl-1-coenzyme A, glutaconyl-coa |
| L-Glutamate | (2S)-2-aminopentanedioic acid, 142-47-2, 19473-49-5, 56-86-0, C5H9NO4, glutamate, glutamic |
| | acid, L-Glu, L-glutamate, L-glutamic acid, monosodium glutamate, potassium glutamate, potassium |
| | L-glutamate, sodium glutamate |
| Maleate | (Z)-but-2-enedioic acid, 110-16-7, 2-butenedioic acid, 2-butenedioic acid (2Z)-, 2-butenedioic acid |
| | (Z)-, 2-butenedioic acid (Z)-(9Cl), C4H4O4, cis-butenedioic acid, toxilic acid |
| PHBC | Acatn, EctA, LAC1, LAG1, PHBC |
| Poly-&beta;-hydroxy- | ((R)-3-hydroxybutanoyl)(n-2), (C4H6O2)n, 29435-48-1, butanoic acid, 3-hydroxy-, (R)-, |
| butyrate | homopolymer, poly(D-beta-hydroxybutyrate), poly-beta-hydroxybutyrate, (R)-isomer |
| Pyruvate | 127-17-3, 2-oxopropanoate, 2-oxopropanoic acid, 57-60-3, C3H4O3, propanoic acid, 2-oxo-, |
| | propanoic acid, 2-oxo-, ion(1-), propanoic acid, 2-oxo-, sodium salt, pyruvate, pyruvic acid, sodium |
| | salt, sodium pyruvate |
| Succinate | 1,2-ethanedicarboxylic acid, 1,4-butanedioic acid, 110-15-6, 56-14-4, amber acid, asuccin, |
| | butanedioate, butanedioic acid, C4H6O4, ethylenesuccinic acid, katasuccin, potassium succinate, |
| | succinate, wormwood acid |
| Succinate | 3-formylpropanoic acid, 4-oxobutanoic acid, 692-29-5, beta-formylpropionic acid, butanoic acid, 4- |
| semialdehyde | oxo-, butanoic acid, 4-oxo-(9Cl), butryaldehydic acid, C4H6O3, gamma-oxybutyric acid, |
| | succinaldehydic acid, succinate semialdehyde |
| Thiamine | 136-09-4, 154-87-0, 23883-45-6, C12H19N4O7P2S+, cocarboxylase, thiamin diphosphate, |
| diphosphate | thiamine diphosphate hydrochloride, thiazolium, 3-((4-amino-2-methyl-5-pyrimidinyl)methyl)-4- |
| | methyl-5-(4,6,6-trihydroxy-3,5-dioxa-4,6-diphosphahex-1-yl)-, chloride, P,P′-dioxide, [2-[3-[(4-amino- |
| | 2-methyl-pyrimidin-5-yl)methyl]-4-methyl-1-thia-3-azoniacyclopenta-2,4-dien-5-yl]ethoxy-hydroxy- |
| | phosphoryl]oxyphosphonic acid |
| Vinylacetyl-CoA | 3-butenoyl-CoA, C25H40N7O17P3S, vinylacetyl-CoA, [(2R,3R,4R,5R)-5-(6-aminopurin-9-yl)-2-[[[[3- |
| | [2-(2-but-3-enoylsulfanylethylcarbamoyl)ethylcarbamoyl]-3-hydroxy-2,2-dimethyl-propoxy]-hydroxy- |
| | phosphoryl]oxy-hydroxy-phosphoryl]oxymethyl]-4-hydroxy-oxolan-3-yl]oxyphosphonic acid |

*Genes/proteins that were used to identify the pathway:
1.1.1.35 (S)-3-hydroxyacyl-CoA:NAD oxidoreductase, 1-specific DPN-linked beta-hydroxybutyric dehydrogenase, 3-hydroxyacetyl-coenzyme A dehydrogenase, 3-hydroxyacyl coenzyme A dehydrogenase, 3-hydroxybutyryl-CoA dehydrogenase, 3-hydroxyisobutyryl-CoA dehydrogenase, 3-keto reductase, 3-L-hydroxyacyl-CoA dehydrogenase, 3beta-hydroxyacyl coenzyme A dehydrogenase, beta-hydroxy acid dehydrogenase, beta-hydroxyacyl CoA dehydrogenase, beta-hydroxyacyl dehydrogenase, beta-hydroxyacyl-coenzyme A synthetase, beta-hydroxyacylcoenzyme A dehydrogenase, beta-hydroxybutyrylcoenzyme A dehydrogenase, beta-keto-reductase, beta-ketoacyl-CoA reductase, L-3-hydroxyacyl CoA dehydrogenase, L-3-hydroxyacyl coenzyme A dehydrogenase
2.3.1.9 2-methylacetoacetyl-CoA thiolase, 3-oxothiolase, acetoacetyl-CoA thiolase, acetyl coenzyme A thiolase, acetyl-CoA acetyltransferase, acetyl-CoA:acetyl-CoA C-acetyltransferase, acetyl-CoA:N-acetyltransferase, beta-acetoacetyl coenzyme A thiolase, thiolase II
2.3.3.10 (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b-methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase
4.2.1.17 (3S)-3-hydroxyacyl-CoA hydro-lyase, 2-enoyl-CoA hydratase, 2-octenoyl coenzyme A hydrase, acyl coenzyme A hydrase, beta-hydroxyacid dehydrase, beta-hydroxyacyl-CoA dehydrase, crotonase, crotonyl hydrase, D-3-hydroxyacyl-CoA dehydratase, ECH, enol-CoA hydratase, enoyl coenzyme A hydrase (D), enoyl coenzyme A hydrase (L), enoyl coenzyme A hydratase, enoyl hydrase, hydratase, enoyl coenzyme A, short chain enoyl coenzyme A hydratase, short-chain enoyl-CoA hydratase, trans-2-enoyl-CoA hydratase, unsaturated acyl-CoA hydratase

TABLE 28
Genes/Proteins Involved in the Valine, leucine and isoleucine degradation pathway.

| Name | Synonyms |
|---|---|
| (R)-3-Methyl-2- | (R)-2-oxoisovalerate, (R)-2-oxoisovaleric acid, (R)-3-methyl-2-oxobutanoate, (R)- |
| oxobutanoate | alpha-ketoisovalerate, (R)-alpha-ketoisovaleric acid, 3-methyl-2-oxo-butanoate, |
| | C5H7O3− |
| (R)-4-Methyl-3- | (R)-3-Oxo-4-methylpentanoate, 4-methyl-3-oxo-pentanoic acid, C6H10O3 |
| oxopentanoate | |
| (R)-Methyl-malonyl- | (2R)-2-[2-[3-[[4-[[[(2R,3R,4R,5R)-5-(6-aminopurin-9-yl)-4-hydroxy-3- |
| CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]propanoic acid, (R)-2- |
| | methyl-3-oxopropanoyl-CoA, (R)-2-methyl-3-oxopropanoyl-coenzyme A, |
| | C25H40N7O19P3S |
| (S)-(3- | 8-(3-methylbutanoylsulfanyl)-6-sulfanyl-octanamide, C13H25NO2S2, S-(8- |
| Methylbutanoyl)- | amino-8-oxo-3-sulfanyloctyl) 3-methylbutanethioate |
| dihydrolipoamide | |
| (S)-3-Amino- | (2S)-3-amino-2-methyl-propanoic acid, (S)-3-amino-2-methyl-propanoic acid, (S)- |
| isobutanoate | 3-aminoisobutyric acid, 4249-19-8, C4H9NO2, L-3-amino-isobutanoate |
| (S)-3-Hydroxy-2- | (2S,3S)-3-hydroxy-2-methylbutanoyl-CoA, (S)-3-hydroxy-2-methylbutyryl-CoA, |
| methylbutyryl-CoA | C26H44N7O18P3S, [(2R,3R,4R,5R)-5-(6-aminopurin-9-yl)-4-hydroxy-2- |
| | [[hydroxy-[hydroxy-[3-hydroxy-3-[2-[2-[(2S,3S)-3-hydroxy-2-methyl- |
| | butanoyl]sulfanylethylcarbamoyl]ethylcarbamoyl]-2,2-dimethyl- |
| | propoxy]phosphoryl]oxy-phosphoryl]oxymethyl]oxolan-3-yl]oxyphosphonic acid |
| (S)-3-Hydroxy-3- | (3S)-4-[2-[3-[[4-[[[(2R,3R,4R,5R)-5-(6-aminopurin-9-yl)-4-hydroxy-3- |
| methylglutaryl-CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-hydroxy-3-methyl- |
| | butanoic acid, (S)-3-hydroxy-3-methylglutaryl-CoA, 1553-55-5, |
| | C27H44N7O20P3S, hydroxymethylglutaryl-CoA, S-(hydrogen 3-hydroxy-3- |
| | methylglutaryl)coenzyme A, S-(hydrogen 3-hydroxy-3-methylpentanedioate)coenzyme A |
| (S)-3- | (2S)-3-hydroxy-2-methyl-propanoic acid, (S)-3-hydroxy-2-methylpropionate, (S)- |
| Hydroxyisobutyrate | 3-hydroxy-2-methylpropionic acid, (S)-3-hydroxyisobutyrate, (S)-3- |
| | hydroxyisobutyric acid, (S)-beta-hydroxyisobutyric acid, 26543-05-5, C4H8O3, |
| | hydracrylic acid, 2-methyl-, L-(+)-, L-(+)-beta-hydroxyisobutyric acid, propanoic |
| | acid, 3-hydroxy-2-methyl-, (S)- |
| (S)-3- | (S)-3-hydroxyisobutyryl-CoA, 3-hydroxyisobutyryl-CoA, C25H44N7O18P3S, |
| Hydroxyisobutyryl- | [(2R,3R,4R,5R)-5-(6-aminopurin-9-yl)-4-hydroxy-2-[[hydroxy-[hydroxy-[3- |
| CoA | hydroxy-3-[[3-hydroxy-3-[2-[(2S)-3-hydroxy-2-methyl- |
| | propanoyl]sulfanylethylamino]propyl]carbamoyl]-2,2-dimethyl- |
| | propoxy]phosphoryl]oxy-phosphoryl]oxymethyl]oxolan-3-yl]oxyphosphonic acid |
| (S)-Methyl-malonate | (2S)-2-methyl-3-oxo-propanoic acid, C4H6O3 |
| semialdehyde | |
| (S)-Methyl-malonyl- | (2S)-2-[2-[3-[[4-[[[(2R,3R,4R,5R)-5-(6-aminopurin-9-yl)-4-hydroxy-3- |
| CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]propanoic acid, (S)-2- |
| | methyl-3-oxopropionyl-CoA, (S)-3-oxo-2-methylpropanoyl-CoA, (S)- |
| | methylmalonyl-CoA, C25H40N7O19P3S |
| 1.1.1.178 | (2S,3S)-3-hydroxy-2-methylbutanoyl-CoA:NAD oxidoreductase, 2-methyl-3- |
| | hydroxy-butyryl CoA dehydrogenase, 2-methyl-3-hydroxybutyryl coenzyme A |
| | dehydrogenase |
| 1.1.1.31 | 3-hydroxy-2-methylpropanoate:NAD oxidoreductase, beta-hydroxyisobutyrate |
| | dehydrogenase |
| 1.2.1.25 | 2-oxoisovalerate dehydrogenase, 3-methyl-2-oxobutanoate:NAD 2- |
| | oxidoreductase (CoA-methyl-propanoylating), alpha-ketoisovalerate |
| | dehydrogenase |
| 1.2.1.27 | 2-methyl-3-oxopropanoate:NAD 3-oxidoreductase (CoA-propanoylating) |
| 1.2.1.3 | aldehyde:NAD oxidoreductase, CoA-independent aldehyde dehydrogenase, m- |
| | methylbenzaldehyde dehydrogenase, NAD-aldehyde dehydrogenase, NAD- |
| | dependent 4-hydroxynonenal dehydrogenase, NAD-dependent aldehyde |
| | dehydrogenase, NAD-linked aldehyde dehydrogenase, propionaldehyde |
| | dehydrogenase |
| 1.2.3.1 | aldehyde:oxygen oxidoreductase, quinoline oxidase |
| 1.2.4.4 | 2-oxoisocaproate dehydrogenase, 2-oxoisovalerate (lipoate) dehydrogenase, 3- |
| | methyl-2-oxobutanoate:lipoamide oxidoreductase (decarboxylating and acceptor- |
| | 2-methylpropanoylating), alpha-keto-alpha-methylvalerate dehydrogenase, |
| | alpha-ketoisocaproate dehydrogenase, alpha-ketoisocaproic dehydrogenase, |
| | alpha-ketoisocaproic-alpha-keto-alpha-methylvaleric dehydrogenase, alpha- |
| | ketoisovalerate dehydrogenase, alpha-oxoisocaproate dehydrogenase, BCKDH, |
| | BCOAD, branched chain keto acid dehydrogenase, branched-chain (-2-oxoacid) |
| | dehydrogenase (BCD), branched-chain 2-keto acid dehydrogenase, branched- |
| | chain 2-oxo acid dehydrogenase, branched-chain alpha-keto acid |
| | dehydrogenase, branched-chain alpha-oxo acid dehydrogenase, |
| | dehydrogenase, 2-oxoisovalerate (lipoate), dehydrogenase, branched chain |
| | alpha-keto acid |
| 1.3.99.10 | 3-methylbutanoyl-CoA:(acceptor) oxidoreductase, isovaleroyl-coenzyme A |
| | dehydrogenase, isovaleryl-coenzyme A dehydrogenase |
| 1.3.99.12 | 2-methyl branched chain acyl-CoA dehydrogenase, 2-methylbutanoyl- |
| | CoA:(acceptor) oxidoreductase, branched-chain acyl-CoA dehydrogenase |
| 1.3.99.2 | 3-hydroxyacyl CoA reductase, butanoyl-CoA:(acceptor) 2,3-oxidoreductase, |
| | butyryl coenzyme A dehydrogenase, butyryl dehydrogenase, enoyl-coenzyme A |
| | reductase, ethylene reductase, short-chain acyl CoA dehydrogenase, short-chain |
| | acyl-coenzyme A dehydrogenase, unsaturated acyl coenzyme A reductase, |
| | unsaturated acyl-CoA reductase |
| 1.3.99.3 | acyl coenzyme A dehydrogenase, acyl dehydrogenase, acyl-CoA:(acceptor) 2,3- |
| | oxidoreductase, fatty acyl coenzyme A dehydrogenase, fatty-acyl-CoA |
| | dehydrogenase, general acyl CoA dehydrogenase, long-chain acyl coenzyme A |
| | dehydrogenase, long-chain acyl-CoA dehydrogenase, medium-chain acyl-CoA |
| | dehydrogenase, medium-chain acyl-coenzyme A dehydrogenase |
| 1.4.1.9 | L-leucine dehydrogenase, L-leucine:NAD oxidoreductase (deaminating), L- |
| | leucine:NAD+ oxidoreductase, deaminating, LeuDH |
| 1.4.3.2 | L-amino-acid:oxygen oxidoreductase (deaminating), ophio-amino-acid oxidase |
| 2-Methylacetoacetyl- | 2-methyl-3-acetoacetyl-CoA, 2-methyl-3-acetoacetyl-coenzyme A, 2- |
| CoA | methylacetoacetyl-CoA, 6712-01-2, C26H42N7O18P3S, coenzyme A, S-(2- |
| | methyl-3-oxobutanoate), [(2R,3R,4R,5R)-5-(6-aminopurin-9-yl)-4-hydroxy-2- |
| | [[hydroxy-[hydroxy-[3-hydroxy-2,2-dimethyl-3-[2-[2-(2-methyl-3-oxo- |
| | butanoyl)sulfanylethylcarbamoyl]ethylcarbamoyl]propoxy]phosphoryl]oxy- |
| | phosphoryl]oxymethyl]oxolan-3-yl]oxyphosphonic acid |
| 2-Methylbutanoyl- | 2-methylbutanoyl-CoA, C26H44N7O17P3S, [(2R,3R,4R,5R)-5-(6-aminopurin-9- |
| CoA | yl)-4-hydroxy-2-[[hydroxy-[hydroxy-[3-hydroxy-2,2-dimethyl-3-[2-[2-(2- |
| | methylbutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| 2-Oxoisopentanoate | 3-methyl-2-oxo-butanoic acid, 3-methyl-2-oxobutanoate, 3-methyl-2-oxobutyric |
| | acid, 3715-29-5, 51828-94-5, 759-05-7, alpha-ketoisovalerate, C5H8O3, calcium |
| | 3-methyl-2-oxobutyrate (1:2), sodium 3-methyl-2-oxobutanoate |
| 2.3.1.- | Acatn, EctA, LAC1, LAG1, PHBC |
| 2.6.1.18 | beta-alanine-alpha-alanine transaminase, beta-alanine-pyruvate |
| | aminotransferase, L-alanine:3-oxopropanoate aminotransferase |
| 2.6.1.22 | (S)-3-amino-2-methylpropanoate:2-oxoglutarate aminotransferase, beta- |
| | aminobutyric transaminase, L-3-aminoisobutyrate transaminase, L-3- |
| | aminoisobutyric aminotransferase |
| 2.6.1.42 | branched-chain amino acid aminotransferase, branched-chain amino acid- |
| | glutamate transaminase, branched-chain aminotransferase, branched-chain- |
| | amino-acid:2-oxoglutarate aminotransferase, glutamate-branched-chain amino |
| | acid transaminase, L-branched chain amino acid aminotransferase, |
| | transaminase B |
| 2.6.1.6 | L-leucine aminotransferase, L-leucine:2-oxoglutarate aminotransferase, leucine |
| | 2-oxoglutarate transaminase, leucine aminotransferase, leucine-alpha- |
| | ketoglutarate transaminase |
| 2.8.3.5 | 3-ketoacid CoA-transferase, 3-ketoacid coenzyme A transferase, 3-oxo-CoA |
| | transferase, 3-oxoacid CoA dehydrogenase, 3-oxoacid coenzyme A-transferase, |
| | acetoacetate succinyl-CoA transferase, acetoacetyl coenzyme A-succinic |
| | thiophorase, succinyl coenzyme A-acetoacetyl coenzyme A-transferase, |
| | succinyl-CoA transferase, succinyl-CoA:3-oxo-acid CoA-transferase |
| 3-Hydroxyisovaleryl- | 3-hydroxyisovaleryl-CoA, C26H44N7O18P3S, [(2R,3S,4R,5R)-5-(6-aminopurin- |
| CoA | 9-yl)-4-hydroxy-2-[[hydroxy-[hydroxy-[3-hydroxy-3-[2-[2-(3-hydroxy-3-methyl- |
| | butanoyl)sulfanylethylcarbamoyl]ethylcarbamoyl]-2,2-dimethyl- |
| | propoxy]phosphoryl]oxy-phosphoryl]oxymethyl]oxolan-3-yl]oxyphosphonic acid |
| 3-Methylbut-2-enoyl- | 3-methylbut-2-enoyl-CoA, 3-methylcrotonoyl-CoA, C26H42N7O17P3S, |
| CoA | [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3-methylbut-2- |
| | enoylsulfanyl)ethylcarbamoyl]ethylcarbamoyl]propoxy]phosphoryl]oxy- |
| | phosphoryl]oxymethyl]oxolan-3-yl]oxyphosphonic acid |
| 3-Methylbutanoyl- | 3-methylbutanoyl-coenzyme A, 6244-91-3, C26H44N7O17P3S, isovaleryl-coa, |
| CoA | S-(3-Methylbutanoate) coenzyme A, [(2R,3R,4R,5R)-5-(6-aminopurin-9-yl)-4- |
| | hydroxy-2-[[hydroxy-[hydroxy-[3-hydroxy-2,2-dimethyl-3-[2-[2-(3- |
| | methylbutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| 3-Methylglutaconyl- | (E)-4-[2-[3-[[4-[[[(2R,3S,4R,5R)-5-(6-aminopurin-9-yl)-4-hydroxy-3- |
| CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-methyl-but-2-enoic acid, |
| | C27H42N7O19P3S, trans-3-methylglutaconyl-CoA |
| 3.1.2.4 | 3-hydroxy-2-methylpropanoyl-CoA hydrolase, HIB CoA deacylase |
| 4-Methyl-2- | 2-oxoisocaproate, 4-methyl-2-oxo-pentanoic acid, 4-methyl-2-oxopentanoate, 4- |
| oxopentanoate | methyl-2-oxopentanoic acid, 4502-00-5, 51828-95-6, 816-66-0, alpha- |
| | ketoisocaproate, C6H10O3, calcium 4-methyl-2-oxovalerate, ketoisocaproate, |
| | sodium 4-methyl-2-oxovalerate |
| 4.1.3.4 | (S)-3-hydroxy-3-methylglutaryl-CoA acetoacetate-lyase, 3-hydroxy-3- |
| | methylglutaryl CoA cleaving enzyme, 3-hydroxy-3-methylglutaryl coenzyme A |
| | lyase, 3-hydroxy-3-methylglutaryl-CoA lyase, hydroxymethylglutaryl coenzyme A |
| | lyase, hydroxymethylglutaryl coenzyme A-cleaving enzyme |
| 4.1.99.- | |
| 4.2.1.18 | (S)-3-hydroxy-3-methylglutaryl-CoA hydro-lyase, 3-methylglutaconyl CoA |
| | hydratase, methylglutaconase, methylglutaconyl coenzyme A hydratase |
| 5.1.99.1 | 2-methyl-3-oxopropanoyl-CoA 2-epimerase, DL-methylmalonyl-CoA racemase, |
| | methylmalonyl coenzyme A racemase, methylmalonyl-CoA racemase |
| 5.4.3.7 | (2S)-alpha-leucine 2,3-aminomutase |
| 5.4.99.2 | (R)-2-methyl-3-oxopropanoyl-CoA CoA-carbonylmutase, (S)-methylmalonyl-CoA |
| | mutase, methylmalonyl coenzyme A carbonylmutase, methylmalonyl coenzyme |
| | A mutase, methylmalonyl-CoA CoA-carbonyl mutase |
| 6.4.1.3 | propanoyl-CoA:carbon-dioxide ligase (ADP-forming) |
| 6.4.1.4 | 3-methylcrotonoyl-CoA:carbon-dioxide ligase (ADP-forming) |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme A, |
| | [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl-propoxy]- |
| | hydroxy-phosphoryl]oxy-hydroxy-phosphoryl]oxymethyl]-5-(6-aminopurin-9-yl)-4- |
| | hydroxy-oxolan-3-yl]oxyphosphonic acid |
| Branched chainfatty | |
| acid | |
| Isobutyryl-CoA | 15621-60-0, 2-methylpropanoyl-CoA, 2-methylpropionyl-CoA, |
| | C25H42N7O17P3S, coenzyme A, S-(2-methylpropanoate), isobutyryl-CoA, |
| | [(2R,3R,4R,5R)-5-(6-aminopurin-9-yl)-4-hydroxy-2-[[hydroxy-[hydroxy-[3- |
| | hydroxy-2,2-dimethyl-3-[2-[2-(2- |
| | methylpropanoylsulfanyl)ethylcarbamoyl]ethylcarbamoyl]propoxy]phosphoryl]oxy- |
| | phosphoryl]oxymethyl]oxolan-3-yl]oxyphosphonic acid |
| L-Isoleucine | (2S,3S)-2-amino-3-methyl-pentanoic acid, 73-32-5, C6H13NO2, isoleucine, L- |
| | isoleucine, valeric acid, 2-amino-3-methyl- |
| L-Leucine | (2S)-2-amino-4-methyl-pentanoic acid, (2S)-alpha-leucine, (S)-2-amino-4- |
| | methylpentanoic acid, 2-amino-4-methylpentanoic acid (L), 61-90-5, 7005-03-0, |
| | C6H13NO2, L-leucine, leucine |
| L-Valine | (2S)-2-amino-3-methyl-butanoic acid, (S)-alpha-amino-beta-methylbutyric acid, |
| | 72-18-4, C5H11NO2, L-alpha-amino-beta-methylbutyric acid, L-valine, valine |
| L-&beta;-Leucine | (3R)-beta-2-amino-4-methylvaleric acid, (3S)-3-amino-4-methyl-pentanoic acid, |
| | C6H13NO2, L-beta-leucine |
| Methylacrylyl-CoA | 2-methylprop-2-enoyl-CoA, C25H40N7O17P3S, methacrylyl-CoA, methylacrylyl- |
| | CoA, [(2R,3R,4R,5R)-5-(6-aminopurin-9-yl)-4-hydroxy-2-[[hydroxy-[hydroxy-[3- |
| | hydroxy-2,2-dimethyl-3-[2-[2-(2-methylprop-2- |
| | enoylsulfanyl)ethylcarbamoyl]ethylcarbamoyl]propoxy]phosphoryl]oxy- |
| | phosphoryl]oxymethyl]oxolan-3-yl]oxyphosphonic acid |
| Methylmalonate | 1,1-ethanedicarboxylic acid, 2-methylmalonic acid, 2-methylpropanedioic acid, |
| | 516-05-2, C4H6O4, isosuccinic acid, methylmalonate, propanedioic acid, methyl-, |
| | propanedioic acid, methyl-(9Cl) |
| Propionyl-CoA | 317-66-8, C24H40N7O17P3S, propanoyl-CoA, propionyl-CoA, S- |
| | propionylcoenzyme A, [(2R,3R,4R,5R)-5-(6-aminopurin-9-yl)-4-hydroxy-2- |
| | [[hydroxy-[hydroxy-[3-hydroxy-2,2-dimethyl-3-[2-(2- |
| | propanoylsulfanylethylcarbamoyl)ethylcarbamoyl]propoxy]phosphoryl]oxy- |
| | phosphoryl]oxymethyl]oxolan-3-yl]oxyphosphonic acid |
| S-(2-Methylbutanoyl)- | 8-(2-methylbutanoylsulfanyl)-6-sulfanyl-octanamide, C13H25NO2S2, S-(8- |
| dihydrolipoamide | amino-8-oxo-3-sulfanyloctyl) 2-methylbutanethioate |
| S-(2- | 8-(2-methylpropanoylsulfanyl)-6-sulfanyl-octanamide, C12H23NO2S2, S-(2- |
| Methylpropanoyl)- | methylpropionyl)-dihydrolipoamide |
| dihydrolipoamide | |
| S-3-Methyl-2- | (3S)-3-methyl-2-oxo-pentanoic acid, (S)-3-methyl-2-oxopentanoate, (S)-3-methyl- |
| oxopentanoate | 2-oxovaleric acid, (S)-alpha-keto-beta-methylvaleric acid, 24809-08-3, 51828-96- |
| | 7, C6H10O3, calcium (S)-3-methyl-2-oxovalerate, L-3-methyl-2-oxopentanoate |
| Succinyl-CoA | 3-[2-[3-[[4-[[[(2R,3R,4R,5R)-5-(6-aminopurin-9-yl)-4-hydroxy-3-phosphonooxy- |
| | oxolan-2-yl]methoxy-hydroxy-phosphoryl]oxy-hydroxy-phosphoryl]oxy-2-hydroxy- |
| | 3,3-dimethyl-butanoyl]amino]propanoylamino]ethylsulfanylcarbonyl]propanoic |
| | acid, 604-98-8, C25H40N7O19P3S, coenzyme A, S-(hydrogen butanedioate), |
| | succinyl-CoA |
| trans-2-Methyl-but-2- | (E)-2-methylcrotonoyl-CoA, 2-methylbut-2-enoyl-CoA, 6247-62-7, |
| enoyl-CoA | C26H42N7O17P3S, coenzyme A, S-(2-methyl-2-butenoate), (E)-, |
| | methylcrotonoyl-CoA, methylcrotonyl-CoA, tigloyl-CoA, tiglyl-CoA, trans-2- |
| | methylbut-2-enoyl-CoA, [(2R,3R,4R,5R)-5-(6-aminopurin-9-yl)-4-hydroxy-2- |
| | [[hydroxy-[hydroxy-[3-hydroxy-2,2-dimethyl-3-[2-[2-(2-methylbut-2- |
| | enoylsulfanyl)ethylcarbamoyl]ethylcarbamoyl]propoxy]phosphoryl]oxy- |
| | phosphoryl]oxymethyl]oxolan-3-yl]oxyphosphonic acid |

*Genes/proteins that were used to identify the pathway:
1.1.1.35 (S)-3-hydroxyacyl-CoA:NAD oxidoreductase, 1-specific DPN-linked beta-hydroxybutyric dehydrogenase, 3-hydroxyacetyl-coenzyme A dehydrogenase, 3-hydroxyacyl coenzyme A dehydrogenase, 3-hydroxybutyryl-CoA dehydrogenase, 3-hydroxyisobutyryl-CoA dehydrogenase, 3-keto reductase, 3-L-ydroxyacyl-CoA dehydrogenase, 3beta-hydroxyacyl coenzyme A dehydrogenase, beta-hydroxy acid dehydrogenase, beta-hydroxyacyl CoA dehydrogenase, beta-hydroxyacyl dehydrogenase, beta-hydroxyacyl- coenzyme A synthetase, beta-hydroxyacylcoenzyme A dehydrogenase, beta-hydroxybutyrylcoenzyme A dehydrogenase, beta-keto-reductase, beta-ketoacyl-CoA reductase, L-3-hydroxyacyl CoA dehydrogenase, L-3-hydroxyacyl coenzyme A dehydrogenase
2.3.1.9 2-methylacetoacetyl-CoA thiolase, 3-oxothiolase, acetoacetyl-CoA thiolase, acetyl coenzyme A thiolase, acetyl-CoA acetyltransferase, acetyl-CoA:acetyl-CoA C-acetyltransferase, acetyl-CoA:N-acetyltransferase, beta-acetoacetyl coenzyme A thiolase, thiolase II
2.3.3.10 (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b-methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase
2.3.1.16 3-ketoacyl CoA thiolase, 3-ketoacyl coenzyme A thiolase, 3-ketoacyl thiolase, 3-ketothiolase, 3-oxoacyl-CoA thiolase, 3-oxoacyl-coenzyme A thiolase, 6-oxoacyl-CoA thiolase, acetoacetyl-CoA beta-ketothiolase, acetyl-CoA acyltransferase, acyl-CoA:acetyl-CoA C-acyltransferase, beta-ketoacyl coenzyme A thiolase, beta-ketoacyl-CoA thiolase, beta-ketoadipyl coenzyme A thiolase, beta-ketoadipyl-CoA thiolase, beta-ketothiolase, KAT, ketoacyl-CoA acyltransferase, ketoacyl-coenzyme A thiolase, long-chain 3-oxoacyl-CoA thiolase, oxoacyl-coenzyme A thiolase, pro-3-ketoacyl-CoA thiolase, thiolase I
4.2.1.17 (3S)-3-hydroxyacyl-CoA hydro-lyase, 2-enoyl-CoA hydratase, 2-octenoyl coenzyme A hydrase, acyl coenzyme A hydrase, beta-hydroxyacid dehydrase, beta-hydroxyacyl-CoA dehydrase, crotonase, crotonyl hydrase, D-3-hydroxyacyl-CoA dehydratase, ECH, enol-CoA hydratase, enoyl coenzyme A hydrase (D), enoyl coenzyme A hydrase (L), enoyl coenzyme A hydratase, enoyl hydrase, hydratase, enoyl coenzyme A, short chain enoyl coenzyme A hydratase, short-chain enoyl-CoA hydratase, trans-2-enoyl-CoA hydratase, unsaturated acyl-CoA hydratase

In addition, pathway analysis using Pathway Studio software based on previously identified differentially expressed genes or proteins associated with low ammonium production led to the identification of the Eda A1 pathway (FIG. 9), Eda-A2 pathway (FIG. 10). Genes/proteins that were used to identify the pathways are indicated in FIGS. 9 and 10. In addition, additional exemplary genes or proteins involved in the above-identified pathways and that may be involved in regulating or indicative of high cell viability are summarized in Table 29 (Eda-A1 pathway) and Table 30 (Eda-A2 pathway).

TABLE 29
Genes/Proteins Involved in the Eda-A1 pathway

| Name | Type | Description |
|---|---|---|
| Apoptosis | Cell Process | |
| CASP8 | Protein | caspase 8, apoptosis-related cysteine |
| | | peptidase |
| EDAR | Protein | ectodysplasin A receptor |
| EDARADD | Protein | EDAR-associated death domain |
| Jnk-mapk | Pathway | |
| NF kappa B | Pathway | |
| RIPK1 | Protein | receptor (TNFRSF)-interacting serine- |
| | | threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |

*Genes/proteins that were used to identify the pathway:
HMGCS1 3-hydroxy-3-methylglutaryl-Coenzyme A synthase 1 (soluble)

TABLE 30
Genes/Proteins Involved in the Eda-A2 pathway

| Name | Type | Description |
|---|---|---|
| Apoptosis | Cell Process | |
| CASP8 | Protein | caspase 8, apoptosis-related cysteine |
| | | peptidase |
| Jnk-mapk | Pathway | |
| NF kappa B | Pathway | |
| p40 MAPK | Pathway | |
| RIPK1 | Protein | receptor (TNFRSF)-interacting serine- |
| | | threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |
| TRAF6 | Protein | TNF receptor-associated factor 6 |
| XEDAR | Protein | microtubule-associated protein 2 |

*Genes/proteins that were used to identify the pathway:
HMGCS1 3-hydroxy-3-methylglutaryl-Coenzyme A synthase 1 (soluble)

### Example 7

### Exemplary Pathways Relating to Low Lactate Production

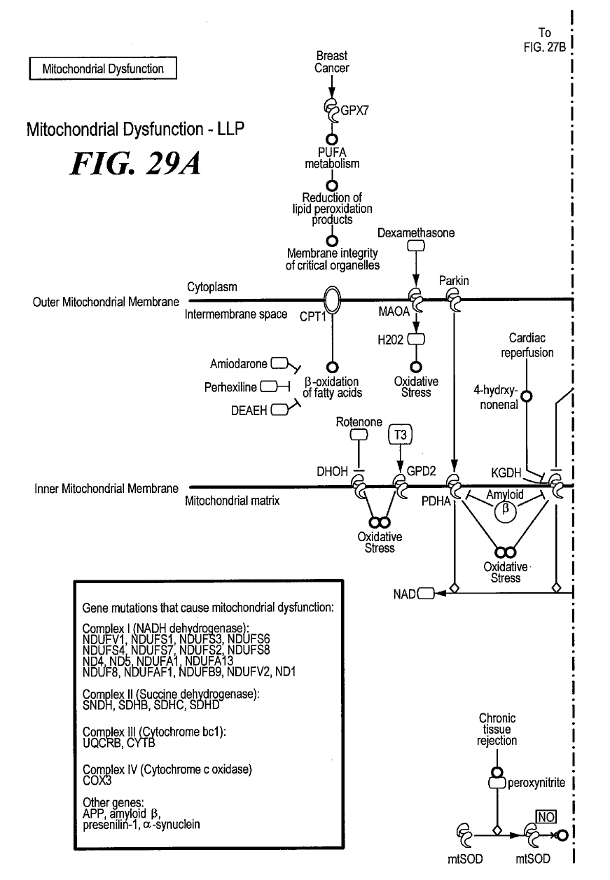

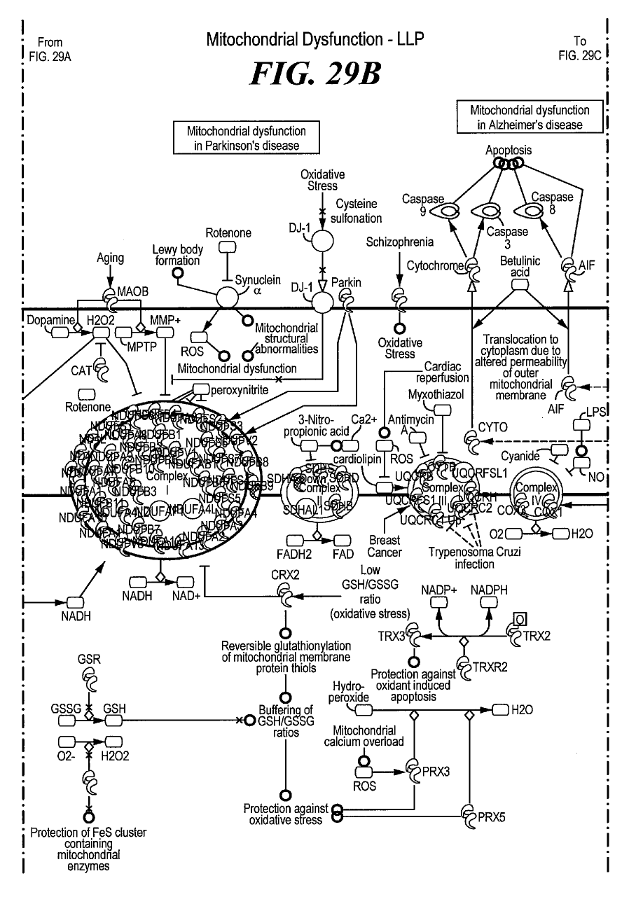

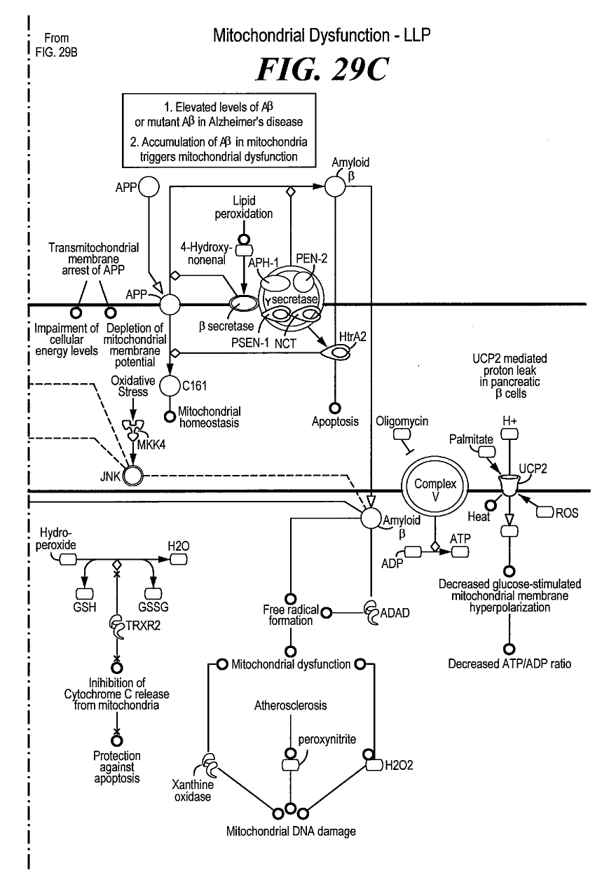

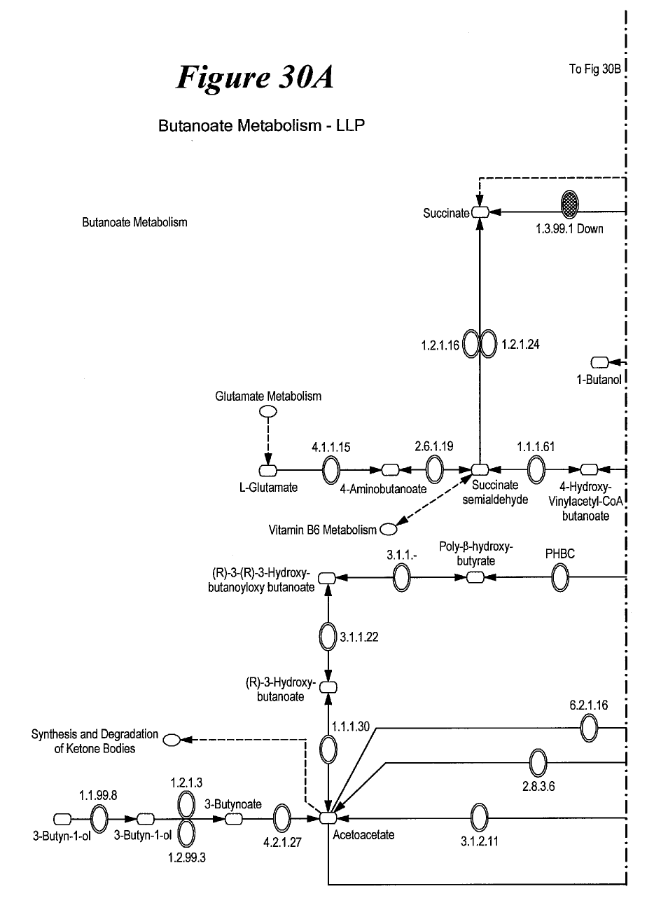

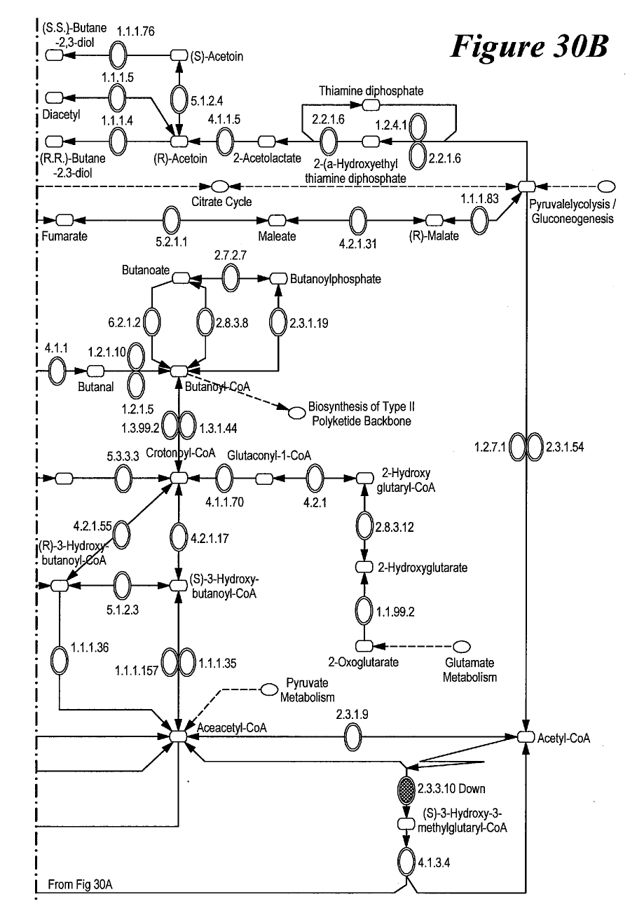

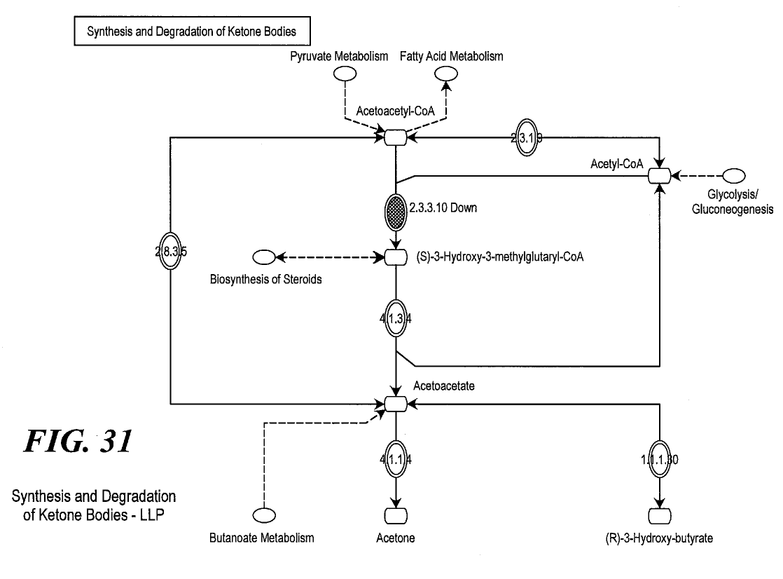

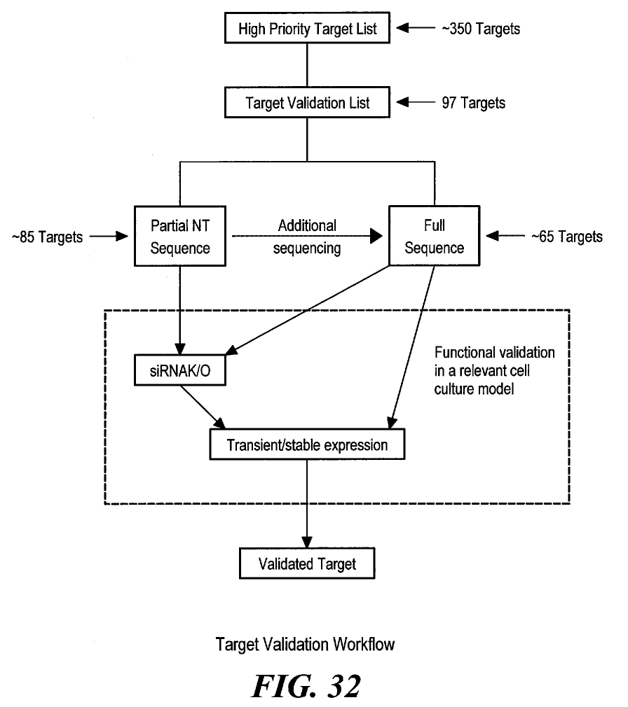

Pathway analysis using Ingenuity software based on previously identified differently expressed genes or proteins associated with low lactate production led to the identification of the oxidative phosphorylation pathway (FIG. 28), the mitochondrial dysfunction pathway (FIG. 29), the butanoate metabolism pathway (FIG. 30), and the synthesis and degradation of ketone bodies pathway (FIG. 31). Genes/proteins that were used to identify relevant pathways are indicated in FIGS. 28-31. In addition, additional exemplary genes or proteins involved in the above-identified pathways and that may be involved in regulating or indicative of low lactate production are summarized in Table 31 (the oxidative phosphorylation pathway), Table 32 (the mitochondrial dysfunction pathway), Table 33 (the butanoate metabolism pathway), and Table 34 (the synthesis and degradation of ketone bodies pathway).

TABLE 31
Genes/Proteins Involved in the Oxidatitve phosphorylation pathway

| Name | Synonyms |
|---|---|
| 1.6.99.5 | D-diaphorase, DPNH-menadione reductase, NADH-quinone oxidoreductase, |
| | NADH2:(quinone-acceptor) oxidoreductase, reduced nicotinamide adenine dinucleotide |
| | (quinone) dehydrogenase |
| 1.9.3.1 | complex IV (mitochondrial electron transport), COX, Cytochrome Aa3, Cytochrome c |
| | oxidase protein, Mitochondrial Complex IV, respiratory chain complex IV |
| 2.7.4.1 | ATP:polyphosphate phosphotransferase, polyphosphoric acid kinase |
| 3.6.1.1 | diphosphate phosphohydrolase |
| 3.6.3.10 | (K+ + H+)-ATPase, ATP phosphohydrolase (H+/K+-exchanging), H+-K+-ATPase, H,K- |
| | ATPase |
| 3.6.3.6 | ATP phosphohydrolase, ATP phosphohydrolase (H+-exporting), proton-translocating |
| | ATPase, yeast plasma membrane ATPase, yeast plasma membrane H+-ATPase |
| ADP | 20398-34-9, 58-64-0, 9-beta-D-arabinofuranosyladenine 5′-diphosphate, adenosine 5′- |
| | (trihydrogen diphosphate), adenosine diphosphate, C10H15N5O10P2, [[(2R,3R,4R,5R)-5- |
| | (6-aminopurin-9-yl)-3,4-dihydroxy-oxolan-2-yl]methoxy-hydroxy-phosphoryl]oxyphosphonic |
| | acid |
| ATP | 56-65-5, 9-beta-D-arabinofuranosyladenine 5′-triphosphate, adenosine 5′-(tetrahydrogen |
| | triphosphate), adenosine 5′-triphosphate, adenosine triphosphate, ATP4-, |
| | C10H16N5O13P3, [[[(2R,3R,4R,5R)-5-(6-aminopurin-9-yl)-3,4-dihydroxy-oxolan-2- |
| | yl]methoxy-hydroxy-phosphoryl]oxy-hydroxy-phosphoryl]oxyphosphonic acid |
| Diphosphate | 14000-31-8, dioxido-oxo-phosphonatooxy-phosphorane, diphosphate, diphosphate(4-), |
| | inorganic pyrophosphate, O7P2-4, PPi, pyrophosphate ion |
| Fumarate | (E)-but-2-enedioic acid, 110-17-8, 2-butenedioic acid (2E)-, C4H4O4, fumarate |
| H+ | 12408-02-5, 12586-59-3, H+, hydrogen ion, hydrogen(+1) cation, proton |
| H2O | 7732-18-5, H2O, oxidane |
| NAD+ | 53-84-9, adenosine 5′-(trihydrogen diphosphate), P′-5′-ester with 3-(aminocarbonyl)-1-beta- |
| | D-ribofuranosylpyridinium, inner salt, beta-NAD+, beta-nicotinamide adenine dinucleotide+, |
| | C21H28N7O14P2+, NAD, [[(2R,3R,4R,5R)-5-(6-aminopurin-9-yl)-3,4-dihydroxy-oxolan-2- |
| | yl]methoxy-hydroxy-phosphoryl]oxy-[[(2R,3R,4R,5R)-5-(5-carbamoylpyridin-1-yl)-3,4- |
| | dihydroxy-oxolan-2-yl]methoxy]phosphinic acid |
| NADH | 58-68-4, adenosine 5′-(trihydrogen diphosphate), P′-5′-ester with 1,4-dihydro-1-beta-D- |
| | ribofuranosyl-3-pyridinecarboxamide, beta-NADH, C21H29N7O14P2, dihydronicotinamide- |
| | adenine dinucleotide, NADH2, nicotinamide dinucleotide, [[(2R,3R,4R,5R)-5-(6-aminopurin- |
| | 9-yl)-3,4-dihydroxy-oxolan-2-yl]methoxy-hydroxy-phosphoryl]oxy-[[(2R,3R,4R,5R)-5-(3- |
| | carbamoyl-4H-pyridin-1-yl)-3,4-dihydroxy-oxolan-2-yl]methoxy]phosphinic acid |
| Oxygen | 7782-44-7, dioxygen, molecular oxygen, O2 |
| Phosphate | 14265-44-2, inorganic phosphate, O4P-3, phosphate, phosphate ion, phosphate(3-), Pi |
| Succinate | 1,2-ethanedicarboxylic acid, 1,4-butanedioic acid, 110-15-6, 56-14-4, amber acid, asuccin, |
| | butanedioate, butanedioic acid, C4H6O4, ethylenesuccinic acid, katasuccin, potassium |
| | succinate, succinate, wormwood acid |
| Triphosphate | 14127-68-5, O10P3-5, oxido-oxo-diphosphonatooxy-phosphorane, triphosphate |
| Ubiquinol | 56275-39-9, C14H20O4(C5H8)n, CoQH2, QH2, ubihydroquinone, ubiquinol, ubiquinone |
| | hydroquinone |
| Ubiquinone | 1339-63-5, C14H18O4(C5H8)n, coenzyme Q, ubiquinones |

*Genes/proteins that were used to identify the pathway:
1.3.5.1 complex II, fumarate reductase complex, menaquinol:fumarate oxidoreductase, succinate dehydrogenase complex, succinate:ubiquinone oxidoreductase, succinic dehydrogenase
1.3.99.1 Complex II, Succinate INT Dehydrogenase
1.6.5.3 coenzyme Q reductase, complex 1 dehydrogenase, complex I (electron transport chain), complex I (mitochondrial electron transport), complex I (NADH:Q1 oxidoreductase), dihydronicotinamide adenine dinucleotide-coenzyme Q reductase, DPNH-coenzyme Q reductase, DPNH-ubiquinone reductase, electron transfer complex I, mitochondrial electron transport complex 1, mitochondrial electron transport complex I, NADH coenzyme Q1 reductase, NADH-coenzyme Q oxidoreductase, NADH-coenzyme Q reductase, NADH-CoQ oxidoreductase, NADH-CoQ reductase, NADH-Q6 oxidoreductase, NADH-ubiquinone oxidoreductase, NADH-ubiquinone reductase, NADH-ubiquinone-1 reductase, NADH2:ubiquinone oxidoreductase, NADH:ubiquinone oxidoreductase complex, reduced nicotinamide adenine dinucleotide-coenzyme Q reductase, type 1 dehydrogenase, ubiquinone reductase
1.6.99.3 beta-NADH dehydrogenase dinucleotide, cytochrome c reductase, diaphorase, dihydrocodehydrogenase I dehydrogenase, dihydronicotinamide adenine dinucleotide dehydrogenase, diphosphopyri3633se, DPNH diaphorase, NADH diaphorase, NADH hydrogenase, NADH oxidoreductase, NADH-menadione oxidoreductase, NADH2:(acceptor) oxidoreductase, NADH:cytochrome c oxidoreductase, reduced diphosphopyridine nucleotide diaphorase, type 1 dehydrogenase
1.10.2.2 coenzyme Q-cytochrome c reductase, coenzyme QH2-cytochrome c reductase, CoQH2- cytochrome c oxidoreductase, dihydrocoenzyme Q-cytochrome c reductase, mitochondrial electron transport complex III, QH2:cytochrome c oxidoreductase, reduced coenzyme Q-cytochrome c reductase, reduced ubiquinone-cytochrome c oxidoreductase, reduced ubiquinone-cytochrome c reductase, complex III (mitochondrial electron transport), ubihydroquinol:cytochrome c oxidoreductase, ubiquinol-cytochrome c oxidoreductase, ubiquinol-cytochrome c-2 oxidoreductase, ubiquinol-cytochrome c1 oxidoreductase, ubiquinol-cytochrome c2 reductase, ubiquinol:ferricytochrome-c oxidoreductase, ubiquinone-cytochrome b-c1 oxidoreductase, ubiquinone-cytochrome c oxidoreductase, ubiquinone-cytochrome c reductase
3.6.3.14 ATP phosphohydrolase (H+-transporting), ATP synthase, bacterial Ca2+/Mg2+ ATPase, chloroplast ATPase, coupling factors (F0, F1 and CF1), F1-ATPase, FoF1-ATPase, H+-transporting ATPase, mitochondrial ATPase

TABLE 32
Genes/Proteins Involved in the Mitochondrial dysfunction pathway

| Name | Synonyms |
|---|---|
| 3-Nitro-propionic | 3-nitropropanoic acid, 504-88-1, beta-nitropropanoate, C3H5NO4, propanoic acid, 3- |
| acid | nitro-, propanoic acid, 3-nitro-(9Cl) |
| 4-hydroxy- | 2-Nonenal, 4-hydroxy-, 29343-52-0, 4-HNE, 4-hydroxy-2-nonenal, 4-hydroxynon-2- |
| nonenal | enal, 75899-68-2, C9H16O2 |
| ABAD | 17b-HSD10, ABAD, Ads9, ERAB, HADH2, HCD2, MHBD, MRX17, MRX31, MRXS10, |
| | SCHAD, XH98G2 |
| ADP | 20398-34-9, 58-64-0, 9-beta-D-arabinofuranosyladenine 5′-diphosphate, adenosine 5′- |
| | (trihydrogen diphosphate), adenosine diphosphate, C10H15N5O10P2, |
| | [[(2R,3R,4R,5R)-5-(6-aminopurin-9-yl)-3,4-dihydroxy-oxolan-2-yl]methoxy-hydroxy- |
| | phosphoryl]oxyphosphonic acid |
| AIF | AIF, AIFsh2, Hq, mAIF, MGC111425, MGC5706, PDCD8 |
| Amiodarone | (2-butylbenzofuran-3-yl)-[4-(2-diethylaminoethoxy)-3,5-diiodo-phenyl]methanone, 1951- |
| | 25-3, 19774-82-4, 2-butyl-3-benzofuryl 4-(2-(diethylamino)ethoxy)-3,5-diiodophenyl |
| | ketone hydrochloride, Amiodarex, amiodarone hydrochloride, Amiohexal, Amiorone, |
| | C25H29I2NO3, Cardarone, Cordarone, Cordarone I.V., Rythmarone |
| Antimycin A | 1397-94-0, antimycin, antimycin A |
| APH-1 | APH1 |
| ATP | 56-65-5, 9-beta-D-arabinofuranosyladenine 5′-triphosphate, adenosine 5′- |
| | (tetrahydrogen triphosphate), adenosine 5′-triphosphate, adenosine triphosphate, |
| | ATP4-, C10H16N5O13P3, [[[(2R,3R,4R,5R)-5-(6-aminopurin-9-yl)-3,4-dihydroxy- |
| | oxolan-2-yl]methoxy-hydroxy-phosphoryl]oxy-hydroxy-phosphoryl]oxyphosphonic acid |
| Betulinicacid | 3-hydroxylup-20(29)-en-28-oic acid, 472-15-1, C30H48O3, lup-20(29)-en-28-oic acid, |
| | 3-hydroxy-, (3beta)-, lup-20(29)-en-28-oic acid, 3beta-hydroxy-(8Cl) |
| C161 | A beta 25-35, A-BETA 40, A-BETA 42, AAA, ABETA, ABPP, AD1, Adap, AL024401, |
| | AMYLOID BETA, AMYLOID BETA 40, AMYLOID BETA 40 HUMAN PROTEIN, |
| | AMYLOID BETA 42, Amyloid beta A4, AMYLOID BETA PEPTIDE 40, Amyloidogenic |
| | glycoprotein, App alpha, APPI, appican, BETAAPP, CTFgamma, CVAP, |
| | E030013M08R1K, Nexin II, P3, PN2, PreA4, PROTEASE NEXIN2 |
| Ca2+ | 14127-61-8, Ca+2, calcium ion, calcium(+2) cation, calcium, ion (Ca2+) |
| cardiolipin | |
| Caspase 3 | A830040C14Rik, Apopain, CASPASE-3, CPP32, CPP32B, Cpp32beta, Ice-like |
| | cysteine protease, Lice, MGC93645, P17, PROCASPASE 3, SCA-1, YAMA |
| Caspase 8 | ALPS2B, CAP4, CASPASE-8, FLICE, FLJ17672, MACH, MCH5, MGC78473 |
| Caspase 9 | AI115399, APAF-3, AW493809, Casp-9-CTD, Casp9 v1, CASPASE-9, CASPASE-9c, |
| | ICE-LAP6, MCH6 |
| CAT | 2210418N07, Cas-1, CATALASE, Catalase1, Cs-1, MGC128112, MGC138422, |
| | MGC138424, RATCAT01, RATCATL |
| ComplexII | Complex II, Succinate INT Dehydrogenase |
| ComplexIII | |
| Complex I | |
| Complex IV | complex IV (mitochondrial electron transport), COX, Cytochrome Aa3, Cytochrome c |
| | oxidase protein, Mitochondrial Complex IV, respiratory chain complex IV |
| Complex V | COMPLEX V, RESPIRATORY CHAIN COMPLEX V |
| COX1 | Co1, COI, COX-I, cytochrome c oxidase I, Cytochrome C Oxidase Subunit 1, |
| | CYTOCHROME OXIDASE SUBUNIT I, CYTOCHROME OXIDASE1, |
| | MITOCHONDRIAL CYTOCHROME OXIDASE SUBUNIT 1, MTCO1 |
| COX3 | CO3 ATPASE 6,8, COIII, COXIII, CY3, Cytochrome C Oxidase Subunit 3, |
| | Mitochondrial cytochrome oxidase III, MTCO3 |
| CPT1 | Cpt-i |
| Cyanide | 57-12-5, CN−, cyanide, cyanide(1-) |
| CYB5R3 | 0610016L08Rik, 2500002N19Rik, B5R, C85115, DIA1, NADH Cytochrome B5 |
| | Reductase, NADHCB5, WU: AL591952.1-001, WU: AL591952.1-002, WU: AL591952.1- |
| | 003, WU: Cyb5r3 |
| CYTB | MITOCHONDRIAL CYTOCHROME B, Mt-cytb, MTCYB |
| Cytochrome C | CYC, Cycs, CYCSA, CYCT, CYCTA, CYTC, CYTOCHROME C, |
| | ENSMUSG00000062038, HCS, MGC93634, T-Cc |
| DEAEH | 2,2′-((1,2-diethylethylene)bis(p-phenyleneoxy))bis(triethyl)amine, 2-[4-[4-[4-(2- |
| | diethylaminoethoxy)phenyl]hexan-3-yl]phenoxy]-N,N-diethyl-ethanamine, 2691-45-4, |
| | 4,4′-bis(beta-diethylaminoethoxy)alpha,beta-diethyldiphenylethane, 4,4′- |
| | diethylaminoethoxyhexestrol, 69-14-7, C30H48N2O2, coralgil, |
| | diethylaminoethoxyhexestrol, trimanyl |
| Dexamethasone | (8S,9R,10S,11S,13S,14S,16R,17R)-9-fluoro-11,17-dihydroxy-17-(2-hydroxyacetyl)- |
| | 10,13,16-trimethyl-6,7,8,11,12,14,15,16-octahydrocyclopenta[a]phenanthren-3-one, 50- |
| | 02-2, 9-fluoro-11b, 17,21-trihydroxy-16a-methylpregna-1,4-diene-3,20-dione, Aeroseb- |
| | Dex, C22H29FO5, Decaderm, Decadron, Decarex, Decaspray, dexamethazone, |
| | Dexone, Dms, glucocorticoid dexamethasone, Hexadrol, Maxidex, Mymethasone |
| DHOH | 2810417D19Rik, AI834883, DHOdehase |
| DJ-1 | CAP1, DJ-1, FLJ27376, FLJ34360, FLJ92274, RNA-BINDING PROTEIN |
| | REGULATORY SUBUNIT, SP22 |
| Dopamine | 1,2-benzenediol, 4-(2-aminoethyl)-, 1,2-benzenediol, 4-(2-aminoethyl)-(9Cl), 4-(2- |
| | aminoethyl)benzene-1,2-diol, 51-61-6, 62-31-7, C8H11NO2, DA, dopamine |
| | hydrochloride, Intropin |
| FAD | 146-14-5, 1H-purin-6-amine, flavin dinucleotide, 1H-purin-6-amine, flavine dinucleotide, |
| | adenine-riboflavin dinucleotide, adenosine 5′-(trihydrogen pyrophosphate), 5′-5′-ester |
| | with riboflavine, C27H33N9O15P2, flavin adenine dinucleotide, flavine adenosine |
| | diphosphate, riboflavin 5′-(trihydrogen diphosphate), 5′-5′-ester with adenosine, |
| | riboflavin 5′-adenosine diphosphate |
| FADH2 | 1,5-dihydro-FAD, 1910-41-4, C27H35N9O15P2 |
| GPD2 | AA408484, AI448216, Alpha-gpd, AU021455, AW494132, GDH2, Gdm1, |
| | Glycerophosphate dehydrogenase, GPDH, Gpdh-m, GPDM, m-GDH, MGPDH, |
| | mtGPDH, TISP38 |
| GPX4 | 1700027O09Rik, Glutathione peroxidase 4, MCSP, MGC103187, MGC118087, |
| | mtPHGPx, PHGPX, phospholipid hydroperoxidase, snGPx, snPHGPx |
| GPX7 | 3110050F08RIK, AI327032, CL683, FLJ14777, GPX6, NPGPX |
| GRX2 | 1700010P22Rik, AI645710, bA101E13.1, CGI-133, GRX2 |
| GSH | (2S)-2-amino-4-[[(1R)-1-(carboxymethylcarbamoyl)-2-sulfanyl-ethyl]carbamoyl]butanoic |
| | acid, 70-18-8, C10H17N3O6S, gamma-Glu-Cys-Gly, gamma-L- |
| | glutamylcysteinylglycine, glutathione-reduced, glycine, N-(N-L-gamma-glutamyl-L- |
| | cysteinyl)-, GSH |
| GSR | AI325518, D8Ertd238e, GLUTATHIONE REDUCTASE, Gr, Gr-1, Gred, GRX, |
| | MGC78522 |
| GSSG | (2S)-2-amino-4-[[(1R)-2-[(2R)-2-[[(4S)-4-amino-4-carboxy-butanoyl]amino]-2- |
| | (carboxymethylcarbamoyl)ethyl]disulfanyl-1- |
| | (carboxymethylcarbamoyl)ethyl]carbamoyl]butanoic acid, 27025-41-8, bis(gamma- |
| | glutamyl-L-cysteinylglycine) disulfide, C20H32N6O12S2, glutathione, oxidized, GSSG, |
| | oxiglutatione |
| H+ | 12408-02-5, 12586-59-3, H+, hydrogen ion, hydrogen(+1) cation, proton |
| H2O | 7732-18-5, H2O, oxidane |
| H2O2 | 7722-84-1, H2O2, hydrogen dioxide, hydrogen peroxide |
| HtrA2 | AI481710, mnd2, OMI, PARK13, PRSS25 |
| Hydro-peroxide | RO2H |
| JNK | Jnk (55 kDa isoform), Jnk p46, Jnk p46 isoform, Jnk p54, Jnk p54 isoform, Jnk p56, Jnk |
| | protein, Jnk/Sapk, p40, p46 jnk/sapk, p47, p54 jnk/sapk, Sapk/Jnk |
| KGDH | 2210403E04RIK, 2210412K19Rik, AA409584, AKGDH, Alpha ketoglutarate |
| | dehydrogenase, d1401, E1k, KIAA4192, LOC360975, mKIAA4192, OGDC, Ogdh e1 |
| LPS | endotoxin, endotoxin protein, LPS |
| MAOA | 1110061B18Rik, AA407771, Mao, MGC27811, Monoamine Oxidase A, NC61C12.R1 |
| MAOB | 6330414K01Rik, MGC26382 |
| MKK4 | JNKK, JNKK1, MAPK/ERK KINASE-1, MAPKK4, MEK4, MKK4, PRKMK4, SAPKK1, |
| | SEK1, SERK1 |
| MMP+ | 1-methyl-4-phenyl-pyridine, 48134-75-4, C12H12N+, cyperquat, MPP+, N-methyl-4- |
| | phenylpyridine, N-methyl-4-phenylpyridinium, pyridinium, 1-methyl-4-phenyl-, |
| | pyridinium, 1-methyl-4-phenyl-(9Cl) |
| MPTP | 1,2,3,6-tetrahydro-1-methyl-4-phenylpyridine, 1-methyl-4-phenyl-3,6-dihydro-2H- |
| | pyridine, 28289-54-5, C12H15N, MPTP, pyridine, 1,2,3,6-tetrahydro-1-methyl-4-phenyl- |
| mtSOD | IPO-B, MANGANESE DEPENDENT SOD, Manganese Superoxide Dismutase 2, |
| | MGC128371, MGC6144, MITOCHONDRIAL SOD, Mn superoxide dismutase, MNSOD |
| Myxothiazol | (2E,4R,5R,6E)-3,5-dimethoxy-4-methyl-7-[2-[2-[(3E,5E)-7-methylocta-3,5-dien-2-yl]- |
| | 1,3-thiazol-4-yl]-1,3-thiazol-4-yl]hepta-2,6-dienamide, 2,6-heptadienamide, 7-(2′- |
| | ((1S,2E,4E)-1,6-dimethyl-2,4-heptadienyl)(2,4′-bithiazol)-4-yl)-3,5-dimethoxy-4-methyl-, |
| | (2E,4R,5S,6E)-, 2,6-heptadienamide, 7-(2′-(1,6-dimethyl-2,4-heptadienyl)(2,4′- |
| | bithiazol)-4-yl)-3,5-dimethoxy-4-methyl-, 76706-55-3, C25H33N3O3S2 |
| NAD+ | 53-84-9, adenosine 5′-(trihydrogen diphosphate), P′-5′-ester with 3-(aminocarbonyl)-1- |
| | beta-D-ribofuranosylpyridinium, inner salt, beta-NAD+, beta-nicotinamide adenine |
| | dinucleotide+, C21H28N7O14P2+, NAD, [[(2R,3R,4R,5R)-5-(6-aminopurin-9-yl)-3,4- |
| | dihydroxy-oxolan-2-yl]methoxy-hydroxy-phosphoryl]oxy-[[(2R,3R,4R,5R)-5-(5- |
| | carbamoylpyridin-1-yl)-3,4-dihydroxy-oxolan-2-yl]methoxy]phosphinic acid |
| NADH | 58-68-4, adenosine 5′-(trihydrogen diphosphate), P′-5′-ester with 1,4-dihydro-1-beta-D- |
| | ribofuranosyl-3-pyridinecarboxamide, beta-NADH, C21H29N7O14P2, |
| | dihydronicotinamide-adenine dinucleotide, NADH2, nicotinamide dinucleotide, |
| | [[(2R,3R,4R,5R)-5-(6-aminopurin-9-yl)-3,4-dihydroxy-oxolan-2-yl]methoxy-hydroxy- |
| | phosphoryl]oxy-[[(2R,3R,4R,5R)-5-(3-carbamoyl-4H-pyridin-1-yl)-3,4-dihydroxy-oxolan- |
| | 2-yl]methoxy]phosphinic acid |
| NADP+ | 1184-16-3, 53-59-8, adenosine 5′-(trihydrogen diphosphate), 2′-(dihydrogen |
| | phosphate), P′-5′-ester with 3-(aminocarbonyl)-1-beta-D-ribofuranosylpyridinium, inner |
| | salt, beta-NADP, C21H29N7O17P3+, NAD phosphate, nicotinamide adenine |
| | dinucleotide phosphate, [(2R,3R,4R,5R)-2-(6-aminopurin-9-yl)-5-[[[[(2R,3R,4R,5R)-5- |
| | (5-carbamoylpyridin-1-yl)-3,4-dihydroxy-oxolan-2-yl]methoxy-hydroxy-phosphoryl]oxy- |
| | hydroxy-phosphoryl]oxymethyl]-4-hydroxy-oxolan-3-yl]oxyphosphonic acid |
| NADPH | 2646-71-1, 53-57-6, adenosine 5′-(trihydrogen diphosphate), 2′-dihydrogen |
| | phosphate), P′-5′-ester with 1,4-dihydro-1-beta-D-ribofuranosyl-3-pyridinecarboxamide, |
| | C21H30N7O17P3, dihydronicotinamide-adenine dinucleotide phosphate, NADPH |
| | tetrasodium salt, [(2R,3R,4R,5R)-2-(6-aminopurin-9-yl)-5-[[[[(2R,3R,4R,5R)-5-(3- |
| | carbamoyl-4H-pyridin-1-yl)-3,4-dihydroxy-oxolan-2-yl]methoxy-hydroxy-phosphoryl]oxy- |
| | hydroxy-phosphoryl]oxymethyl]-4-hydroxy-oxolan-3-yl]oxyphosphonic acid |
| NCT | 9430068N19Rik, AA727311, APH2, D1Dau13e, KIAA0253, mKIAA0253, NCT, |
| | NICASTRIN, RP11-517F10.1 |
| ND4 | MTND4, Nadh Dehydrogenase Subunit 4, Nadh ubiquinone oxidoreductase chain 4, |
| | Urf4 |
| ND4L | MTND4L, Urf4l |
| ND5 | 0610010I05RIK, MTND5, Nadh5, Urf5 |
| NDUFA10 | 2900053E13Rik, CI-42 KD, Complex I-42 KD, MGC5103, NDUFA10, Ndufa10I1 |
| NDUFA11 | 11, 14.7 kDa, 2010012C24Rik, AV006275, B14.7, Complex I-B14.7, ZINC FINGER |
| | PROTEIN |
| NDUFA12 | 13 KD DIFFERENTIATION-ASSOCIATED PROTEIN, 2410011G03Rik, AW112974, |
| | B17.2, DAP13, MGC107642, MGC7999, NADH-ubiquinone oxidoreductase b17.2, |
| | RGD1311462 |
| NDUFA13 | 2700054G14Rik, AU022060, B16.6, CDA016, CGI-39, GRIM-19, RGD1565358 |
| NDUFA2 | AV000592, B8, C1-B8, CD14, Complex I-B8, Nadh dehydrogenase(ubiquinone) 1 |
| | alpha subcomplex 7, Nadh dehydrogenase(ubiquinone) 1 subcomplex 2 |
| NDUFA3 | 1010001M12Rik, 1700022J01Rik, B9, LOC683547, LOC6910011 |
| NDUFA4 | CI-MLRQ, Complex I-MLRQ, FLJ27440, LOC679503, LOC681024, MGC104422, |
| | MGC126843, MGC126845, MLRQ |
| NDUFA4L | bcm1723 |
| NDUFA4L2 | BC064011, FLJ26118, LOC56901, MGC117746, NUOMS |
| NDUFA5 | 2900002J19Rik, B13, CI-13KD-B, DKFZp781K1356, FLJ12147, MGC72911, |
| | NADHUO, NUFM, UQOR13 |
| NDUFA6 | 14 kDa, 2700038D15Rik, B14, B230217P19Rik, CI-B14, LYRM6, NADH |
| | DEHYDROGENASE 1 ALPHA, NADHB14 |
| NDUFA7 | 14.5 kDa, 2400007M02Rik, B14.5a, MGC188160 |
| NDUFA8 | 0610033L03Rik, Aa2-258, AW261656, CI-19 KD, CI-PGIV, Complex I-19 KD, Complex |
| | I-PGIV, MGC793, PGIV |
| NDUFA9 | 1010001N11Rik, 39 Kda, ci, Complex I-39 KD, MGC111043, NADH UBIQUINONE |
| | OXYDOREDUCTASE, ND39, NDUFA9, NDUFS2L |
| NDUFAB1 | 100043472, 2210401F17Rik, 2310039H15Rik, 2610003B19Rik, 8 kDa, |
| | 9130423F15Rik, ACP, FASN2A, MGC65095, Ndufab1 predicted, Ndufab1_predicted, |
| | SDAP |
| NOUFAF1 | 2410001M24Rik, CGI-65, CIA30 |
| NDUFB1 | CI-SGDH, MNLL |
| NDUFB10 | 0610011B04Rik, 22 kDa, Complex I-PDSW, LOC681418, NDUFB10, PDSW |
| NDUFB11 | D5Bwg0566e, D5Bwg0577e, ESSS, FLJ20494, MGC111182, Ndufb11 predicted, |
| | Ndufb11_predicted, Neuronal protein 15.6, NP15, NP15.6, NP17.3, P17.3, |
| | RGD1563698 |
| NDUFB2 | 1810011O0Rik, 8 kDa, AGGG, AI325567, CI-AGGG, MGC70788, Ndufb2 predicted, |
| | Ndufb2_predicted |
| NDUFB3 | 2700033I16Rik, AI415450, B12, Ndufb3 predicted, Ndufb3_predicted |
| NDUFB4 | 0610006N12Rik, 1300010H20Rik, B15, CI-B15, Complex I-B15, LOC687500, |
| | LOC687888, LOC688963, LOC690550, LOC691675, MGC5105, RGD1560088 |
| | predicted, RGD1560088_predicted, RGD1560413 predicted, RGD1560413_predicted |
| NDUFB5 | 0610007D05Rik, AU015782, CI-SGDH, Complex I-SGDH, DKFZp686N02262, |
| | FLJ30597, MGC111204, MGC12314, Nadh, SGDH |
| NDUFB6 | 17 Kda, ci, B17, CI, Gm137, MGC13675, NADH UBIQUINONE OXIDOREDUCTASE |
| | B17 |
| NDUFB7 | 1110002H15Rik, B18, CI-B18, MGC2480, NADH DEHYDROGENASE (UBIQUINONE) |
| | 1 BETA SUBCOMPLEX, 7, 18 KDA, Ndufb7 predicted Ndufb7_predicted, SQM1, |
| | UBIQUINONE OXIDOREDUCTASE COMPLEX |
| NDUFB8 | 2900010I05Rik, AI987932, ASHI, CI-ASHI, Complex-I Ubiquinone Oxidoreductase |
| | Subunit Ashi |
| NDUFS1 | 5830412M15RIK, 9930026A05Rik, CI-75 Kd, MGC19199, MGC26839, MGC7850, |
| | MGC93795, MITOCHONDRIAL COMPLEX I 75-KDA SUBUNIT, NADH |
| | dehydrogenase precursor, 75 kDa subunit, Nadh Ubiquinone Oxidoreductase 75 Kda |
| | Subunit, NADH-coenzyme Q reductase, PRO1304 |
| NDUFS2 | AL033311, Complex I-49 KD, MGC27667, Nadh Ubiquinone Oxidoreductase 49 Kda |
| | Subunit, Nadh-coenzyme q reductase |
| NDUFS3 | 0610010M09Rik, 30 Kda, ci, NADH Dehydrogenase 30 kDa, NADH-UBIQUINONE |
| | REDUCTASE, Ndufs3 predicted Ndufs3_predicted, OTTMUSG00000005734 |
| NDUFS4 | 18 kda subunit of complex i, 6720411N02RIK, AQDQ, C1-18k, CI-18 kDa, Complex I- |
| | 18 kDa |
| NDUFS5 | AA407369, AI256693, Nadh-Q Reductase, Ndufs5b |
| NDUFS6 | BC059730, EG623286, IP13, Ip13dis, LOC679739, MGC107676, Ndub13, Ndufs6, |
| | RATIp13dis |
| NDUFS7 | 1010001M04Rik, CI-20 KD, FLJ45860, FLJ46880, MGC105684, MGC120002, MY017, |
| | NADH-coenzyme Q reductase, PSST |
| NDUFS8 | BC021616, MGC101957, MGC37950, Ndufs8 predicted, Ndufs8_predicted, TYKY |
| NDUFV1 | CI-51 kD, MGC94599, MITOCHONDRIAL COMPLEX 1-51 KDA SUBUNIT, NADH |
| | DEHYDROGENASE 51 KD, ND51, UQOR1 |
| NDUFV2 | 24 KDA SUBUNIT-MITOCHONDRIAL COMPLEX 1, 2900010C23Rik |
| NDUFV3 | 1500032D16Rik, CI-9 KD, MGC72817, Mipp65, MITOCHONDRIAL COMPLEX 1- |
| | 10 KDA SUBUNIT, NDUFV3, Ndufv3I |
| NO | 10102-43-9, EDRF, nitric oxide, nitric oxide gas radical, nitrogen monoxide, nitrogen |
| | oxide (NO), NO |
| O2 | 7782-44-7, dioxygen, molecular oxygen, O2 |
| O2− | 11062-77-4, O2−, superoxide, superoxide anion, superoxide radical |
| Oligomycin | 1404-19-9, C45H74O11 |
| Palmitate | 143-20-4, 57-10-3, c16 fatty acid, C16:0 fatty acid, C16H32O2, hexadecanoic acid, |
| | palmitate |
| Parkin | AR-JP, LPRS2, MGC130518, Park, PARKIN, PDJ, PRKN |
| PDHA | E1 ALPHA PDH, MGC114215, MGC94854, Pdh e1alpha, PDHA, Pdha1, PDHCE1A, |
| | PHE1A |
| PEN-2 | 1700023M09RIK, MDS033, MGC102026, MSTP064, OTTMUSG00000006606, PEN-2, |
| | RGD1312037 |
| Perhexiline | 2-(2,2-dicyclohexylethyl)piperidine, 6621-47-2, C19H35N, piperidine, 2-(2,2- |
| | dicyclohexylethyl)- |
| peroxynitrite | 19059-14-4, NO3−, oxido nitrite, peroxynitrite |
| PRX3 | AOP-1, AW822249, D0Tohi1, Ef2I, MER5, MGC104387, MGC24293, PRO1748, PRX |
| | III, Prx3, SP-22, TDXM, THIOREDUCTASE DEPENDANT PEROXIDE REDUCTASE |
| PRX5 | ACR1, AOEB166, AOPP, B166, MGC117264, MGC142283, MGC142285, |
| | Peroxiredoxin 5, PLP, PMP20, PRDX6, PRXV, SBBI10, THIOREDOXIN PEROXIDASE 5 |
| PSEN-1 | AD3, Ad3h, FAD, PRESENILIN 1, PS-1, S182 |
| ROS | oxygen and reactive oxygen species, reactive oxygen metabolites, ROI, ROS |
| Rotenone | (1)Benzopyrano(3,4-b)furo(2,3-h)(1)benzopyran-6(6aH)-one, 1,2,12,12a-tetrahydro-8,9- |
| | dimethoxy-2-(1-methylethenyl)-, (2R-(2alpha,6aalpha, 12aalpha))-, (2R,6aS,12aS)- |
| | 1,2,6,6a,12,12a-hexahydro-2-isopropenyl-8,9-dimethoxychromeno(3,4-b)furo(2,3- |
| | h)chromen-6-one, 83-79-4, C23H22O6, rot |
| SDHAL1 | LOC255812 |
| SDHB | 0710008N11Rik, FLJ92337, IP, PCHC, PGL4, SDH, SDH1, SDHIP, Succinate |
| | Dehydrogenase Cytochrome B Subunit, Succinate Dehydrogenase Ip Cytochrome B |
| | Subunit |
| SDHC | 0610010E03Rik, AI316496, AU019277, CYB560, CYBL, MGC103103, MGC95158, |
| | PGL3, QPS1, SDH3 |
| SDHD | 311001M13RIK, AVLL5809, C78570, CBT1, MGC72971, PGL, PGL1, PRO19626, |
| | SDH4 |
| Synuclein&alpha; | AD AMYLOID, ALPHA SYNUCLEIN, ALPHASYN, MGC105443, MGC110988, NACP, |
| | PARK1, PARK4, PD1, SYNUCLEIN ALPHA |
| T3 | |
| TRX2 | 2510006J11Rik, AI788873, MGC137598, MGC93312, MT-TRX, MTRX, TRX2 |
| TRXR2 | AA118373, ESTM573010, MGC93435, SELZ, TGR, TR, TR-BETA, TR3, TRXR2, |
| | Trxrd2 |
| UCP2 | SLC25A8, UCPH |
| UQCRB | 2210415M14Rik, FLJ92016, FLJ97033, LOC685596, LOC687741, LOC690049, |
| | MGC107639, MGC35665, QCR7, QP-C, UQBC, UQBP, Uqcrb predicted, |
| | Uqcrb_predicted, UQPC |
| UQCRC2 | 1500004O06Rik, AURA11, MGC94368, Mitochondrial Core Protein2, QCR2, Ubiquinol |
| | Cytochrome C Reductase Core Protein 2, Ubiquinol Cytochrome C Reductase Core |
| | Protein 2 Precursor, UQCR2 |
| UQCRFS1 | 4430402G14Rik, AI875505, FE-S COX3 SUBUNIT, Fes subunit of complex iii, |
| | LRRGT00195, MGC105530, RIP1, RIS1, RISP, UBIQUINOL CYTOCHROME C |
| | REDUCTASE, UQCR5 |
| UQCRFSL1 | |
| UQCRH | ENSMUSG00000037438, MGC111572, QCR6 |
| Xanthineoxidase | XANTHINE OXIDASE, XO, XOR, Xox-1 |
| &beta; secretase | |
| &gamma; | Gamma Secretase |
| secretase | |

*Genes/proteins that were used to identify the pathway:
NDUFB9 1190008J14Rik, B22, Complex I-B22, DKFZp566O173, FLJ22885, LYRM3, NADH Dehydrogenase (Ubiquinone) 1 Beta B22 Subunit, Nadh Ubiquinone Oxidoreductase B22 Subunit, Nadh-Q Oxidoreductase B22, UQOR22
SDHA 2310034D06Rik, 4921513A11, C81073, FP, SDH2, SDHF, Succinate dehydrogenase complex, subunit A flavoprotein (Fp), Succinate-ubiquinone oxidoreductase 70-kda subunit
UQCRC1 1110032G10Rik, COR1, D3S3191, MGC93712, MGC97899, QCR1, Ubiquinol cytochrome c reductase 1, Ubiquinol Cytochrome C Reductase Core 1, UQCR1

TABLE 32
Genes/Proteins Involved in the Butanoate metabolism pathway

| Name | Synonyms |
|---|---|
| (R)-3-((R)-3-Hydroxy-butanoyloxy)butanoate | (3R)-3-[(3R)-3-hydroxybutanoyl]oxybutanoic acid, (R)-3-((R)-3- |
| | hydroxybutanoyloxy)-butanoate, C8H14O5 |
| (R)-3-Hydroxy-butanoate | (3R)-3-hydroxybutanoic acid, (R)-(−)-3-hydroxybutyric acid sodium salt, (R)- |
| | 3-hydroxybutanoic acid, (R)-3-hydroxybutyric acid, 13613-65-5, 625-72-9, |
| | C4H8O3, D-beta-hydroxybutyrate, R-3-hydroxybutanoate, sodium (R)-3- |
| | hydroxybutyrate |
| (R)-3-Hydroxy-butanoyl-CoA | (R)-3-hydroxybutanoyl-CoA, (R)-3-hydroxybutyryl-coenzyme A, 21804-29-5, |
| | C25H42N7O18P3S, [(2R,3R,4R,5R)-5-(6-aminopurin-9-yl)-4-hydroxy-2- |
| | [[hydroxy-[hydroxy-[3-hydroxy-3-[2-[2-[(3R)-3- |
| | hydroxybutanoyl]sulfanylethylcarbamoyl]ethylcarbamoyl]-2,2-dimethyl- |
| | propoxy]phosphoryl]oxy-phosphoryl]oxymethyl]oxolan-3-yl]oxyphosphonic |
| | acid |
| (R)-Acetoin | (3R)-3-hydroxybutan-2-one, (R)-2-acetoin, (R)-3-hydroxy-2-butanone, (R)-3- |
| | hydroxybutan-2-one, (R)-dimethylketol, C4H8O2 |
| (R)-Malate | (2R)-2-hydroxybutanedioic acid, (R)-malate, 636-61-3, C4H6O5, D-malate, |
| | malic acid, L(+)- |
| (R,R)-Butane-2,3-diol | (2R,3R)-butane-2,3-diol, (R,R)-(−)-butane-2,3-diol, (R,R)-2,3-butanediol, |
| | (R,R)-butane-2,3-diol, 24347-58-8, C4H10O2, r,r-butane-2,3-diol |
| (S)-3-Hydroxy-butanoyl-CoA | (S)-3-hydroxybutanoyl-CoA, (S)-3-hydroxybutyryl-CoA, (S)-3-hydroxybutyryl- |
| | coenzyme A, 22138-45-0, C25H42N7O18P3S, [(2R,3R,4R,5R)-5-(6- |
| | aminopurin-9-yl)-4-hydroxy-2-[[hydroxy-[hydroxy-[3-hydroxy-3-[2-[2-[(3S)-3- |
| | hydroxybutanoyl]sulfanylethylcarbamoyl]ethylcarbamoyl]-2,2-dimethyl- |
| | propoxy]phosphoryl]oxy-phosphoryl]oxymethyl]oxolan-3-yl]oxyphosphonic |
| | acid |
| (S)-3-Hydroxy-3-methylglutaryl-CoA | (3S)-4-[2-[3-[[4-[[[(2R,3R,4R,5R)-5-(6-aminopurin-9-yl)-4-hydroxy-3- |
| | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-hydroxy-3-methyl- |
| | butanoic acid, (S)-3-hydroxy-3-methylglutaryl-CoA, 1553-55-5, |
| | C27H44N7O20P3S, hydroxymethylglutaryl-CoA, S-(hydrogen 3-hydroxy-3- |
| | methylglutaryl)coenzyme A, S-(hydrogen 3-hydroxy-3-methylpentanedioate)coenzyme A |
| (S)-Acetoin | (3S)-3-hydroxybutan-2-one, C4H8O2 |
| (S,S)-Butane-2,3-diol | (2S,3S)-butane-2,3-diol, (S,S)-butane-2,3-diol, 19132-06-0, 2,3-butanediol, |
| | (S—(R*,R*))—, C4H10O2 |
| 1-Butanol | 1-butanol, 1-hydroxybutane, 71-36-3, butan-1-ol, butanol, butyl alcohol, |
| | C4H10O, n-butanol |
| 1.1.1.- | |
| 1.1.1.157 | (S)-3-hydroxybutanoyl-CoA:NADP oxidoreductase, beta-hydroxybutyryl |
| | coenzyme A dehydrogenase, beta-hydroxybutyryl-CoA dehydrogenase, |
| | BHBD, dehydrogenase, L-3-hydroxybutyryl coenzyme A (nicotinamide |
| | adenine dinucleotide phosphate), L(+)-3-hydroxybutyryl-CoA dehydrogenase |
| 1.1.1.30 | (R)-3-hydroxybutanoate:NAD oxidoreductase, 3-D-hydroxybutyrate |
| | dehydrogenase, beta-hydroxybutyrate dehydrogenase, beta-hydroxybutyric |
| | acid dehydrogenase, beta-hydroxybutyric dehydrogenase, D-(−)-3- |
| | hydroxybutyrate dehydrogenase, D-3-hydroxybutyrate dehydrogenase, D- |
| | beta-hydroxybutyrate dehydrogenase, hydroxybutyrate oxidoreductase, |
| | NAD-beta-hydroxybutyrate dehydrogenase |
| 1.1.1.35 | (S)-3-hydroxyacyl-CoA:NAD oxidoreductase, 1-specific DPN-linked beta- |
| | hydroxybutyric dehydrogenase, 3-hydroxyacetyl-coenzyme A |
| | dehydrogenase, 3-hydroxyacyl coenzyme A dehydrogenase, 3- |
| | hydroxybutyryl-CoA dehydrogenase, 3-hydroxyisobutyryl-CoA |
| | dehydrogenase, 3-keto reductase, 3-L-hydroxyacyl-CoA dehydrogenase, |
| | 3beta-hydroxyacyl coenzyme A dehydrogenase, beta-hydroxy acid |
| | dehydrogenase, beta-hydroxyacyl CoA dehydrogenase, beta-hydroxyacyl |
| | dehydrogenase, beta-hydroxyacyl-coenzyme A synthetase, beta- |
| | hydroxyacylcoenzyme A dehydrogenase, beta-hydroxybutyrylcoenzyme A |
| | dehydrogenase, beta-keto-reductase, beta-ketoacyl-CoA reductase, L-3- |
| | hydroxyacyl CoA dehydrogenase, L-3-hydroxyacyl coenzyme A |
| | dehydrogenase |
| 1.1.1.36 | (R)-3-hydroxyacyl-CoA dehydrogenase, (R)-3-hydroxyacyl-CoA:NADP |
| | oxidoreductase, acetoacetyl coenzyme A reductase, beta-ketoacyl-CoA |
| | reductase, D(−)-beta-hydroxybutyryl CoA-NADP oxidoreductase, D-3- |
| | hydroxyacyl-CoA reductase, hydroxyacyl coenzyme-A dehydrogenase, |
| | NADP-linked acetoacetyl CoA reductase, NADPH:acetoacetyl-CoA |
| | reductase, short chain beta-ketoacetyl(acetoacetyl)-CoA reductase |
| 1.1.1.4 | (R)-2,3-butanediol dehydrogenase, (R)-diacetyl reductase, (R,R)-butane-2,3- |
| | diol:NAD oxidoreductase, 1-amino-2-propanol dehydrogenase, 1-amino-2- |
| | propanol oxidoreductase, 2,3-butanediol dehydrogenase, aminopropanol |
| | oxidoreductase, butylene glycol dehydrogenase, D-(−)-butanediol |
| | dehydrogenase, D-1-amino-2-propanol dehydrogenase, D-1-amino-2- |
| | propanol:NAD+ oxidoreductase, D-aminopropanol dehydrogenase, D- |
| | butanediol dehydrogenase, diacetyl (acetoin) reductase |
| 1.1.1.5 | acetoin:NAD oxidoreductase, diacetyl reductase |
| 1.1.1.61 | 4-hydroxybutanoate:NAD oxidoreductase, g-hydroxybutyrate |
| | dehydrogenase |
| 1.1.1.76 | (S,S)-butane-2,3-diol:NAD oxidoreductase, L(+)-2,3-butanediol |
| | dehydrogenase (L-acetoin forming), L-BDH, L-butanediol dehydrogenase |
| 1.1.1.83 | (R)-malate:NAD oxidoreductase (decarboxylating), bifunctional L(+)-tartrate |
| | dehydrogenase-D(+)-malate (decarboxylating), D-malate dehydrogenase, D- |
| | malic enzyme |
| 1.1.99.2 | (S)-2-hydroxyglutarate:(acceptor) 2-oxidoreductase, alpha-hydroxyglutarate |
| | dehydrogenase, alpha-hydroxyglutarate dehydrogenase (NAD+ specific), |
| | alpha-hydroxyglutarate oxidoreductase, alpha-ketoglutarate reductase, |
| | hydroxyglutaric dehydrogenase, L-alpha-hydroxyglutarate dehydrogenase, |
| | L-alpha-hydroxyglutarate:NAD+ 2-oxidoreductase |
| 1.1.99.8 | alcohol:(acceptor) oxidoreductase, MDH, primary alcohol dehydrogenase, |
| | quinohemoprotein alcohol dehydrogenase, quinoprotein alcohol |
| | dehydrogenase, quinoprotein ethanol dehydrogenase |
| 1.2.1.10 | acetaldehyde:NAD oxidoreductase (CoA-acetylating), aldehyde |
| | dehydrogenase (acylating) |
| 1.2.1.16 | succinate semialdehyde dehydrogenase (nicotinamide adenine dinucleotide |
| | (phosphate)), succinate-semialdehyde:NAD(P) oxidoreductase |
| 1.2.1.24 | succinate semialdehyde:NAD+ oxidoreductase, succinate- |
| | semialdehyde:NAD oxidoreductase, succinic semialdehyde dehydrogenase, |
| | succinyl semialdehyde dehydrogenase |
| 1.2.1.3 | aldehyde:NAD oxidoreductase, CoA-independent aldehyde dehydrogenase, |
| | m-methylbenzaldehyde dehydrogenase, NAD-aldehyde dehydrogenase, |
| | NAD-dependent 4-hydroxynonenal dehydrogenase, NAD-dependent |
| | aldehyde dehydrogenase, NAD-linked aldehyde dehydrogenase, |
| | propionaldehyde dehydrogenase |
| 1.2.1.57 | butanal:NAD(P) oxidoreductase (CoA-acylating) |
| 1.2.4.1 | MtPDC (mitochondrial pyruvate dehydogenase complex), PDH, pyruvate |
| | decarboxylase, pyruvate dehydrogenase, pyruvate dehydrogenase complex, |
| | pyruvate:lipoamide 2-oxidoreductase (decarboxylating and acceptor- |
| | acetylating), pyruvic acid dehydrogenase, pyruvic dehydrogenase |
| 1.2.7.1 | pyruvate oxidoreductase, pyruvate synthetase, pyruvate:ferredoxin 2- |
| | oxidoreductase (CoA-acetylating), pyruvate:ferredoxin oxidoreductase, |
| | pyruvic-ferredoxin oxidoreductase |
| 1.2.99.3 | aldehyde dehydrogenase (acceptor), aldehyde:(pyrroloquinoline-quinone) |
| | oxidoreductase |
| 1.3.1.44 | acyl-CoA:NAD trans-2-oxidoreductase |
| 1.3.99.2 | 3-hydroxyacyl CoA reductase, butanoyl-CoA:(acceptor) 2,3-oxidoreductase, |
| | butyryl coenzyme A dehydrogenase, butyryl dehydrogenase, enoyl- |
| | coenzyme A reductase, ethylene reductase, short-chain acyl CoA |
| | dehydrogenase, short-chain acyl-coenzyme A dehydrogenase, unsaturated |
| | acyl coenzyme A reductase, unsaturated acyl-CoA reductase |
| 2-(&alpha;-hydroxyethyl)-thiamine | 2-(1-hydroxyethyl)thiamine pyrophosphate, C14H23N4O8P2S+, [2-[3-[(4- |
| diphosphate | amino-2-methyl-pyrimidin-5-yl)methyl]-2-(1-hydroxyethyl)-4-methyl-1-thia-3- |
| | azoniacyclopenta-2,4-dien-5-yl]ethoxy-hydroxy-phosphoryl]oxyphosphonic |
| | acid |
| 2-Acetolactate | 2-acetoxypropanoic acid, 2-acetyloxypropanoic acid, 535-17-1, acetyllactic |
| | acid, alpha-acetolactate, alpha-acetoxypropionic acid, C5H8O4, propanoic |
| | acid, 2-(acetyloxy)- |
| 2-Hydroxy-glutaryl-CoA | 2-hydroxyglutaryl-1-coa, 4-[2-[3-[[4-[[[(2R,3R,4R,5R)-5-(6-aminopurin-9-yl)-4- |
| | hydroxy-3-phosphonooxy-oxolan-2-yl]methoxy-hydroxy-phosphoryl]oxy- |
| | hydroxy-phosphoryl]oxy-2-hydroxy-3,3-dimethyl- |
| | butanoyl]amino]propanoylamino]ethylsulfanylcarbonyl]-4-hydroxy-butanoic |
| | acid, C26H42N7O20P3S, coenzyme A, S-(5-hydrogen 2- |
| | hydroxypentanedioate), (R)- |
| 2-Hydroxyglutarate | 2-hydroxyglutarate, 2-hydroxyglutaric acid, 2-hydroxypentanedioic acid, |
| | 2889-31-8, C5H8O5, pentanedioic acid, 2-hydroxy- |
| 2-Oxoglutarate | 2-ketoglutarate, 2-oxoglutarate, 2-oxopentanedioic acid, 328-50-7, alpha- |
| | ketoglutarate, alpha-ketoglutaric acid, alphaKG, C5H6O5, glutaric acid, 2- |
| | oxo-, glutaric acid, 2-oxo-(8Cl), pentanedioic acid, 2-oxo- |
| 2.2.1.6 | acetohydroxy acid synthetase, acetohydroxyacid synthase, acetolactate |
| | pyruvate-lyase (carboxylating), acetolactic synthetase, alpha-acetohydroxy |
| | acid synthetase, alpha-acetohydroxyacid synthase, alpha-acetolactate |
| | synthase, alpha-acetolactate synthetase |
| 2.3.1.19 | butanoyl-CoA:phosphate butanoyltransferase, phosphotransbutyrylase |
| 2.3.1.54 | acetyl-CoA:formate C-acetyltransferase, formate acetyltransferase, pyruvate |
| | formate-lyase, pyruvic formate-lyase |
| 2.3.1.9 | 2-methylacetoacetyl-CoA thiolase, 3-oxothiolase, acetoacetyl-CoA thiolase, |
| | acetyl coenzyme A thiolase, acetyl-CoA acetyltransferase, acetyl- |
| | CoA:acetyl-CoA C-acetyltransferase, acetyl-CoA:N-acetyltransferase, beta- |
| | acetoacetyl coenzyme A thiolase, thiolase II |
| 2.6.1.19 | 4-aminobutanoate:2-oxoglutarate aminotransferase, 4-aminobutyrate |
| | aminotransferase, 4-aminobutyrate-2-ketoglutarate aminotransferase, 4- |
| | aminobutyrate-2-oxoglutarate aminotransferase, 4-aminobutyrate-2- |
| | oxoglutarate transaminase, 4-aminobutyric acid 2-ketoglutaric acid |
| | aminotransferase, 4-aminobutyric acid aminotransferase, aminobutyrate |
| | aminotransferase, aminobutyrate transaminase, beta-alanine |
| | aminotransferase, beta-alanine-oxoglutarate aminotransferase, beta- |
| | alanine-oxoglutarate transaminase, g-aminobutyrate aminotransaminase, g- |
| | aminobutyrate transaminase, g-aminobutyrate-alpha-ketoglutarate |
| | aminotransferase, g-aminobutyrate-alpha-ketoglutarate transaminase, g- |
| | aminobutyrate:alpha-oxoglutarate aminotransferase, g-aminobutyric acid |
| | aminotransferase, g-aminobutyric acid pyruvate transaminase, g- |
| | aminobutyric acid transaminase, g-aminobutyric acid-2-oxoglutarate |
| | transaminase, g-aminobutyric acid-alpha-ketoglutarate transaminase, g- |
| | aminobutyric acid-alpha-ketoglutaric acid aminotransferase, g-aminobutyric |
| | transaminase, GABA aminotransferase, GABA transaminase, GABA |
| | transferase, GABA-2-oxoglutarate aminotransferase, GABA-2-oxoglutarate |
| | transaminase, GABA-alpha-ketoglutarate aminotransferase, GABA-alpha- |
| | ketoglutarate transaminase, GABA-alpha-ketoglutaric acid transaminase, |
| | GABA-alpha-oxoglutarate aminotransferase, GABA-oxoglutarate |
| | aminotransferase, GABA-oxoglutarate transaminase, glutamate-succinic |
| | semialdehyde transaminase |
| 2.7.2.7 | ATP:butanoate 1-phosphotransferase |
| 2.8.3.12 | (E)-glutaconate CoA-transferase |
| 2.8.3.5 | 3-ketoacid CoA-transferase, 3-ketoacid coenzyme A transferase, 3-oxo-CoA |
| | transferase, 3-oxoacid CoA dehydrogenase, 3-oxoacid coenzyme A- |
| | transferase, acetoacetate succinyl-CoA transferase, acetoacetyl coenzyme |
| | A-succinic thiophorase, succinyl coenzyme A-acetoacetyl coenzyme A- |
| | transferase, succinyl-CoA transferase, succinyl-CoA:3-oxo-acid CoA- |
| | transferase |
| 2.8.3.8 | acetate coenzyme A-transferase, acyl-CoA:acetate CoA-transferase, butyryl |
| | CoA:acetate CoA transferase, butyryl coenzyme A transferase, succinyl- |
| | CoA:acetate CoA transferase |
| 3-Butyn-1-al | 52844-23-2, but-3-ynal, C4H4O |
| 3-Butyn-1-ol | 1-butyn-4-ol, 2-hydroxyethylacetylene, 3-butyne-1-ol, 3-butynol, 3-butynyl |
| | alcohol, 4-hydroxy-1-butyne, 927-74-2, but-3-yn-1-ol, C4H6O |
| 3-Butynoate | 2345-51-9, 3-butynoate, 3-butynoic acid, but-3-ynoic acid, C4H4O2 |
| 3.1.1.- | |
| 3.1.1.22 | (R)-3-((R)-3-hydroxybutanoyloxy)butanoate hydroxybutanoylhydrolase, D-(−)- |
| | 3-hydroxybutyrate-dimer hydrolase |
| 3.1.2.11 | acetoacetyl CoA deacylase, acetoacetyl coenzyme A deacylase, acetoacetyl |
| | coenzyme A hydrolase |
| 4-Aminobutanoate | 4-aminobutanoic acid, 4-aminobutyrate, 4-aminobutyric acid, 56-12-2, |
| | butanoic acid, 4-amino-, C4H9NO2, gamma-amino-N-butyric acid, gamma- |
| | aminobutyric acid |
| 4-Hydroxy-butanoate | 4-hydroxybutanoate, 4-hydroxybutanoic acid, 4-hydroxybutyrate, 4- |
| | hydroxybutyric acid, 591-81-1, butanoic acid, 4-hydroxy-, C4H8O3, gamma- |
| | hydroxybutyrate, gamma-hydroxybutyric acid |
| 4.1.1.15 | aspartate 1-decarboxylase, aspartic alpha-decarboxylase, cysteic acid |
| | decarboxylase, g-glutamate decarboxylase, Glutamate decarboxylase, L- |
| | aspartate-alpha-decarboxylase, L-glutamate 1-carboxy-lyase, L-glutamate |
| | alpha-decarboxylase, L-glutamic acid decarboxylase, L-glutamic |
| | decarboxylase |
| 4.1.1.5 | (S)-2-hydroxy-2-methyl-3-oxobutanoate carboxy-lyase, alpha-acetolactate |
| | decarboxylase |
| 4.1.1.70 | glutaconyl coenzyme A decarboxylase, pent-2-enoyl-CoA carboxy-lyase |
| 4.1.3.4 | (S)-3-hydroxy-3-methylglutaryl-CoA acetoacetate-lyase, 3-hydroxy-3- |
| | methylglutaryl CoA cleaving enzyme, 3-hydroxy-3-methylglutaryl coenzyme |
| | A lyase, 3-hydroxy-3-methylglutaryl-CoA lyase, hydroxymethylglutaryl |
| | coenzyme A lyase, hydroxymethylglutaryl coenzyme A-cleaving enzyme |
| 4.2.1.- | EctC, HPAH, hydratase |
| 4.2.1.17 | (3S)-3-hydroxyacyl-CoA hydro-lyase, 2-enoyl-CoA hydratase, 2-octenoyl |
| | coenzyme A hydrase, acyl coenzyme A hydrase, beta-hydroxyacid |
| | dehydrase, beta-hydroxyacyl-CoA dehydrase, crotonase, crotonyl hydrase, |
| | D-3-hydroxyacyl-CoA dehydratase, ECH, enol-CoA hydratase, enoyl |
| | coenzyme A hydrase (D), enoyl coenzyme A hydrase (L), enoyl coenzyme A |
| | hydratase, enoyl hydrase, hydratase, enoyl coenzyme A, short chain enoyl |
| | coenzyme A hydratase, short-chain enoyl-CoA hydratase, trans-2-enoyl-CoA |
| | hydratase, unsaturated acyl-CoA hydratase |
| 4.2.1.27 | 3-oxopropanoate hydro-lyase, acetylmonocarboxylic acid hydrase |
| 4.2.1.31 | (R)-malate hydro-lyase, D-malate hydro-lyase, malease |
| 4.2.1.55 | (3R)-3-hydroxybutanoyl-CoA hydro-lyase, D-3-hydroxybutyryl coenzyme A |
| | dehydratase, D-3-hydroxybutyryl-CoA dehydratase, enoyl coenzyme A |
| | hydrase (D) |
| 5.1.2.3 | 3-hydroxyacyl-CoA epimerase, 3-hydroxybutanoyl-CoA 3-epimerase, 3- |
| | hydroxybutyryl coenzyme A epimerase |
| 5.1.2.4 | acetylmethylcarbinol racemase |
| 5.2.1.1 | maleate cis-trans-isomerase |
| 5.3.3.3 | D3-cis-D2-trans-enoyl-CoA isomerase, vinylacetyl coenzyme A D- |
| | isomerase, vinylacetyl coenzyme A isomerase, vinylacetyl-CoA D3-D2- |
| | isomerase |
| 6.2.1.16 | acetoacetate:CoA ligase (AMP-forming), acetoacetyl-CoA synthetase |
| 6.2.1.2 | acyl-activating enzyme, butanoate:CoA ligase (AMP-forming), butyryl-CoA |
| | synthetase, fatty acid thiokinase (medium chain) |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme |
| | A, [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |
| Butanal | 1-butanal, 123-72-8, aldehyde C4, butal, butalyde, butanal, butyraldehyde, |
| | butyric aldehyde, C4H8O, n-butyraldehyde |
| Butanoate | 107-92-6, 156-54-7, 461-55-2, butanoic acid, butyrate, C4 SCFA, C4H8O2, |
| | n-butyrate, sodium butyrate |
| Butanoyl-CoA | 2140-48-9, butanoyl-coenzyme A, butyryl-CoA, C25H42N7O17P3S, |
| | [(2R,3R,4R,5R)-5-(6-aminopurin-9-yl)-2-[[[[3-[2-(2- |
| | butanoylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-4-hydroxy- |
| | oxolan-3-yl]oxyphosphonic acid |
| Butanoylphosphate | butanoyloxyphosphonic acid, C4H9O5P |
| Crotonoyl-CoA | 102680-35-3, 2-butenoyl-CoA, but-2-enoyl-CoA, C25H40N7O17P3S, |
| | crotonoyl-CoA, crotonyl-CoA, [(2R,3R,4R,5R)-5-(6-aminopurin-9-yl)-2-[[[[3- |
| | [2-(2-but-2-enoylsulfanylethylcarbamoyl)ethylcarbamoyl]-3-hydroxy-2,2- |
| | dimethyl-propoxy]-hydroxy-phosphoryl]oxy-hydroxy-phosphoryl]oxymethyl]- |
| | 4-hydroxy-oxolan-3-yl]oxyphosphonic acid |
| Diacetyl | 2,3-butanedione, 431-03-8, butane-2,3-dione, C4H6O2 |
| Fumarate | (E)-but-2-enedioic acid, 110-17-8, 2-butenedioic acid (2E)-, C4H4O4, |
| | fumarate |
| Glutaconyl-1-CoA | 4-[2-[3-[[4-[[[5-(6-aminopurin-9-yl)-4-hydroxy-3-phosphonooxy-oxolan-2- |
| | yl]methoxy-hydroxy-phosphoryl]oxy-hydroxy-phosphoryl]oxy-2-hydroxy-3,3- |
| | dimethyl-butanoyl]amino]propanoylamino]ethylsulfanylcarbonyl]but-3-enoic |
| | acid, 6712-05-6, C26H40N7O19P3S, coenzyme A, glutaconyl-, coenzyme A, |
| | S-(5-hydrogen 2-pentenedioate), glutaconyl-1-CoA, glutaconyl-1-coenzyme |
| | A, glutaconyl-coa |
| L-Glutamate | (2S)-2-aminopentanedioic acid, 142-47-2, 19473-49-5, 56-86-0, C5H9NO4, |
| | glutamate, glutamic acid, L-Glu, L-glutamate, L-glutamic acid, monosodium |
| | glutamate, potassium glutamate, potassium L-glutamate, sodium glutamate |
| Maleate | (Z)-but-2-enedioic acid, 110-16-7, 2-butenedioic acid, 2-butenedioic acid |
| | (2Z)-, 2-butenedioic acid (Z)-, 2-butenedioic acid (Z)-(9Cl), C4H4O4, cis- |
| | butenedioic acid, toxilic acid |
| PHBC | Acatn, EctA, LAC1, LAG1, PHBC |
| Poly-&beta;-hydroxy-butyrate | ((R)-3-hydroxybutanoyl)(n-2), (C4H6O2)n, 29435-48-1, butanoic acid, 3- |
| | hydroxy-, (R)-, homopolymer, poly(D-beta-hydroxybutyrate), poly-beta- |
| | hydroxybutyrate, (R)-isomer |
| Pyruvate | 127-17-3, 2-oxopropanoate, 2-oxopropanoic acid, 57-60-3, C3H4O3, |
| | propanoic acid, 2-oxo-, propanoic acid, 2-oxo-, ion(1-), propanoic acid, 2- |
| | oxo-, sodium salt, pyruvate, pyruvic acid, sodium salt, sodium pyruvate |
| Succinate | 1,2-ethanedicarboxylic acid, 1,4-butanedioic acid, 110-15-6, 56-14-4, amber |
| | acid, asuccin, butanedioate, butanedioic acid, C4H6O4, ethylenesuccinic |
| | acid, katasuccin, potassium succinate, succinate, wormwood acid |
| Succinate semialdehyde | 3-formylpropanoic acid, 4-oxobutanoic acid, 692-29-5, beta-formylpropionic |
| | acid, butanoic acid, 4-oxo-, butanoic acid, 4-oxo-(9Cl), butryaldehydic acid, |
| | C4H6O3, gamma-oxybutyric acid, succinaldehydic acid, succinate |
| | semialdehyde |
| Thiamine diphosphate | 136-09-4, 154-87-0, 23883-45-6, C12H19N4O7P2S+, cocarboxylase, |
| | thiamin diphosphate, thiamine diphosphate hydrochloride, thiazolium, 3-((4- |
| | amino-2-methyl-5-pyrimidinyl)methyl)-4-methyl-5-(4,6,6-trihydroxy-3,5-dioxa- |
| | 4,6-diphosphahex-1-yl)-, chloride, P,P′-dioxide, [2-[3-[(4-amino-2-methyl- |
| | pyrimidin-5-yl)methyl]-4-methyl-1-thia-3-azoniacyclopenta-2,4-dien-5- |
| | yl]ethoxy-hydroxy-phosphoryl]oxyphosphonic acid |
| Vinylacetyl-CoA | 3-butenoyl-CoA, C25H40N7O17P3S, vinylacetyl-CoA, [(2R,3R,4R,5R)-5-(6- |
| | aminopurin-9-yl)-2-[[[[3-[2-(2-but-3- |
| | enoylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-4-hydroxy- |
| | oxolan-3-yl]oxyphosphonic acid |
| *Genes/proteins that were used to identify the pathway: | |
| 1.3.99.1 Complex II, Succinate INT Dehydrogenase | |
| 2.3.3.10 (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b-methylglutaryl-CoA synthase, beta-hydroxy-beta-methylglutaryl-CoA | |
| synthase, Hmgcs, hydroxymethylglutaryl coenzyme A synthase, hydroxymethylglutaryl coenzyme A-condensing enzyme, hydroxymethylglutaryl-CoA synthase | |

TABLE 33
Genes/Proteins Involved in the Synthesis and degradation of ketone bodies pathway.

| Name | Synonyms |
|---|---|
| (R)-3-Hydroxy-butyrate | (3R)-3-hydroxybutanoic acid, (R)-(−)-3-hydroxybutyric acid sodium salt, (R)-3- |
| | hydroxybutanoic acid, (R)-3-hydroxybutyric acid, 13613-65-5, 625-72-9, |
| | C4H8O3, D-beta-hydroxybutyrate, R-3-hydroxybutanoate, sodium (R)-3- |
| | hydroxybutyrate |
| (S)-3-Hydroxy-3- | (3S)-4-[2-[3-[[4-[[[(2R,3R,4R,5R)-5-(6-aminopurin-9-yl)-4-hydroxy-3- |
| methylglutaryl-CoA | phosphonooxy-oxolan-2-yl]methoxy-hydroxy-phosphoryl]oxy-hydroxy- |
| | phosphoryl]oxy-2-hydroxy-3,3-dimethyl- |
| | butanoyl]amino]propanoylamino]ethylsulfanylcarbonyl]-3-hydroxy-3-methyl- |
| | butanoic acid, (S)-3-hydroxy-3-methylglutaryl-CoA, 1553-55-5, |
| | C27H44N7O20P3S, hydroxymethylglutaryl-CoA, S-(hydrogen 3-hydroxy-3- |
| | methylglutaryl)coenzyme A, S-(hydrogen 3-hydroxy-3-methylpentanedioate)coenzyme A |
| 1.1.1.30 | (R)-3-hydroxybutanoate:NAD oxidoreductase, 3-D-hydroxybutyrate |
| | dehydrogenase, beta-hydroxybutyrate dehydrogenase, beta-hydroxybutyric |
| | acid dehydrogenase, beta-hydroxybutyric dehydrogenase, D-(−)-3- |
| | hydroxybutyrate dehydrogenase, D-3-hydroxybutyrate dehydrogenase, D- |
| | beta-hydroxybutyrate dehydrogenase, hydroxybutyrate oxidoreductase, NAD- |
| | beta-hydroxybutyrate dehydrogenase |
| 2.3.1.9 | 2-methylacetoacetyl-CoA thiolase, 3-oxothiolase, acetoacetyl-CoA thiolase, |
| | acetyl coenzyme A thiolase, acetyl-CoA acetyltransferase, acetyl-CoA:acetyl- |
| | CoA C-acetyltransferase, acetyl-CoA:N-acetyltransferase, beta-acetoacetyl |
| | coenzyme A thiolase, thiolase II |
| 2.8.3.5 | 3-ketoacid CoA-transferase, 3-ketoacid coenzyme A transferase, 3-oxo-CoA |
| | transferase, 3-oxoacid CoA dehydrogenase, 3-oxoacid coenzyme A- |
| | transferase, acetoacetate succinyl-CoA transferase, acetoacetyl coenzyme A- |
| | succinic thiophorase, succinyl coenzyme A-acetoacetyl coenzyme A- |
| | transferase, succinyl-CoA transferase, succinyl-CoA:3-oxo-acid CoA- |
| | transferase |
| 4.1.1.4 | acetoacetate carboxy-lyase, acetoacetic acid decarboxylase |
| 4.1.3.4 | (S)-3-hydroxy-3-methylglutaryl-CoA acetoacetate-lyase, 3-hydroxy-3- |
| | methylglutaryl CoA cleaving enzyme, 3-hydroxy-3-methylglutaryl coenzyme A |
| | lyase, 3-hydroxy-3-methylglutaryl-CoA lyase, hydroxymethylglutaryl coenzyme |
| | A lyase, hydroxymethylglutaryl coenzyme A-cleaving enzyme |
| Acetoacetate | 3-oxobutanoic acid, 541-50-4, acetoacetate, butanoic acid, 3-oxo-, C4H6O3 |
| Acetoacetyl-CoA | 1420-36-6, acetoacetyl CoA, C25H40N7O18P3S, S-acetoacetylcoenzyme A, |
| | [(2R,3R,4R,5R)-5-(6-aminopurin-9-yl)-4-hydroxy-2-[[hydroxy-[hydroxy-[3- |
| | hydroxy-2,2-dimethyl-3-[2-[2-(3- |
| | oxobutanoylsulfanyl)ethylcarbamoyl]ethylcarbamoyl]propoxy]phosphoryl]oxy- |
| | phosphoryl]oxymethyl]oxolan-3-yl]oxyphosphonic acid |
| Acetone | 2-Propanone, 67-64-1, acetone, C3H6O, dimethyl ketone, |
| | dimethylformaldehyde, dimethylketal, propanone |
| Acetyl-CoA | 72-89-9, acetyl-CoA, C23H38N7O17P3S, coenzyme A, S-acetate, S-acetyl |
| | coenzyme A, [(2R,3R,4R,5R)-2-[[[[3-[2-(2- |
| | acetylsulfanylethylcarbamoyl)ethylcarbamoyl]-3-hydroxy-2,2-dimethyl- |
| | propoxy]-hydroxy-phosphoryl]oxy-hydroxy-phosphoryl]oxymethyl]-5-(6- |
| | aminopurin-9-yl)-4-hydroxy-oxolan-3-yl]oxyphosphonic acid |

*Genes/proteins that were used to identify the pathway:
2.3.3.10 (S)-3-hydroxy-3-methylglutaryl-CoA acetoacetyl-CoA-lyase (CoA-acetylating), 3-hydroxy-3-methylglutaryl CoA synthetase, 3-Hydroxy-3-methylglutaryl coenzyme A synthase, 3-hydroxy-3-methylglutaryl coenzyme A synthetase, 3-hydroxy-3-methylglutaryl-CoA synthase, acetoacetyl coenzyme A transacetase, acetyl-CoA:acetoacetyl-CoA C-acetyltransferase (thioester-hydrolysing, carboxymethyl-forming), b-hydroxy-b-methylglutaryl-CoA synthase, beta-hydroxy-beta-methylglutaryl-CoA synthase, Hmgcs, hydroxymethylglutaryl coenzyme A synthase, hydroxymethylglutaryl coenzyme A-condensing enzyme, hydroxymethylglutaryl-CoA synthase

In addition, pathway analysis using Pathway Studio software based on previously identified differentially expressed genes or proteins associated with low lactate production led to the identification of the Eda A1 pathway (FIG. 9), Eda-A2 pathway (FIG. 10). Genes/proteins that were used to identify relevant pathways are indicated in the figures. In addition, additional exemplary genes or proteins involved in the above-identified pathways and that may be involved in regulating or indicative of low lactate production are summarized in Table 34 (Eda-A1 pathway) and Table 35 (Eda-A2 pathway).

TABLE 34
Genes/Proteins Involved in the Eda-A1 pathway

| Name | Type | Description |
|---|---|---|
| Apoptosis | Cell Process | |
| CASP8 | Protein | caspase 8, apoptosis-related cysteine |
| | | peptidase |
| EDAR | Protein | ectodysplasin A receptor |
| EDARADD | Protein | EDAR-associated death domain |
| Jnk-mapk | Pathway | |
| NF kappa B | Pathway | |
| RIPK1 | Protein | receptor (TNFRSF)-interacting serine- |
| | | threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |

*Genes/proteins that were used to identify the pathway:
HMGCS1 3-hydroxy-3-methylglutaryl-Coenzyme A synthase 1 (soluble)

TABLE 35
Genes/Proteins Involved in the Eda-A2 pathway

| Name | Type | Description |
|---|---|---|
| Apoptosis | Cell Process | |
| CASP8 | Protein | caspase 8, apoptosis-related cysteine |
| | | peptidase |
| Jnk-mapk | Pathway | |
| NF kappa B | Pathway | |
| p40 MAPK | Pathway | |
| RIPK1 | Protein | receptor (TNFRSF)-interacting serine- |
| | | threonine kinase 1 |
| RIPK2 | Protein | receptor-interacting serine-threonine kinase 2 |
| TRAF2 | Protein | TNF receptor-associated factor 2 |
| TRAF3 | Protein | TNF receptor-associated factor 3 |
| TRAF6 | Protein | TNF receptor-associated factor 6 |
| XEDAR | Protein | microtubule-associated protein 2 |

*Genes/proteins that were used to identify the pathway:
HMGCS1 3-hydroxy-3-methylglutaryl-Coenzyme A synthase 1 (soluble)

### Example 8

### Target Validation: siRNA

The proteins or genes identified herein can be used to engineer cells to improve a cell line. FIG. 9 illustrates an exemplary target validation workflow.

The ability of the genes and proteins identified herein to affect a cellular phenotype is first verified by overexpression of a nucleic acid inhibiting the expression of the relevant gene using methods known in the art. Exemplary methods based on interfering RNA constructs are described below.

Design and Synthesis of siRNA

Typically, targets that are candidates for siRNA mediated gene knockdown are sequenced, and the sequences verified. Full-length cDNA sequence information is preferred (although not required) to facilitate siRNAs design. The target sequence that is a candidate for gene knockdown is compared to gene sequences available on public or proprietary databases (e.g., BLAST search). Sequences within the target gene that overlap with other known sequences (for example, 16-17 contiguous basepairs of homology) are generally not suitable targets for specific siRNA-mediated gene knockdown.

siRNAs may be designed using, for example, online design tools, over secure internet connections, such as the one available on the Ambion® website (http://www.ambion.com/techlib/misc/siRNA_finder.html). Alternatively, custom siRNAs may also be requested from Ambion®, which applies the Cenix algorithm for designing effective siRNAs. The standard format for siRNAs is typically 5 nmol, annealed and with standard purity in plates. Upon receipt of synthesized siRNAs, the siRNAs are prepared according to the instructions provided by the manufacture and stored at the appropriate temperature (−20° C.)

Standard procedures were used for siRNA transfections. Cells to be transfected were typically pre-passaged on the day before transfection to ensure that the cells are in logarithmic growth phase. Typically, an siRNA Fed-Batch assay was used. Exemplary materials, conditions and methods for transfections are as follows.

Transfection (D0)

Per Spin Tube (50 ml)

100 uL R1

2 uL Transit-TKO transfection reagent (Mirus)

10 uL 10 uM siRNA

2 mL 1e5 cells/mL in AS1 medium

Following Transfection

37° C.: 72 hrs

31° C.: 96 hrs

Feed: AQ3 on day 3 (D3)

Sample taken on day 1 (D1), day 3 (D3), day 7 (D7)

24 Well Suspension Transfections

For each experiment, 100,000 cells (e.g., 3C7 cells) in 1 mL total volume, and 50 nM siRNA were used. To make a mix for 3 reactions, 150 μL R1 and 70 μL Mirus TKO reagent were mixed and incubated for 10 minutes at room temperature. 15 μL of 10 μM siRNA was added and the mix was incubated for 10 minutes at room temperature. 57.3 μL of the mix was transferred into each of 3 wells. 942.7 μL of R5CD1 (containing 100,000 cells) was added and the plate was incubated on rocker at 37° C. for 72 hrs.

Spin Tube siRNA Transfection

For each experiment, 100,000 cells (e.g., 3C7 cells) in 1 mL total volume were used. For each transfection, 100 μL R1 and 2 μL Mirus TKO reagent were mixed and incubated for 10 minutes at room temperature. 10 μL of 10 μM siRNA was added and the mix was incubated for 15 minutes at room temperature, mixed occasionally. 1.9 mL culture was transferred to each spin tube. siRNA mix (112 uL) was added to each spin tube. The culture was initially incubated at 37° C. and then the temperature was shifted to 31° C. on day 3. Spin tube cultures were shaken rapidly (250 RPM). Samples were taken on days 1, 3, and 7. Cultures were terminated on day 7.

Growth and productivity controls were included on each plate. An exemplary productivity control is DHFR (selectable marker on bicistronic mRNA). Treatment with DHFR siRNA reproducibly decreases amount of antibody in the CM-FcIGEN (antibody production control). An exemplary growth control is CHO1 (kinesin) (see Matuliene et al. (2002) Mol. Cell. Biol. 13:1832-45) (typically, about 20-30% growth inhibition was observed with CHO1 treatment). Other standard controls such as no siRNA treatment (transfection reagents only) and non-targeting siRNA treatment (non-specific siRNA) were also included. Plates were then subjected to cell counting (for example, in a 96-well cell counting instrument) to assess growth and to, for example, an automated 96-well titer assay, to assess productivity. Genes whose modulation, singly or in combination, are sufficient to modify useful cellular phenotypes were thereby validated and such changes can be engineered, singly or in combination, into a mammalian cell line to modify its properties.

### Example 9

### Target Validation: Overexpression

The ability of genes and proteins identified herein to affect a cellular phenotype is verified by overexpression of a nucleic acid encoding the relevant gene using methods known in the art. Exemplary methods are described below.

For example, nucleic acids overexpressing specific targets can be introduced into CHO cells by transient transfections and then the impact of over-expression on cellular growth and productivity are monitored.

Growth and productivity controls are typically used for overexpression assays. For example, positive growth/viability control used in this experiment included Ha-Ras and Bcl-xL. Negative growth control used included p27. Other suitable growth and productivity controls are known in the art and can be used for overexpression assays. Additional standard controls such as no nucleic acid control (transfection reagents only) were also included.

Target genes and the control genes are cloned into the pexpressl vector and introduced into various cell lines using methods known in the art.

### Example 10

### Engineering Cell Lines to Improve Cell Phenotypes Based on the Verified Target Genes

The verified target genes are used to effect a cell phenotype, particularly a phenotype characterized by increased and efficient production of a recombinant transgene, increased cell growth rate, high peak cell density, sustained high cell viability, high maximum cellular productivity, sustained high cellular productivity, low ammonium production, and low lactate production, etc. Exemplary target genes are disclosed above, for example, in Tables 1 through 35.

Standard cell engineering methods are used to modify target genes to effect desired cell phenotypes. As discussed above, target genes are modified to achieve desired CHO cell phenotypes by interfering RNA, conventional gene knockout or overexpression methods. Typically, knockout methods or stable transfection methods with overexpression constructs are used to engineer modified CHO cell lines. Other suitable methods are discussed in the general description section and known in the art.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

### INCORPORATION BY REFERENCE

The genes and proteins identified herein are well known and their sequences are available in several public databases (e.g., GenBank, SWISS-PROT, etc). The sequences associated with each of the genes and proteins identified herein that are available in public databases (e.g., GenBank, SWISS-PROT, etc) as of the filing date of the present application are incorporate by reference herein. All sequence accession numbers, publications and patent documents cited in this application are incorporated by reference in their entirety for all purposes to the same extent as if the contents of each individual publication or patent document was incorporated herein.

What is claimed is:

1. An engineered cell line characterized by an improved cell culture phenotype as compared to the corresponding wild type or parental cell line comprising a population of engineered cells, each of which comprising an engineered construct up-regulating or down-regulating one or more genes or proteins selected from Tables 1-35, wherein said up-regulating or down-regulating one or more genes or proteins confers the improved cell culture phenotype.

2. The engineered cell line of claim 1, wherein the improved cell culture phenotype is selected from the group consisting of improved peak cell density, improved cell growth rate, improved sustained high cell viability, improved maximum cellular productivity, improved sustained high cellular productivity, reduced lactate production, reduced ammonia production, and combinations thereof.

3. The engineered cell line of claim 1, wherein the one or more genes or proteins are selected from Tables 10 and 11, and wherein said improved cell culture phenotype is improved peak cell density.

4. The engineered cell line of claim 1, wherein the one or more genes or proteins are selected from Table 12, and wherein said improved cell culture phenotype is improved cell growth rate.

5. The engineered cell line of claim 1, wherein the one or more genes or proteins are selected from Tables 1-9, and wherein said improved cell culture phenotype is improved sustained high cell viability.

6. The engineered cell line of claim 1, wherein the one or more genes or proteins are selected from Tables 13-20, and wherein said improved cell culture phenotype is improved maximum cellular productivity.

7. The engineered cell line of claim 1, wherein the one or more genes or proteins are selected from Tables 21-24, and wherein said improved cell culture phenotype is improved sustained high cellular productivity.

8. The engineered cell line of claim 1, wherein the one or more genes or proteins are selected from Tables 25-30, and wherein said improved cell culture phenotype is reduced ammonium production.

9. The engineered cell line of claim 1, wherein the one or more genes or proteins are selected from Tables 31-35, and wherein said improved cell culture phenotype is reduced lactate production.

10-13. (canceled)

14. A method of producing a protein of interest, the method comprising:
providing an engineered cell line of claim 1 that carries a nucleic acid encoding a protein of interest;
culturing the engineered cell line under conditions that allow expression of the protein of interest; and
harvesting the protein of interest.

15. (canceled)

16. A protein produced using the method of claim 14.

17. A method of improving a cell line, the method comprising modifying one or more pathways selected from FIGS. 1-31.

18. The method of claim 17, wherein the one or more pathways are involved in alanine and aspartate metabolism, glutamate metabolism, or combinations thereof, and wherein the modification confers improved peak cell density as compared to the corresponding unmodified cell line.

19. The method of claim 17, wherein the one or more pathways are involved in G1/S checkpoint regulation, ATM signaling, Eda-A1 signaling, Eda-A2 signaling, p53 signaling, JNK-MAPK signaling pathway, mitochondrial control of apoptosis, Rb tumor suppressor signaling, or combinations thereof, and wherein the modification confers improved maximum cellular productivity as compared to the corresponding unmodified cell line.

20. The method of claim 17, wherein the one or more pathways are involved in synthesis and degradation of ketone bodies, and wherein the modification confers improved cell growth rate as compared to the corresponding unmodified cell line.

21. The method of claim 17, wherein the one or more pathways are involved in synthesis and degradation of ketone bodies, butanoate metabolism, valine, leucine, and isoleucine degradation, Eda-A1 signaling, Eda-A2 signaling, or combinations thereof, and wherein the modification confers reduced ammonia production as compared to the corresponding unmodified cell line.

22. The method of claim 17, wherein the one or more pathways are involved in oxidative phosphorylation, mitochondrial dysfunction, butanoate metabolism, synthesis and degradation of ketone bodies, Eda-A1 signaling, Eda-A2 signaling, or combinations thereof, and wherein the modification confers reduced lactate production as compared to the corresponding unmodified cell line.

23. The method of claim 17, wherein the one or more pathways are involved in citrate cycle, butanoate metabolism, glutathione metabolism, NRF2-mediated oxidative stress response, LPS-IL-1 mediated inhibition of RXR function, synthesis and degradation of ketone bodies, Eda-A1 signaling, Eda-A2 signaling, or combinations thereof, and wherein the modification confers improved sustained high cell viability as compared to the corresponding unmodified cell line.

24. The method of claim 17, wherein the one or more pathways are involved in inositol metabolism, glycolysis, gluconeogenesis, NRF2-mediated oxidative stress response, purine metabolism, or combinations thereof, and wherein the modification confers improved sustained high cellular productivity as compared to the corresponding unmodified cell line.

25-28. (canceled)

29. A cell line improved by the method of claim 17.

30. A method of producing a protein of interest, the method comprising:
providing an improved cell line of claim 29 that carries a nucleic acid encoding a protein of interest;
culturing the improved cell line under conditions that allow expression of the protein of interest; and
harvesting the protein of interest.

31. (canceled)

32. A protein produced using the method of claim 30.

33. A method of evaluating a cell culture phenotype of a cell line, the method comprising:
detecting, in a sample of cultured cells, an expression level of at least one protein or gene selected from Tables 1-35;
comparing the expression level to a reference level;
wherein the comparison is indicative of the cell culture phenotype.

34-40. (canceled)

41. A method of evaluating a cell culture phenotype of a cell line, the method comprising:
determining, in a sample of cultured cells, a signaling strength of at least one pathway selected from FIGS. 1-31;
comparing the signaling strength to a reference;
wherein the comparison is indicative of the cell culture phenotype.
